(19) United States
(12) Patent Application Publication
(10) Pub. No.: US 2023/0250134 A1
(43) Pub. Date: Aug. 10, 2023

(54) SARS-COV-2 inhibitors

(71) Applicants: University of Washington, Seattle, WA (US); NORTHWESTERN UNIVERSITY, Evanston, IL (US); WASHINGTON UNIVERSITY, St. Louis, MO (US)

(72) Inventors: Longxing CAO, Seattle, WA (US); Brian COVENTRY, Seattle, WA (US); Inna GORESHNIK, Seattle, WA (US); Lauren MILLER, Seattle, WA (US); David BAKER, Seattle, WA (US); Lisa KOZODOY, Seattle, WA (US); John BOWEN, Seattle, WA (US); Lauren CARTER, Seattle, WA (US); James Brett CASE, Seattle, WA (US); Michael DIAMOND, Seattle, WA (US); Natasha EDMAN, Seattle, WA (US); Andrew HUNT, Seattle, WA (US); Michael Christopher JEWETT, Seattle, WA (US); Cassandra Jean OGOHARA, Seattle, WA (US); Young-jun PARK, Seattle, WA (US); Rashmi RAVICHANDRAN, Seattle, WA (US); Lance Joseph STEWART, Seattle, WA (US); David VEESLER, Seattle, WA (US); Bastian VOGELI, Seattle, WA (US); Alexandra C. WALLS, Seattle, WA (US); Kejia WU, Seattle, WA (US); Scott BOYKEN, Seattle, WA (US); George UEDA, Seattle, WA (US)

(21) Appl. No.: 18/004,545

(22) PCT Filed: May 25, 2021
(86) PCT No.: PCT/US2021/034069

§ 371 Date: Jan. 6, 2023

Related U.S. Application Data
(60) Provisional application No. 63/067,593, filed on Aug. 19, 2020.
(60) Provisional application No. 63/051,474, filed on Jul. 14, 2020.

(51) Int. Cl. C07K 14/00; A61P 31/14; C12N 15/63

(52) U.S. Cl. CPC C07K 14/001; A61P 31/14; C12N 15/63

(57) ABSTRACT

Polypeptide inhibitors of SARS-COV-2 are disclosed comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-101, and their use for treating and limiting development of SARS-COV-2 infection.

### CROSS REFERENCE

This application claims priority to U.S. Provisional Patent Application Serial Nos. 63/051,474 filed Jul. 14, 2020 and 63/067,593 filed Aug. 19, 2020, each incorporated by reference herein in its entirety.

### FEDERAL FUNDING STATEMENT

This invention was made with government support under Grant No. FA8750-17-C-0219, awarded by the Defense Advanced Research Projects Agency and Grant Nos. HHSN272201700059C and R01 GM120553, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on May 25, 2021, having the file name “20-1074-WO_SeqList_ST25” and is 1,112 kb in size.

### BACKGROUND

SARS-COV-2 infection is thought to often start in the nose, with virus replicating there for several before spreading to the broader respiratory system. Delivery of a high concentration of a viral inhibitor into the nose and into the respiratory system generally could therefore potentially provide prophylactic protection, and therapeutic efficacy early in infection, and could be particularly useful for health care workers and others coming into frequent contact with infected individuals.

### SUMMARY

In a first aspect the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-17, 19-21, 23-34 and 100-101, wherein the polypeptide binds to SARS-COV-2 Spike glycoprotein receptor binding domain (RBD). In one embodiment, amino acid substitutions relative to the reference polypeptide amino acid sequence are selected from the exemplary amino acid substitutions provided in Table 1. In another embodiment, interface residues are identical to those in the reference polypeptide or are conservatively substituted relative to interface residues in the reference polypeptide. In a further embodiment the polypeptides comprise two or more copies of the amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-101. In one embodiment, the polypeptide comprises the formula Z1-Z2-Z3, wherein:

Z1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164;

Z2 comprises an optional amino acid linker; and

Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164;

wherein Z1 and Z3 may be identical or different.

In another embodiment, the polypeptides comprises the formula B1-B2-Z1-Z2-Z3-B3-B4, wherein:

Z1, Z2, and Z3 are as defined;

B2 and B3 comprise optional amino acid linkers; and one or both of B1 and B4 independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164, wherein one of B1 and B4 may be absent.

In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:47-60, 193-355 and 454-588, and a genus selected from those recited in the right hand column of Table 8 wherein genus positions X1, X2, X3, and X4 may be present or absent, and when present may be any sequence of 1 or more amino acids.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 356-453 and 595-692, and a genus selected from those recited in the middle column of Table 9 wherein genus positions X1, X2, X3, and X4 may be present or absent, and when present may be any sequence of 1 or more amino acids.

In a further embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 65-96, wherein in embodiments where a secretion signal is present (MARAWIFFLLCLAGRALA; SEQ ID NO:63) it can be replaced with any other secretion signal.

In other aspects, the disclosure provides nucleic acids encoding the polypeptide of the disclosure, expression vectors comprising the nucleic acids operatively linked to a promoter, host cell comprising a polypeptide, nucleic acid, and/or expression vector of the disclosure, oligomers of the polypeptides of the disclosure, compositions comprising 2, 3, 4, or more copies of the polypeptide any embodiment of the disclosure attached to a support, including but not limited to a polypeptide particle support, and pharmaceutical compositions, comprising a polypeptide, nucleic acid, expression vector, host cell, oligomer, and/or composition of the disclosure, and a pharmaceutically acceptable carrier.

In another aspect, the disclosure provides methods for treating or limiting development of a severe acute respiratory syndrome (SARS) coronavirus infection (including SARS-Co-V and SARS-COV-2), comprising administering to a subject in need thereof an amount of the polypeptide, the nucleic acid, the expression vector, the host cell, the oligomer, the composition, and/or the pharmaceutical composition of the disclosure, effective to treat or limit development of the infection.

### DESCRIPTION OF THE FIGURES

FIG. 1. Designed Minibinder Proteins For the SARS-CoV-2 Spike Receptor Binding Domain Designs for approach 1, and approach 2, were encoded in long oligonucleotides, and screened for binding to fluorescently tagged RBD on the yeast cell surface. Deep sequencing identified 3 Ace2 helix scaffolded designs (approach 1), and 150 de novo interface designs (approach 2) that were clearly enriched following FACS sorting for RBD binding. Designs were expressed in E. coli and purified, and many were found to have soluble expression, to bind RBD in biolayer interferometry experiments, and could effectively compete with ACE-2 for binding to RBD (example shown in FIG. 2). Based on BLI data (e.g. See FIG. 2) the RBD binding affinities of minbinders are: LCB1<1 nM, LCB3<1 nM. The affinities of LCB2, LCB4, LCB5, LCB6, LCB7,LCB8 range from 1˜20 nM, with relative strength of different binders being LCB4>LCB2>LCB9=LCB5>LCB6>LCB7.

FIG. 2. High Affinity Binding of De novo Designed Minibinder to SARS-COV-2 Spike RBD. Biotinylated Spike RBD protein was loaded to a streptavidin biolayer interferometry (BLI) tip (ForteBio Octet™) and after washing, the tip was dipped into purified Combo 1 anti-RBD minibinder at different concentrations. After loading the tips were placed into buffer alone. (Left and middle) Response curves indicate ˜Kd of 300 pM affinity. (Right) If ACE-2 is loaded to RBD tips and then Combo 1 is added, the minibinder rapidly displaces ACE-2 off of the BLI tip.

FIG. 3. De novo Designed Minibinder to SARS-COV-2 Spike RBD is Heat Stable. Purified Combo 1 minibinder was measured for in a circular dichroism spectrometer at 25 C, 95 C and at 25 C after heating to 95 C. The CD spectra were all very similar in shape indicating that the protein remains folded in all conditions.

FIG. 4. De novo Designed Minibinder to SARS-COV-2 Spike RBD are Potent in Virus Neutralization Assays. SARS-COV-2 strain 2019 n-COV/USA_WA1/2020 was obtained from the Centers for Disease Control and Prevention (gift of Natalie Thornburg). Virus stocks were produced in Vero CCL81 cells (ATCC) and titrated by focus-forming assay on Vero E6 cells. Serial dilutions of mAbs or minibinder were incubated with 102 focus-forming units (FFU) of SARS-COV-2 for 1 h at 37 C. RBD minibinder (or mAb)-virus complexes were added to Vero E6 cell monolayers in 96-well plates and incubated at 37C for 1 h. Subsequently, cells were overlaid with 1% (w/v) methylcellulose in MEM supplemented with 2% FBS. Plates were harvested 30 h later by removing overlays and fixed with 4% PFA in PBS for 20 min at room temperature. Plates were washed and sequentially incubated with 1 μg/mL of CR3022 ([1]) anti-S antibody and HRP-conjugated goat anti-human IgG in PBS supplemented with 0.1% saponin and 0.1% BSA. SARS-COV-2-infected cell foci were visualized using TrueBlue™ peroxidase substrate (KPL) and quantitated on an ImmunoSpot™ microanalyzer (Cellular Technologies). Data were processed using Prism software (GraphPad Prism™ M 8.0).

FIG. 5(A-J). LCB1-Fc prophylaxis protects against SARS-CoV-2 infection. (A) Molecular surface representation of three LCB1v1.3 miniproteins bound to individual protomers of the SARS-COV-2 spike protein trimer (left: side view; right: top view). (B) Binding curves of purified LCB1v1.3 and LCB1-Fc to SARS-COV-2 RBD as monitored by biolayer interferometry (one experiment performed in technical duplicate). (C) Neutralization curves of LCB1v1.3, LCB1-Fc, or control binder against a SARS-COV-2 WA1/2020 isolate (EC 50 values: 14.4 pM, 71.8 pM, and >10,000 nM respectively; average of two experiments, each performed in duplicate). (D-J) 7 to 8-week-old female and male K18-hACE2 transgenic mice received 250 μg of LCB1-Fc or control binder by i.p. injection one day prior to i.n. inoculation with 10³ PFU of SARS-COV-2. Tissues were collected at 4 and 7 dpi. (D) Weight change following LCB1-Fc administration (mean+SEM; n=8, two experiments: two-way ANOVA with Sidak's post-test: *** P<0.001, **** P<0.0001). (E) Infectious virus measured by plaque assay at 4 or 7 dpi in the lung (n=8, two experiments: Mann-Whitney test; *** P<0.001). (F-J) Viral RNA levels at 4 or 7 dpi in the lung, heart, spleen, brain, or nasal wash (n=8, two experiments: Mann-Whitney test: ns, not significant, * P<0.05, ** P<0.01, *** P<0.001, **** P<0.0001).

FIG. 6(A-C). LCB1-Fc prophylaxis prevents SARS-COV-2-mediated lung disease. (A) Respiratory mechanics parameters: inspiratory capacity, resistance, elastance tissue damping, quasi-static compliance, and pressure-volume loops measured at 7 dpi (n=3-6, two experiments: two-way ANOVA with Tukey's post-test: ns, not significant, * P<0.05, ** P<0.01, *** P<0.001 between indicated groups). (B) Hematoxylin and eosin staining of lung sections from mice treated at D-1 and collected at 7 dpi with SARS-COV-2. Images show low (left) and high (right; boxed region from left) magnification. Scale bars for all images, 100 μm. Representative images from n=3 mice per group. (C) Heat-map of cytokine mRNA levels from lung tissues of SARS-COV-2 infected mice at 4 dpi. For each cytokine, the fold-change was calculated relative to age-matched naïve control animals after normalization to Gapdh and the Log₂(fold change) was plotted (n=8 mice/group relative to n=3 naïve controls).

FIG. 7(A-J). Post-exposure delivery of anti-RBD binders reduces SARS-COV-2 burden. (A-G) 7 to 8-week-old female and male K18-hACE2 transgenic mice received 250 μg of LCB1-Fc or control binder by i.p. injection one day after i.n. inoculation with 103 PFU of SARS-COV-2. Tissues were collected at 4 or 7 dpi. (A) Weight change following LCB1-Fc administration (mean+SEM; n=6, two experiments: two-way ANOVA with Sidak's post-test: ** P<0.01, **** P<0.0001). (B) Infectious virus in the lung measured by plaque assay at 4 or 7 dpi in the lung (n=6, two experiments: ** P<0.01). (C-G) Viral RNA levels at 4 or 7 dpi in the lung, heart, spleen, brain, or nasal wash (n=6, two experiments: Mann-Whitney test: ns, not significant, * P<0.05, ** P<0.01). (H) Hematoxylin and eosin staining of lung sections from mice treated at D+1 and collected at 7 dpi with SARS-COV-2. Images show low (left) and high (right; boxed region from left) magnification. Scale bars for all images, 100 μm. Representative images from n=3 mice per group. (I-J) 7 to 8-week-old male K18-hACE2 transgenic mice received a single 50 μg i.n. dose of LCB1v1.3 or control binder at one- or two-days post-inoculation with 103 PFU of SARS-COV-2. Viral RNA levels at 7 dpi in the lung (I) or nasal wash (J) (n=6, two experiments: one-way ANOVA: ns, not significant, * P<0.05, **** P<0.0001).

FIG. 8(A-K). Intranasal administration of LCB1v1.3 reduces viral infection even when given 5 days prior to SARS-COV-2 exposure. (A-D) 7 to 8-week-old female K18-hACE2 transgenic mice received a single i.n. 50 μg dose of LCB1v1.3 or control binder at the indicated time prior to i.n. inoculation with 10³ PFU of SARS-COV-2. Tissues were collected at 4 or 7 dpi and viral RNA levels were determined (n=5-6 animals per group, two-experiments: two-way ANOVA with Sidak's post-test: ns, not significant, * P<0.05, ** P<0.01, *** P<0.001, **** P<0.0001). (E-J) 7 to 8-week-old female K18-hACE2 transgenic mice received the indicated i.n. dose of LCB 1v1.3 or control binder at one day prior to i.n. inoculation with 10³ PFU of SARS-COV-2. (E) Weight change following LCB1v1.3 or control binder administration (mean+SEM; n=6, two experiments: two-way ANOVA with Sidak's post-test compared to the control binder treated group: ** P<0.01,**** P<0.0001). (F-J) Viral RNA levels at 7 dpi in the lung, heart, spleen, brain, or nasal wash (n=6, two experiments: Kruskal-Wallis ANOVA with Dunn's post-test: * P<0.05. ** P<0.01, *** P<0.001). (K) Hematoxylin and eosin staining of lung sections from mice treated with a single i.n. 50 μg dose of LCB1v1.3 or control binder at D-1 and collected at 7 dpi with SARS-COV-2. Images show low (left) and high (right; boxed region from left) magnification. Scale bars for all images, 100 μm. Representative images from n=3 mice per group.

FIG. 9(A-H). Immunogenicity of LCB1v1.3 and protection from challenge. (A) Scheme of experimental details. K18-hACE2 transgenic mice (n=10 to 12 per group) were treated every 3 days with 50 μg of LCB1v1.3 or control binder by i.n. administration. On day 18 post-treatment, animals were bled and anti-LCB1v1.3 antibodies were measured. The following day, animals were challenged with 10³ PFU of SARS-COV-2, and tissues were collected at 7 dpi. (B) Binding of serum antibodies to LCB1v1.3 as measured by ELISA (three experiments). Dashed line indicated limit of detection of the assay. (C) Weight change following LCB1v1.3 or control binder administration (mean+SEM; two experiments: two-way ANOVA with Sidak's post-test: **** P<0.0001). (D-H) Viral RNA levels at 7 dpi in the lung, heart, spleen, brain, or nasal wash (two experiments: Mann-Whitney test: * P<0.05, ** P<0.01, **** P<0.0001).

FIG. 10(A-M). LCB1v1.3 protects mice against B.1.1.7 variant and WA1/2020 E484K/N501Y/D614G strains. (A) Neutralization of LCB1v1.3 against B.1.1.7 or WA1/2020 E484K/N501Y/D614G SARS-COV-2 (EC₅₀ values: 802 pM and 667 pM, respectively; mean of two experiments, each performed in duplicate). (B-G) 7 to 8-week-old female K18-hACE2 transgenic mice were treated with a single 50 μg i.n. dose of LCB1v1.3 or control binder at 1 day prior to i.n. inoculation with 10³ PFU of B.1.1.7. (B) Weight change following LCB1v1.3 or control binder administration (mean+SEM; n=6, two experiments: two-way ANOVA with Sidak's post-test: *** P<0.001, **** P<0.0001). (C-G) Viral RNA levels at 6 dpi in the lung, heart, spleen, nasal wash, or brain (n=6, two experiments: Mann-Whitney test: * P<0.05, ** P<0.01). (H-M) 8-week-old male K18-hACE2 transgenic mice were treated with a single 50 μg i.n. dose of LCB1v1.3 or control binder at 1 day prior to i.n. inoculation with 10³ PFU of WA1/2020 E484K/N501Y/D614G. (H) Weight change following LCB1v1.3 or control binder administration (mean+SEM; n=6, two experiments: two-way ANOVA with Sidak's post-test: * P<0.05, **** P<0.0001). (I-M) Viral RNA levels at 6 dpi in the lung, heart, spleen, nasal wash, or brain (n=6, two experiments: Mann-Whitney test: * P<0.05, ** P<0.01).

FIG. 11. Cytokine and chemokine induction following SARS-CoV-2 infection. Individual plots for cytokine and chemokine RNA levels in the lungs of SARS-COV-2 infected mice at 4 dpi following treatment with control or LCB1-Fc binders (n=8 per group, two experiments: Mann-Whitney test: ns, not significant, * P<0.05, ** P<0.01, *** P<0.001). Data were used to generate the heat-map in FIG. 6C.

FIG. 12(A-C). Intranasal delivery of LCB1v1.3 at 1 or 2 days post-SARS-CoV-2 infection reduces viral burden, Related to FIG. 7. (A-C) 7 to 8-week-old male K18-hACE2 transgenic mice received a single 50 μg i.n. dose of LCB1v1.3 or control binder at one- or two-days post-inoculation with 10³ PFU of SARS-COV-2. Viral RNA levels at 7 dpi in the heart (A), spleen (B), or brain (C) (n=6, two experiments: one-way ANOVA: * P<0.05, ** P<0.01).

FIG. 13. Intranasal prophylaxis of LCB1v1.3 reduces weight loss, Related to FIG. 8. 7 to 8-week-old female K18-hACE2 transgenic mice received a single 50 μg i.n. dose of LCB1v1.3 or control binder at the indicated time prior to i.n. inoculation with 10³ PFU of SARS-COV-2. Weight change was recorded daily (mean+SEM; n=6, two experiments: two-way ANOVA with Sidak's post-test:* P<0.05, ** P<0.01, *** P<0.001, **** P<0.0001).

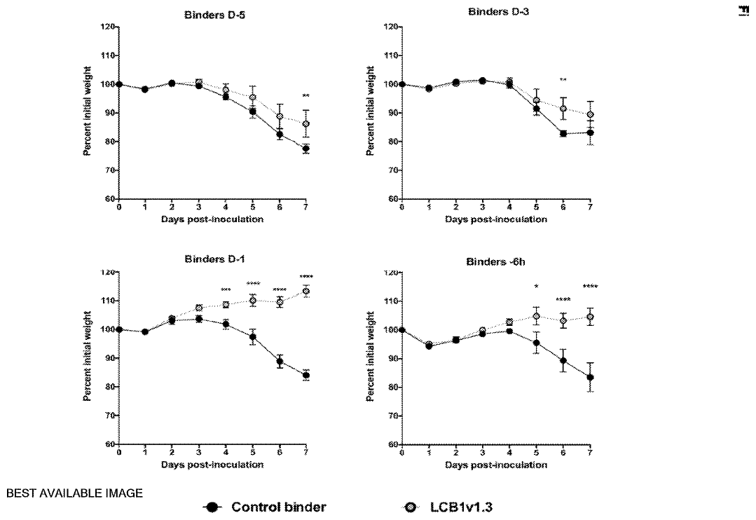

FIG. 14(A-B). Multivalent minibinders simultaneously engage multiple epitopes on the pre-fusion SARS-COV-2 spike protein resulting in extremely slow dissociation rates. (A,B) Dissociation of the minibinder construct and the receptor binding domain (RBD) (A) or the hexapro spike protein (S6P) (B) complex was monitored via competition with 100-fold molar excess of untagged M1 using AlphaLISA™ (Mean+SEM, n=3).

FIG. 15(A-F). Cryo-EM structures of multivalent minibinders in complex with the SARS-COV-2 S glycoprotein. (A) Ribbon diagram representations of all three minibinders bound to the RBD. (B) Cryo-EM map of F31-G10 in complex with two RBDs. (C) Cryo-EM map of F231-P24 in complex with three RBDs. (D) Design model of H2-1 bound to the S glycoprotein. (E) Cryo-EM map of H2-1 in complex with the S glycoprotein in two orthogonal orientations. (F) Cryo-EM map showing the interacting residues of the H2-1 and S glycoprotein interface.

FIG. 16(A-F). Multivalency enhances both the breadth and potency of neutralization against SARS-COV-2 variants by minibinders. (A) Dissociation of minibinder constructs from S6P variants after 24 hours was measured via competition with untagged H2-0 using AlphaLISA (mean, n=3). Cells containing an X indicate insufficient signal in the no competitor condition to quantify the fraction of protein bound. (B) Competition of minibinder constructs with ACE2 for S6P was measured via ELISA (mean, n=2). (C) Neutralization curves of minibinder constructs against SARS-COV-2 pseudovirus variants (mean, n=2) (D)) Table summarizing neutralization potencies of multivalent minibinder constructs against SARS-COV-2 pseudovirus variants. N/A indicates an IC₅₀ value above the tested concentration range and an IC₅₀ greater than 50,000 pM. (E) Neutralization curves of minibinder constructs against authentic SARS-COV-2 isolates (mean, n=2). (F) Table summarizing neutralization potencies of multivalent minibinder constructs against authentic SARS-COV-2 isolates.

FIG. 17(A-C). Top multivalent minibinder candidates are escape resistant and protect mice from SARS-COV-2 infection via pre-exposure intranasal administration. (A) Plaque assays were performed to isolate VSV-SARS-COV-2 chimera virus escape mutants against a control neutralizing antibody (2B04) and the F231-P12 and H2-1 multivalent minibinders. Images are representative of 35 replicate wells per multivalent minibinder. Large plaques, highlighted by black arrows, are indicative of escape. (B, C) K18-hACE2-transgenic mice (n=6/timepoint) were dosed with 50 μg H2-0 by intranasal administration (i.n., 2×25 μl doses per nostril, 100 μl total) 24 h prior (T-24 h) to infection with 10³ plaque forming units of SARS-COV-2 Variants B.1.1.7, B1.351, B.1.1.24 intranasally at Day 0. (B) Daily weight change following infection (mean+SEM; n=6, two-way ANOVA with Sidak's post-test: * P<0.05, *** P<0.001, **** P<0.0001). (C) After days post infection (6 dpi) animals (n=6/timepoint) were sacrificed and analyzed for the presence of SARS-COV-2 viral RNA by quantitative real time RT-PCR in the lung, heart, spleen, brain, or nasal wash (n=6: Mann-Whitney test: ns, not significant, * P<0.05, ** P<0.01).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, an N-terminal methionine residue is optional (i.e.: may be present or absent).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-101, wherein the polypeptide binds to SARS-COV-2 Spike glycoprotein receptor binding domain (RBD).

>LCB1-1
(SEQ ID NO: 1)
DKEWILQKIYEIMRLLDELGHAEASMRVSDLIYEF
MKKGDERLLEEAERLLEEVER
>LCB1-2
(SEQ ID NO: 2)
DKEEILNKIYEIMRLLDELGNAEASMRVSDLILEF
MKKGDERLLEEAERLLEEVER
>LCB1-3
(SEQ ID NO: 3)
DKEWILQKIYEIMRLLDELGHAEASMRVSDLIYEF
MKQGDERLLEEAERLLEEVER
>LCB1-4
(SEQ ID NO: 4)
DKENILQKIYEIMKTLDQLGHAEASMQVSDLIYEF
MKQGDERLLEEAERLLEEVER
>LCB1-5
(SEQ ID NO: 5)
DKENILQKIYEIMKTLDQLGHAEASMNVSDLIYEF
MKQGDERLLEEAERLLEEVER
(SEQ ID NO: 6)
LCB1_v1.1_Cys
DKENILQKIYEIMKTLDQLGHAEASMQVSDLIYEF
MKQGDERLLEEAERLLEEVERC
>LCB1_v1.2
(SEQ ID NO: 7)
DKENILQKIYEIMKTLDQLGHAEASMYVSDLIYEF
MKQGDERLLEEAERLLEEVER
>LCB1_v1.3
(SEQ ID NO: 8)
DKENILQKIYEIMKTLEQLGHAEASMQVSDLIYEF
MKQGDERLLEEAERLLEEVER
>LCB1_v1.4
(SEQ ID NO: 9)
DKENILQKIYEIMKTLEQLGHAEASMQVSDLIYEF
MKQGDENLLEEAEQLLQEVER
>LCB1_v1._5
(LCB1_v1._3 with N-link Glycosylation)
(SEQ ID NO: 10)
DKENILQKIYEIMKTLEQLGHAEASMNVSDLIYEF
MKQGDERLLEEAERLLEEVER
>LCB2-1
(SEQ ID NO: 11)
SDDEDSVRYLLYMAELRYEQGNPEKAKKILEMAEF
IAKRNNNEELERLVREVKKRL
>LCB2-2
(SEQ ID NO: 12)
SDDEDAVRYLLYMAELLYKQGNPEEAKKLLELAEF
IAKRNNNEELERLVREVKKRL
>LCB3-1
(SEQ ID NO: 13)
NDDELHMLMTDLVYEALHFAKDEEIKKRVFQLFEL
ADKAYKNNDRQKLEKVVEELKELLERLLS
>LCB3-2
(SEQ ID NO: 14)
NDDELLMLVTDLVAEALLFAKDEEIKKRVFTLFEL
ADKAYKNNDRDTLSKVVSELKELLERLQ
> LCB3_v1.2
(SEQ ID NO: 15)
NDDELHMQMTDLVYEALHFAKDEEIQKHVFQLFEK
ATKAYKNKDRQKLEKVVEELKELLERLLS
>LCB3-4
(SEQ ID NO: 16)
NDDELHMQMTDLVYEALHFAKDEEIQKHVFQLFEN
ATKAYKNKDRQKLEKVVEELKELLERLLS
>LCB3_v1.1
(SEQ ID NO: 17)
NDDELHMQMTDLVYEALHFAKDEEFQKHVFQLFEK
ATKAYKNNDRQKLEKVVEELKELLERLLS
>LCB3_v1.3
(SEQ ID NO: 19)
NDDELHMQMTDLVYEALHFAKDEEFQKHVFQLFEK
ATKAYKNKDRQKLEKVVEELKELLERLLS
>LCB3_v1.4
(SEQ ID NO: 20)
NDDELHMQMTDLVYEALHKAKDEEFQKHVFQLFEK
ATKARKNKDRQKLEKVVEELKELLERLLS
>LCB3_v1.5
(SEQ ID NO: 21)
NDDELHMQMTDLVYEALHKAKDEEMQKRVFQLFEQ
ADKAYKTKDRQKLEKVVEELKELLERLLS
>LCB4-1
(SEQ ID NO: 23)
QREKRLKOLEMLLEYAIERNDPYLMFDVAVEMLRL
AEENNDERIIERAKRILEEYE
>LCB4-2
(SEQ ID NO: 24)
DREERLKYLEMLLELAVERNDPYLIFDVAIELLRL
AEENNDERIYERAKRILEEVE
>LCB5-1
(SEQ ID NO: 25)
SLEELKEQVKELKKELSPEMRRLIEEALRFLEEGN
PAMAMMVLSDLVYQLGDPRVIDLYMLVTKT
>LCB5-2
(SEQ ID NO: 26)
SLEEVKEILKELKKELSPEDRRLIEEALRLLEEGN
PAMASMVLSDLVFLLGDPRVIELLMLVTKT
>LCB6-1
(SEQ ID NO: 27)
DREQRLVRFLVRLASKFNLSPEQILQLFEVLEELL
ERGVSEEEIRKOLEEVAKELG
>LCB6-2
(SEQ ID NO: 28)
DREQRLVRFLVRLASKFNLSMEQILILFDVLEELL
ERGVSEEEIRKILEEVAKEL
>LCB7-1
(SEQ ID NO: 29)
DDDIRYLIYMAKLRLEQGNPEEAEKVLEMARFLAE
RLGMEELLKEVRELLRKIEELR
>LCB7-2
(SEQ ID NO: 30)
DDDVRYLIYMAKLLLEQGNPEEAEKVLESARFAAE
LLGNEELLKEVRELLRKIEELR
>LCB8-1
(SEQ ID NO: 31)
PIIELLREAKEKNDEFAISDALYLVNELLQRTGDP
RLEEVLYLIWRALKEKDPRLLDRAIELFER
>LCB8-2
(SEQ ID NO: 32)
PVTELLREAKEKNDPMAISDALFLVFELAQRTGDP
RLEEVLFLIWRALKEKDPRLLDRAIELFER
>AHB1-1
(SEQ ID NO: 33)
DEDLEELERLYRKAEEVAKEAKDASRRGDDERAKE
QMERAMRLFDQVFELAQELQEKQTDGNRQKATHLD
KAVKEAADELYQRVR
>AHB1-2
(SEQ ID NO: 34)
AADELYQRVR
>AHB2-1
(SEQ ID NO: 100)
ELEERVMHLLDQVSELAHELLHKLTGEELQRATHF
DKWANEAILELIKSDDEREIREIEEEARRILEHLE
ELARK
>AHB2-2
(SEQ ID NO: 101)
ELEEQVMHVLDQVSELAHELLHKLTGEELERAAYF
NWWATEMMLELIKSDDEREIREIEEEARRILEHLE
ELARK

As detailed in the examples that follows, the polypeptides bind with high affinity to the SARS-COV-2 Spike glycoprotein receptor binding domain (RBD).

In all of embodiments herein, the percent identity requirement does not include any additional functional domain that may be incorporated in the polypeptide. In one embodiment, 1, 2, or 3 amino acids may be deleted from the N and/or C terminus.

The polypeptides have been subjected to extensive mutational analysis as described in the examples that follow, permitting determination of allowable substitutions at each residue within the polypeptide. Exemplary substitutions are as shown in Table 1 (The number denotes the residue number, and the letters denote the single letter amino acids that can be present at that residue). Thus, in one embodiment, amino acid substitutions relative to the reference polypeptide amino acid sequence (i.e.: one of SEQ ID NOS: 1-17, 19-21, 23-34 and 100-101) are selected from the exemplary amino acid substitutions provided in Table 1.

TABLE 1
Exemplary substitutions:

```
LCB1 (SEQ ID NOS: 1-10 and 102-136)
  1 -- A, C, D, E, F, G, H, I, K, L, M, N, P, Q, R, S, T, V, W, Y
  2 -- A, D, E, F, G, H, I, K, L, M, N, P, Q, R, S, T, V, W, Y
  3 -- A, D, E, F, G, H, K, L, M, N, P, Q, R, S, T, V, W, Y
  4 -- A, C, D, E, F, G, H, I, K, L, M, N, P, Q, R, S, T, V, W, Y
  5 -- A, C, D, E, F, G, H, I, K, L, M, N, P, Q, R, S, T, V, W, Y
  6 -- A, C, I, L, M, Q, T, V
  7 -- A, C, D, E, F, G, H, M, N, P, Q, R, S, V, W, Y
  8 -- A, C, D, E, F, G, H, I, K, L, M, N, Q, R, S, T, V, W, Y
  9 -- C, I, L, M, N, Q, T, V
10 -- C, F, V, W, Y
11 -- A, C, D, E, F, G, H, I, K, L, M, N, Q, R, S, T, V, W, Y
12 -- A, C, D, H, I, L, M, N, S, T, V, Y
13 -- C, I, M, Q
14 -- A, C, D, E, F, G, H, I, K, L, M, N, Q, R, S, T, V, W, Y
15 -- A, C, D, E, F, G, H, I, K, L, M, N, Q, R, S, T, V, W, Y
16 -- C, F, I, L, M, T, V
17 -- A, C, D, E, F, G, H, I, K, L, M, N, Q, R, S, T, V, W, Y
18 -- A, C, D, E, F, G, H, I, K, L, M, N, Q, R, S, T, V, W, Y
19 -- A, C, D, E, F, G, H, I, K, L, M, N, Q, R, S, T, V, W, Y
20 -- A, C, D, E, F, G, H, K, L, M, N, Q, R, S, T, W
21 -- A, C, D, E, F, G, H, I, K, L, M, N, Q, R, S, T, V, W, Y
22 -- A, C, D, F, G, H, I, L, M, N, P, Q, S, T, V, W, Y
23 -- C, E, M, N, P, Q, S, T, V
24 -- A, C, D, E, F, G, H, K, L, M, N, P, Q, R, S, T, V, W, Y
25 -- A, C, G, M, N, Q, S, T, V
26 -- M, N, V
27 -- A, C, D, E, F, G, H, I, K, L, M, N, Q, R, S, T, V, W, Y
28 -- A, C, G, I, L, S, T, V
29 -- A, C, S, V, W
30 -- D
31 -- A, C, D, F, G, H, I, K, L, M, N, Q, R, S, T, V, W, Y
32 -- C, F, H, I, L, M, N, P, T, V
33 -- A, C, D, E, F, G, H, I, K, L, M, N, P, Q, R, S, T, V, W, Y
34 -- A, C, D, E, F, G, H, I, K, L, M, N, P, Q, R, S, T, V, W, Y
35 -- A, C, D, F, H, M, Q, V, W, Y
36 -- A, C, D, E, G, H, I, L, M, N, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, Q, R, S, T, V, W, Y
39 -- A, C, D, E, F, G, H, K, L, M, N, P, Q, R, S, T, V, W, Y
40 -- D, E, G, H, N, P, Q
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, Q, R, S, V, W, Y
45 -- A, C, D, E, F, G, H, I, K, L, M, N, P, Q, R, S, T, V, W, Y
46 -- A, C, D, E, F, G, H, I, K, L, M, N, P, Q, R, S, T, V, W, Y
47 -- A, C, G, P, S, T, V
48 -- A, C, D, E, F, G, H, I, K, L, M, N, Q, R, S, T, V, W, Y
49 -- A, C, D, E, F, G, H, I, K, L, M, N, Q, R, S, T, V, W, Y
50 -- A, C, D, E, F, G, H, I, K, L, M, N, Q, R, S, T, V, W, Y
51 -- A, C, E, F, G, H, I, K, L, M, N, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- A, C, D, E, F, G, H, I, K, L, M, N, P, Q, R, S, T, V, W, Y
LCB2 (SEQ ID NOS: 11-12)
  1 -- A, C, D, E, G, N, P, S, T
  2 -- D, M, P, Q, Y
  3 --A, D, E, N, Q
  4 -- C, D, E, V
  5 -- D
  6 -- A, C, D, E, G, N, Q, S, T, V
  7 -- A, C, G, I, L, M, P, S, T, V
  8 -- A, C, E, F, G, H, I, K, L, M, N, Q, R, S, T, V, W, Y
  9 -- D, N, Y
10 -- I, L, T
11 -- C, E, G, I, L, M, W
12 -- F, H, Y
13 -- E, M, Q, R, V
14 -- A, C, E, F, G, H, I, K, L, M, N, Q, R, S, T, V, W, Y
15 -- A, C, D, E, G, H, I, K, L, M, N, Q, R, S, T, V
16 -- C, H, L, T
17 -- A, C, D, E, F, G, H, I, K, L, M, N, P, Q, R, S, T, V, W, Y
18 -- A, C, D, E, F, G, H, I, K, L, M, N, Q, R, S, T, V, W, Y
19 -- A, C, D, E, F, G, H, I, K, L, M, N, P, Q, R, S, T, V, W, Y
20 -- A, C, E, F, G, H, I, K, L, M, N, P, Q, R, S, T, V, Y
21 -- A, C, D, E, F, G, H, I, K, L, M, N, P, Q, R, S, T, V, W, Y
22 -- A, C, D, E, G, I, K, L, N, P, Q, R, S, T, V
23 -- A, C, D, E, F, G, H, I, K, L, M, N, P, Q, R, S, T, V, W, Y
24 -- A, C, D, E, F, G, H, I, K, L, M, N, P, Q, R, S, T, V, W, Y
25 -- A, C, E, G, H, I, K, N, P, Q, R, S, T, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- H, K, R, T, Y
29 -- C, D, E, H, I, K, L, M, N, P, Q, R, S, T, V, Y
30 -- A, C, D, E, F, G, H, I, K, L, M, N, P, Q, S, T, V, W, Y
31 -- A, C, D, E, F, G, H, I, K, L, M, N, P, Q, R, S, T, V, Y
32 -- F, H, I, K, L, M, P, Q, R, Y
33 -- A, C, G, P, S, T
34 -- A, C, D, E, F, G, H, I, K, L, M, N, Q, R, S, T, V, W, Y
35 -- F, H, Y
36 -- A, C, E, H, I, L, M, S, V
37 -- A, C, E, G, H, L, M, Q, R, S, T, V, W
38 -- A, C, D, E, F, G, H, I, K, L, M, N, P, Q, R, S, T, V, W, Y
39 -- A, C, D, E, G, H, I, K, L, M, N, P, Q, R, S, T, V
40 -- A, C, D, E, G, H, I, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, E, F, I, L, M, P, S, T, V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- A, C, D, E, F, G, H, I, K, L, M, N, P, Q, R, S, T, V, W, Y
49 -- A, C, E, F, G, H, I, K, L, M, N, P, Q, R, S, T, V, W, Y
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- A, C, D, E, F, G, H, I, K, L, M, N, P, Q, R, S, T, V, W, Y
LCB3 (SEQ ID NOS: 13-17, 19-21 and 137-163)
  1 -- C, E, F, I, M, N, T, W
  2 -- A, C, D, E, F, G, H, I, K, L, M, N, P, Q, R, S, T, V, W, Y
  3 -- D, G, L, M, N, S, Y
  4 -- A, C, E, F, H, K, Q, T
  5 -- A, C, E, F, G, H, I, K, L, M, N, Q, R, S, T, V, W, Y
  6 -- A, C, D, E, F, G, H, I, K, L, M, N, P, Q, R, S, T, V, W, Y
  7 -- A, C, D, F, I, L, M, P, R, S, V, W
  8 -- A, C, D, E, F, G, H, I, K, L, M, N, Q, R, S, T, V, W, Y
  9 -- A, C, E, F, G, H, I, L, M, N, Q, R, S, T, V, Y
10 -- A, C, F, G, H, K, M, N, Q, R, S, T, Y
11 -- D, F, H, L, M, N, Q
12 -- A, C, D, E, F, G, H, I, K, L, M, N, P, Q, R, S, T, V, W, Y
13 -- A, F, I, L, M, N, Q, S, T, V
14 -- A, C, F, G, H, I, K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, L, M, N, P, Q, R, S, T, V, W, Y
17 -- A, C, D, F, G, H, I, K, L, M, N, P, Q, R, S, T, V, W
18 -- A, C, D, E, F, G, H, I, K, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, C, D, E, F, G, H, I, K, L, M, N, P, Q, R, S, T, V, W, Y
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- A, C, D, E, F, G, H, I, K, L, M, N, P, Q, R, S, T, V, W, Y
29 -- A, C, D, E, F, G, I, L, M, N, P, S, T, V, W, Y
30 -- C, E, F, H, L, N, S,W, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, C, E, F, I, K, P, Q, S, V, W, Y
34 -- A, D, E, F, G, H, M, N, P, Q, R, S, V, W, Y
35 -- A, C, D, E, F, G, H, I, K, L, M, N, P, Q, R, S, T, V, W, Y
36 -- A, C, E, G, H, I, M, N, Q, S, T, V
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, D, E, F, G, H, I, K, L, M, N, P, Q, R, S, T , V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- A, C, E, F, G, I, K, L, M, N, P, Q, S, T, V, W
49 -- A, C, D, E, F, G, H, I, K, L, M, N, P, Q, R, S, T, V, W, Y
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, E, F, G, H, I, K, L, M, N, Q, S, T, V, W, Y
56 -- A, C, D, E, F, G, H, I, K, L, M, N, P, Q, R, S, T, V, W, Y
57 -- A, C, D, E, F, G, H, I, K, L, M, N, P, Q, R, S, T, V, W, Y
58 -- A, C, D, E, F, G, H, I, K, L, M, N, P, Q, R, S, T, V, W, Y
59 -- A, C, D, E, F, G, H, I, K, L, M, N, P, Q, R, S, T, V, W, Y
60 -- A, C, D, E, F, G, H, I, K, L, M, N, P, Q, R, S, T, V, W, Y
61 -- A, C, D, E, F, G, H, I, K, L, M, N, P, Q, R, S, T, V, W, Y
62 -- A, C, D, E, F, G, H, I, K, L, M, N, P, Q, R, S, T , V, W, Y
63 -- A, C, D, E, F, G, H, I, K, L, M, N, P, Q, R, S, T, V, W, Y
64 -- A, C, D, E, F, G, H, I, K, L, M, N, P, Q, R, S, T, V, W, Y
LCB4 (SEQ ID NO: 23-24)
  1 -- A, C, D, E, F, G, H, I, K, L, M, N, P, Q, R, S, T, V, W, Y
  2 -- A, C, D, E, F, G, H, I, K, L, M, N, P, Q, R, S, T, V, W, Y
  3 -- A, C, D, E, F, G, H, I, K, L, M, N, P, Q, R, S, T, V, W, Y
  4 -- A, C, D, E, F, G, H, I, K, L, M, N, P, Q, R, S, T, V, W, Y
  5 -- C, D, H, K, N, Q, R, Y
  6 -- A, C, F, G, I, K, L, M, P, Q, R, S, T, V, Y
  7 -- A, C, D, E, F, G, H, I, K, L, M, N, P, Q, R, S, T, V, W, Y
  8 -- A, C, H, I, M, N, Q, R, S, T, V, Y
  9 -- A, C, D, G, H, I, K, L, M, N, Q, R, S, T, V, Y
10 -- A, C, D, E, M, N, P, Q, S, T, V
11 -- C, D, G, H, I, K, L, M, N, P, R, S, T, V
12 -- F, G, I, L
13 -- F, I, L, M, S, V, Y
14 -- A, C, D, E, G, L, M, N, Q, R, S, T, V
15 -- C, E, F, G, H, I, L, M, S, V, W, Y
16 -- A, G, T, Y
17 -- A, C, D, E, F, G, H, I, K, L, M, N, Q, R, S, T, V, W, Y
18 -- A, C, D, E, F, G, H, I, K, L, M, N, Q, R, S, T, V, W, Y
19 -- A, C, D, E, F, G, H, I, K, L, M, N, Q, R, S, T, V, W, Y
20 -- A, C, D, E, F, G, H, I, K, L, M, N, P, Q, R, S, T, V, W, Y
21 -- C, D, Q, Y
22 -- A, C, D, E, F, G, H, I, K, L, M, N, P, Q, R, S, T, V, W, Y
23 -- E, F, H, Y
24 -- A, F, G, I, L, M, W
25 -- A, C, E, G, H, I, K, L, M, N, Q, R, S, T, V, Y
26 -- C, F, H, I, L, N, S, T, V, W
27 -- D, Q, W, Y
28 -- A, C, D, I, L, V, Y
29 -- A, C, E, G, K, L, N, Q, R, S, T
30 -- C, I, L, M, P, T, V
31 -- C, D, E
32 -- A, C, E, I, L, M, Q, S, T, V, Y
33 -- A, C, E, F, G, H, I, K, L, M, Q, R, S, T, V, Y
34 -- C, D, F, G, H, L, M, N, P, R, S, T, W, Y
35 -- A, C, E, F, G, H, I, K, L, N, P, R, T, V, W
36 -- A, C, G, S, T, V
37 -- A, C, D, E, G, H, I, K, L, M, N, P, Q, R, S, T, V, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, I, K, L, M, N, Q, R, S, T, V, Y
41 -- A, C, D, E, G, H, K, N, Q, S, W
42 -- A, C, D, E, F, G, H, I, K, L, M, N, P, Q, R, S, T, V, W, Y
43 -- A, C, D, E, F, G, H, I, K, L, M, N, P, Q, R, S, T, V, Y
44 -- A, E, F, G, H, I, K, L, M, N, Q, R, S, T, V
45 -- A, C, E, F, G, H, I, K, L, M, N, Q, R, S, T, V, W, Y
46 -- A, C, D, E, F, G, H, I, K, L, M, N, Q, R, S, T, V, W, Y
47 -- A, C, D, E, F, G, H, I, K, L, M, N, Q, R, S, T, V, W, Y
48 -- A, C, M, S, T, V
49 -- A, H, I, K, L, M, N, Q, R, S, T, V, W, Y
50 -- A, C, D, E, F, G, H, I, K, L, M, N, P, Q, R, S, T, V, W, Y
51 -- A, F, I, K, L, M, R, T, V, W, Y
52 -- F, I, K, L, M, V
53 -- A, C, D, E, F, G, H, I, K, L, M, N, Q, R, S, T, V, W, Y
54 -- A, C, D, E, F, G, H, I, K, L, M, N, P, Q, R, S, T, V, W, Y
55 -- A, C, F, G, H, I, K, L, M, N, Q, R, S, T, V, W, Y
56 -- A, C, D, E, F, G, H, I, K, L, M, N, P, Q, R, S, T, V, W, Y
LCB5 (SEQ ID NO: 25-26)
  1 -- A, C, D, E, F, G, H, I, K, L, M, N, P, Q, R, S, T, V, W, Y
  2 -- A, C, D, E, F, G, H, I, K, L, M, N, P, Q, R, S, T, V, W, Y
  3 -- A, C, D, E, F, G, H, I, K, L, M, N, P, Q, R, S, T, V, W, Y
  4 -- A, C, D, E, F, G, H, I, K, L, M, N, P, Q, R, S, T, V, W, Y
  5 -- A, C, D, E, F, G, H, I, K, L, M, N, P, Q, R, S, T, V, W, Y
  6 -- A, C, D, E, F, G, H, I, K, L, M, N, P, Q, R, S, T, V, W, Y
  7 -- A, C, D, E, F, G, H, I, K, L, M, N, P, Q, R, S, T, V, W, Y
  8 -- A, C, D, E, F, G, H, I, K, L, M, N, P, Q, R, S, T, V, W, Y
  9 -- A, C, E, F, G, H, I , L, M, N, Q, S, T, V, W, Y
10 -- A, C, D, E, F, G, H, I, K, L, M, N, P, Q, R, S, T, V, W, Y
11 -- A, C, D, E, F, G, H, I, K, L, M, N, P, Q, R, S, T, V, W, Y
12 -- A, C, D, E, F, G, H, I, L, M, N, P, Q, R, S, T, V, W, Y
13 -- A, C, D, E, F, G, H, I, K, L, M, N, P, Q, R, S, T, V, W, Y
14 -- A, C, D, E, F, G, H, I , K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, K, L, M, N, P, Q, R, S, T, V, W, Y
17 -- A, C, D, E, F, G, H, I, K, L, M, N, P, Q, R, S, T, V, W, Y
18 -- A, C, D, E, F, G, H, I, K, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I , K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, C, D, E, F, G, H, I, L, M, N, P, Q, R, S, T, W, Y
24 -- A, C, D, E, F, G, H, I, L, M, N, P, Q, S, T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- A, C, G, H, I, S, T, V
28 -- A, C, D, E, F, G, H, I, K, L, M, N, Q, R, S, T, V, W, Y
29 -- A, C, D, E, F, G, H, I, K, L, M, N, P, Q, R, S, T, V, W, Y
30 -- A, C, D, E, F, G, H, I, K, L, M, N, P, Q, R, S, T, V, W, Y
31 -- A, C, E, F, H, I, K, L, M, N, Q, S, T, V, W, Y
32 -- A, C, D, E, F, G, H, I, K, L, M, N, P, Q, R, S, T, V, W, Y
33 -- A, C, D, E, F, G, H, I, K, L, M, N, P, Q, R, S, T, V, W, Y
34 -- A, C, D, E, F, G, H, I, K, L, M, N, P, Q, R, S, T, V, W, Y
35 -- A, C, D, E, F, G, H, I, K, L, M, N, Q, R, S, T, V, W, Y
36 -- A, C, D, E, F, G, H, I, K, L, M, N, P, Q, R, S, T, V, W, Y
37 -- A, C, D, E, F, G, H, I, L, M, N, P, Q, R, S, T, V, W, Y
38 -- A, C, D, E, G, I, L, M, N, P, Q, S, T, V, W
39 -- A, C, F, G, L, M, N, S, T, V, W
40 -- A, C, E, F, G, H, I, K, L, M, N, Q, R, S, T, V, Y
41 -- C, H, I, L, M, P, R
42 -- A, C, E, G, H, I, L, M, P, T, V, Y
43 -- C, I, L, M, Q, T, V
44 -- A, C, D, F, G, H, I, M, S, T
45 -- D, Y
46 -- A, C, D, F, I, L, R, V
47 -- C, E, G, I, V
48 -- F, I, V, W, Y
49 -- A, C, D, E, F, G, H, I, K, L, M, N, Q, R, S, T, V, W, Y
50 -- A, C, E, F, G, H, I, K, L, M, N, Q, R, S, T, V, W, Y
51 -- A, C, D, E, F, G, H, I, K, L, M, N, P, Q, R, S, T, V, W, Y
52 -- C, D, E, H, I, K, N, P, Q, R, S, T, Y
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- F, I, L, M, T, V, W
57 -- A, C, D, E, F, G, H, N, P, Q, R, S, T, W, Y
58 -- A, C, D, E, F, G, H, I, K, L, M, N, P, Q, R, S, T, V, W, Y
59 -- A, C, F, I, L, M, T, V, Y
60 -- C, F, M, N, V, Y
61 -- A, C, D, E, F, G, H, I, K, L, M, N, Q, R, S, T, V, W, Y
62 -- A, C, F, G, I, L, M, S, T, V, W
63 -- A, C, E, F, G, H, I, K, L, M, N, Q, R, S, T, V, W, Y
64 -- A, C, E, F, G, H, K, L, N, P, R, S, T, W, Y
65 -- A, C, D, E, F, G, H, I, K, L, M, N, P, Q, R, S, T, V, W, Y
LCB6 (SEQ ID NO: 27-28)
  1 -- A, C, D, E, F, G, H, I, K, L, M, N, P, Q, R, S, T, V, W, Y
  2 -- A, C, D, E, F, G, H, I, K, L, M, N, P, Q, R, S, T, V, W, Y
  3 -- E, W
  4 -- A, C, D, E, F, G, H, I , K, L, M, N, P, Q, R, S, T, V, W, Y
  5 -- A, C, D, E, F, G, H, I, K, L, M, N, P, Q, R, S, T, V, W, Y
  6 -- F, L, M, R, S
  7 -- H, T, V
  8 -- A, C, D, E, F, G, H, I , K, L, M, N, P, Q, R, S, T, V, W, Y
  9 -- F, M
10 -- A, K, L, W
11 -- D, E, G, V, Y
12 -- A, C, D, E, F, G, H, I, K, L, M, N , P, Q, R, S, T, V, W, Y
13 -- E, L
14 -- A, C, D, E, F, G, H, I, K, L, M, N, P, Q, R, S, T, V, W, Y
15 -- A, C, D, E, F, G, H, I, K, L, M, N, P, Q, R, S, T, V, W, Y
16 -- A, C, D, E, F, G, H, I, K, L, M, N, P, Q, R, S, T, V, W, Y
17 -- F, N, P, S
18 -- A, C, D, E, F, G, H, I, K, L, M, N, P, Q, R, S, T, V, W, Y
19 -- L, N, Q, V
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- C, D, P, Q, R, W
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- A, C, D, E, F, G, H, I, K, L, M, N, P, Q, R, S, T, V, W, Y
27 -- D, H, L, S, W
28 -- A, C, D, E, F, G, H, I, K, L, M, N, P, Q, R, S, T, V, W, Y
29 -- A, C, D, E, F, G, H, I, K, L, M, N, P, Q, R, S, T, V, W, Y
30 -- L, Q, V, W
31 -- I, K, L, S
32 -- A, C, D, E, F, G, H, I, K, L, M, N, P, Q, R, S, T, V, W, Y
33 -- A, C, D, E, F, G, H, I, K, L, M, N, P, Q, R, S, T, V, W, Y
34 -- A, F, L, T, V
35 -- C, D, G, H, K, L, N, T
36 -- A, C, D, E, F, G, H, I, K, L, M, N, P, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, D, E, F, G, H, I, K, L, M, N, P, Q, R, S, T, V, W, Y
40 -- A, C, D, E, F, G, H, I, K, L, M, N, P, Q, R, S, T, V, W, Y
41 -- A, C, D, E, F, G, H, I, K, L, M, N, P, Q, R, S, T, V, W, Y
42 -- A, C, D, E, F, G, H, I, K, L, M, N, P, Q, R, S, T, V, W, Y
43 -- A, C, D, E, F, G, H, I, K, L, M, N, P, Q, R, S, T, V, W, Y
44 -- F, I
45 -- A, C, D, E, F, G, H, I, K, L, M, N, P, Q, R, S, T, V, W, Y
46 -- A, C, D, E, F, G, H, I, K, L, M, N, P, Q, R, S, T, V, W, Y
47 -- A, C, D, E, F, G, H, I, K, L, M, N, P, Q, R, S, T, V, W, Y
48 -- L, Q, R, T
49 -- A, C, D, E, F, G, H, I, K, L, M, N, P, Q, R, S, T, V, W, Y
50 -- A, C, D, E, F, G, H, I, K, L, M, N, P, Q, R, S, T, V, W, Y
51 -- C, V, Y
52 -- A, E, H, K
53 -- A, C, D, E, F, G, H, I, K, L, M, N, P, Q, R, S, T, V, W, Y
54 -- A, C, D, E, F, G, H, I, K, L, M, N, P, Q, R, S, T, V, W, Y
55 -- C, F, H, L, P, W, Y
56 -- A, C, D, E, F, G, H, I, K, L, M, N, P, Q, R, S, T, V, W, Y
LCB7 (SEQ ID NO: 29-30)
  1 -- A, C, D, E, F, G, H, I, K, L, M, N, P, Q, R, S, T, V, W, Y
  2 -- A, C, D, E, F, G, H, I, K, L, M, N, P, Q, R, S, T, V, W, Y
  3 -- A, C, D, E, F, G, H, I, K, L, M, N, P, Q, R, S, T, V, W, Y
  4 -- I, T, V
  5 -- A, C, D, E, F, G, H , I, K, L, M, N, P, Q, R, S, T, V, W, Y
  6 -- A, C, D, E, F, G, H, I, K, L, M, N, P, Q, R, S, T, V, W, Y
  7 -- L, P, Y
  8 -- A, C, D, E, F, G, H, I, K, L, M, N, P, Q, R, S, T, V, W, Y
  9 -- A, C, D, E, F, G, H, I, K, L, M, N, P, Q, R, S, T, V, W, Y
10 -- A, C, D, E, F, G, H, I, K, L, M, N, P, Q, R, S, T, V, W, Y
11 -- A
12 -- A, C, D, E, F, G, H, I, K, L, M, N, P, Q, R, S, T, V, W, Y
13 -- A, L, P
14 -- H, L, R, T, Y
15 -- A, C, D, E, F, G, H, I, K, L, M, N, P, Q, R, S, T, V, W, Y
16 -- A, C, D, E, F, G, H, I, K, L, M, N, P, Q, R, S, T, V, W, Y
17 -- A, C, D, E, F, G, H, I, K, L, M, N, P, Q, R, S, T, V, W, Y
18 -- A, C, D, E, F, G, H, I, K, L, M, N, P, Q, R, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, D, E, F, G, H, I, K, L, M, N, P, Q, R, S, T, V, W, Y
22 -- A, C, D, E, F, G, H, I, K, L, M, N, P, Q, R, S, T, V, W, Y
23 -- A, S
24 -- A, C, D, E, F, G, H, I, K, L, M, N, P, Q, R, S, T, V, W, Y
25 -- A, C, D, E, F, G, H, I, K, L, M, N, P, Q, R, S, T, V, W, Y
26 -- C, G, S, V, Y
27 -- K, L, M, W
28 -- A, C, D, E, F, G, H, I, K, L, M, N, P, Q, R, S, T, V, W, Y
29 -- A, C, D, E, F, G, H, I, K, L, M, N, P, Q, R, S, T, V, W, Y
30 -- A, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, C, F, I, K, L, V, W
34 -- A, H, L
35 -- A, C, D, E, F, G, H, I, K, L, M, N, P, Q, R, S, T, V, W, Y
36 -- A, C, D, E, F, G, H, I, K, L, M, N, P, Q, R, S, T, V, W, Y
37 -- A, C, D, E, F, G, H, I, K, L, M, N, P, Q, R, S, T, V, W, Y
38 -- A, C, D, E, F, G, H, I, K, L, M, N, P, Q, R, S, T, V, W, Y
39 -- A, C, K, L, M, N
40 -- A, C, D, E, F, G, H, I, K, L, M, N, P, Q, R, S, T, V, W, Y
41 -- A, C, D, E, F, G, H, I, K, L, M, N, P, Q, R, S, T, V, W, Y
42 -- A, C, D, L, V
43 -- A, C, D, E, F, G, H, I, K, L, M, N, P, Q, R, S, T, V, W, Y
44 -- A, C, D, E, F, G, H, I, K, L, M, N, P, Q, R, S, T, V, W, Y
45 -- A, C, D, E, F, G, H, I, K, L, M, N, P, Q, R, S, T, V, W, Y
46 -- Q, S, V
47 -- A, C, D, E, F, G, H, I, K, L, M, N, P, Q, R, S, T, V, W, Y
48 -- A, C, D, E, F, G, H, I, K, L, M, N, P, Q, R, S, T, V, W, Y
49 -- E, L
50 -- A, C, D, E, F, G, H, I, K, L, M, N, P, Q, R, S, T, V, W, Y
51 -- A, C, D, E, F, G, H, I, K, L, M, N, P, Q, R, S, T, V, W, Y
52 -- A, C, D, E, F, G, H, I, K, L, M, N, P, Q, R, S, T, V, W, Y
53 -- I
54 -- A, C, D, E, F, G, H, I, K, L, M, N, P, Q, R, S, T, V, W, Y
55 -- A, C, D, E, F, G, H, I, K, L, M, N, P, Q, R, S, T, V, W, Y
56 -- L, M, N, R
57 -- A, C, D, E, F, G, H, I, K, L, M, N, P, Q, R, S, T, V, W, Y
LCB8 (SEQ ID NO: 31-32)
  1 -- A, C, D, E, F, G, H, I, K, L, M, N, P, Q, R, S, T, V, W, Y
  2 -- C, F, I, L, M, S, V, W, Y
  3 -- A, C, E, F, G, H, I, K, L, M, N, P, Q, R, S, T, V, W, Y
  4 -- A, C, D, E, F, G, H, I, K, L, M, N, Q, R, S, T, V, W, Y
  5 -- A, C, F, G, I, K, L, M, Q, S, T, V, W, Y
  6 -- H, I, K, L,M
  7 -- A, H, I, K, L, M, N, P, Q, R, W, Y
  8 -- A, C, D, E, F, G, H, I, K, L, M, N, Q, R, S, T, V, W, Y
  9 -- A, C, F, G, I, L, M, S, Y
10 -- A, F, H, K, L, M, Q, R, S
11 -- A, C, D, E, F, G, H, I, K, L, M, N, Q, R, S, T, V, W, Y
12 -- A, C, D, E, G, H, I, K, L, M, N, Q, R, S, T, V, W, Y
13 -- A, C, D, E, F, G, H, M, N, Q, S, W, Y
14 -- C, D, E, H, N, Q, S
15 -- A, D, E, F, H, I, L, M, N, P, Q, S, T, V, W, Y
16 -- C, F, M, N, R, Y
17 -- A, C, I, L, M, Q, R, V
18 -- A, C, F, H, I, L, M, T, V, Y
19 -- I, Q, S
20 -- D, N
21 -- A, C, G, S, V
22 -- A, C, I, L, M, V
23 -- C, F, R, T, W, Y
24 -- A, C, D, E, F, G, H, I, L, M, N, Q, R, S, T, V, W, Y
25 -- C, E, S, T, V, Y
26 -- A, C, D, E, F, G, H, N, Q, S, T
27 -- A, C, D, E, G, H, I, K, L, M, N, Q, R, S, T, V
28 -- C, E, F, G, H, I, K, L, M, Q, R, W, Y
29 -- A, C, F, G, H, I, K, L, M, N, Q, R, S, T, V, Y
30 -- A, C, E, G, H, K, M, N, P, Q, R, T
31 -- A, C, D, E, F, G, H, I, K, L, M, N, Q, R, S, T, V, Y
32 -- A, C, D, E, G, H, I, K, N, Q, R, S, T, W
33 -- A, C, E, G, H, K, M, N, P, Q, R, S, W, Y
34 -- C, D, E, F, H, M, N, W, Y
35 -- A, C, D, E, F, G, H, I, K, L, M, N, P, Q, R, S, T, V, Y
36 -- A, C, D, E, F, G, H, K, L, M, N, Q, R, S, T, V, W, Y
37 -- F, G, H, I, L, M, S, T, Y
38 -- D, E, H, Q, W, Y
39 -- C, D, E, F, G, H, K, L, M, N, P, Q, R, S, T, V, W, Y
40 -- A, C, E, G, H, I, K, M, P, V, Y
41 -- C, F, H, I, K, L, M, R, S, T, V
42 -- E, F, I, T, W, Y
43 -- A, C, D, E, F, H, I, L, M, N, Q, R, S, T, V, W, Y
44 -- C, G, I, K, L, M, T, V, Y
45 -- G, S, W, Y
46 -- C, I, K, L, M, N, Q, R, S, T
47 -- A, C, E, N, Q, S,T,V
48 -- C, D, E, F, H, I, L, M,W
49 -- C, D, F, H, K, L, M, N, Q, R, T
50 -- A, C, D, E, N, Y
51 -- A, C, D, E, F, G, H, I, K, L, M, N, P, Q, R, S, T , V, W, Y
52 -- A, C, D, E, G, H, K, L, M, N, Q, R, S, T
53 -- A, C, D, E, F, G, H, I, L, M, N, P, Q, S, T, V, W, Y
54 -- A, C, D, E, F, G, H, I, K, L, M, N, P, Q, R, S, T, V, W, Y
55 -- A, C, D, E, F, G, H, I , K, L, M, N, P, Q, S, T, V, W, Y
56 -- C, I, L, M
57 -- A, C, D, E, G, I, N, Q, S, T
58 -- A, C, D, E, F, G, H, I, K, L, M, N, P, Q, R, S, T, V, W, Y
59 -- A, C, G, P, S
60 -- A, C, E, F, G, I, L, M, N, Q, S, T, V
61 -- A, C, D, E, F, G, H, I, K, L, M, N, P, Q, R, S, T, V, W, Y
62 -- A, C, D, E, F, G, H, I, K, L, M, N, Q, R, S, T, V, W, Y
63 -- A, C, E, F, G, H, I, L, M, N, Q, S, T, V, W, Y
64 -- A, C, D, E, G, H, I, K, L, M, N, P, Q, S, T, V
65 -- A, C, D, E, G, H, I, K, L, M, N, P, Q, R, S, T, W, Y
AHB1 (SEQ ID NOS: 33-34)
  1 -- A, C, D, E, F, G, H, I, K, L, M, N, P, Q, R, S, T, V, W, Y
  2 -- A, C, D, E, F, G, H, I, K, L, M, N, P, Q, R, S, T, V, W, Y
  3 -- A, C, D, E, F, G, H, I, K, L, M, N, P, Q, R, S, T, V, W, Y
  4 -- A, C, D, E, F, G, H, I, K, L, M, N, P, Q, R, S, T, V, W, Y
  5 -- A, C, D, E, F, G, H, I, K, L, M, N, P, Q, R, S, T, V, W, Y
  6 -- A, C, D, E, F, G, H, I, K, L, M, N, P, Q, R, S, T, V, W, Y
  7 -- A, C, D, E, F, G, H, I, K, L, M, N, P, Q, R, S, T, V, W, Y
  8 -- A, C, D, E, F, G, H, I, K, L, M, N, P, Q, R, S, T, V, W, Y
  9 -- A, C, D, E, F, G, H, I, K, L, M, N, P, Q, R, S, T, V, W, Y
10 -- A, C, F, H, I, K, L, M, N, Q, R, S, T, V, W, Y
11 -- F, N, Y
12 -- A, C, D, E, F, G, H, I, K, L, M, N, P, Q, R, S, T, V, W, Y
13 -- A, C, D, E, F, G, H, I, K, L, M, N, P, Q, R, S, T, V, W, Y
14 -- A, D, G
15 -- A, C, D, E, G, H, I, K, L, M, N, Q, R, S, T, V
16 -- A, C, D, E, F, G, H, I, K, L, M, N, P, Q, R, S, T, V, W, Y
17 -- A, C, D, E, F, G, H, I, K, L, M, N, Q, R, S, T, V, W, Y
18 -- A, C, D, E, F, G, H, I, L, M, N, Q, S, T, V, W, Y
19 -- A, C, D, E, F, G, H, I, K, L, M, N, P, Q, R, S, T, V, W, Y
20 -- A, C, D, E, F, G, H, I, K, L, M, N, P, Q, R, S, T, V, W, Y
21 -- A, C, E, G, S, V
22 -- A, C, D, E, F, G, H, I, K, L, M, N, P, Q, R, S, T, V, W, Y
23 -- A, C, D, E, F, G, H, I, K, L, M, N, P, Q, R, S, T, V, W, Y
24 -- A, C, D, E, F, H, K, L, M, N, Q, R, S, T, V, Y
25 -- A, C, D, F, G, H, L, M, N, Q, R, S, T, V, W, Y
26 -- A, C, D, E, F, G, H, I, K, L, M, N, P, Q, R, S, T, V, W, Y
27 -- A, C, D, E, F, G, H, I, K, L, M, N, P, Q, R, S, T, V, W, Y
28 -- A, C, D, E, F, G, H, K, L, M, N, P, Q, R, S, T, Y
29 -- A, C, D, E, F, G, H, I, K, L, M, N, Q, R, S, T, V, W, Y
30 -- A, D, E, F, G, H, I, K, L, M, N, P, Q, R, S, T, V, W, Y
31 -- A, C, D, E, F, G, H, I, K, L, M, N, P, Q, R, S, T, V, W, Y
32 -- A, C, D, E, F, G, H, I, K, L, M, N, P, Q, R, S, T, V, W, Y
33 -- A, G, S
34 -- A, C, D, E, F, G, H, I, K, L, M, N, P, Q, R, S, T, V, W, Y
35 -- A, C, D, E, F, G, H, I, K, L, M, N, P, Q, R, S, T, V, W, Y
36 -- A, C, D, E, F, G, H, K, L, M, N, P, Q, R, S, T, V, W, Y
37 -- A, C, D, E, F, G, H, I, K, L, M, N, P, Q, R, S, T, V, W, Y
38 -- A, C, E, G, H, M, P, Q
39 -- A, C, D, E, F, G, H, I, K, L, M, N, P, Q, R, S, T, V, W, Y
40 -- A, C, D, E, G, K, N, Q, R, S, T
41 -- A, C, D, E, F, G, H, I, L, M, N, P, Q, S, T, V, W, Y
42 -- A, C, D, E, F, G, H, I, K, L, M, N, P, Q, R, S, T, V, W, Y
43 -- A, C, D, E, F, G, H, I, K, L, M, N, Q, S, T, V, W, Y
44 -- E, F, H,Q, S, W, Y
45 -- D, N
46 -- A, C, D, E, F, G, H, I, K, L, M, N, P, Q, R, S, T, V, W, Y
47 -- C, T, V
48 -- F, S, W, Y
49 -- A, C, D, E, F, G, H, I, K, L, M, N, Q, R, S, T, V, W, Y
50 -- A, C, F, H, I, K, L, M, N, Q, R, S, T, V, W, Y
51 -- A, D, G, H, N, S
52 -- H, K, Q, R
53 -- A, C, D, E, F, G, H, I, K, L, M, N, P, Q, R, S, T, V, W, Y
54 -- A, C, H, I, K, L, M, N, P, Q, R, S, T, V
55 -- A, C, E, G, H, K, N, Q, R, S, T
56 -- A, C, D, E, F, G, H, I, K, L, M, N, P, Q, R, S, T, V, W, Y
57 -- A, C, D, E, F, G, H, I, K, L, M, N, P, Q, R, S, T, V, W, Y
58 -- A, C, D, E, F, G, H, I, K, L, M, N, P, Q, R, S, T, V, W, Y
59 -- A, C, D, E, F, G, H, I, K, L, M, N, P, Q, R, S, T, V, W, Y
60 -- A, C, D, E, F, G, H, I, K, L, M, N, P, Q, R, S, T, V, W, Y
61 -- A, D, E, F, G, H, I, K, L, M, N, P, Q, R, S, T, V, W, Y
62 -- A, C, D, E, F, G, H, I, K, L, M, N, P, Q, R, S, T, V, W, Y
63 -- A, C, D, E, F, G, H, I, K, L, M, N, P, Q, R, S, T, V, W, Y
64 -- A, C, D, E, F, G, H, I, K, L, M, N, P, Q, R, S, T, V, W, Y
65 -- A, C, D, E, F, G, H, I, K, L, M, N, P, Q, R, S, T, V, W, Y
66 -- A, C, D, E, F, G, H, I, K, L, M, N, P, Q, R, S, T, V, W, Y
67 -- A, C, D, E, F, G, H, I, K, L, M, N, P, Q, R, S, T, V, W, Y
68 -- A, C, D, E, F, G, H, I, K, L, M, N, P, Q, R, S, T, V, W, Y
69 -- A, C, D, E, F, G, H, I, K, L, M, N, P, Q, R, S, T, V, W, Y
70 -- A, C, D, E, F, G, H, I, K, L, M, N, P, Q, R, S, T, V, W, Y
71 -- A, C, D, E, F, G, H, I, K, L, M, N, P, Q, R, S, T, V, W, Y
72 -- A, C, D, E, F, G, H, I, K, L, M, N, P, Q, R, S, T, V, W, Y
73 -- A, C, D, E, F, G, H, I, K, L, M, N, P, Q, R, S, T, V, W, Y
74 -- A, C, D, E, F, G, H, I, K, L, M, N, P, Q, R, S, T, V, W, Y
75 -- A, C, D, E, F, G, H, I, K, L, M, N, P, Q, R, S, T, V, W, Y
76 -- A, C, D, E, F, G, H, I, K, L, M, N, P, Q, R, S, T, V, W, Y
77 -- A, C, D, E, F, G, H, I, K, L, M, N, P, Q, R, S, T, V, W, Y
78 -- A, C, D, E, F, G, H, I, K, L, M, N, P, Q, R, S, T, V, W, Y
79 -- A, C, D, E, F, G, H, I, K, L, M, N, P, Q, R, S, T, V, W, Y
80 -- A, C, D, E, F, G, H, I, K, L, M, N, P, Q, R, S, T, V, W, Y
81 -- A, C, D, E, F, G, H, I, K, L, M, N, P, Q, R, S, T, V, W, Y
82 -- A, C, D, E, F, G, H, I, K, L, M, N, P, Q, R, S, T, V, W, Y
83 -- A, C, D, E, F, G, H, I, K, L, M, N, P, Q, R, S, T, V, W, Y
84 -- A, C, D, E, F, G, H, I, K, L, M, N, P, Q, R, S, T, V, W, Y
85 -- A, C, D, E, F, G, H, I, K, L, M, N, P, Q, R, S, T, V, W, Y
AHB2 (SEQ ID NO: 101-102 and 164)
  1 -- C, G, A, V, F, Y, W, S, Q, D, E, R, K
  2 -- C, P, G, V, I, M, L, F, Y,W, S, N, Q, D, E, R, H
  3 -- C, G, A,V, I, F, S, T, D, E, K
  4 -- C, P, G, A,V, I, M, L, F, Y, W, S, T, N, Q, D, E, R, K, H
  5 -- C, P, G, A, V, M, L, Y, W, S, N, Q, D, E, R, K, H
  6 -- G, A, V, I, F, S, T, D, H
  7 -- C, P, G,V, I, M, L, F, W, S, T, N, Q, E, R, K, H
  8 -- C, P, G, A, V, M, L, Y, W, S, T, N, Q, D, E, R, K, H
  9 -- C, P, G, A,V, I, M, L, F, W, S, T, N, Q, D, E, R, K, H
10 -- C, P, G, A, V, I, L, Y, W, S, T, N, E, R, K
11 -- C, P, G, A, V, I, M, L, F, Y, W, S, T, N, Q, D, E, R, H
12 -- C, P, G, A, V, I, L, F, Y, W, S, T, N, Q, D, E, R, K, H
13 -- C, G, A, V, M, L, F, W, S, T, N, E, H
14 -- C, P, G, A, V, I, Y, S, T, N, D, E, R, H
15 -- C, G, A, V, I, M, L, F, Y, W, S, T, N, Q, D, E, R, K
16 -- C, P, G, A, V, I, M, L, F, Y, W, S, T, N, Q, D, E, R, K, H
17 -- C, P, G, A, V, L, Y, W, S, T, Q, D, E, R
18 -- C, P, A, V, I, M, F, Y, N, Q, R, K, H
19 -- C, P, G, A, V, I, M, L, F, Y, W, S, T, N, Q, D, E, R, K, H
20 -- C, P, G, A, V, M, L, Y, W, N, Q, E, R, K, H
21 -- C, P, G, A, V, I, M, L, F, Y, W, S, N, Q, E, R, K, H
22 -- C, P, G, A, V, M, L, F, Y, S, T, N, Q, D, E, R, K, H
23 -- C, P, G, A, V, I, M, L, F, Y, W, S, T, N, Q, E, R, K
24 -- C, P, G, A, V, I, M, L, F, Y, W, S, Q, E, R, H
25 -- C, P, G, A, V, I, M, L, F, Y, W, S, T, N, Q, D, R, H
26 -- C, G, A, V, L, Y, S, N, D, R, K, H
27 -- C, P, G, A, V, I, M, L, F, Y, W, S, T, N, Q, D, E, R, K, H
28 -- C, P, G, A, V, I, M, L, F, Y, W, S, T, N, Q, D, E, R, K, H
29 -- C, P, G, V, I, M, L, F, Y, W, S, T, N, Q, D, R, K, H
30 -- C, P, G, A, V, I, M, L, F, Y, W, S, T, N, Q, D, E, R, K, H
31 -- C, G, A,V, I, M, L, F, Y, W, S, T, Q, D, E, R, K, H
32 -- P, G, A, V, I, L, W, S, T, D, R, H
33 -- C, P, G, A,V, I, M, L, F, Y, W, S, T, N, Q, E, R, K, H
34 -- C, G, A, V, I, M, L, F, Y, W, S, T, N, Q, D, E, R, K, H
35 -- C, P, G, A, V, I, M, L, F, Y, W, S, T, N, Q, D, E, R, K, H
36 -- C, P, G, A, V, I, L, F, Y, S, T, N, Q, D, E, R, H
37 -- C, G, A, V, I, M, L, F, Y, W, S, T, N, Q, D, E, R, K, H
38 -- C, P, G, A, V, I, M, L, F, Y, W, S, T, Q, E, R, K
39 -- C, P, G, A, V, I, W, S, Q, E, R, H
40 -- C, P, G, A, V, I, L, Y, W, S, T, N, D, E, R, K, H
41 -- C, P, G, A, V, I, M, L, Y, W, S, T, N, Q, D, E, R, K, H
42 -- C, P, G, A, V, M, L, Y, W, S, T, N, Q, D, E, R, K, H
43 -- C, G, A, V, I, M, L, F, Y, W, S, T, N, Q, D, E, R, K, H
44 -- C, P, G, A, V, I, M, L, F, W, S, T, Q, D, E, R, H
45 -- C, G, A, V, I, M, L, F, Y, W, S, T, N, Q, D, E, R, K, H
46 -- C, P, G, A, V, I, M, L, F, S, T, Q, E, R, K
47 -- C, G, A, V, I, M, L, F, W, S, T, N, Q, D, E, R, H
48 -- C, P, G, A, V, I, M, L, F, Y, W, S, N, Q, E, R, K
49 -- C, P, G, A, V, M, L, F, Y, W, S, T, N, Q, D, E, R, K, H
50 -- C, P, G, A, V, I, M, L, F, Y, W, S, T, N, Q, D, E, R, K, H
51 -- C, G, A, V, I, M, L, F, Y, W, S, T, N, Q, D, E, R, K, H
52 -- C, P, G, A, V, I, M, L, F, Y, W, S, T, N, Q, D, E, R, K, H
53 -- C, P, G, A, V, I, M, L, F, Y, W, S, T, N, D, E, R, K, H
54 -- C, P, G, A, V, I, M, L, F, Y, W, S, T, N, Q, D, E, R, K, H
55 -- C, P, G, A, V, I, M, L, F, Y, S, T, N, Q, D, E, R, K, H
56 -- C, P, G, A, V, I, M, L, F, Y, W, S, T, N, Q, D, E, R, K, H
57 -- C, P, G, A, V, I, M, L, F, Y, W, S, T, N, Q, D, E, R, K, H
58 -- C, G, A, V, I, M, L, F, Y, W, S, T, N, E, R, K, H
59 -- C, P, G, A, V, I, M, L, F, Y, W, S, T, N, Q, D, E, R, K, H
60 -- C, G, A, V, I, M, L, F, Y, W, S, T, Q, D, E, R, K
61 -- C, P, G, A, V, I, M, L, F, Y, W, S, N, Q, D, E, R, K, H
62 -- C, G, A, V, L, S, T, N, D, E, K, H
63 -- C, P, G, A, V, I, L, F, Y, W, S, T, N, Q, D, E, R, K, H
64 -- C, P, G, A, V, I, M, L, F, Y, W, S, T, N, Q, D, E, R, H
65 -- C, G, A, V, I, M, L, F, Y, S, T, N, R, K, H
66 -- C, P, G, A, V, I, M, L, W, T, Q, E, R
67 -- C, P, G, A, V, I, M, L, F, Y, W, S, T, N, Q, D, E, R, K, H
68 -- C, P, G, A, V, I, L, F, Y, W, S, T, N, Q, D, E, R, H
69 -- P, G, V, I, M, L, Y, W, S, T, Q, R, K
70 -- C, P, G, A, V, I, M, L, F, Y, W, S, T, N, Q, D, E, R, K, H
71 -- C, G, A, V, L, F, W, S, Q, D, E, R, K
72 -- C, V, I, L, S
73 -- P, G, A, V, S, T, E
74 -- C, A, L, F , Y, S, T, R, H
75 -- C, P, G,V, I, L, F, W, S, N, D, E, R, K
```

The residue numbers of the interface residues which are within 8A to the RBD target are listed in Table 2 for the various design types. In another embodiment, amino acid residues at the interface residues listed in Table 2 are either identical at that residue to the reference sequence, or may be substituted by a conservative amino acid substitution. Such conservative amino acid substitutions involve replacing a residue by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) 35 acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

TABLE 2
Interface residues

| | |
|---|---|
| ‘LCB1’: | [3, 6, 7, 10, 13, 17, 20, 22, 23, 25, 26, 29, 32, 33, 36], |
| ‘LCB2’: | [1, 2, 5, 6, 9, 12, 13, 16, 20, 32, 35, 39], |
| ‘LCB3’: | [1, 3, 4, 6, 7, 10, 11, 13, 14, 15, 18, 27, 30, 33, 34, 37], |
| ‘LCB4’: | [8, 11, 12, 15, 23, 24, 26, 27, 28, 30, 31, 34, 56], |
| ‘LCB5’: | [35, 37, 38, 40, 41, 44, 47, 48, 53, 56, 60, 63], |
| ‘LCB6’: | [3, 4, 7, 8, 11, 12, 14, 15, 21, 24, 25, 28, 31, 32, 35], |
| ‘LCB7’: | [2, 3, 6, 7, 9, 10, 13, 17, 29, 32, 33, 36], |
| ‘LCB8’: | [14, 15, 16, 19, 22, 23, 26, 29, 30, 38, 41, 42, 45], |
| ‘AHB1’, | [34, 38, 41, 45, 48, 49, 52, 63, 64, 67, 68, 70, 71, 74, 78, |
| | 81, 82, 85], |
| ‘AHB2’, | [4, 7, 11, 14, 15, 18, 21, 26, 29, 30, 33, 34, 36, 37, 40, 43, |
| | 44, 47, 48]. |

In one embodiment, amino acid residues at the interface residues listed in Table 2 are identical at that residue to the reference sequence.

In another embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-10, 13-17, 19-21, 33-34, and 100-101.

In one embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-10 and 102-136 (see Table 3).

TABLE 3
LCB1 exemplary variants

| Name | Binder Protein |
|---|---|
| LCB1_4N | DKENILQKIYEIMRLLDELGHAEASMRVSDLIYEFMKKGDERLLEEAERLLEEVER (SEQ |
| | ID NO: 102) |
| LCB1_4K | DKEKILQKIYEIMRLLDELGHAEASMRVSDLIYEFMKKGDERLLEEAERLLEEVER (SEQ |
| | ID NO: 103) |
| LCB1_14K | DKEWILQKIYEIMKLLDELGHAEASMRVSDLIYEFMKKGDERLLEEAERLLEEVER (SEQ |
| | ID NO: 104) |
| LCB1_15T | DKEWILQKIYEIMRTLDELGHAEASMRVSDLIYEFMKKGDERLLEEAERLLEEVER (SEQ |
| | ID NO: 105) |
| LCB1_18Q | DKEWILQKIYEIMRLLDQLGHAEASMRVSDLIYEFMKKGDERLLEEAERLLEEVER (SEQ |
| | ID NO: 106) |
| LCB1_18K | DKEWILQKIYEIMRLLDKLGHAEASMRVSDLIYEFMKKGDERLLEEAERLLEEVER (SEQ |
| | ID NO: 107) |
| LCB1_27Q | DKEWILQKIYEIMRLLDELGHAEASMQVSDLIYEFMKKGDERLLEEAERLLEEVER (SEQ |
| | ID NO: 108) |
| LCB1_27Y | DKEWILQKIYEIMRLLDELGHAEASMYVSDLIYEFMKKGDERLLEEAERLLEEVER (SEQ |
| | ID NO: 109) |
| LCB1_17E | DKEWILQKIYEIMRLLEELGHAEASMRVSDLIYEFMKKGDERLLEEAERLLEEVER (SEQ |
| | ID NO: 110) |
| LCB1_17R | DKEWILQKIYEIMRLLRELGHAEASMRVSDLIYEFMKKGDERLLEEAERLLEEVER (SEQ |
| | ID NO: 111) |
| LCB1_42N | DKEWILQKIYEIMRLLDELGHAEASMRVSDLIYEFMKKGDENLLEEAERLLEEVER (SEQ |
| | ID NO: 112) |
| LCB1_49Q | DKEWILQKIYEIMRLLDELGHAEASMRVSDLIYEFMKKGDERLLEEAEQLLEEVER (SEQ |
| | ID NO: 113) |
| LCB1_52Q | DKEWILQKIYEIMRLLDELGHAEASMRVSDLIYEFMKKGDERLLEEAERLLQEVER (SEQ |
| | ID NO: 114) |
| LCB1_32L | DKEWILQKIYEIMRLLDELGHAEASMRVSDLLYEFMKKGDERLLEEAERLLEEVER (SEQ |
| | ID NO: 115) |
| LCB1_28A | DKEWILQKIYEIMRLLDELGHAEASMRASDLIYEFMKKGDERLLEEAERLLEEVER (SEQ |
| | ID NO: 116) |
| LCB1_v1.3_ACH1 | DKENILQKIYEIMKTLEQLGHAEASMYVSDLIYEFMKQGDERLLEEAERLLEEVER (SEQ |
| | ID NO: 117) |
| LCB1_v1.3_ACH2 | DKENILQKIYEIMKTLEQLGHAEASMQVSDLIYEFMKQGDENLLEEAERLLEEVER (SEQ |
| | ID NO: 118) |
| LCB1_v1.3_ACH3 | DKENILQKIYEIMKTLEQLGHAEASMQVSDLIYEFMKQGDERLLEEAEQLLEEVER (SEQ |
| | ID NO: 119) |
| LCB1_v1.3_ACH4 | DKENILQKIYEIMKTLEQLGHAEASMYVSDLIYEFMKQGDENLLEEAEQLLEEVER (SEQ |
| | ID NO: 120) |
| LCB1_v1.3_ACH5 | DKENILQKIYEIMKTLEQLGHAEASMQVSDLIYEFMKQGDENLLEEAEQLLEEVER (SEQ |
| | ID NO: 121) |
| LCB1_v1.3_1 | DRENILQKIYEIMKELEKLGHAEASMQVSDLIYEFMQDKDERLLEEAERLLEEVKR (SEQ |
| | ID NO: 122) |
| LCB1_v1.3_2 | DRENILQKIYEIMKELRQLGHAEASMQVSDLIYEFMKTKDKRLLEEAERLLEEVKR (SEQ |
| | ID NO: 123) |
| LCB1_v1.3_3 | DRENILQKIYEIMKTLRRLGHAEASMQVSDLIYEFMQDKDKRLLEEAERLLEEVQR (SEQ |
| | ID NO: 124) |
| LCB1_v1.3_4 | DKENVLQKIYEIMKELERLGHAEASMQVSDLIYEFMKTKDERLLEEAERLLEEVKR (SEQ |
| | ID NO: 125) |
| LCB1_v1.3_5 | DRENILQKIYEIMKTLEKLGHAEASMQASDLIYEFMKTKDERLLEEAERLLEEVQR (SEQ |
| | ID NO: 126) |
| LCB1_v1.3_6 | DKENILQKIYEIMKTLRALGHAEASMQVSDLIYEFMQTKDERLLEEAERLLEEVKR (SEQ |
| | ID NO: 127) |
| LCB1_v1.3_7 | DKENVLQKIYEIMKTLEKLGHAEASMQVSDLIYEFMQTKDKRLLEEAERLLEEVQR (SEQ |
| | ID NO: 128) |
| LCB1_v1.3_15 | DRENILQKIYEIMKELEKLGHAEASMQVSDLIYEFMQDKDENLLEEAERLLEEVKR (SEQ |
| | ID NO: 129) |
| LCB1_v1.3_16 | DRENILQKIYEIMKELRQLGHAEASMQVSDLIYEFMKTKDKNLLEEAERLLEEVKR (SEQ |
| | ID NO: 130) |
| LCB1_v1.3_17 | DRENILQKIYEIMKTLRRLGHAEASMQVSDLIYEFMQDKDKNLLEEAERLLEEVQR (SEQ |
| | ID NO: 131) |
| LCB1_v1.3_19 | DRENILQKIYEIMKTLEKLGHAEASMQASDLIYEFMKTKDENLLEEAERLLEEVQR (SEQ |
| | ID NO: 132) |
| LCB1_v1.3_20 | DKENILQKIYEIMKTLRALGHAEASMQVSDLIYEFMQTKDENLLEEAERLLEEVKR (SEQ |
| | ID NO: 133) |
| LCB1_v1.3_21 | DKENVLQKIYEIMKTLEKLGHAEASMQVSDLIYEFMQTKDKNLLEEAERLLEEVQR (SEQ |
| | ID NO: 134) |
| LCB1_v2.2 | DKENVLQKIYEIMKELERLGHAEASMQVSDLIYEFMKTKDENLLEEAERLLEEVKR (SEQ |
| | ID NO: 135) |
| LCB1_v2.2_ | DKENVLQKIYEIMKELERLGHAEASMQVSDLIYEFMKTKDENLLEEAERLLEEVTR (SEQ |
| ompT | ID NO: 136) |

The polypeptides may contain a substantial number of mutations while retaining binding activity, as detailed in the examples that follow. In one embodiment, the polypeptide comprises an amino acid substitution relative to the amino acid sequence of SEQ ID NO:1 at 1, 2, 3, 4, 5, 6, 7, 8, 9, 10, 11, 12, 13, 14, 15, 16, 17, or all 18 residues selected from the group consisting of 2, 4, 5, 14, 15, 17, 18, 27, 28, 32, 37, 38, 39, 41, 42, 49, 52, and 55. In another embodiment, the substitutions are selected from the substitutions listed in Table 4, either individually (i.e.: any single mutation listed in the Table) or in combinations in a given row.

TABLE 4
Exemplary LCB1 substitutions

| Name | Parent | Mutations from WT | | | | | | | | | |
|---|---|---|---|---|---|---|---|---|---|---|---|
| LCB1_1 | LCB1 | W4N | R14K | L15T | E18Q | R27Q | K38Q | | | | |
| LCB1_2 | LCB1 | W4N | R14K | L15T | E18Q | R27Y | K38Q | | | | |
| LCB1_3 | LCB1 | W4N | R14K | L15T | D17E | E18Q | R27Q | K38Q | | | |
| LCB1_4 | LCB1 | W4N | R14K | L15T | D17E | E18Q | R27Q | K38Q | R42N | R49Q | E529 |
| LCB1_4N | LCB1 | W4N | | | | | | | | | |
| LCB1_4K | LCB1 | W4K | | | | | | | | | |
| LCB1_14K | LCB1 | R14K | | | | | | | | | |
| LCB1_15T | LCB1 | L15T | | | | | | | | | |
| LCB1_18Q | LCB1 | E18Q | | | | | | | | | |
| LCB1_18K | LCB1 | E18K | | | | | | | | | |
| LCB1_27Q | LCB1 | R27Q | | | | | | | | | |
| LCB1_27Y | LCB1 | R27Y | | | | | | | | | |
| LCB1_38Q | LCB1 | K38Q | | | | | | | | | |
| LCB1_17E | LCB1 | D17E | | | | | | | | | |
| LCB1_17R | LCB1 | D17R | | | | | | | | | |
| LCB1_42N | LCB1 | R42N | | | | | | | | | |
| LCB1_49Q | LCB1 | R49Q | | | | | | | | | |
| LCB1_52Q | LCB1 | E52Q | | | | | | | | | |
| LCB1_32L | LCB1 | I32L | | | | | | | | | |
| LCB1_28A | LCB1 | V28A | | | | | | | | | |
| LCB1_v1.3 | LCB1 | W4N | R14K | L15T | D17E | E18Q | R27Q | K38Q | | | |
| LCB1_v1.3_ACH1 | LCB1_v1.3 | W4N | R14K | L15T | D17E | E18Q | R27Y | K38Q | | | |
| LCB1_v1.3_ACH2 | LCB1_v1.3 | W4N | R14K | L15T | D17E | E18Q | R27Q | K38Q | R42N | | |
| LCB1_v1.3_ACH3 | LCB1_v1.3 | W4N | R14K | L15T | D17E | E18Q | R27Q | K38Q | R49Q | | |
| LCB1_v1.3_ACH4 | LCB1_v1.3 | W4N | R14K | L15T | D17E | E18Q | R27Y | K38Q | R42N | R49Q | |
| LCB1_v1.3_ACH5 | LCB1_v1.3 | W4N | R14K | L15T | D17E | E18Q | R27Q | K38Q | R42N | R49Q | |
| LCB1_v1.3_1 | LCB1_v1.3 | K2R | W4N | R14K | L15E | D17E | E18K | R27Q | K37Q | K38D | G39K |
| | E55K | | | | | | | | | | |
| LCB1_v1.3_2 | LCB1_v1.3 | K2R | W4N | R14K | L15E | D17R | E18Q | R27Q | K38T | G39K | E41K |
| | E55K | | | | | | | | | | |
| LCB1_v1.3_3 | LCB1_v1.3 | K2R | W4N | R14K | L15T | D17R | E18R | R27Q | K37Q | K38D | G39K |
| | E41K | E55Q | | | | | | | | | |
| LCB1_v1.3_4 | LCB1_v1.3 | W4N | I5V | R14K | L15E | D17E | E18R | R27Q | K38T | G39K | E55K |
| LCB1_v1.3_5 | LCB1_v1.3 | K2R | W4N | R14K | L15T | D17E | E18K | R27Q | V28A | K38T | G39K |
| | E55Q | | | | | | | | | | |
| LCB1_v1.3_6 | LCB1_v1.3 | W4N | R14K | L15T | D17R | E18A | R27Q | K37Q | K38T | G39K | E55K |
| LCB1_v1.3_7 | LCB1_v1.3 | W4N | I5V | R14K | L15T | D17E | E18K | R27Q | K37Q | K38T | G39K |
| | E41K | E55Q | | | | | | | | | |
| LCB1_v1.3_15 | LCB1_v1.3 | K2R | W4N | R14K | L15E | D17E | E18K | R27Q | K37Q | K38D | G39K |
| | R42N | E55K | | | | | | | | | |
| LCB1_v1.3_16 | LCB1_v1.3 | K2R | W4N | R14K | L15E | D17R | E18Q | R27Q | K38T | G39K | E41K |
| | R42N | E55K | | | | | | | | | |
| LCB1_v1.3_17 | LCB1_v1.3 | K2R | W4N | R14K | L15T | D17R | E18R | R27Q | K37Q | K38D | G39K |
| | E41K | R42N | E55Q | | | | | | | | |
| LCB1_v1.3_19 | LCB1_v1.3 | K2R | W4N | R14K | L15T | D17E | E18K | R27Q | V28A | K38T | G39K |
| | R42N | E55Q | | | | | | | | | |
| LCB1_v1.3_20 | LCB1_v1.3 | W4N | R14K | L15T | D17R | E18A | R27Q | K37Q | K38T | G39K | R42N |
| | E55K | | | | | | | | | | |
| LCB1_v1.3_21 | LCB1_v1.3 | W4N | I5V | R14K | L15T | D17E | E18K | R27Q | K37Q | K38T | G39K |
| | E41K | R42N | E55Q | | | | | | | | |
| LCB1_v2.2 | LCB1_v1.3 | W4N | I5V | R14K | L15E | D17E | E18R | R27Q | K38T | G39K | R42N |
| | E55K | | | | | | | | | | |
| LCB1_v2.2_ompT | LCB1-v1.3 | W4N | I5V | R14K | L15E | D17E | E18R | R27Q | K38T | G39K | R42N |
| | E55T | | | | | | | | | | |

In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163 (see Table 5).

TABLE 5
LCB3 exemplary variants

| Name | Binder Protein |
|---|---|
| LCB3_8Q | NDDELHMQMTDLVYEALHFAKDEEIKKRVFQLFELADKAYKNNDRQKLEKVVEELKELLE |
| | RLLS (SEQ ID NO: 137) |
| LCB3_8T | NDDELHMTMTDLVYEALHFAKDEEIKKRVFQLFELADKAYKNNDRQKLEKVVEELKELLE |
| | RLLS (SEQ ID NO: 138) |
| LCB3_19K | NDDELHMLMTDLVYEALHKAKDEEIKKRVFQLFELADKAYKNNDRQKLEKVVEELKELLE |
| | RLLS (SEQ ID NO: 139) |
| LCB3_19I | NDDELHMLMTDLVYEALHIAKDEEIKKRVFQLFELADKAYKNNDRQKLEKVVEELKELLE |
| | RLLS (SEQ ID NO: 140) |
| LCB3_25F | NDDELHMLMTDLVYEALHFAKDEEFKKRVFQLFELADKAYKNNDRQKLEKVVEELKELLE |
| | RLLS (SEQ ID NO: 141) |
| LCB3_25M | NDDELHMLMTDLVYEALHFAKDEEMKKRVFQLFELADKAYKNNDRQKLEKVVEELKELLE |
| | RLLS (SEQ ID NO: 142) |
| LCB3_26Q | NDDELHMLMTDLVYEALHFAKDEEIQKRVFQLFELADKAYKNNDRQKLEKVVEELKELLE |
| | RLLS (SEQ ID NO: 143) |
| LCB3_28H | NDDELHMLMTDLVYEALHFAKDEEIKKHVFQLFELADKAYKNNDRQKLEKVVEELKELLE |
| | RLLS (SEQ ID NO: 144) |
| LCB3_35K | NDDELHMLMTDLVYEALHFAKDEEIKKRVFQLFEKADKAYKNNDRQKLEKVVEELKELLE |
| | RLLS (SEQ ID NO: 145) |
| LCB3_37T | NDDELHMLMTDLVYEALHFAKDEEIKKRVFQLFELATKAYKNNDRQKLEKVVEELKELLE |
| | RLLS (SEQ ID NO: 146) |
| LCB3_40R | NDDELHMLMTDLVYEALHFAKDEEIKKRVFQLFELADKARKNNDRQKLEKVVEELKELLE |
| | RLLS (SEQ ID NO: 147) |
| LCB3_43K | NDDELHMLMTDLVYEALHFAKDEEIKKRVFQLFELADKAYKNKDRQKLEKVVEELKELLE |
| | RLLS (SEQ ID NO: 148) |
| LCB3_34G | NDDELHMLMTDLVYEALHFAKDEEIKKRVFQLFGLADKAYKNNDRQKLEKVVEELKELLE |
| | RLLS (SEQ ID NO: 149) |
| LCB3_34Y | NDDELHMLMTDLVYEALHFAKDEEIKKRVFQLFYLADKAYKNNDRQKLEKVVEELKELLE |
| | RLLS (SEQ ID NO: 150) |
| LCB3_34T | NDDELHMLMTDLVYEALHFAKDEEIKKRVFQLFTLADKAYKNNDRQKLEKVVEELKELLE |
| | RLLS (SEQ ID NO: 151) |
| LCB3_49K | NDDELHMLMTDLVYEALHFAKDEEIKKRVFQLFELADKAYKNNDRQKLKKVVEELKELLE |
| | RLLS (SEQ ID NO: 152) |
| LCB3_v1.2_AC | NDDELHMQMTDLVYEALHFAKDEEIQKHVFQLFGKATKAYKNKDRQKLEKVVEELKELLE |
| H1 | RLLS (SEQ ID NO: 153) |
| LCB3_v1.2_AC | NDDELHMQMTDLVYEALHFAKDEEIQKHVFQLFYKATKAYKNKDRQKLEKVVEELKELLE |
| H2 | RLLS (SEQ ID NO: 154) |
| LCB3_v2.2 | NLDELHMQMTDLVYEALHFAKDEEFQKHVFQLFEKATKAYKNKDRQKLEKVVEELKELLE |
| | RLLS (SEQ ID NO: 155) |
| LCB3_v1.3_2 | NDDELHMQMTDLVYEALHFAKTEEFQKHVFQLFEKATKAYKNKDRQKLEKVVEELKELLE |
| | RLLS (SEQ ID NO: 156) |
| LCB3_v1.3_3 | NDDELHMQMTDLVYEALHFAKDEEFQKHVFQLFEKARKAYKNKDRQKLEKVVEELKELLE |
| | RLLS (SEQ ID NO: 157) |
| LCB3_v1.3_4 | NDDELHMQMTDLVWEALHFAKDEEFQKHVFQLFEKARKAYKNKDRQKLEKVVEELKELLE |
| | RLLS (SEQ ID NO: 158) |
| LCB3_v1.3_5 | NDDELHMQMTDLVWEALHFAKDEEFQKHVFQLFEKATKAYKNKDRQKLEKVVEELKELLE |
| | RLLS (SEQ ID NO: 159) |
| LCB3_v1.3_6 | NEDELHMQMTDLVWEALHFAKDEEFQKHVFQLFEKATKAYKNKDRQKLEKVVEELKELLE |
| | RLLS (SEQ ID NO: 160) |
| LCB3_v1.3_7 | NDDELHMQMTDLVWEALHFAKTEEFQKHVFQLFEKATKAYKNKDRQKLEKVVEELKELLE |
| | RLLS (SEQ ID NO: 161) |
| LCB3_v1.3_15 | NLDELHMQMTDLVYEALHFAKTEEFQKHVFQLFEKATKAYKNKDRQKLEKVVEELKELLE |
| | RLLS (SEQ ID NO: 162) |
| LCB3_v2.3 | NIDELLMQVTDLIYEALHFAKDEEFQKHAFQLFEKATKAYKNKDKQKLEKVVEELKELLE |
| | RILS (SEQ ID NO: 163) |

The polypeptides may contain a substantial number of mutations while retaining binding activity, as detailed in the examples that follow. In one embodiment, the polypeptide comprises an amino acid substitution relative to the amino acid sequence of SEQ ID NO:13 at 1, 2, 3, 4, 5, 6, 7, 8, 9, 10, 11, 12, 13, 14, 15, 16, 17, 18, 19, or all 20 residues selected from the group consisting 2, 6, 8, 9, 13, 14, 19, 22, 25, 26, 28, 29, 34, 35, 37, 40, 43, 45, 49, and 62. In another embodiment, the substitutions are selected from the substitutions listed in Table 6, either individually or in combinations in a given row.

TABLE 6
Exemplary LCB3 substitutions

| Name | Parent | Mutations from WT | | | | | | | |
|---|---|---|---|---|---|---|---|---|---|
| LCB3_1 | LCB3 | L8Q | I25F | K26Q | R28H | L35K | D37T | | |
| LCB3_2 | LCB3 | L8Q | K26Q | R28H | L35K | D37T | N43K | | |
| LCB3_3 | LCB3 | L8Q | I25F | K26Q | R28H | L35K | D37T | N43K | |
| LCB3_4 | LCB3 | L8Q | F19K | I25F | K26Q | R28H | L35K | D37T | Y40R, |
| | | | | | | | | | N43K |
| LCB3_8Q | LCB3 | L8Q | | | | | | | |
| LCB3_8T | LCB3 | L8T | | | | | | | |
| LCB3_19K | LCB3 | F19K | | | | | | | |
| LCB3_19I | LCB3 | F19I | | | | | | | |
| LCB3_25F | LCB3 | I25F | | | | | | | |
| LCB3_25M | LCB3 | I25M | | | | | | | |
| LCB3_26Q | LCB3 | K26Q | | | | | | | |
| LCB3_28H | LCB3 | R28H | | | | | | | |
| LCB3_35K | LCB3 | L35K | | | | | | | |
| LCB3_37T | LCB3 | D37T | | | | | | | |
| LCB3_40R | LCB3 | Y40R | | | | | | | |
| LCB3_43K | LCB3 | N43K | | | | | | | |
| LCB3_34G | LCB3 | E34G | | | | | | | |
| LCB3_34Y | LCB3 | E34Y | | | | | | | |
| LCB3_34T | LCB3 | E34T | | | | | | | |
| LCB3_49K | LCB3 | E49K | | | | | | | |
| LCB3_v1.2 | LCB3 | L8Q | K26Q | R28H | L35K | D37T | N43K | | |
| LCB3_v1.2_ACH1 | LCB3_v1.2 | L8Q | K26Q | R28H | E34G | L35K | D37T | N43K | |
| LCB3_v1.2_ACH2 | LCB3_v1.2 | L8Q | K26Q | R28H | E34Y | L35K | D37T | N43K | |
| LCB3_v2.2 | LCB3_v1.3 | D2L | L8Q | I25F | K26Q | R28H | L35K | D37T | N43K |
| LCB3_v1.3_2 | LCB3_v1.3 | L8Q | D22T | I25F | K26Q | R28H | L35K | D37T | N43K |
| LCB3_v1.3_3 | LCB3_v1.3 | L8Q | I25F | K26Q | R28H | L35K | D37R | N43K | |
| LCB3_v1.3_4 | LCB3_v1.3 | L8Q | Y14W | I25F | K26Q | R28H | L35K | D37R | N43K |
| LCB3_v1.3_5 | LCB3_v1.3 | L8Q | Y14W | I25F | K26Q | R28H | L35K | D37T | N43K |
| | | | | | | | | | D37T, |
| LCB3_v1.3_6 | LCB3_v1.3 | D2E | L8Q | Y14W | I25F | K26Q | R28H | L35K | N43K |
| LCB3_v1.3_7 | LCB3_v1.3 | L8Q | Y14W | D22T | I25F | K26Q | R28H | L35K | D37T, |
| | | | | | | | | | N43K |
| LCB3_v1.3 | LCB3_v1.2 | L8Q | I25F | K26Q | R28H | L35K | D37T | N43K | |
| LCB3_v1.3_15 | LCB3_v1.3 | D2L | L8Q | D22T | I25F | K26Q | R28H | L35K | D37T, |
| | | | | | | | | | N43K |
| | | | | | | | | | R28H, |
| LCB3_v2.3 | LCB1_v2.1 | D2I | H6L | L8Q | M9V | V13I | I25F | K26Q | V29A, |
| | | | | | | | | | D37T, |
| | | | | | | | | | N43K, |
| | | | | | | | | | R45K, |
| | | | | | | | | | L62I |
| | | | | | | | | | L35K, |

In a further embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:33-34 and 100-101 and 164 (see Table 7). 5

TABLE 7
AHB2 exemplary variant

| | |
|---|---|
| AHB2v2 | ELEEQVMHVLDQVSELAHELLHKLTGEELERAAYFNWWATEMMLELIKSDDEREIREIEEEAARILEH |
| | LEELART (SEQ ID NO: 164) |

In one embodiment, the polypeptide comprises an amino acid substitution relative to the amino acid sequence of SEQ ID NO: 101 at or both residues selected from the group consisting 63 and 75. In a further embodiment, the substitutions comprise R63A and/or K75T.

In all embodiments disclosed herein, the polypeptides may comprise one or more additional functional groups or residues as deemed appropriate for an intended use. In one embodiment, the polypeptides may further comprise one or more added cysteine residues at the N-terminus and/or C-terminus. In another embodiment, the polypeptides may further comprise an N-linked glycosylation site (i.e.: NX(S/T), where X is any amino acid).

In another embodiment, the polypeptides may comprise two or more (i.e.: 2, 3, 4, 5, or more) copies of the amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-101. In this embodiment, 2 or more of the binders are linked. In one embodiment, the two or more copies of the polypeptide are all identical; in another embodiment, the two or more copies of the polypeptide are not all identical. In any of these embodiments, the two or more copies of the polypeptide may be separated by amino acid linker sequences, though such linkers are not required. The amino acid linkers may be of any length and amino acid composition as suitable for an intended purpose. In one embodiment, the amino acid linkers are independently between 2-100 or 3-100 amino acids in length.

In another embodiment, the amino acid linker sequences comprise Gly-Ser rich (at least 90%, 91%, 92%, 93%, 94%, 95%, 96%, 97%, 98%, 99%, or 100% Gly-Ser residues) amino acid linkers. In a further embodiment, the Gly-Ser rich linkers comprise an amino acid sequence selected from the group consisting of GG and SEQ ID NOs:35-46 and 165-171

(SEQ ID NO: 35)
GSGS
(SEQ ID NO: 36)
GGSGGS
(SEQ ID NO: 37)
SGGSGGSGGSG
(SEQ ID NO:38)
GGSGGSGSGGSG
(SEQ ID NO:39)
GGSGSSGGSGSGSG
(SEQ ID NO: 40)
GGSGSGGSGSGSGGS
(SEQ ID NO: 41)
SGGSGSGSGGSGSGS
(SEQ ID NO: 42)
GGGSGGGSSGGSGGSSGGGSGGGS
(SEQ ID NO:43)
GGGSGGGGSGGGGSGGGGSGGGGSGGGGSG
(SEQ ID NO:44)
GGGSGGGSGGSGGSGGGSGGGSGSGGSGGGGSGGGS
(SEQ ID NO: 45)
GGGGSGGGGSGGGGSGGGGSGGGGSGGGGSGGGGSG
GGGSGGGGSGGGGSGGGGSGGGGSGGGGSGGGGSGG
GGS
(SEQ ID NO: 46)
SGGGGSGGGGSGGGGSGGGGSGGGGSGGGGSGGGGS
GGGGSGGGGSGGGGSGGGGSGGGGSGGGGSGGGGSG
GGGS
(SEQ ID NO: 165)
GGSGGGGSGGGGSGGGGSGG
(SEQ ID NO: 166)
GGSGGGGSGGGGSGG
(SEQ ID NO: 167)
GGSGGGGSGG
(SEQ ID NO: 168)
GGGGSGGGG
(SEQ ID NO: 169)
GGGSGGG
(SEQ ID NO: 170)
GGSGG
(SEQ ID NO: 171)
GGSGSSG

In another embodiment, the amino acid linker sequences may comprise Pro-rich (at least 15%, 20%, 25%, or greater Pro residues) amino acid linkers. Non-limiting and exemplary embodiments may comprise an amino acid sequence selected from the group consisting of SEQ ID NOs:97-98 and 172-176.

(SEQ ID NO: 97)
AGSGGSGGSGGSPVPSTPPTPSPSTPPTPSPSPVPSTP
PTPSPSTPPTPSPSPVPSTPPTPSPSTPPTPSPSASG
(SEQ ID NO: 98)
GSGGSGGSGGSPVPSTPPTPSPSTPPTPSPSGGSGNSS
GSGGSPVPSTPPTPSPSTPPTPSPSAS
(SEQ ID NO: 172)
GGASPAAPAPASPAAPAPSAPAGG
(SEQ ID NO: 173)
GGASPAAPAPASPAGG
(SEQ ID NO: 174)
GGASPAAPAPGG
(SEQ ID NO: 175)
GGASPAAPAGG
(SEQ ID NO: 176)
GGSSGPSTPPTPSPSTPPTPSPSPGGSSG

In further non-limiting embodiments, the amino acid linkers may comprise the amino acid sequence selected from the group consisting of SEQ ID NOS: 99 and 177-178.

(SEQ ID NO: 177)
GGSSAGSPTSTGTSSATPSGSGTGG
(SEQ ID NO: 178)
GGSSGEAAAKEAAAKEAAAKGSSGG
(SEQ ID NO: 99)
GGSSGQIFVKTLTGKTITLEVEPSDTIENVKAKIQDKEGIPPDQ
QRLIFAGKQLEDGRTLSDYNIQKESTLHLVL
RLRGGGGSSG

In one embodiment, the polypeptide comprises the formula Z1-Z2-Z3, wherein:

Z1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164;

Z2 comprises an optional amino acid linker; and

Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164;

wherein Z1 and Z3 may be identical or different. In one embodiment, Z1 and Z3 are identical; in another embodiment Z1 and Z3 are different. In embodiments where Z1 and Z3 differ, each may be a variant of a given starting monomer (ex: Z1 comprises the amino acid sequence of SEQ ID NO:1 (LCB1), and Z3 comprises the amino acid sequence of SEQ ID NO: 102-136. Any such combination of the monomers disclosed herein may be used. It will further be understood that the polypeptides may comprise 2, 3, 4, 5, or more monomers of any embodiment disclosed herein. In embodiments where there are 3 or more monomers, all 3 monomers may be identical; 2 monomers may be identical and one may differ, or all 3 monomers may be different.

In one embodiment employing LCB1 and variants thereof, Z1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136; and

Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136.

In another embodiment employing LCB3 and variants thereof,

Z1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163; and

Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163.

In another embodiment employing AHB and variants thereof,

Z1 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-101, and 164; and

Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-101, and 164.

In one embodiment, one of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136; and

the other of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163.

In another embodiment, one of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136; and

the other of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-100, and 164.

In a further embodiment, one of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting SEQ ID NOS: 13-17, 19-21 and 137-163; and

the other of Z1 and Z3 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-100, and 164.

In another embodiment of any of the other embodiments disclosed herein, the polypeptide comprises at least 3 monomers (i.e.: 3, 4, 5, or more). In one such embodiment, the polypeptide comprises the formula B1-B2-Z1-Z2-Z3-B3-B4, wherein:

Z1, Z2, and Z3 are as defined above;

B2 and B3 comprise optional amino acid linkers; and

one or both of B1 and B4 independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: SEQ ID NOS: 1-17, 19-21, 23-34 and 100-164, wherein one of B1 and B4 may be absent. In one embodiment, one of B1 and B4 is absent. In another embodiment, both B1 and B4 are present. In one embodiment, B1 and B4 independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164. In this embodiment, B1 and B4 may be identical or may be different. In one embodiment, B1 when present and B4 when present, are identical to one or both of Z1 and Z3. In another embodiment, B1 when present and B4 when present, are not identical to either of Z1 and Z3.

In one embodiment, B1 when present, and B4 when present, independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10, 13-17, 19-21, 33-34, 100-101, and 102-164.

In another embodiment, B1 when present, and B4 when present, independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136.

In a further embodiment, B1 when present, and B4 when present, independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163.

In a still further embodiment, B1 when present, and B4 when present, independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-101, and 164.

In various embodiments when both B1 and B4 are present,
one of B1 and B4 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136, and the other comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163;
one of B1 and B4 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-10 and 102-136, and the other comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-101, and 164, or
one of B1 and B4 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 13-17, 19-21 and 137-163, and the other comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 33-34, 100-101, and 164.

In various non-limiting embodiments, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:47-60, 193-355, and 454-588 and a genus selected from those recited in the right hand column of Table 8 wherein genus positions X1, X2, X3, and X4 may be present or absent, and when present may be any sequence of 1 or more amino acids. In all embodiments, any N-terminal methionine residues may be present or absent in the polypeptide. In one embodiment, any N-terminal methionine residues are absent in the polypeptide.

>LCB1-6GS-LCB1
(SEQ ID NO: 47)
DKEWILQKIYEIMRLLDELGHAEASMRVSDLIYEFMKKGDERLLEEAERLLEEVERGGSGGSDKEWILQKIYEIMRLLDELGH
AEASMRVSDLIYEFMKKGDERLLEEAERLLEEVER
>LCB1-12GS-LCB1
(SEQ ID NO: 48)
DKEWILQKIYEIMRLLDELGHAEASMRVSDLIYEFMKKGDERLLEEAERLLEEVERGGSGGSGSGGSGDKEWILQKIYEIMRL
LDELGHAEASMRVSDLIYEFMKKGDERLLEEAERLLEEVER
>LCB1-24GS-LCB1
(SEQ ID NO: 49)
DKEWILQKIYEIMRLLDELGHAEASMRVSDLIYEFMKKGDERLLEEAERLLEEVERGGGSGGGSSGGSGGSSGGGSGGGSDKE
WILQKIYEIMRLLDELGHAEASMRVSDLIYEFMKKGDERLLEEAERLLEEVER
>LCB1-36GS-LCB1
(SEQ ID NO: 50)
GGGGSGGGSDKEWILQKIYEIMRLLDELGHAEASMRVSDLIYEFMKKGDERLLEEAERLLEEVER
>LCB1_v1.1-GSLCB1_v1.1(1GS1)
(SEQ ID NO: 51)
DKENILQKIYEIMKTLDOLGHAEASMQVSDLIYEFMKQGDERLLEEAERLLEEVERGGGGSGGGGSGGGGSGGGGSGGGGSGG
GGSGGGGSGGGGSGGGGSGGGGSGGGGSGGGGSGGGGSGGGGSGGGGSDKENILQKIYEIMKTLDQLGHAEASMQVSDLIYEF
MKQGDERLLEEAERLLEEVER
>LCB1_v1.1-PRO-LCB1_v1.1(1PRO1)
(SEQ ID NO: 52)
DKENILQKIYEIMKTLDQLGHAEASMQVSDLIYEFMKQGDERLLEEAERLLEEVERAGSGGSGGSGGSPVPSTPPTPSPSTPP
TPSPSPVPSTPPTPSPSTPPTPSPSPVPSTPPTPSPSTPPTPSPSASGDKENILQKIYEIMKTLDQLGHAEASMQVSDLIYEF
MKQGDERLLEEAERLLEEVER
>LCB3_v1.2-GS3-LCB3_v1.2(3GS3)
(SEQ ID NO: 53)
NDDELHMQMTDLVYEALHFAKDEEIQKHVFQLFEKATKAYKNKDRQKLEKVVEELKELLERLLSGGGGSGGGGSGGGGSGGGG
SGGGGSGGGGSGGGGSGGGGSGGGGSGGGGSGGGGSGGGGSGGGGSGGGGSGGGGSNDDELHMQMTDLVYEALHFAKDEEIQK
HVFQLFEKATKAYKNKDRQKLEKVVEELKELLERLLS
>LCB3_v1.2-PRO-LCB3_v1.2(3PRO3)
(SEQ ID NO: 54)
NDDELHMQMTDLVYEALHFAKDEEIQKHVFQLFEKATKAYKNKDRQKLEKVVEELKELLERLLSAGSGGSGGSGGSPVPSTPP
TPSPSTPPTPSPSPVPSTPPTPSPSTPPTPSPSPVPSTPPTPSPSTPPTPSPSASGNDDELHMQMTDLVYEALHFAKDEEIQK
HVFQLFEKATKAYKNKDRQKLEKVVEELKELLERLLS
>LCB1_v1.1-GS-LCB3_v1.2(1GS3)
(SEQ ID NO: 55)
DKENILQKIYEIMKTLDQLGHAEASMQVSDLIYEFMKQGDERLLEEAERLLEEVERGGGGSGGGGSGGGGSGGGGSGGGGSGG
GGSGGGGSGGGGSGGGGSGGGGSGGGGSGGGGSGGGGSGGGGSGGGGSNDDELHMQMTDLVYEALHFAKDEEIQKHVFQLFEK
ATKAYKNKDRQKLEKVVEELKELLERLLS
>LCB3_v1.2-GS-LCB1_v1.1(3GS1)
(SEQ ID NO: 56)
NDDELHMQMTDLVYEALHFAKDEEIQKHVFQLFEKATKAYKNKDRQKLEKVVEELKELLERLLSGGGGSGGGGSGGGGSGGGG
SGGGGSGGGGSGGGGSGGGGSGGGGSGGGGSGGGGSGGGGSGGGGSGGGGSGGGGSDKENILQKIYEIMKTLDQLGHAEASMQ
VSDLIYEFMKOGDERLLEEAERLLEEVER
>LCB3_v1.2-10GS-LCB1_v1.1(LCB3-GS10-LCB1)
(SEQ ID NO: 57)
NDDELHMQMTDLVYEALHFAKDEEIQKHVFQLFEKATKAYKNKDRQKLEKVVEELKELLERLLSGGSGGSGGSGDKENILQKI
YEIMKTLDQLGHAEASMQVSDLIYEFMKQGDERLLEEAERLLEEVER
>LCB1_v1.1-PRO-LCB3_v1.2(1PRO3)
(SEQ ID NO: 58)
DKENILQKIYEIMKTLDQLGHAEASMQVSDLIYEFMKQGDERLLEEAERLLEEVERAGSGGSGGSGGSPVPSTPPTPSPSTPP
TPSPSPVPSTPPTPSPSTPPTPSPSPVPSTPPTPSPSTPPTPSPSASGNDDELHMQMTDLVYEALHFAKDEEIQKHVFQLFEK
ATKAYKNKDRQKLEKVVEELKELLERLLS
>LCB3_v1.2-PRO-LCB1_v1.1(3PRO1)
(SEQ ID NO: 59)
NDDELHMQMTDLVYEALHFAKDEEIQKHVFQLFEKATKAYKNKDRQKLEKVVEELKELLERLLSAGSGGSGGSGGSPVPSTPP
TPSPSTPPTPSPSPVPSTPPTPSPSTPPTPSPSPVPSTPPTPSPSTPPTPSPSASGDKENILQKIYEIMKTLDQLGHAEASMQ
VSDLIYEFMKQGDERLLEEAERLLEEVER
>36175(5_LCB1_linker14)
(SEQ ID NO : 60)
DKEWILQKIYEIMRLLDELGHAEASMRVSDLIYEFMKKGDERLLEEAERLLEEVERGGSGSSGGSGSGSGDKEWILQKIYEIM
RLLDELGHAEASMRVSDLIYEFMKKGDERLLEEAERLLEEVERGGSGSSGGSGSGSGDKEWILQKIYEIMRLLDELGHAEASM
RVSDLIYEFMKKGDERLLEEAERLLEEVERGGSGSSGGSGSGSGDKEWILQKIYEIMRLLDELGHAEASMRVSDLIYEFMKKG
DERLLEEAERLLEEVERGGSGSSGGSGSGSGDKEWILQKIYEIMRLLDELGHAEASMRVSDLIYEFMKKGDERLLEEAERLLE
EVER

Daisy Chain Designs

| | | Annotated: X1, X2, X3, and X4 may be |
|---|---|---|
| | | present or absent, and when present |
| | | may be any sequence of 1 or more |
| Name | Protein | amino acids |
| 1GS1 | MEKKIGSSAWSHPQFEKGGGSGGGSGGSAWSHPQFE | X1- |
| | KGGSGSSGGGGDKENILQKIYEIMKTLDQLGHAEAS | DKENILQKIYEIMKTLDQLGHAEASMQVSDLIYEF |
| | MQVSDLIYEFMKQGDERLLEEAERLLEEVERGGGGS | MKQGDERLLEEAERLLEEVER(SEQ ID NO: 4)- |
| | GGGGSGGGGSGGGGSGGGGSGGGGSGGGGSGGGGSG | X2- |
| | GGGSGGGGSGGGGSGGGGSGGGGSGGGGSGGGGSDK | DKENILQKIYEIMKTLDQLGHAEASMQVSDLIYEF |
| | ENILQKIYEIMKTLDQLGHAEASMQVSDLIYEFMKQ | MKQGDERLLEEAERLLEEVER(SEQ ID NO: 4) |
| | GDERLLEEAERLLEEVER(SEQ ID NO: 193) | |
| 1PR01 | MEKKIGSSAWSHPQFEKGGGSGGGSGGSAWSHPQFE | X1- |
| | KGGSGSSGGGGDKENILQKIYEIMKTLDQLGHAEAS | DKENILQKIYEIMKTLDQLGHAEASMQVSDLIYEF |
| | MQVSDLIYEFMKQGDERLLEEAERLLEEVERAGSGG | MKQGDERLLEEAERLLEEVER(SEQ ID NO: 4)- |
| | SGGSGGSPVPSTPPTPSPSTPPTPSPSPVPSTPPTP | X2- |
| | SPSTPPTPSPSPVPSTPPTPSPSTPPTPSPSASGDK | DKENILQKIYEIMKTLDQLGHAEASMQVSDLIYEF |
| | ENILQKIYEIMKTLDQLGHAEASMQVSDLIYEFMKQ | MKQGDERLLEEAERLLEEVER(SEQ ID NO: 4) |
| | GDERLLEEAERLLEEVER(SEQ ID NO: 194) | |
| 3GS3 | MEKKIGSSAWSHPQFEKGGGSGGGSGGSAWSHPQFE | X1- |
| | KGGSGSSGGGGNDDELHMQMTDLVYEALHFAKDESI | NDDELHMQMTDLVYEALHFAKDEEIQKHVFQLFEK |
| | QKHVFQLFEKATKAYKNKDRQKLEKVVEELKELLSR | ATKAYKNKDRQKLEKVVEELKELLERLLS |
| | LLSGGGGSGGGGSGGGGSGGGGSGGGGSGGGGSGGG | (SEQ ID NO: 15)-X2- |
| | GSGGGGSGGGGSGGGGSGGGGSGGGGSGGGGSGGGG | NDDELHMQMTDLVYEALHFAKDEEIQKHVFQLFEK |
| | SGGGGSNDDELHMQMTDLVYEALHFAKDEEIQKHVF | ATKAYKNKDRQKLEKVVEELKELLERLLS |
| | QLFEKATKAYKNKDRQKLEKVVEELKELLERLLS | (SEQ ID NO: 15) |
| | (SEQ ID NO: 195) | |
| 3PR03 | MEKKIGSSAWSHPQFEKGGGSGGGSGGSAWSHPQFE | X1- |
| | KGGSGSSGGGGNDDELHMQMTDLVYEALHFAKDE2I | NDDELHMQMTDLVYEALHFAKDEEIQKHVFQLFEK |
| | QKHVFQLFEKATKAYKNKDRQKLEKVVEELKELL2R | ATKAYKNKDRQKLEKVVEELKELLERLLS |
| | LLSAGSGGSGGSGGSPVPSTPPTPSPSTPPTPSPSP | (SEQ ID NO: 15)-X2- |
| | VPSTPPTPSPSTPPTPSPSPVPSTPPTPSPSTPPTP | NDDELHMQMTDLVYEALHFAKDEEIQKHVFQLFEK |
| | SPSASGNDDELHMQMTDLVYEALHFAKDEEIQKHVF | ATKAYKNKDRQKLEKVVEELKELLERLLS |
| | QLFEKATKAYKNKDRQKLEKVVEELKELLERLLS | (SEQ ID NO: 15) |
| | (SEQ ID NO: 196) | |
| 1GS3 | MEKKIGSSAWSHPQFEKGGGSGGGSGGSAWSHPQFE | X1- |
| | KGGSGSSGGGGDKENILQKIYEIMKTLDQLGHAEAS | DKENILQKIYEIMKTLDQLGHAEASMQVSDLIYEF |
| | MQVSDLIYEFMKQGDERLLEEAERLLEEVERGGGGS | MKQGDERLLEEAERLLEEVER(SEQ ID NO: 4)- |
| | GGGGSGGGGSGGGGSGGGGSGGGGSGGGGSGGGGSG | X2- |
| | GGGSGGGGSGGGGSGGGGSGGGGSGGGGSGGGGSND | NDDELHMQMTDLVYEALHFAKDEEIQKHVFQLFEK |
| | DELHMQMTDLVYEALHFAKDEEIQKHVFQLFEKATK | ATKAYKNKDRQKLEKVVEELKELLERLLS(SEQ |
| | AYKNKDRQKLEKVVEELKELLERLLS | ID NO: 15) |
| | (SEQ ID NO: 197) | |
| 3GS1 | MEKKIGSSAWSHPQFEKGGGSGGGSGGSAWSHPQFE | X1- |
| | KGGSGSSGGGGNDDELHMQMTDLVYEALHFAKDESI | NDDELHMQMTDLVYEALHFAKDEEIQKHVFQLFEK |
| | QKHVFQLFEKATKAYKNKDRQKLEKVVEELKELLER | ATKAYKNKDRQKLEKVVEELKELLERLLS(SEQ |
| | LLSGGGGSGGGGSGGGGSGGGGSGGGGSGGGGSGGG | ID NO: 15)-X2- |
| | GSGGGGSGGGGSGGGGSGGGGSGGGGSGGGGSGGGG | DKENILQKIYEIMKTLDQLGHAEASMQVSDLIYEF |
| | SGGGGSDKENILQKIYEIMKTLDQLGHAEASMQVSD | MKQGDERLLEEAERLLEEVER(SEQ ID NO: 4) |
| | LIYEFMKQGDERLLEEAERLLEEVER | |
| | (SEQ ID NO: 198) | |
| 1PR03 | MEKKIGSSAWSHPQFEKGGGSGGGSGGSAWSHPQFE | X1- |
| | KGGSGSSGGGGDKENILQKIYEIMKTLDQLGHAEAS | DKENILQKIYEIMKTLDQLGHAEASMQVSDLIYEF |
| | MQVSDLIYEFMKQGDERLLEEAERLLEEVERAGSGG | MKQGDERLLEEAERLLEEVER(SEQ ID NO: 4)- |
| | SGGSGGSPVPSTPPTPSPSTPPTPSPSPVPSTPPTP | X2- |
| | SPSTPPTPSPSPVPSTPPTPSPSTPPTPSPSASGND | NDDELHMQMTDLVYEALHFAKDEEIQKHVFQLFEK |
| | DELHMQMTDLVYEALHFAKDEEIQKHVFQLFEKATK | ATKAYKNKDRQKLEKVVEELKELLERLLS(SEQ |
| | AYKNKDRQKLEKVVEELKELLERLLS | ID NO: 15) |
| | (SEQ ID NO: 199) | |
| 3PR01 | MEKKIGSSAWSHPQFEKGGGSGGGSGGSAWSHPQFE | X1- |
| | KGGSGSSGGGGNDDELHMQMTDLVYEALHFAKDEEI | NDDELHMQMTDLVYEALHFAKDEEIQKHVFQLF |
| | QKHVFQLFEKATKAYKNKDRQKLEKVVEELKELLERL | EKATKAYKNKDRQKLEKVVEELKELLERLLS |
| | LSAGSGGSGGSGGSPVPSTPPTPSPSTPPTPSPSPV | (SEQ ID NO: 15)- |
| | PSTPPTPSPSTPPTPSPSPVPSTPPTPSPSTPPTPS | X2- |
| | PSASGDKENILQKIYEIMKTLDQLGHAEASMQVSDL | DKENILQKIYEIMKTLDQLGHAEASMQVSDLIY |
| | IYEFMKQGDERLLEEAERLLEEVER | EFMKQGDERLLEEAERLLEEVER |
| | (SEQ ID NO: 200) | (SEQ ID NO: 4) |
| CSL- | MEKKISAWSHPQFEKGGGSGGGSGGSAWSHPQFEKG | X1- |
| LCB1- | GSGSSGDKEWILQKIYEIMRLLDELGHAEASMRVSD | DKEWILQKIYEIMRLLDELGHAEASMRVSDLIY |
| GS15- | LIYEFMKKGDERLLEEAERLLEEVERGGGGSGGGGS | EFMKKGDERLLEEAERLLEEVER |
| LCB1 | GGGGSDKEWILQKIYEIMRLLDELGHAEASMRVSDL | (SEQ ID NO: 1)- |
| | IYEFMKKGDERLLEEAERLLEEVER | X2- |
| | (SEQ ID NO: 201) | DKEWILQKIYEIMRLLDELGHAEASMRVSD |
| | | LIYEFMKKGDERLLEEAERLLEEVER |
| | | (SEQ ID NO: 1) |
| CSL- | MEKKISAWSHPQFEKGGGSGGGSGGSAWSHPQFEKG | X1- |
| LCB3- | GSGSSGNDDELHMLMTDLVYEALHFAKDEEIKKRVF | NDDELHMLMTDLVYEALHFAKDEEIKKRVFQLF |
| GS15- | QLFELADKAYKNNDRQKLEKVVEELKELLERLLSGGG | ELADKAYKNNDRQKLEKVVEELKELLERLLS |
| LCB3 | GSGGGGSGGGGSNDDELHMLMTDLVYEALHFAKDEE | (SEQ ID NO: 13)- |
| | IKKRVFQLFELADKAYKNNDRQKLEKVVEELKELLER | X2- |
| | LLS | NDDELHMLMTDLVYEALHFAKDEEIKKRVFQLF |
| | (SEQ ID NO: 202) | ELADKAYKNNDRQKLEKVVEELKELLERLLS |
| | | (SEQ ID NO: 13) |
| CSL- | MEKKISAWSHPQFEKGGGSGGGSGGSAWSHPQFEKG | X1- |
| LCB1- | GSGSSGDKEWILQKIYEIMRLLDELGHAEASMRVSD | DKEWILQKIYEIMRLLDELGHAEASMRVSDLIY |
| GS20- | LIYEFMKKGDERLLEEAERLLEEVERGGGGSGGGGS | EFMKKGDERLLEEAERLLEEVER |
| LCB1 | GGGGSGGGGSDKEWILQKIYEIMRLLDELGHAEASM | (SEQ ID NO: 1)- |
| | RVSDLIYEFMKKGDERLLEEAERLLEEVER | X2- |
| | (SEQ ID NO: 203) | DKEWILQKIYEIMRLLDELGHAEASMRVSD |
| | | LIYEFMKKGDERLLEEAERLLEEVER |
| | | (SEQ ID NO: 1) |
| CSL- | MEKKISAWSHPQFEKGGGSGGGSGGSAWSHPQFEK | X1- |
| LCB3- | GGSGSSGNDDELHMLMTDLVYE1ALHFAKDEEIKKR | NDDELHMLMTDLVYEALHFAKDEEIKKRVFQLF |
| GS20- | VFQLFELADKAYKNNDRQKLEKVVEELKELLERLLSG | ELADKAYKNNDRQKLEKVVEELKELLERLLS |
| LCB3 | GGGSGGGGSGGGGSGGGGSNDDELHMLMTDLVYEAL | (SEQ ID NO: 13)- |
| | HFAKDEEIKKRVFQLFELADKAYKNNDRQKLEKVVEE | X2- |
| | LKELLERLLS | NDDELHMLMTDLVYEALHFAKDEEIKKRVFQLF |
| | (SEQ ID NO: 204) | ELADKAYKNNDRQKLEKVVEELKELLERLLS |
| | | (SEQ ID NO: 13) |
| CSL- | MEKKISAWSHPQFEKGGSGSSGDKEWILQKIYEIMR | X1- |
| LCB1- | LLDELGHAEASMRVSDLIYEFMKKGDERLLEEAERL | DKEWILQKIYEIMRLLDELGHAEASMRVSDLIY |
| GS20- | LEEVERGSSGSGSSGSGSSGSGSSGSDKEWILQKIY | EFMKKGDERLLEEAERLLEEVER |
| LCB1- | EIMRLLDELGHAEASMRVSDLIYEFMKKGDERLLEE | (SEQ ID NO: 1)- |
| GS20- | AERLLEEVERGSSSGGSSSGGSSSGGSSSGDKEWIL | X2- |
| LCB1 | QKIYEIMRLLDELGHAEASMRVSDLIYEFMKKGDER | DKEWILQKIYEIMRLLDELGHAEASMRVSD |
| | LLEEAERLLEEVER | LIYEFMKKGDERLLEEAERLLEEVER |
| | (SEQ ID NO: 205) | (SEQ ID NO: 1)- |
| | | X3- |
| | | DKEWILQKIYEIMRLLDELGHAEASMR |
| | | VSDLIYEFMKKGDERLLEEAERLLEEVER |
| | | (SEQ ID NO: 1) |
| CSL- | MEKKISAWSHPQFEKGGSGSSGNDDELHMLMTDLVY | X1- |
| LCB3- | EALHFAKDEEIKKRVFQLFELADKAYKNNDRQKLEK | NDDELHMLMTDLVYEALHFAKDEEIKKRVFQLF |
| GS20- | WEELKELLERLLSGSSGSGSSGSGSSGSGSSGSNDD | ELADKAYKNNDRQKLEKVVEELKELLERLLS |
| LCB3- | ELHMLMTDLVYEALHFAKDEEIKKRVFQLFELADKA | (SEQ ID NO: 13)- |
| GS20- | YKNNDRQKLEKVVEELKELLERLLSGSSSGGSSSGGS | X2- |
| LCB3 | SSGGSSSGNDDELHMLMTDLVYEALHFAKDEEIKKR | DDELHMLMTDLVYEALHFAKDEEIKKRVFQLFEL |
| | VFQLFELADKAYKNNDRQKLEKVVEELKELLERLLS | ADKAYKNNDRQKLEKVVEELKELLERLLS |
| | (SEQ ID NO: 206) | (SEQ ID NO: 13)- |
| | | X3- |
| | | NDDELHMLMTDLVYEALHFAKDEEIKKRVFQLF |
| | | ELADKAYKNNDRQKLEKVVEELKELLERLLS |
| | | (SEQ ID NO: 13) |
| CSL- | MEKKISAWSHPQFEKGGGSGGGSGGSAWSHPQFEKG | X1- |
| LCB1- | GSGSSGDKEWILQKIYEIMRLLDELGHAEASMRVSD | DKEWILQKIYEIMRLLDELGHAEASMRVSDLIY |
| XTENx2 | LIYEFMKKGDERLLEEAERLLEEVERGSAGGSPAGS | EFMKKGDERLLEEAERLLEEVER |
| | PTSTGTSTSGDKEWILQKIYEIMRLLDELGHAEASM | (SEQ ID NO: 1)- |
| | RVSDLIYEFMKKGDERLLEEAERLLEEVER | X2- |
| | (SEQ ID NO: 207) | DKEWILQKIYEIMRLLDELGHAEASMRVSD |
| | | LIYEFMKKGDERLLEEAERLLEEVER |
| | | (SEQ ID NO: 1) |
| CSL- | MEKKISAWSHPQFEKGGGSGGGSGGSAWSHPQFEK | X1- |
| LCB1- | GGSGSSGDKEWILQKIYEIMRLLDELGHAEASMRVS | DKEWILQKIYEIMRLLDELGHAEASMRVSDLIY |
| XTENx | DLIYEFMKKGDERLLEEAERLLEEVERGSAGGSPAG | EFMKKGDERLLEEAERLLEEVER |
| 3 | S | (SEQ ID NO: 1) |
| | PTSTGTSGSGDKEWILQKIYEIMRLLDELGHAEASM | -X2- |
| | RVSDLIYEFMKKGDERLLEEAERLLEEVERGSAGGS | DKEWILQKIYEIMRLLDELGHAEASMRVSDLIYEF |
| | PAGSPTSTGTSGSGDKEWILQKIYEIMRLLDELGHA | MKKGDERLLEEAERLLEEVER(SEQ ID NO: 1) |
| | EASMRVSDLIYEFMKKGDERLLEEAERLLEEVER | -X3- |
| | (SEQ ID NO: 208) | DKEWILQKIYEIMRLLDELGHAEASMRVSDLIYEF |
| | | MKKGDERLLEEAERLLEEVER(SEQ ID NO: 1) |
| CSL- | MEKKISAWSHPQFEKGGGSGGGSGGSAWSHPQFEKG | X1-NDDELHMLMTDLVYEALHFAKDEEIKKRVFQLFEL |
| LCB3- | GSGSSGNDDELHMLMTDLVYEALHFAKDEEIKKRVF | ADKAYKNNDRQKLEKVVEELKELLERLLS |
| XTENx | QLFELADKAYKNNDRQKLEKVVEELKELLERLLSGS | (SEQ ID NO: 13) |
| 2 | AGGSPAGSPTSTGTSGSGNDDELHMLMTDLVYEALH | -X2- |
| | FAKDEEIKKRVFQLFELADKAYKNNDRQKLEKVVEE | NDDELHMLMTDLVYEALHFAKDEEIKKRVFQLFEL |
| | LKELLERLLS | ADKAYKNNDRQKLEKVVEELKELLERLLS |
| | (SEQ ID NO: 209) | (SEQ ID NO: 13) |
| CSL- | MEKKISAWSHPQFEKGGGSGGGSGGSAWSHPQFEKG | X1-NDDELHMLMTDLVYEALHFAKDEEIKKRVFQLFEL |
| LCB3- | GSGSSGNDDELHMLMTDLVYEALHFAKDEEIKKRVF | ADKAYKNNDRQKLEKVVEELKELLERLLS |
| XTENx | QLFELADKAYKNNDRQKLEKVVEELKELLERLLSGS | (SEQ ID NO: 13) |
| 3 | AGGSPAGSPTSTGTSGSGNDDELHMLMTDLVYEALH | -X2- |
| | FAKDEEIKKRVFQLFELADKAYKNNDRQKLEKVVEE | NDDELHMLMTDLVYEALHFAKDEEIKKRVFQLFEL |
| | LKELLERLLSGSAGGSPAGSPTSTGTSGSGNDDELH | ADKAYKNNDRQKLEKVVEELKELLERLLS |
| | MLMTDLVYEALHFAKDEEIKKRVFQLFELADKAYKN | (SEQ ID NO: 13) |
| | NDRQKLEKVVEELKELLERLLS | -X3- |
| | (SEQ ID NO: 210) | NDDELHMLMTDLVYEALHFAKDEEIKKRVFQLFEL |
| | | ADKAYKNNDRQKLEKVVEELKELLERLLS |
| | | (SEQ ID NO: 13) |
| C- | MEKKISSGDKEWILQKIYEIMRLLDELGHAEASMRV | X1-DKEWILQKIYEIMRLLDELGHAEASMRVSDLIYEF |
| LCB1- | SDLIYEFMKKGDERLLEEAERLLEEVERGSSGSGSS | MKKGDERLLEEAERLLEEVER |
| GS20- | GSGSSGSGSSGSDKEWILQKIYEIMRLLDELGHAEA | (SEQ ID NO: 1) |
| LCB1- | SMRVSDLIYEFMKKGDERLLEEAERLLEEVERGSSS | -X2- |
| GS20- | GGSSSGGSSSGGSSSGDKEWILQKIYEIMRLLDELG | DKEWILQKIYEIMRLLDELGHAEASMRVSDLIYEF |
| LCB1- | HAEASMRVSDLIYEFMKKGDERLLEEAERLLEEVER | MKKGDERLLEEAERLLEEVER |
| LS | GGSGSSGSAWSHPQFEK(SEQ ID NO: 211) | (SEQ ID NO: 1) |
| | | -X3- |
| | | DKEWILQKIYEIMRLLDELGHAEASMRVSDLIYEF |
| | | MKKGDERLLEEAERLLEEVER |
| | | (SEQ ID NO: 1)-X4 |
| C- | MEKKISSGNDDELHMLMTDLVYEALHFAKDEEIKKR | X1-NDDELHMLMTDLVYEALHFAKDEEIKKRVFQLFEL |
| LCB3- | VFQLFELADKAYKNNDRQKLEKVVEELKELLERLLS | ADKAYKNNDRQKLEKVVEELKELLERLLS |
| GS2Q- | GSSGSGSSGSGSSGSGSSGSNDDELHMLMTDLVYEA | (SEQ ID NO: 13) |
| LCB3- | LHFAKDEEIKKRVFQLFELADKAYKNNDRQKLEKW | -X2- |
| GS20- | EELKELLERLLSGSSSGGSSSGGSSSGGSSSGNDDE | NDDELHMLMTDLVYEALHFAKDEEIKKRVFQLFEL |
| LCB3- | LHMLMTDLVYEALHFAKDEEIKKRVFQLFELADKAY | ADKAYKNNDRQKLEKVVEELKELLERLLS |
| LS | KNNDRQKLEKVVEELKELLERLLSGGSGSSGSAWSH | (SEQ ID NO: 13) |
| | PQFEK | -X3- |
| | (SEQ ID NO: 212) | NDDELHMLMTDLVYEALHFAKDEEIKKRVFQLFEL |
| | | ADKAYKNNDRQKLEKVVEELKELLERLLS |
| | | (SEQ ID NO: 13)-X4 |
| CSL- | MEKKISAWSHPQFEKGGGSGGGSGGSAWSHPQFEKG | X1-DKEWILQKIYEIMRLLDELGHAEASMRVSDLIYEF |
| LCB1- | GSGSSGDKEWILQKIYEIMRLLDELGHAEASMRVSD | MKKGDERLLEEAERLLEEVER |
| XTEN2 | LIYEFMKKGDERLLEEAERLLEEVERGGSSAGSPTS | (SEQ ID NO: 1) |
| 5x3 | TGTSSATPSGSGTGGDKEWILQKIYEIMRLLDELGH | -X2- |
| | AEASMRVSDLIYEFMKKGDERLLEEAERLLEEVERG | DKEWILQKIYEIMRLLDELGHAEASMRVSDLIYEF |
| | GSSAGSPTSTGTSSATPSGSGTGGDKEWILQKIYEI | MKKGDERLLEEAERLLEEVER |
| | MRLLDELGHAEASMRVSDLIYEFMKKGDERLLEEAE | (SEQ ID NO: 1)-X3 |
| | RLLEEVER | DKEWILQKIYEIMRLLDELGHAEASMRVSDLIYEF |
| | (SEQ ID NO: 213) | MKKGDERLLEEAERLLEEVER |
| | | (SEQ ID NO: 1) |
| CSL- | MEKKISAWSHPQFEKGGGSGGGSGGSAWSHPQFEKG | X1-NDDELHMLMTDLVYEALHFAKDEEIKKRVFQLF |
| LCB3- | GSGSSGNDDELHMLMTDLVYEALHFAKDEEIKKRVF | ELADKAYKNNDRQKLEKVVEELKELLERLLS |
| XTEN2 | QLFELADKAYKNNDRQKLEKVVEELKELLERLLSGG | (SEQ ID NO: 13) |
| 5x3 | SSAGSPTSTGTSSATPSGSGTGGNDDELHMLMTDLV | -X2- |
| | YEALHFAKDEEIKKRVFQLFELADKAYKNNDRQKLE | NDDELHMLMTDLVYEALHFAKDEEIKKRVFQLFEL |
| | KVVEELKELLERLLSGGSSAGSPTSTGTSSATPSGS | ADKAYKNNDRQKLEKVVEELKELLERLLS |
| | GTGGNDDELHMLMTDLVYEALHFAKDEEIKKRVFQL | (SEQ ID NO: 13) |
| | FELADKAYKNNDRQKLEKVVEELKELLERLLS | -X3- |
| | (SEQ ID NO: 214) | NDDELHMLMTDLVYEALHFAKDEEIKKRVFQLFEL |
| | | ADKAYKNNDRQKLEKVVEELKELLERLLS |
| | | (SEQ ID NO: 13) |
| LCB1 | MEKKISAWSHPQFEKGGGSGGGSGGSAWSHPQFEKG | X1-DKENILQKIYEIMKTLEQLGHAEASMQVSDLIYEF |
| v1.3_ | GSGSSGDKENILQKIYEIMKTLEQLGHAEASMQVSD | MKQGDERLLEEAERLLEEVER |
| GS_2X | LIYEFMKQGDERLLEEAERLLEEVERGGSSGGGSSG | (SEQ ID NO: 8) |
| | GGSSGGGSSGGGSSGDKENILQKIYEIMKTLEQLGH | -X2- |
| | AEASMQVSDLIYEFMKQGDERLLEEAERLLEEVER | DKENILQKIYEIMKTLEQLGHAEASMQVSDLIYEF |
| | (SEQ ID NO: 215) | MKQGDERLLEEAERLLEEVER(SEQ ID NO: 8) |
| LCB1 | MEKKISAWSHPQFEKGGGSGGGSGGSAWSHPQFEKG | X1- |
| _v1.3 | GSGSSGDKENILQKIYEIMKTLEQLGHAEASMQVSD | DKENILQKIYEIMKTLEQLGHAEASMQVSDLIYEF |
| XTEN | LIYEFMKQGDERLLEEAERLLEEVERGGSSAGSPTS | MKQGDERLLEEAERLLEEVER(SEQ ID NO: 8)- |
| 2X | TGTSSATPSGSGTGGDKENILQKIYEIMKTLEQLGH | X2- |
| | AEASMQVSDLIYEFMKQGDERLLEEAERLLEEVER | DKENILQKIYEIMKTLEQLGHAEASMQVSDLIYEF |
| | (SEQ ID NO: 216) | MKQGDERLLEEAERLLEEVER(SEQ ID NO: 8) |
| LCB1 | MEKKISAWSHPQFEKGGGSGGGSGGSAWSHPQFEKG | X1- |
| _v1.3 | GSGSSGDKENILQKIYEIMKTLEQLGHAEASMQVSD | DKENILQKIYEIMKTLEQLGHAEASMQVSDLIYEF |
| EAAAK | LIYEFMKQGDERLLEEAERLLEEVERGGSSGEAAAK | MKQGDERLLEEAERLLEEVER(SEQ ID NO: 8)- |
| _2X | EAAAKEAAAKGSSGGDKENILQKIYEIMKTLEQLGH | X2- |
| | AEASMQVSDLIYEFMKQGDERLLEEAERLLEEVER | DKENILQKIYEIMKTLEQLGHAEASMQVSDLIYEF |
| | (SEQ ID NO: 217) | MKQGDERLLEEAERLLEEVER(SEQ ID NO: 8) |
| LCB1 | MEKKISAWSHPQFEKGGGSGGGSGGSAWSHPQFEKG | X1- |
| _v1.3 | GSGSSGDKENILQKIYEIMKTLEQLGHAEASMQVSD | DKENILQKIYEIMKTLEQLGHAEASMQVSDLIYEF |
| Pro_2 | LIYEFMKQGDERLLEEAERLLEEVERGGSSGPSTPP | MKQGDERLLEEAERLLEEVER(SEQ ID NO: 8)- |
| X | TPSPSTPPTPSPSPGGSSGDKENILQKIYEIMKTLE | X2- |
| | QLGHAEASMQVSDLIYEFMKQGDERLLEEAERLLEE | DKENILQKIYEIMKTLEQLGHAEASMQVSDLIYEF |
| | VER(SEQ ID NO: 218) | MKQGDERLLEEAERLLEEVER(SEQ ID NO: 8) |
| LCB1_ | MEKKISAWSHPQFEKGGGSGGGSGGSAWSHPQFEKG | X1- |
| _v1.3 | GSGSSGDKENILQKIYEIMKTLEQLGHAEASMQVSD | DKENILQKIYEIMKTLEQLGHAEASMQVSDLIYEF |
| Ub_2X | LIYEFMKQGDERLLEEAERLLEEVERGGSSGQIFVK | MKQGDERLLEEAERLLEEVER(SEQ ID NO: 8)- |
| | TLTGKTITLEVEPSDTIENVKAKIQDKEGIPPDQQR | X2- |
| | LIFAGKQLEDGRTLSDYNIQKESTLHLVLRLRGGGG | DKENILQKIYEIMKTLEQLGHAEASMQVS DLIYEF |
| | SSGDKENILQKIYEIMKTLEQLGHAEASMQVSDLIY | MKQGDERLLEEAERLLEEVER(SEQ ID NO: 8) |
| | EFMKQGDERLLEEAERLLEEVER | |
| | (SEQ ID NO: 219) | |
| LCB1_ | MEKKISAWSHPQFEKGGGSGGGSGGSAWSHPQFEKG | X1-DKENILQKIYEIMKTLEQLGHAEASMQVSDLIYEF |
| V1.3 | GSGSSGDKENILQKIYEIMKTLEQLGHAEASMQVSD | MKQGDERLLEEAERLLEEVER(SEQ ID NO: 8)- |
| XTEN | LIYEFMKQGDERLLEEAERLLEEVERGGSSAGSPTS | X2- |
| 3X | TGTSSATPSGSGTGGDKENILQKIYEIMKTLEQLGH | DKENILQKIYEIMKTLEQLGHAEASMQVSDLIYEF |
| | AEASMQVSDLIYEFMKQGDERLLEEAERLLEEVERG | MKQGDERLLEEAERLLEEVER(SEQ ID NO: 8)- |
| | GSSAGSPTSTGTSSATPSGSGTGGDKENILQKIYEI | X3- |
| | MKTLEQLGHAEASMQVSDLIYEFMKQGDERLLEEAE | DKENILQKIYEIMKTLEQLGHAEASMQVSDLIYEF |
| | RLLEEVER(SEQ ID NO: 220) | MKQGDERLLEEAERLLEEVER(SEQ ID NO: 8) |
| LCB1 | MEKKISAWSHPQFEKGGGSGGGSGGSAWSHPQFEKG | X1-DKENILQKIYEIMKTLEQLGHAEASMQVSDLIYEF |
| V1.3_ | GSGSSGDKENILQKIYEIMKTLEQLGHAEASMQVSD | MKQGDERLLEEAERLLEEVER(SEQ ID NO: 8)- |
| EAAAK | LIYEFMKQGDERLLEEAERLLEEVERGGSSGEAAAK | X2- |
| _3X | EAAAKEAAAKGSSGGDKENILQKIYEIMKTLEQLGH | DKENILQKIYEIMKTLEQLGHAEASMQVSDLIYEF |
| | AEASMQVSDLIYEFMKQGDERLLEEAERLLEEVERG | MKQGDERLLEEAERLLEEVER(SEQ ID NO: 8)- |
| | GSSGEAAAKEAAAKEAAAKGSSGGDKENILQKIYEI | X3- |
| | MKTLEQLGHAEASMQVSDLIYEFMKQGDERLLEEAE | DKENILQKIYEIMKTLEQLGHAEASMQVSDLIYEF |
| | RLLEEVER(SEQ ID NO: 221) | MKQGDERLLEEAERLLEEVER(SEQ ID NO: 8) |
| LCB1 | MEKKISAWSHPQFEKGGGSGGGSGGSAWSHPQFEKG | X1-DKENILQKIYEIMKTLEQLGHAEASMQVSDLIYEF |
| _v1.3_ | GSGSSGDKENILQKIYEIMKTLEQLGHAEASMQVSD | MKQGDERLLEEAERLLEEVER(SEQ ID NO: 8)- |
| Pro_3 | LIYEFMKQGDERLLEEAERLLEEVERGGSSGPSTPP | X2- |
| X | TPSPSTPPTPSPSPGGSSGDKENILQKIYEIMKTLE | DKENILQKIYEIMKTLEQLGHAEASMQVSDLIYEF |
| | QLGHAEASMQVSDLIYEFMKQGDERLLEEAERLLEE | MKQGDERLLEEAERLLEEVER(SEQ ID NO: 8)- |
| | VERGGSSGPSTPPTPSPSTPPTPSPSPGGSSGDKEN | X3- |
| | ILQKIYEIMKTLEQLGHAEASMQVSDLIYEFMKQGD | DKENILQKIYEIMKTLEQLGHAEASMQVSDLIYEF |
| | ERLLEEAERLLEEVER(SEQ ID NO: 222) | MKQGDERLLEEAERLLEEVER(SEQ ID NO: 8) |
| LCB1 | MEKKISAWSHPQFEKGGGSGGGSGGSAWSHPQFEKG | X1 |
| V1.3 | GSGSSGDKENILQKIYEIMKTLEQLGHAEASMQVSD | DKENILQKIYEIMKTLEQLGHAEASMQVSDLIY |
| Ub_3X | LIYEFMKQGDERLLEEAERLLEEVERGGSSGQIFVK | EFMKQGDERLLEEAERLLEEVER |
| | TLTGKTITLEVEPSDTIENVKAKIQDKEGIPPDQQR | (SEQ ID NO: 8}-X2- |
| | LIFAGKQLEDGRTLSDYNIQKESTLHLVLRLRGGGG | DKENILQKIYEIMKTLEQLGHAEASMQVSDLIYEF |
| | SSGDKENILQKIYEIMKTLEQLGHAEASMQVSDLIY | MKQGDERLLEEAERLLEEVER(SEQ ID NO: 8)- |
| | EFMKQGDERLLEEAERLLEEVERGGSSGQIFVKTLT | X3- |
| | GKTITLEVEPSDTIENVKAKIQDKEGIPPDQQRLIF | DKENILQKIYEIMKTLEQLGHAEASMQVSDLIYEF |
| | AGKQLEDGRTLSDYNIQKESTLHLVLRLRGGGGSSG | MKQGDERLLEEAERLLEEVER(SEQ ID NO: 8) |
| | DKENILQKIYEIMKTLEQLGHAEASMQVSDLIYEFM | |
| | KQGDERLLEEAERLLEEVER(SEQ ID NO: 223) | |
| 1GS1_ | MEKKISAWSHPQFEKGGGSGGGSGGSAWSHPQFEKG | X1- |
| NTS | GSGSSGDKENVLQKIYEIMKELERLGHAEASMQVSD | DKENVLQKIYEIMKELERLGHAEASMQVSDLIYEF |
| | LIYEFMKTKDENLLEEAERLLEEVKRGGSSGGGSSG | MKTKDENLLEEAERLLEEVKR(SEQ ID NO: 135) |
| | GGSSGGGSSGGGSSGDKENVLQKIYEIMKELERLGH | -X2- |
| | AEASMQVSDLIYEFMKTKDENLLEEAERLLEEVKR | DKENVLQKIYEIMKELERLGHAEASMQVSDLIYEF |
| | (SEQ ID NO: 224) | MKTKDENLLEEAERLLEEVKR(SEQ ID NO: 135) |
| 1Pro1 | MEKKISAWSHPQFEKGGGSGGGSGGSAWSHPQFEKG | X1- |
| _NTS | GSGSSGDKENVLQKIYEIMKELERLGHAEASMQVSD | DKENVLQKIYEIMKELERLGHAEASMQVSDLIYEF |
| | LIYEFMKTKDENLLEEAERLLEEVKRGGSSGPSTPP | MKTKDENLLEEAERLLEEVKR(SEQ ID NO: 135) |
| | TPSPSTPPTPSPSPGGSSGDKENVLQKIYEIMKELE | -X2- |
| | RLGHAEASMQVSDLIYEFMKTKDENLLEEAERLLEE | DKENVLQKIYEIMKELERLGHAEASMQVSDLIYEF |
| | VKR | MKTKDENLLEEAERLLEEVKR(SEQ ID NO: 135) |
| | (SEQ ID NO: 225) | |
| 3GS3_ | MEKKISAWSHPQFEKGGGSGGGSGGSAWSHPQFEKG | X1- |
| NTS | GSGSSGNLDELHMQMTDLVYEALHFAKDEEFQKHVF | NLDELHMQMTDLVYEALHFAKDEEFQKHVFQLFEK |
| | QLFEKATKAYKNKDRQKLEKVVEELKELLERLLSGG | ATKAYKNKDRQKLEKVVEELKELLERLLS |
| | SSGGGSSGGGSSGGGSSGGGSSGNLDELHMQMTDLV | (SEQ ID NO: 155) |
| | YEALHFAKDEEFQKHVFQLFEKATKAYKNKDRQKLE | -X2- |
| | KVVEELKELLERLLS | NLDELHMQMTDLVYEALHFAKDEEFQKHVFQLFEK |
| | (SEQ ID NO: 226) | ATKAYKNKDRQKLEKVVEELKELLERLLS |
| | | (SEQ ID NO: 155) |
| 3Pro3 | MEKKISAWSHPQFEKGGGSGGGSGGSAWSHPQFEKG | X1- |
| _NTS | GSGSSGNLDELHMQMTDLVYEALHFAKDEEFQKHVF | NLDELHMQMTDLVYEALHFAKDEEFQKHVFQLFEK |
| | QLFEKATKAYKNKDRQKLEKVVEELKELLERLLSGG | ATKAYKNKDRQKLEKVVEELKELLERLLS |
| | SSGPSTPPTPSPSTPPTPSPSPGGSSGNLDELHMQM | (SEQ ID NO: 155) |
| | TDLVYEALHFAKDEEFQKHVFQLFEKATKAYKNKDR | -X2- |
| | QKLEKVVEELKELLERLLS | NLDELHMQMTDLVYEALHFAKDEEFQKHVFQLFEK |
| | (SEQ ID NO: 227) | ATKAYKNKDRQKLEKVVEELKELLERLLS |
| | | (SEQ ID NO: 155) |
| 1GS1_ | MEKKIDKENVLQKIYEIMKELERLGHAEASMQVSDL | X1- |
| CTS | IYEFMKTKDENLLEEAERLLEEVKRGGSSGGGSSGG | DKENVLQKIYEIMKELERLGHAEASMQVSDLIYEF |
| | GSSGGGSSGGGSSGDKENVLQKIYEIMKELERLGHA | MKTKDENLLEEAERLLEEVKR(SEQ ID NO: 135) |
| | EASMQVSDLIYEFMKTKDENLLEEAERLLEEVKRGG | -X2- |
| | SGSSGSAWSHPQFEKGGGSGGGSGGSAWSHPQFEK | DKENVLQKIYEIMKELERLGHAEASMQVSDLIYEF |
| | (SEQ ID NO: 228) | MKTKDENLLEEAERLLEEVKR |
| | | (SEQ ID NO: 135)-X3 |
| 1Pro1 | MEKKIDKENVLQKIYEIMKELERLGHAEASMQVSDL | X1- |
| _CTS | IYEFMKTKDENLLEEAERLLEEVKRGGSSGPSTPPT | DKENVLQKIYEIMKELERLGHAEASMQVSDLIYEF |
| | PSPSTPPTPSPSPGGSSGDKENVLQKIYEIMKELER | MKTKDENLLEEAERLLEEVKR(SEQ ID NO: 135) |
| | LGHAEASMQVSDLIYEFMKTKDENLLEEAERLLEEV | -X2- |
| | KRGGSGSSGSAWSHPQFEKGGGSGGGSGGSAWSHPQ | DKENVLQKIYEIMKELERLGHAEASMQVSDLIYEF |
| | FEK | MKTKDENLLEEAERLLEEVKR |
| | (SEQ ID NO: 229) | (SEQ ID NO: 135)-X3 |
| 3GS3 | MEKKINLDELHMQMTDLVYEALHFAKDEEFQKHVFQ | X1- |
| _CTS | LFEKATKAYKNKDRQKLEKVVEELKELLERLLSGGS | NLDELHMQMTDLVYEALHFAKDEEFQKHVFQLFEK |
| | SGGGSSGGGSSGGGSSGGGSSGNLDELHMQMTDLVY | ATKAYKNKDRQKLEKVVEELKELLERLLS |
| | EALHFAKDEEFQKHVFQLFEKATKAYKNKDRQKLEK | (SEQ ID NO: 155) |
| | WEELKELLERLLSGGSGSSGSAWSHPQFEKGGGSG | -X2- |
| | GGSGGSAWSHPQFEK | NLDELHMQMTDLVYEALHFAKDEEFQKHVFQLFEK |
| | (SEQ ID NO: 230) | ATKAYKNKDRQKLEKVVEELKELLERLLS |
| | | (SEQ ID NO: 155)-X3 |
| 3Pro3 | MEKKINLDELHMQMTDLVYEALHFAKDEEFQKHVFQ | X1- |
| _CTS | LFEKATKAYKNKDRQKLEKVVEELKELLERLLSGGS | NLDELHMQMTDLVYEALHFAKDEEFQKHVFQLFEK |
| | SGPSTPPTPSPSTPPTPSPSPGGSSGNLDELHMQMT | ATKAYKNKDRQKLEKVVEELKELLERLLS |
| | DLVYEALHFAKDEEFQKHVFQLFEKATKAYKNKDRQ | (SEQ ID NO: 155) |
| | KLEKVVEELKELLERLLSGGSGSSGSAWSHPQFEKG | -X2- |
| | GGSGGGSGGSAWSHPQFEK | NLDELHMQMTDLVYEALHFAKDEEFQKHVFQLFEK |
| | (SEQ ID NO: 231) | ATKAYKNKDRQKLEKVVEELKELLERLLS |
| | | (SEQ ID NO: 155)-X3 |
| 1GS1G | MEKKISAWSHPQFEKGGSGSSGDKENVLQKIYEIMK | X1- |
| S1_NT | ELERLGHAEASMQVSDLIYEFMKTKDENLLEEAERL | DKENVLQKIYEIMKELERLGHAEASMQVSDLIYEF |
| S | LEEVKRGGSSGGGSSGGGSSGGGSSGGGSSGDKENV | MKTKDENLLEEAERLLEEVKR |
| | LQKIYEIMKELERLGHAEASMQVSDLIYEFMKTKDE | (SEQ ID NO: 135)-X2- |
| | NLLEEAERLLEEVKRGGSSGGGSSGGGSSGGGSSGG | DKENVLQKIYEIMKELERLGHAEASMQVSDLIYEF |
| | GSSGDKENVLQKIYEIMKELERLGHAEASMQVSDLI | MKTKDENLLEEAERLLEEVKR |
| | YEFMKTKDENLLEEAERLLEEVKR | (SEQ ID NO: 135)-X3- |
| | (SEQ ID NO: 232) | DKENVLQKIYEIMKELERLGHAEASMQVSDLIYEF |
| | | MKTKDENLLEEAERLLEEVKR |
| | | (SEQ ID NO: 135) |
| 1Pro1 | MEKKISAWSHPQFEKGGSGSSGDKENVLQKIYEIMK | X1- |
| Pro1_ | ELERLGHAEASMQVSDLIYEFMKTKDENLLEEAERL | DKENVLQKIYEIMKELERLGHAEASMQVSDLIYEF |
| NTS | LEEVKRGGSSGPSTPPTPSPSTPPTPSPSPGGSSGD | MKTKDENLLEEAERLLEEVKR |
| | KENVLQKIYEIMKELERLGHAEASMQVSDLIYEFMK | (SEQ ID NO: 135)-X2- |
| | TKDENLLEEAERLLEEVKRGGSSGPSTPPTPSPSTP | DKENVLQKIYEIMKELERLGHAEASMQVSDLIYEF |
| | PTPSPSPGGSSGDKENVLQKIYEIMKELERLGHAEA | MKTKDENLLEEAERLLEEVKR |
| | SMQVSDLIYEFMKTKDENLLEEAERLLEEVKR | (SEQ ID NO: 135)-X3- |
| | (SEQ ID NO: 233) | DKENVLQKIYEIMKELERLGHAEASMQVSDLIYEF |
| | | MKTKDENLLEEAERLLEEVKR |
| | | (SEQ ID NO: 135) |
| 3GS3G | MEKKISAWSHPQFEKGGSGSSGNLDELHMQMTDLVY | X1- |
| S3_NTS | EALHFAKDEEFQKHVFQLFEKATKAYKNKDRQKLEK | NLDELHMQMTDLVYEALHFAKDEEFQKHVFQLFEK |
| | VVEELKELLERLLSGGSSGGGSSGGGSSGGGSSGGG | ATKAYKNKDRQKLEKVVEELKELLERLLS |
| | SSGNLDELHMQMTDLVYEALHFAKDEEFQKHVFQLF | (SEQ ID NO: 155)-X2- |
| | EKATKAYKNKDRQKLEKVVEELKELLERLLSGGSSG | NLDELHMQMTDLVYEALHFAKDEEFQKHVFQLFEK |
| | GGSSGGGSSGGGSSGGGSSGNLDELHMQMTDLVYEA | ATKAYKNKDRQKLEKVVEELKELLERLLS |
| | LHFAKDEEFQKHVFQLFEKATKAYKNKDRQKLEKW | (SEQ ID NO: 155)-X3- |
| | EELKELLERLLS | NLDELHMQMTDLVYEALHFAKDEEFQKHVFQLFEK |
| | (SEQ ID NO: 234) | ATKAYKNKDRQKLEKVVEELKELLERLLS |
| | | (SEQ ID NO: 155) |
| 3Pro3 | MEKKISAWSHPQFEKGGSGSSGNLDELHMQMTDLVY | X1- |
| Pro3_ | EALHFAKDEEFQKHVFQLFEKATKAYKNKDRQKLEK | NLDELHMQMTDLVYEALHFAKDEEFQKHVFQLFEK |
| NTS | VVEELKELLERLLSGGSSGPSTPPTPSPSTPPTPSP | ATKAYKNKDRQKLEKVVEELKELLERLLS |
| | SPGGSSGNLDELHMQMTDLVYEALHFAKDEEFQKHV | (SEQ ID NO: 155)-X2- |
| | FQLFEKATKAYKNKDRQKLEKVVEELKELLERLLSG | NLDELHMQMTDLVYEALHFAKDEEFQKHVFQLFEK |
| | GSSGPSTPPTPSPSTPPTPSPSPGGSSGNLDELHMQ | ATKAYKNKDRQKLEKVVEELKELLERLLS |
| | MTDLVYEALHFAKDEEFQKHVFQLFEKATKAYKNKD | (SEQ ID NO: 155)-X3- |
| | RQKLEKVVEELKELLERLLS | NLDELHMQMTDLVYEALHFAKDEEFQKHVFQLFEK |
| | (SEQ ID NO: 235) | ATKAYKNKDRQKLEKVVEELKELLERLLS |
| | | (SEQ ID NO: 155) |
| 1GS1G | MEKKIDKENVLQKIYEIMKELERLGHAEASMQVSDL | X1- |
| S1_CT | IYEFMKTKDENLLEEAERLLEEVKRGGSSGGGSSGG | DKENVLQKIYEIMKELERLGHAEASMQVSDLIYEF |
| S | GSSGGGSSGGGSSGDKENVLQKIYEIMKELERLGHA | MKTKDENLLEEAERLLEEVKR |
| | EASMQVSDLIYEFMKTKDENLLEEAERLLEEVKRGG | (SEQ ID NO: 135)-X2- |
| | SSGGGSSGGGSSGGGSSGGGSSGDKENVLQKIYEIM | DKENVLQKIYEIMKELERLGHAEASMQVSDLIYEF |
| | KELERLGHAEASMQVSDLIYEFMKTKDENLLEEAER | MKTKDENLLEEAERLLEEVKR |
| | LLEEVKRGGSGSSGSAWSHPQFEK | (SEQ ID NO: 135)-X3- |
| | (SEQ ID NO: 236) | DKENVLQKIYEIMKELERLGHAEASMQVSDLIYEF |
| | | MKTKDENLLEEAERLLEEVKR |
| | | (SEQ ID NO: 135)-X4 |
| 1Pro1 | MEKKIDKENVLQKIYEIMKELERLGHAEASMQVSDL | X1- |
| Pro1_ | IYEFMKTKDENLLEEAERLLEEVKRGGSSGPSTPPT | DKENVLQKIYEIMKELERLGHAEASMQVSDLIYEF |
| CTS | PSPSTPPTPSPSPGGSSGDKENVLQKIYEIMKELER | MKTKDENLLEEAERLLEEVKR |
| | LGHAEASMQVSDLIYEFMKTKDENLLEEAERLLEEV | (SEQ ID NO: 135)-X2- |
| | KRGGSSGPSTPPTPSPSTPPTPSPSPGGSSGDKENV | DKENVLQKIYEIMKELERLGHAEASMQVSDLIYEF |
| | LQKIYEIMKELERLGHAEASMQVSDLIYEFMKTKDE | MKTKDENLLEEAERLLEEVKR |
| | NLLEEAERLLEEVKRGGSGSSGSAWSHPQFEK | (SEQ ID NO: 135)-X3- |
| | (SEQ ID NO: 237) | DKENVLQKIYEIMKELERLGHAEASMQVSDLIYEF |
| | | MKTKDENLLEEAERLLEEVKR |
| | | (SEQ ID NO: 135)-X4 |
| 3GS3G | MEKKINLDELHMQMTDLVYEALHFAKDEEFQKHVFQ | X1- |
| S3_CT | LFEKATKAYKNKDRQKLEKVVEELKELLERLLSGGS | NLDELHMQMTDLVYEALHFAKDEEFQKHVFQLFEK |
| S | SGGGSSGGGSSGGGSSGGGSSGNLDELHMQMTDLVY | ATKAYKNKDRQKLEKVVEELKELLERLLS |
| | EALHFAKDEEFQKHVFQLFEKATKAYKNKDRQKLEK | (SEQ ID NO: 155)-X2- |
| | VVEELKELLERLLSGGSSGGGSSGGGSSGGGSSGGG | NLDELHMQMTDLVYEALHFAKDEEFQKHVFQLFEK |
| | SSGNLDELHMQMTDLVYEALHFAKDEEFQKHVFQLF | ATKAYKNKDRQKLEKVVEELKELLERLLS |
| | EKATKAYKNKDRQKLEKVVEELKELLERLLSGGSGS | (SEQ ID NO: 155)-X3- |
| | SGSAWSHPQFEK(SEQ ID NO: 238) | NLDELHMQMTDLVYEALHFAKDEEFQKHVFQLFEK |
| | | ATKAYKNKDRQKLEKVVEELKELLERLLS |
| | | (SEQ ID NO: 155)-X4 |
| | | X1- |
| 3Pro3 | MEKKINLDELHMQMTDLVYEALHFAKDEEFQKHVFQ | NLDELHMQMTDLVYEALHFAKDEEFQKHVFQLFEK |
| Pro3_ | LFEKATKAYKNKDRQKLEKVVEELKELLERLLSGGS | ATKAYKNKDRQKLEKVVEELKELLERLLS |
| CTS | SGPSTPPTPSPSTPPTPSPSPGGSSGNLDELHMQMT | (SEQ ID NO: 155)-X2- |
| | DLVYEALHFAKDEEFQKHVFQLFEKATKAYKNKDRQ | NLDELHMQMTDLVYEALHFAKDEEFQKHVFQLFEK |
| | KLEKVVEELKELLERLLSGGSSGPSTPPTPSPSTPP | ATKAYKNKDRQKLEKVVEELKELLERLLS |
| | TPSPSPGGSSGNLDELHMQMTDLVYEALHFAKDEEF | (SEQ ID NO: 155)-X3- |
| | QKHVFQLFEKATKAYKNKDRQKLEKVVEELKELLER | NLDELHMQMTDLVYEALHFAKDEEFQKHVFQLFEK |
| | LLSGGSGSSGSAWSHPQFEK(SEQ ID NO: 239) | ATKAYKNKDRQKLEKVVEELKELLERLLS |
| | | (SEQ ID NO: 155)-X4 |
| 1GS3_ | MEKKISAWSHPQFEKGGGSGGGSGGSAWSHPQFEKG | X1- |
| NTS | GSGSSGDKENVLQKIYEIMKELERLGHAEASMQVSD | DKENVLQKIYEIMKELERLGHAEASMQVSDLIYEF |
| | LIYEFMKTKDENLLEEAERLLEEVKRGGSSGGGSSG | MKTKDENLLEEAERLLEEVKR(SEQ ID NO: 135) |
| | GGSSGGGSSGGGSSGNLDELHMQMTDLVYEALHFAK | -X2- |
| | DEEFQKHVFQLFEKATKAYKNKDRQKLEKVVEELKE | NLDELHMQMTDLVYEALHFAKDEEFQKHVFQLFEK |
| | LLERLLS(SEQ ID NO: 240) | ATKAYKNKDRQKLEKVVEELKELLERLLS |
| | | (SEQ ID NO: 155) |
| 1Pro3_ | MEKKISAWSHPQFEKGGGSGGGSGGSAWSHPQFEKG | X1- |
| NTS | GSGSSGDKENVLQKIYEIMKELERLGHAEASMQVSD | DKENVLQKIYEIMKELERLGHAEASMQVSDLIYEF |
| | LIYEFMKTKDENLLEEAERLLEEVKRGGSSGPSTPP | MKTKDENLLEEAERLLEEVKR(SEQ ID NO: 135) |
| | TPSPSTPPTPSPSPGGSSGNLDELHMQMTDLVYEAL | X-2- |
| | HFAKDEEFQKHVFQLFEKATKAYKNKDRQKLEKWE | NLDELHMQMTDLVYEALHFAKDEEFQKHVFQLFEK |
| | ELKELLERLLS(SEQ ID NO: 241) | ATKAYKNKDRQKLEKVVEELKELLERLLS |
| | | (SEQ ID NO: 155) |
| 3GS1_ | MEKKISAWSHPQFEKGGGSGGGSGGSAWSHPQFEKG | X1- |
| NTS | GSGSSGNLDELHMQMTDLVYEALHFAKDEEFQKHVF | NLDELHMQMTDLVYEALHFAKDEEFQKHVFQLFEK |
| | QLFEKATKAYKNKDRQKLEKVVEELKELLERLLSGG | ATKAYKNKDRQKLEKVVEELKELLERLLS |
| | SSGGGSSGGGSSGGGSSGGGSSGDKENVLQKIYEIM | (SEQ ID NO: 155)-X2- |
| | KELERLGHAEASMQVSDLIYEFMKTKDENLLEEAER | DKENVLQKIYEIMKELERLGHAEASMQVSDLIYEF |
| | LLEEVKR(SEQ ID NO: 242) | MKTKDENLLEEAERLLEEVKR(SEQ ID NO: 135) |
| 3Pro1_ | MEKKISAWSHPQFEKGGGSGGGSGGSAWSHPQFEKG | X1- |
| NTS | GSGSSGNLDELHMQMTDLVYEALHFAKDEEFQKHVF | NLDELHMQMTDLVYEALHFAKDEEFQKHVFQLFEK |
| | QLFEKATKAYKNKDRQKLEKVVEELKELLERLLSGG | ATKAYKNKDRQKLEKVVEELKELLERLLS |
| | SSGPSTPPTPSPSTPPTPSPSPGGSSGDKENVLQKI | (SEQ ID NO: 155)-X2- |
| | YEIMKELERLGHAEASMQVSDLIYEFMKTKDENLLE | DKENVLQKIYEIMKELERLGHAEASMQVSDLIYEF |
| | EAERLLEEVKR(SEQ ID NO: 243) | MKTKDENLLEEAERLLEEVKR(SEQ ID NO: 135) |
| 1GS3_ | MEKKIDKENVLQKIYEIMKELERLGHAEASMQVSDL | X1- |
| CTS | IYEFMKTKDENLLEEAERLLEEVKRGGSSGGGSSGG | DKENVLQKIYEIMKELERLGHAEASMQVSDLIYEF |
| | GSSGGGSSGGGSSGNLDELHMQMTDLVYEALHFAKD | MKTKDENLLEEAERLLEEVKR(SEQ ID NO: 135)- |
| | EEFQKHVFQLFEKATKAYKNKDRQKLEKVVEELKEL | X2- |
| | LERLLSGGSGSSGSAWSHPQFEKGGGSGGGSGGSAW | NLDELHMQMTDLVYEALHFAKDEEFQKHVFQLFEK |
| | SHPQFEK(SEQ ID NO: 244) | ATKAYKNKDRQKLEKVVEELKELLERLLS |
| | | (SEQ ID NO: 155)-X3 |
| 1Pro3_ | MEKKIDKENVLQKIYEIMKELERLGHAEASMQVSDL | X1- |
| CTS | IYEFMKTKDENLLEEAERLLEEVKRGGSSGPSTPPT | DKENVLQKIYEIMKELERLGHAEASMQVSDLIYEF |
| | PSPSTPPTPSPSPGGSSGNLDELHMQMTDLVYEALH | MKTKDENLLEEAERLLEEVKR(SEQ ID NO: 135)- |
| | FAKDEEFQKHVFQLFEKATKAYKNKDRQKLEKVVEE | X2- |
| | LKELLERLLSGGSGSSGSAWSHPQFEKGGGSGGGSG | NLDELHMQMTDLVYEALHFAKDEEFQKHVFQLFEK |
| | GSAWSHPQFEK(SEQ ID NO: 245) | ATKAYKNKDRQKLEKVVEELKELLERLLS |
| | | (SEQ ID NO: 155)-X3 |
| 3GS1_ | MEKKINLDELHMQMTDLVYEALHFAKDEEFQKHVFQ | X1- |
| CTS | LFEKATKAYKNKDRQKLEKVVEELKELLERLLSGGS | NLDELHMQMTDLVYEALHFAKDEEFQKHVFQLFEK |
| | SGGGSSGGGSSGGGSSGGGSSGDKENVLQKIYEIMK | ATKAYKNKDRQKLEKVVEELKELLERLLS |
| | ELERLGHAEASMQVSDLIYEFMKTKDENLLEEAERL | (SEQ ID NO: 155)-X2- |
| | LEEVKRGGSGSSGSAWSHPQFEKGGGSGGGSGGSAW | DKENVLQKIYEIMKELERLGHAEASMQVSDLIYEF |
| | SHPQFEK | MKTKDENLLEEAERLLEEVKR |
| | (SEQ ID NO: 246) | (SEQ ID NO: 135)-X3 |
| 3Pro1 | MEKKINLDELHMQMTDLVYEALHFAKDEEFQKHVFQ | X1- |
| _CTS | LFEKATKAYKNKDRQKLEKVVEELKELLERLLSGGS | NLDELHMQMTDLVYEALHFAKDEEFQKHVFQLFEK |
| | SGPSTPPTPSPSTPPTPSPSPGGSSGDKENVLQKIY | ATKAYKNKDRQKLEKVVEELKELLERLLS |
| | EIMKELERLGHAEASMQVSDLIYEFMKTKDENLLEE | (SEQ ID NO: 155)-X2- |
| | AERLLEEVKRGGSGSSGSAWSHPQFEKGGGSGGGSG | DKENVLQKIYEIMKELERLGHAEASMQVSDLIYEF |
| | GSAWSHPQFEK | MKTKDENLLEEAERLLEEVKR |
| | (SEQ ID NO: 247) | (SEQ ID NO: 135)-X3 |
| 3- | MEKKISAWSHPQFEKGGGSGGGSGGSAWSHPQFEKG | X1- |
| GS10- | GSGSSGNLDELHMQMTDLVYEALHFAKDEEFQKHVF | NLDELHMQMTDLVYEALHFAKDEEFQKHVFQLFEK |
| 1- | QLFEKATKAYKNKDRQKLEKVVEELKELLERLLSGG | ATKAYKNKDRQKLEKVVEELKELLERLLS |
| L_NTS | GSGGGSGGDKENVLQKIYEIMKELERLGHAEASMQV | (SEQ ID NO: 155)-X2- |
| | SDLIYEFMKTKDENLLEEAERLLEEVKR | DKENVLQKIYEIMKELERLGHAEASMQVSDLIYEF |
| | (SEQ ID NO: 248) | MKTKDENLLEEAERLLEEVKR |
| | | (SEQ ID NO: 135) |
| 3- | MEKKISAWSHPQFEKGGGSGGGSGGSAWSHPQFEKG | X1- |
| GS10- | GSGSSGNLDELHMQMTDLVYEALHFAKDEEFQKHVF | )NLDELHMQMTDLVYEALHFAKDEEFQKHVFQLFE |
| 1_NTS | QLFEKATKAYKNKDRQKLEKVVEELKELLERLLSGG | KATKAYKNKDRQKLEKVVEELKELLERLLS |
| | SSGGGSSGDKENVLQKIYEIMKELERLGHAEASMQV | (SEQ ID NO: 155)-X2- |
| | SDLIYEFMKTKDENLLEEAERLLEEVKR | DKENVLQKIYEIMKELERLGHAEASMQVSDLIYEF |
| | (SEQ ID NO: 249) | MKTKDENLLEEAERLLEEVKR |
| | | (SEQ ID NO: 135) |
| 3- | MEKKISAWSHPQFEKGGGSGGGSGGSAWSHPQFEKG | X1- |
| GS15- | GSGSSGNLDELHMQMTDLVYEALHFAKDEEFQKHVF | NLDELHMQMTDLVYEALHFAKDEEFQKHVFQLFEK |
| 1_NTS | QLFEKATKAYKNKDRQKLEKVVEELKELLERLLSGG | ATKAYKNKDRQKLEKVVEELKELLERLLS |
| | SSGGGSSGGGSSGDKENVLQKIYEIMKELERLGHAE | (SEQ ID NO: 155)-X2- |
| | ASMQVSDLIYEFMKTKDENLLEEAERLLEEVKR | DKENVLQKIYEIMKELERLGHAEASMQVSDLIYEF |
| | (SEQ ID NO: 250) | MKTKDENLLEEAERLLEEVKR |
| | | (SEQ ID NO: 135) |
| 3- | MEKKISAWSHPQFEKGGGSGGGSGGSAWSHPQFEKG | X1- |
| GS20- | GSGSSGNLDELHMQMTDLVYEALHFAKDEEFQKHVF | NLDELHMQMTDLVYEALHFAKDEEFQKHVFQLFEK |
| 1_NTS | QLFEKATKAYKNKDRQKLEKVVEELKELLERLLSGG | ATKAYKNKDRQKLEKVVEELKELLERLLS |
| | SSGGGSSGGGSSGGGSSGDKENVLQKIYEIMKELER | (SEQ ID NO: 155)-X2- |
| | LGHAEASMQVSDLIYEFMKTKDENLLEEAERLLEEV | DKENVLQKIYEIMKELERLGHAEASMQVSDLIYEF |
| | KR | MKTKDENLLEEAERLLEEVKR |
| | (SEQ ID NO: 251) | (SEQ ID NO: 135) |
| 1- | MEKKISAWSHPQFEKGGGSGGGSGGSAWSHPQFEKG | X1- |
| GS10- | GSGSSGDKENVLQKIYEIMKELERLGHAEASMQVSD | DKENVLQKIYEIMKELERLGHAEASMQVSDLIYEF |
| 1_NTS | LIYEFMKTKDENLLEEAERLLEEVKRGGSSGGGSSG | MKTKDENLLEEAERLLEEVKR |
| | DKENVLQKIYEIMKELERLGHAEASMQVSDLIYEFM | (SEQ ID NO: 135)-X2- |
| | KTKDENLLEEAERLLEEVKR | DKENVLQKIYEIMKELERLGHAEASMQVSDLIYEF |
| | (SEQ ID NO: 252) | MKTKDENLLEEAERLLEEVKR |
| | | (SEQ ID NO: 135) |
| 1- | MEKKISAWSHPQFEKGGGSGGGSGGSAWSHPQFEKG | X1- |
| GS15- | GSGSSGDKENVLQKIYEIMKELERLGHAEASMQVSD | DKENVLQKIYEIMKELERLGHAEASMQVSDLIYEF |
| 1_NTS | LIYEFMKTKDENLLEEAERLLEEVKRGGSSGGGSSG | MKTKDENLLEEAERLLEEVKR |
| | GGSSGDKENVLQKIYEIMKELERLGHAEASMQVSDL | (SEQ ID NO: 135)-X2- |
| | IYEFMKTKDENLLEEAERLLEEVKR | DKENVLQKIYEIMKELERLGHAEASMQVSDLIYEF |
| | (SEQ ID NO: 253) | MKTKDENLLEEAERLLEEVKR |
| | | (SEQ ID NO: 135) |
| 1- | MEKKISAWSHPQFEKGGGSGGGSGGSAWSHPQFEKG | X1-DKENVLQKIYEIMKELERLGHAEASMQVSDLIYEF |
| GS20- | GSGSSGDKENVLQKIYEIMKELERLGHAEASMQVSD | MKTKDENLLEEAERLLEEVKR |
| 1_NTS | LIYEFMKTKDENLLEEAERLLEEVKRGGSSGGGSSG | (SEQ ID NO: 135)-X2- |
| | GGSSGGGSSGDKENVLQKIYEIMKELERLGHAEASM | DKENVLQKIYEIMKELERLGHAEASMQVSDLIYEF |
| | QVSDLIYEFMKTKDE1NLLEEAERLLEEVKR | MKTKDENLLEEAERLLEEVKR |
| | (SEQ ID NO: 254) | (SEQ ID NO: 135) |
| 2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS10 | RAAYFNWWATEMMLELIKSDDEREIREIEEEARRIL | NWWATEMMLELIKSDDEREIREIEEEARRILEHLE |
| | EHLEELARKGGSSGGGSSGDKENVLQKIYEIMKELE | ELARK |
| | | (SEQ ID NO: 101)-X2- |
| | RLGHAEASMQVSDLIYEFMKTKDERLLEEAERLLEE | DKENVLQKIYEIMKELERLGHAEASMQVSDLIYEF |
| | VKR(SEQ ID NO: 255) | MKTKDERLLEEAERLLEEVKR |
| | | (SEQ ID NO: 125) |
| 2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS15 | RAAYFNWWATEMMLELIKSDDEREIREIEEEARRIL | NWWATEMMLELIKSDDEREIREIEEEARRILEHLE |
| | EHLEELARKGGSSGGGSSGGGSSGDKENVLQKIYEI | ELARK |
| | MKELERLGHAEASMQVSDLIYEFMKTKDERLLEEAE | (SEQ ID NO: 101)- |
| | RLLEEVKR(SEQ ID NO: 256) | X2-DKENVLQKIYEIMKELERLGHAEASMQVSDLIYEF |
| | | MKTKDERLLEEAERLLEEVKR |
| | | (SEQ ID NO: 125) |
| 2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS20 | RAAYFNWWATEMMLELIKSDDEREIREIEEEARRIL | NWWATEMMLELIKSDDEREIREIEEEARRILEHLE |
| | EHLEELARKGGSSGGGSSGGGSSGGGSSGDKENVLQ | ELARK |
| | KIYEIMKELERLGHAEASMQVSDLIYEFMKTKDERL | (SEQ ID NO: 101)- |
| | LEEAERLLEEVKR | X2-DKENVLQKIYEIMKELERLGHAEASMQVSDLIYEF |
| | (SEQ ID NO: 257) | MKTKDERLLEEAERLLEEVKR |
| | | (SEQ ID NO: 125) |
| 2-2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS10 | RAAYFNWWATEMMLELIKSDDEREIREIEEEARRIL | NWWATEMMLELIKSDDEREIREIEEEARRILEHLE |
| | EHLEELARKGGSSGGGSSGELEEQVMHVLDQVSELA | ELARK(SEQ ID NO: 101)- |
| | HELLHKLTGEELERAAYFNWWATEMMLELIKSDDER | X2-ELEEQVMHVLDQVSELAHELLHKLTGEELERAAYF |
| | ElREIEEEARRILEHLEELARKGGSSGGGSSGDKEN | NWWATEMMLELIKSDDEREIREIEEEARRILEHLE |
| | VLQKIYEIMKELERLGHAEASMQVSDLIYEFMKTKD | ELARK(SEQ ID NO: 101)- |
| | ERLLEEAERLLEEVKR(SEQ ID NO: 258) | X3 |
| 2-2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS15 | RAAYFNWWATEMMLELIKSDDEREIREIEEEARRIL | NWWATEMMLELIKSDDEREIREIEEEARRILEHLE |
| | EHLEELARKGGSSGGGSSGGGSSGELEEQVMHVLDQ | ELARK(SEQ ID NO: 101)- |
| | VSELAHELLHKLTGEELERAAYFNWWATEMMLELIK | X2-ELEEQVMHVLDQVSELAHELLHKLTGEELERAAYF |
| | SDDEREIREIEEEARRILEHLEELARKGGSSGGGSS | NWWATEMMLELIKSDDEREIREIEEEARRILEHLE |
| | GGGSSGDKENVLQKIYEIMKELERLGHAEASMQVSD | ELARK(SEQ ID NO: 101)- |
| | LIYEFMKTKDERLLEEAERLLEEVKR | X3 |
| | (SEQ ID NO: 259) | |
| 2-2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS20 | RAAYFNWWATEMMLELIKSDDEREIREIEEEARRIL | NWWATEMMLELIKSDDEREIREIEEEARRILEHLE |
| | EHLEELARKGGSSGGGSSGGGSSGGGSSGELEEQVM | ELARK(SEQ ID NO: 101)- |
| | HVLDQVSELAHELLHKLTGEELERAAYFNWWATEMM | X2-ELEEQVMHVLDQVSELAHELLHKLTGEELERAAYF |
| | LELIKSDDEREIREIEEEARRILEHLEELARKGGSS | NWWATEMMLELIKSDDEREIREIEEEARRILEHLE |
| | GGGSSGGGSSGGGSSGDKENVLQKIYEIMKELERLG | ELARK(SEQ ID NO: 101)- |
| | HAEASMQVSDLIYEFMKTKDERLLEEAERLLEEVKR | X3 |
| | (SEQ ID NO: 260) | |
| 2-2- | MSKIKSAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYF |
| GS10 | RAAYFNWWATEMMLELIKSDDEREIREIEEEARRIL | NWWATEMMLELIKSDDEREIREIEEEARRILEHLE |
| | EHLEELARKGGSSGGGSSGELEEQVMHVLDQVSELA | ELARK(SEQ ID NO: 101)- |
| | HELLHKLTGEELERAAYFNWWATEMMLELIKSDDER | X2-ELEEQVMHVLDQVSELAHELLHKLTGEELERAAYF |
| | EIREIEEEARRILEHLEELARK(SEQ ID NO: | NWWATEMMLELIKSDDEREIREIEEEARRILEHLE |
| | 261) | ELARK(SEQ ID NO: 101) |
| 2- | MSKIKSAWSHPQFEKGGGSGGGSGGSAWSHPQFEKG | X1- |
| 2_ | GSGSSGELEEQVMHVLDQVSELAHELLHKLTGEELE | ELEEQVMHVLDQVSELAHELLHKLTGEELERAAYF |
| GS15 | RAAYFNWWATEMMLELIKSDDEREIREIEEEARRIL | NWWATEMMLELIKSDDEREIREIEEEARRILEHLE |
| | EHLEELARKGGSSGGGSSGGGSSGELEEQVMHVLDQ | ELARK(SEQ ID NO: 101)- |
| | VSELAHELLHKLTGEELERAAYFNWWATEMMLELIK | X2-ELEEQVMHVLDQVSELAHELLHKLTGEELERAAYF |
| | SDDEREIREIEEEARRILEHLEELARK | NWWATEMMLELIKSDDEREIREIEEEARRILEHLE |
| | (SEQ ID NO: 262) | ELARK(SEQ ID NO: 101) |
| 2- | MSKIKSAWSHPQFEKGGGSGGGSGGSAWSHPQFEKG | X1- |
| 2_ | GSGSSGELEEQVMHVLDQVSELAHELLHKLTGEELE | ELEEQVMHVLDQVSELAHELLHKLTGEELERAAYF |
| GS10 | RAAYFNWWATEMMLELIKSDDEREIREIEEEARRIL | NWWATEMMLELIKSDDEREIREIEEEARRILEHLE |
| | EHLEELARKGGSSGGGSSGGGSSGGGSSGELEEQVM | ELARK(SEQ ID NO: 101)- |
| | HVLDQVSELAHELLHKLTGEELERAAYFNWWATEMM | X2-ELEEQVMHVLDQVSELAHELLHKLTGEELERAAYF |
| | LELIKSDDEREIREIEEEARRILEHLEELARK(SEQ | NWWATEMMLELIKSDDEREIREIEEEARRILEHLE |
| | ID NO: 263) | ELARK(SEQ ID NO: 101) |
| 2-2-2_ | MSKIKSAWSHPQFEKGGGSGGGSGGSAWSHPQFEKG | X1-ELEEQVMHVLDQVSELAHELLHKLTGEELERAA |
| GS10 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEARRIL | EELARK(SEQ ID NO: 101)-X2-ELEEQVMHVL |
| | EHLEELARKGGSSGGGSSGELEEQVMHVLDQVSELA | DQVSELAHELLHKLTGEELERAAYFNWWATEMMLEL |
| | HELLHKLTGEELERAAYFNWWATEMMLELIKSDDER | IKSDDEREIREIEEEARRILEHLEELARK(SEQ ID |
| | EIREIEEEARRILEHLEELARKGGSSGGGSSGELEE | NO: 101)-X3-ELEEQVMHVLDQVSELAHELLHK |
| | QVMHVLDQVSELAHELLHKLTGEELERAAYFNWWAT | LTGEELERAAYFNWWATEMMLELIKSDDEREIREIE |
| | EMMLELIKSDDEREIREIEEEARRILEHLEELARK | EEARRILEHLEELARK(SEQ ID NO: 101) |
| | (SEQ ID NO: 264) | |
| 2-2-2_ | MSKIKSAWSHPQFEKGGGSGGGSGGSAWSHPQFEKG | X1-ELEEQVMHVLDQVSELAHELLHKLTGEELERAA |
| GS15 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEIARR | EELARK(SEQ ID NO: 101)-X2-ELEEQVMHVL |
| | ILEHLEELARKGGSSGGGSSGGGSSGELEEQVMHVL | DQVSELAHELLHKLTGEELERAAYFNWWATEMMLEL |
| | DQVSELAHELLHKLTGEELERAAYFNWWATEMMLEL | IKSDDEREIREIEEEARRILEHLEELARK(SEQ ID |
| | IKSDDEREIREIEEEARRILEHLEELARKGGSSGGG | NO: 101)-X3-ELEEQVMHVLDQVSELAHELLHK |
| | SSGGGSSGELEEQVMHVLDQVSELAHELLHKLTGEE | LTGEELERAAYFNWWATEMMLELIKSDDEREIREIE |
| | LERAAYFNWWATEMMLELIKSDDEREIREIEEEARR | EEARRILEHLEELARK(SEQ ID NO: 101) |
| | ILEHLEELARK(SEQ ID NO: 265) | |
| 2-2-2_ | MSKIKSAWSHPQFEKGGGSGGGSGGSAWSHPQFEKG | X1-ELEEQVMHVLDQVSELAHELLHKLTGEELERAA |
| GS20 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEARRIL | EELARK(SEQ ID NO: 101)-X2-ELEEQVMHVL |
| | EHLEELARKGGSSGGGSSGGGSSGGGSSGELEEQVM | DQVSELAHELLHKLTGEELERAAYFNWWATEMMLEL |
| | HVLDQVSELAHELLHKLTGEELERAAYFNWWATEMM | IKSDDEREIREIEEEARRILEHLEELARK(SEQ ID |
| | LELIKSDDEREIREIEEEARRILEHLEELARKGGSS | NO: 101)-X3-ELEEQVMHVLDQVSELAHELLHK |
| | GGGSSGGGSSGGGSSGELEEQVMHVLDQVSELAHEL | LTGEELERAAYFNWWATEMMLELIKSDDEREIREIE |
| | LHKLTGEELERAAYFNWWATEMMLELIKSDDEREIR | EEARRILEHLEELARK(SEQ ID NO: 101) |
| | ElEEEIARRILEHLEELARK(SEQ ID NO: 266) | |
| AHB2- | MEKKISAWSHPQFEKGGGSGGGSGGSAWSHPQFEKG | X1-ELEEQVMHVLDQVSELAHELLHKLTGEELERAA |
| AHB2_ | GSGSSGELEEQVMHVLDQVSELAHELLHKLTGEELE | YFNWWATEMMLELIKSDDEREIREIEEEARRILEHL |
| PAS | RAAYFNWWATEMMLELIKSDDEREIREIEEEARRIL | EELARK(SEQ ID NO: 101)-X2-ELEEQVMHVL |
| | EHLEELARKGGASPAAPAPASPAAPAPSAPAGGELE | DQVSELAHELLHKLTGEELERAAYFNWWATEMMLEL |
| | EQVMHVLDQVSELAHELLHKLTGEELERAAYFNWWA | IKSDDEREIREIEEEARRILEHLEELARK(SEQ ID |
| | TEMMLELIKSDDEREIREIEEEIARRILEHLEELAR | NO: 101) |
| | K(SEQ ID NO: 267) | |
| LCB3- | MEKKISAWSHPQFEKGGGSGGGSGGSAWSHPQFEKG | X1-NLDELHMQMTDLVYEALHFAKDEEFQKHVFQLF |
| LCB1_ | GSGSSGNLDELHMQMTDLVYEALHFAKDEEFQKHVF | EKATKAYKNKDRQKLEKVVEELKELLERLLS(SEQ |
| PAS | QLFEKATKAYKNKDRQKLEKVVEELKELLERLLSGG | ID NO: 155)-X2-DKENVLQKIYEIMKELERLGH |
| | ASPAAPAPASPAAPAPSAPAGGDKENVLQKIYEIMK | AEASMQVSDLIYEFMKTKDERLLEEAERLLEEVKR( |
| | ELERLGHAEASMQVSDLIYEFMKTKDERLLEEAERL | SEQ ID NO: 125) |
| | LEEVKR(SEQ ID NO: 268) | |
| AHB2- | MEKKISAWSHPQFEKGGGSGGGSGGSAWSHPQFEKG | X1-ELEEQVMHVLDQVSELAHELLHKLTGEELERAA |
| LCB1_ | GSGSSGELEEQVMHVLDQVSELAHELLHKLTGEELE | YFNWWATEMMLELIKSDDEREIREIEEEARRILEHL |
| PAS | RAAYFNWWATEMMLELIKSDDEREIREIEEEARRIL | EELARK(SEQ ID NO: 101)-X2-DKENVLQKIY |
| | EHLEELARKGGASPAAPAPASPAAPAPSAPAGGDKE | EIMKELERLGHAEASMQVSDLIYEFMKTKDERLLEE |
| | NVLQKIYEIMKELERLGHAEASMQVSDLIYEFMKTK | AERLLEEVKR(SEQ ID NO: 125) |
| | DERLLEEAERLLEEVKR(SEQ ID NO: 269) | |
| AHB2_ | MEKKISAWSHPQFEKGGGSGGGSGGSAWSHPQFEKG | X1-ELEEQVMHVLDQVSELAHELLHKLTGEELERAA |
| 3x_ | GSGSSGELEEQVMHVLDQVSELAHELLHKLTGEELE | YFNWWATEMMLELIKSDDEREIREIEEEARRILEHL |
| AS | RAAYFNWWATEMMLELIKSDDEREIREIEEEARRIL | EELARK(SEQ ID NO: 101)-X2-ELEEQVMHVL |
| | EHLEELARKGGASPAAPAPASPAAPAPSAPAGGELE | DQVSELAHELLHKLTGEELERAAYFNWWATEMMLEL |
| | EQVMHVLDQVSELAHELLHKLTGEELERAAYFNWWA | IKSDDEREIREIEEEIARRILEHLEELARK(SEQ I |
| | TEMMLELIKSDDEREIREIEEEARRILEHLEELARK | D NO: 101)-X3-ELEEQVMHVLDQVSELAHELLH |
| | GGASPAAPAPASPAAPAPSAPAGGELEEQVMHVLDQ | KLTGEELERAAYFNWWATEMMLELIKSDDEREIREI |
| | VSELAHELLHKLTGEELERAAYFNWWATEMMLELIK | EEEARRILEHLEELARK(SEQ ID NO: 101) |
| | SDDEREIREIEEEARRILEHLEELARK | |
| | (SEQ ID NO: 270) | |
| 3-2-1_ | MEKKISAWSHPQFEKGGGSGGGSGGSAWSHPQFEKG | X1-NLDELHMQMTDLVYEALHFAKDEEFQKHVFQLF |
| PAS | GSGSSGNLDELHMQMTDLVYEALHFAKDEEFQKHVF | EKATKAYKNKDRQKLEKVVEELKELLERLLS(SEQ |
| | QLFEKATKAYKNKDRQKLEKVVEELKELLERLLSGG | ID NO: 155)-X2- |
| | ASPAAPAPASPAAPAPSAPAGGELEEQVMHVLDQVS | ELEEQVMHVLDQVSELAHELL |
| | ELAHELLHKLTGEELERAAYFNWWATEMMLELIKSD | HKLTGEELERAAYFNWWATEMMLELIKSDDEREIRE |
| | DEREIREIEEEARRILEHLEELARKGGASPAAPAPA | IEEEARRILEHLEELARK |
| | SPAAPAPSAPAGGDKENVLQKIYEIMKELERLGHAE | (SEQ ID NO: 101)-X3 |
| | ASMQVSDLIYEFMKTKDERLLEEAERLLEEVKR | |
| | (SEQ ID NO: 271) | |
| 2-3- | MEKKISAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERA |
| PAS | RAAYFNWWATEMMLELIKSDDEREIREIEEEARRIL | AYFNWWATEMMLELIKSDDEREIREIEEEARRILEH |
| | EHLEELARKGGASPAAPAPASPAAPAPSAPAGGNLD | LEELARK |
| | ELHMQMTDLVYEALHFAKDEEFQKHVFQLFEKATKA | (SEQ ID NO: 101)-X2- |
| | YKNKDRQKLEKVVEELKELLERLLSGGASPAAPAPA | NLDELHMQ |
| | SPAAPAPSAPAGGDKENVLQKIYEIMKELERLGHAE | MTDLVYEALHFAKDEEFQKHVFQLFEKATKAYKNKD |
| | ASMQVSDLIYEFMKTKDERLLEEAERLLEEVKR | RQKLEKVVEELKELLERLLS |
| | (SEQ ID NO: 272) | (SEQ ID NO: 155)-X3 |
| 1-1-1_ | MEKKISAWSHPQFEKGGGSGGGSGGSAWSHPQFEKG | X1- |
| PAS | GSGSSGDKENVLQKIYEIMKELERLGHAEASMQVSD | DKENVLQKIYEIMKELERLGHAEASMQVSDLI |
| 24 | LIYEFMKTKDENLLEEAERLLEEVKRGGASPAAPAP | YEFMKTKDENLLEEAERLLEEVKR |
| | ASPAAPAPSAPAGGDKENVLQKIYEIMKELERLGHA | (SEQ ID NO: 135)-X2- |
| | EASMQVSDLIYEFMKTKDENLLEEAERLLEEVKRGG | DKENVLQKIYEIMKELERLGHAEASMQ |
| | ASPAAPAPASPAAPAPSAPAGGDKENVLQKIYEIMK | VSDLIYEFMKTKDENLLEEAERLLEEVKR |
| | ELERLGHAEASMQVSDLIYEFMKTKDENLLEEAERL | (SEQ ID NO: 135)-X3- |
| | LEEVKR | DKENVLQKIYEIMKELERLGHA |
| | (SEQ ID NO: 273) | EASMQVSDLIYEFMKTKDENLLEEAERLLEEVKR |
| | | (SEQ ID NO: 135) |
| 2-2-2_ | MEKKISAWSHPQFEKGGGSGGGSGGSAWSHPQFEKG | X1- |
| PAS | GSGSSGELEEQVMHVLDQVSELAHELLHKLTGEELE | ELEEQVMHVLDQVSELAHELLHKLTGEELERA |
| 24 | RAAYFNWWATEMMLELIKSDDEREIREIEEEARRIL | AYFNWWATEMMLELIKSDDEREIREIEEEARRILEH |
| | EHLEELARKGGASPAAPAPASPAAPAPSAPAGGELE | LEELARK |
| | EQVMHVLDQVSELAHELLHKLTGEELERAAYFNWWA | (SEQ ID NO: 101)-X2- |
| | TEMMLELIKSDDEREIREIEEEARRILEHLEELARK | ELEEQVMH |
| | GGASPAAPAPASPAAPAPSAPAGGELEEQVMHVLDQ | VLDQVSELAHELLHKLTGEELERAAYFNWWATEMML |
| | VSELAHELLHKLTGEELERAAYFNWWATEMMLELIK | ELIKSDDEREIREIEEEARRILEHLEELARK |
| | SDDEREIREIEEE1ARRILEHLEELARK | (SEQ ID NO: 101)-X3- |
| | (SEQ ID NO: 274) | ELEEQVMHVLDQVSELAHEL |
| | | LHKLTGEELERAAYFNWWATEMMLELIKSDDEREIR |
| | | EIEEEARRILEHLEELARK |
| | | (SEQ ID NO: 101) |
| 3-3-3_ | MEKKISAWSHPQFEKGGGSGGGSGGSAWSHPQFEKG | X1- |
| PAS | GSGSSGNLDELHMQMTDLVYEALHFAKDEEFQKHVF | NLDELHMQMTDLVYEALHFAKDEEFQKHVFQL |
| 24 | QLFEKATKAYKNKDRQKLEKVVEELKELLERLLSGG | FEKATKAYKNKDRQKLEKVVEELKELLERLLS |
| | ASPAAPAPASPAAPAPSAPAGGNLDELHMQMTDLVY | (SEQ ID NO: 155)-X2- |
| | EALHFAKDEEFQKHVFQLFEKATKAYKNKDRQKLEK | NLDELHMQMTDLVYEALHF |
| | WEELKELLERLLSGGASPAAPAPASPAAPAPSAPAG | AKDEEFQKHVFQLFEKATKAYKNKDRQKLEKVVEEL |
| | GNLDELHMQMTDLVYEALHFAKDEEFQKHVFQLFEK | KELLERLLS |
| | ATKAYKNKDRQKLEKVVEELKELLERLLS | (SEQ ID NO: 155)-X3- |
| | (SEQ ID NO: 275) | NLDELH |
| | | MQMTDLVYEALHFAKDEEFQKHVFQLFEKATKAYKN |
| | | KDRQKLEKVVEELKELLERLLS |
| | | (SEQ ID NO: 155) |
| 2-2-1_ | MEKKISAWSHPQFEKGGGSGGGSGGSAWSHPQFEKG | X1- |
| PAS | GSGSSGELEEQVMHVLDQVSELAHELLHKLTGEELE | ELEEQVMHVLDQVSELAHELLHKLTGEELERA |
| 24 | RAAYFNWWATEMMLELIKSDDEREIREIEEEARRIL | AYFNWWATEMMLELIKSDDEREIREIEEEARR |
| | EHLEELARKGGASPAAPAPASPAAPAPSAPAGGELE | ILEHLEELARK |
| | EQVMHVLDQVSELAHELLHKLTGEELERAAYFNWWA | (SEQ ID NO: 101)-X2- |
| | TEMMLELIKSDDEREIREIEEEARRILEHLEELARK | ELEEQVMHVLDQVSELAHELLHKLTGEELERAAYFN |
| | GGASPAAPAPASPAAPAPSAPAGGDKENVLQKIYEl | WWATEMMLELIKSDDE-REIREIEEEARRILEHLEE |
| | MKELERLGHAEASMQVSDLIYEFMKTKDENLLEEAE | LARK |
| | RLLEEVKR | (SEQ ID NO: 101) |
| | (SEQ ID NO: 276) | X3-DKENVLQKIYEIMKELERLG |
| | | HAEASMQVSDLIYEFMKTKDENLLEEAERLLEEVKR |
| | | (SEQ ID NO: 135) |
| 2- | MEKKISAWSHPQFEKGGGSGGGSGGSAWSHPQFEKG | X1- |
| 2_ | GSGSSGELEEQVMHVLDQVSELAHELLHKLTGEELE | ELEEQVMHVLDQVSELAHELLHKLTGEELERA |
| PAS | RAAYFNWWATEMMLELIKSDDEREIREIEEEARRIL | AYFNWWATEMMLELIKSDDEREIREIEEEARRILEH |
| 16 | EHLEELARKGGASPAAPAPASPAGGELEEQVMHVLD | LEELARK |
| | QVSELAHELLHKLTGEELERAAYFNWWATEMMLELI | (SEQ ID NO: 101)-X2- |
| | KSDDEREIREIEEEARRILEHLEELARK | ELEEQVMH |
| | (SEQ ID NO: 277 | VLDQVSELAHELLHKLTGEELERAAYFNWWATEMML |
| | | ELIKSDDEREIREIEEEARRILEHLEELARK |
| | | (SEQ ID NO: 101) |
| 2-2_PAS | MEKKISAWSHPQFEKGGGSGGGSGGSAWSHPQFEKG | X1-ELEEQVMHVLDQVSELAHELLHKLTGEELERAA |
| 11 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEARRIL | EELARK |
| | EHLEELARKGGASPAAPAGGELEEQVMHVLDQVSEL | (SEQ ID NO: 101) |
| | AHELLHKLTGEELERAAYFNWWATEMMLELIKSDDE | -X2-ELEEQVMH |
| | REIREIEEEARRILEHLEELARK | VLDQVSELAHELLHKLTGEELERAAYFNWWATEMML |
| | (SEQ ID NO: | ELIKSDDEREIREIEEEARRILEHLEELARK |
| | 278) | (SEQ ID NO: 101) |
| 3-1_PAS | MEKKISAWSHPQFEKGGGSGGGSGGSAWSHPQFEKG | X1-NLDELHMQMTDLVYEALHFAKDEEFQKHVFQLF |
| 16 | GSGSSGNLDELHMQMTDLVYEALHFAKDEEFQKHVF | EKATKAYKNKDRQKLEKVVEELKELLERLLS |
| | QLFEKATKAYKNKDRQKLEKVVEELKELLERLLSGGA | (SEQ ID NO: 155) |
| | SPAAPAPASPAGGDKENVLQKIYEIMKELERLGHAE | -X2-DKENVLQKIYEIMKELERLG |
| | ASMQVSDLIYEFMKTKDERLLEEAERLLEEVKR | HAEASMQVSDLIYEFMKTKDERLLEEAERLLEEVKR |
| | (SEQ ID NO: 279) | (SEQ ID NO: 125) |
| 3-1_PAS | MEKKISAWSHPQFEKGGGSGGGSGGSAWSHPQFEKG | X1-NLDELHMQMTDLVYEALHFAKDEEFQKHVFQLF |
| 11 | GSGSSGNLDELHMQMTDLVYEALHFAKDEEFQKHVF | EKATKAYKNKDRQKLEKVVEELKELLERLLS |
| | QLFEKATKAYKNKDRQKLEKVVEELKELLERLLSGGA | (SEQ ID NO: 155) |
| | SPAAPAGGDKENVLQKIYEIMKELERLGHAEASMQV | -X2- |
| | SDLIYEFMKTKDERLLEEAERLLEEVKR | DKENVLQKIYEIMKELERLG |
| | (SEQ ID NO: 280) | HAEASMQVSDLIYEFMKTKDERLLEEAERLLEEVKR |
| | | (SEQ ID NO: 125) |
| 2-1_PAS | MEKKISAWSHPQFEKGGGSGGGSGGSAWSHPQFEKG | X1-ELEEQVMHVLDQVSELAHELLHKLTGEELERAA |
| 16 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEARRIL | EELARK |
| | EHLEELARKGGASPAAPAPASPAGGDKENVLQKIYE | (SEQ ID NO: 101) |
| | IMKELERLGHAEASMQVSDLIYEFMKTKDERLLEEA | -X2-DKENVLQK |
| | ERLLEEVKR | IYEIMKELERLGHAEASMQVSDLIYEFMKTKDERLL |
| | (SEQ ID NO: 281) | EEAERLLEEVKR |
| | | (SEQ ID NO: 125) |
| 2-1_PAS | MEKKISAWSHPQFEKGGGSGGGSGGSAWSHPQFEKG | X1-ELEEQVMHVLDQVSELAHELLHKLTGEELERAA |
| 11 | GSGSSGELEEQVMHVLDQVSELAHELLHKLTGEELE | YFNWWATEMMLELIKSDDEREIREIEEEARRILEHL |
| | RAAYFNWWATEMMLELIKSDDEREIREIEEEARRIL | EELARK(SEQ ID NO: 101) |
| | EHLEELARKGGASPAAPAGGDKENVLQKIYEIMKEL | -X2-DKENVLQKIYEI |
| | ERLGHAEASMQVSDLIYEFMKTKDERLLEEAERLLE | MKELERLGHAEASMQVSDLIYEFMKTKDERLLEEAE |
| | EVKR | RLLEEVKR |
| | (SEQ ID NO: 282) | (SEQ ID NO: 125) |
| 3v2.3- | MEKKISAWSHPQFEKGGGSGGGSGGSAWSHPQFEKG | X1-NIDELLMQVTDLIYEALHFAKDEEFQKHAFQLF |
| 2-1_PAS | GSGSSGNIDELLMQVTDLIYEALHFAKDEEFQKHAF | EKATKAYKNKDKQKLEKVVEELKELLERILS |
| 24 | QLFEKATKAYKNKDKQKLEKVVEELKELLERILSGGA | (SEQ ID NO: 163) |
| | SPAAPAPASPAAPAPSAPAGGELEEQVMHVLDQVSE | -X2- |
| | LAHELLHKLTGEELERAAYFNWWATEMMLELIKSDD | ELEEQVMHVLDQVSELAHEL |
| | EREIREIEEEARRILEHLEELARKGGASPAAPAPAS | LHKLTGEELERAAYFNWWATEMMLELIKSDDEREIR |
| | PAAPAPSAPAGGDKENVLQKIYEIMKELERLGHAEA | EIEEEARRILEHLEELARK |
| | SMQVSDLIYEFMKTKDERLLEEAERLLEEVKR | (SEQ ID NO: 101) |
| | (SEQ ID NO: 283) | -X3 |
| 2-3v2.3- | MEKKISAWSHPQFEKGGGSGGGSGGSAWSHPQFEKG | X1-ELEEQVMHVLDQVSELAHELLHKLTGEELERAA |
| 1_PAS | GSGSSGELEEQVMHVLDQVSELAHELLHKLTGEELE | YFNWWATEMMLELIKSDDEREIREIEEEARRILEHL |
| 24 | RAAYFNWWATEMMLELIKSDDEREIREIEEEARRIL | EELARK |
| | EHLEELARKGGASPAAPAPASPAAPAPSAPAGGNID | (SEQ ID NO: 101) |
| | ELLMQVTDLIYEALHFAKDEEFQKHAFQLFEKATKA | -X2-NIDELLMQ |
| | YKNKDKQKLEKVVEELKELLERILSGGASPAAPAPAS | VTDLIYEALHFAKDEEFQKHAFQLFEKATKAYKNKD |
| | PAAPAPSAPAGGDKENVLQKIYEIMKELERLGHAEA | KQKLEKVVEELKELLERILS |
| | SMQVSDLIYEFMKTKDERLLEEAERLLEEVKR | (SEQ ID NO: 163) |
| | (SEQ ID NO: 284) | -X3 |
| 2-2-2_PAS | MEKKISAWSHPQFEKGGGSGGGSGGSAWSHPQFEKG | X1-ELEEQVMHVLDQVSELAHELLHKLTGEELERAA |
| 24_ompT | GSGSSGELEEQVMHVLDQVSELAHELLHKLTGEELE | YFNWWATEMMLELIKSDDEREIREIEEEAARILEHL |
| | RAAYFNWWATEMMLELIKSDDEREIREIEEEAARIL | EELART |
| | EHLEELARTGGASPAAPAPASPAAPAPSAPAGGELE | (SEQ ID NO: 164) |
| | EQVMHVLDQVSELAHELLHKLTGEELERAAYFNWWA | -X2- |
| | TEMMLELIKSDDEREIREIEEEAARILEHLEELART | ELEEQVMH |
| | GGASPAAPAPASPAAPAPSAPAGGELEEQVMHVLDQ | VLDQVSELAHELLHKLTGEELERAAYFNWWATEMML |
| | VSELAHELLHKLTGEELERAAYFNWWATEMMLELIK | ELIKSDDEREIREIEEEAARILEHLEELART |
| | SDDEREIREIEEEAARILEHLEELART | (SEQ ID NO: 164) |
| | (SEQ ID NO: 285) | -X3-ELEEQVMHVLDQVSELAHE |
| | | LLHKLTGEELERAAYFNWWATEMMLELIKSDDEREI |
| | | REIEEEAARILEHLEELART |
| | | (SEQ ID NO: 164) |
| 1-1-1_ | MEKKISAWSHPQFEKGGGSGGGSGGSAWSHPQFEKG | X1- |
| _PAS | GSGSSGDKENVLQKIYEIMKELERLGHAEASMQV5D | DKENVLQKIYEIMKELERLGHAEASMQVSDLIYEFM |
| 24_ | LIYEFMKTKDENLLEEAERLLEEVTRGGASPAAPAP | KTKDERLLEEAERLLEEVKR |
| ompT | ASPAAPAPSAPAGGDKENVLQKIYEIMKELERLGHA | (SEQ ID NO: 125) |
| | EASMQVSDLIYEFMKTKDENLLEEAERLLEEVTRGG | -X2- |
| | ASPAAPAPASPAAPAPSAPAGGDKENVLQKIYEIMK | DKENVLQKIYEIMKELERLGHAEASMQVSDLIYEFM |
| | ELERLGHAEASMQVSDLIYEFMKTKDENLLEEAERL | KTKDERLLEEAERLLEEVKR |
| | LEEVTR | (SEQ ID NO: 125) |
| | (SEQ ID NO: 286) | -X3- |
| | | DKENVLQKIYEIMKELERLGHAEASMQVSDLIYEFM |
| | | KTKDERLLEEAERLLEEVKR |
| | | (SEQ ID NO: 125) |
| 3v2.3-2- | MEKKISAWSHPQFEKGGGSGGGSGGSAWSHPQFEKG | X1- |
| 1_PAS | GSGSSGNIDELLMQVTDLIYEALHFAKDEEFQKHAF | NIDELLMQVTDLIYEALHFAKDEEFQKHAFQLFEKA |
| 24_ | QLFEKATKAYKNKDKQKLEKVVEELKELLERILSGGA | TKAYKNKDKQKLEKVVEELKELLERILS |
| ompT | SPAAPAPASPAAPAPSAPAGGELEEQVMHVLDQVSE | (SEQ ID NO: 163) |
| | LAHELLHKLTGEELERAAYFNWWATEMMLELIKSDD | -X2- |
| | EREIREIEEEAARILEHLEELARTGGASPAAPAPAS | ELEEQVMHVLDQVSELAHELLHKLTGEELERAAYFN |
| | PAAPAPSAPAGGDKENVLQKIYEIMKELERLGHAEA | WWATEMMLELIKSDDEREIREIEEEAARILEHLEEL |
| | SMQVSDLIYEFMKTKDERLLEEAERLLEEVKR | ART |
| | (SEQ ID NO: 287) | (SEQ ID NO: 164) |
| | | -X3 |
| 2-3v2.3- | MEKKISAWSHPQFEKGGGSGGGSGGSAWSHPQFEKG | X1- |
| _PAS | GSGSSGELEEQVMHVLDQVSELAHELLHKLTGEELE | ELEEQVMHVLDQVSELAHELLHKLTGEELERAAYFN |
| 24_ | RAAYFNWWATEMMLELIKSDDEREIREIEEEAARIL | WWATEMMLELIKSDDEREIREIEEEAARILEHLEEL |
| ompT | EHLEELARTGGASPAAPAPASPAAPAPSAPAGGNID | ART |
| | ELLMQVTDLIYEALHFAKDEEFQKHAFQLFEKATKA | (SEQ ID NO: 164) |
| | YKNKDKQKLEKVVEELKELLERILSGGASPAAPAPAS | -X2- |
| | PAAPAPSAPAGGDKENVLQKIYEIMKELERLGHAEA | NIDELLMQVTDLIYEALHFAKDEEFQKHAFQLFEKA |
| | SMQVSDLIYEFMKTKDERLLEEAERLLEEVKR | TKAYKNKDKQKLEKVVEELKELLERILS |
| | (SEQ ID NO: 288) | (SEQ ID NO: 163) |
| | | -X3 |
| 3v2.3- | MEKKISAWSHPQFEKGGGSGGGSGGSAWSHPQFEKG | X1- |
| 1_2-1_ | GSGSSGNIDELLMQVTDLIYEALHFAKDEEFQKHAF | NIDELLMQVTDLIYEALHFAKDEEFQKHAFQLFEKA |
| _PAS | QLFEKATKAYKNKDKQKLEKVVEELKELLERILSGG | TKAYKNKDKQKLEKVVEELKELLERILS |
| 24 | ASPAAPAPASPAAPAPSAPAGGELEEQVMHVLDQVS | (SEQ ID NO: 163) |
| | ELAHELLHKLTGEELERAAYFNWWATEMMLELIKSD | -X2- |
| | DEREIREIEEEARRILEHLEELARKGGASPAAPAPA | ELEEQVMHVLDQVSELAHELLHKLTGEELERAAYFN |
| | SPAAPAPSAPAGGDKENVLQKIYEIMKELERLGHAE | WWATEMMLELIKSDDEREIREIEEEARRILEHLEEL |
| | ASMQVSDLIYEFMKTKDENLLEEAERLLEEVKR | ARK |
| | (SEQ ID NO: 289) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3v2.3- | MEKKISAWSHPQFEKGGGSGGGSGGSAWSHPQFEK | X1- |
| 1_ | GGSGSSGELEEQVMHVLDQVSELAHELLHKLTGEEL | ELEEQVMHVLDQVSELAHELLHKLTGEELERAAYFN |
| _PAS | ERAAYFNWV/ATEMMLELIKSDDEREIREIEEEARR | WWATEMMLELIKSDDEREIREIEEEARRILEHLEEL |
| 24 | ILEHLEELARKGGASPAAPAPASPAAPAPSAPAGGN | ARK |
| | IDELLMQVTDLIYEALHFAKDEEFQKHAFQLFEKAT | (SEQ ID NO: 101) |
| | KAYKNKDKQKLEKVVEELKELLERILSGGASPAAPAP | -X2- |
| | ASPAAPAPSAPAGGDKENVLQKIYEIMKELERLGHA | NIDELLMQVTDLIYEIALHFAKDEEFQKHAFQLFEK |
| | EASMQVSDLIYEFMKTKDENLLEEAERLLEEVKR | ATKAYKNKDKQKLEKVVEELKELLERILS |
| | (SEQ ID NO: 290) | (SEQ ID NO: 163) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 3v2.3- | MEKKISAWSHPQFEKGGGSGGGSGGSAWSHPQFEKG | X1- |
| 2-1_ | GSGSSGNIDELLMQVTDLIYEALHFAKDEEFQKHAF | NIDELLMQVTDLIYEIALHFAKDEEFQKHAFQLFEK |
| _PAS | QLFEKATKAYKNKDKQKLEKVVEELKELLERILSGGA | ATKAYKNKDKQKLEKVVEELKELLERILS |
| 24_ | SPAAPAPASPAAPAPSAPAGGELEEQVMHVLDQVSE | (SEQ ID NO: 163) |
| ompT | LAHELLHKLTGEELERAAYFNWWATEMMLELIKSDD | -X2- |
| | EREIREIEEEAARILEHLEELARTGGASPAAPAPAS | ELEEQVMHVLDQVSELAHELLHKLTGEELERAAYFN |
| | PAAPAPSAPAGGDKENVLQKIYEIMKELERLGHAEA | WWATEMMLELIKSDDEREIREIEEEAARILEHLEEL |
| | SMQVSDLIYEFMKTKDENLLEEAERLLEEVKR | ART |
| | (SEQ ID NO: 291) | (SEQ ID NO: 164) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3v2.3- | MEKKISAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYFN |
| _PAS | RAAYFNWWATEMMLELIKSDDEREIREIEEEAARIL | WWATEMMLELIKSDDEREIREIEEEAARILEHLEEL |
| 24_ | EHLEELARTGGASPAAPAPASPAAPAPSAPAGGNID | ART |
| ompT | ELLMQVTDLIYEALHFAKDEEFQKHAFQLFEKATKA | (SEQ ID NO: 164) |
| | YKNKDKQKLEKVVEELKELLERILSGGASPAAPAPA | -X2- |
| | SPAAPAPSAPAGGDKENVLQKIYEIMKELERLGHAE | NIDELLMQVTDLIYEALHFAKDEEFQKHAFQLFEKA |
| | ASMQVSDLIYEFMKTKDENLLEEAERLLEEVKR | TKAYKNKDKQKLEKVVEELKELLERILS |
| | (SEQ ID NO: 292) | (SEQ ID NO: 163) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3v2.3- | MEKKIHHHHHHSGENLYFQSGGSGSSGELEEQVMHV | X1- |
| 1_PAS | LDQVSELAHELLHKLTGEELERAAYFNWWATEMMLE | ELEEQVMHVLDQVSELAHELLHKLTGEELERAAYFN |
| 24__N- | LIKSDDEREIREIEEEARRILEHLEELARKGGASPA | WWATEMMLELIKSDDEREIREIEEEARRILEHLEEL |
| His-TEV | APAPASPAAPAPSAPAGGNIDELLMQVTDLIYEALH | ARK |
| | FAKDEEFQKHAFQLFEKATKAYKNKDKQKLEKVVEE | (SEQ ID NO: 101) |
| | LKELLERILSGGASPAAPAPASPAAPAPSAPAGGDK | -X2- |
| | ENVLQKIYEIMKELERLGHAEASMQVSDLIYEFMKT | NIDELLMQVTDLIYEALHFAKDEEFQKHAFQLFEKA |
| | KDENLLEEAERLLEEVKR | TKAYKNKDKQKLEKVVEELKELLERILS |
| | (SEQ ID NO: 293) | (SEQ ID NO: 163) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3v2.3-1_ | MEKKIHHHHHHSGENLYFQSGGSGSSGELEEQVMHV | X1- |
| PAS | LDQVSELAHELLHKLTGEELERAAYFNWWATEMMLE | ELEEQVMHVLDQVSELAHELLHKLTGEELERAAYFN |
| 24_ompTN- | LIKSDDEREIREIEEEAARILEHLEELARTGGASPA | WWATEMMLELIKSDDEREIREIEEEAARILEHLEEL |
| His-TEV | APAPASPAAPAPSAPAGGNIDELLMQVTDLIYEALH | ART |
| | FAKDEEFQKHAFQLFEKATKAYKNKDKQKLEKVVEE | (SEQ ID NO: 164) |
| | LKELLERILSGGASPAAPAPASPAAPAPSAPAGGDK | -X2- |
| | ENVLQKIYEIMKELERLGHAEASMQVSDLIYEFMKT | NIDELLMQVTDLIYEALHFAKDEEFQKHAFQLFEKA |
| | KDENLLEEAERLLEEVKR | TKAYKNKDKQKLEKVVEELKELLERILS |
| | (SEQ ID NO: 294) | (SEQ ID NO: 163) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_PAS | MEKKIDYKDDDDKGSGSSAWSHPQFEKGGGSGGGSG | X1- |
| 24_NTSF | GSAWSHPQFEKGGSGSSGELEEQVMHVLDQVSELAH | ELEEQVMHVLDQVSELAHELLHKLTGEELERAAYFN |
| | ELLHKLTGEELERAAYFNWWATEMMLELIKSDDERE | WWATEMMLELIKSDDEREIREIEEEARRILEHLEEL |
| | IREIEEEARRILEHLEELARKGGASPAAPAPASPAA | ARK |
| | PAPSAPAGGNIDELLMQVTDLIYEALHFAKDEEFQK | (SEQ ID NO: 101) |
| | HAFQLFEKATKAYKNKDKQKLEKVVEELKELLERIL | -X2- |
| | SGGASPAAPAPASPAAPAPSAPAGGDKENVLQKIYE | NIDELLMQVTDLIYEALHFAKDEEFQKHAFQLFEKA |
| | IMKELERLGHAEASMQVSDLIYEFMKTKDENLLEEA | TKAYKNKDKQKLEKVVEELKELLERILS |
| | ERLLEEVKR | (SEQ ID NO: 163) |
| | (SEQ ID NO: 295) | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_PAS | MEKKIDYKDHDGDYKDHDIDYKDDDDKGSGSSAWSH | X1- |
| 24_NTS3F | PQFEKGGGSGGGSGGSAWSHPQFEKGGSGSSGELEE | ELEEQVMHVLDQVSELAHELLHKLTGEELERAAYFN |
| | QVMHVLDQVSELAHELLHKLTGEELERAAYFNWWAT | WWATEMMLELIKSDDEREIREIEEEARRILEHLEEL |
| | EMMLELIKSDDEREIREIEEEARRILEHLEELARKG | ARK |
| | GASPAAPAPASPAAPAPSAPAGGNIDELLMQVTDLI | (SEQ ID NO: 101) |
| | YEALHFAKDEEFQKHAFQLFEKATKAYKNKDKQKLE | -X2- |
| | KVVEELKELLERILSGGASPAAPAPASPAAPAPSAP | NIDELLMQVTDLIYEALHFAKDEEFQKHAFQLFEKA |
| | AGGDKENVLQKIYEIMKELERLGHAEASMQVSDLIY | TKAYKNKDKQKLEKVVEELKELLERILS |
| | EFMKTKDENLLEEAERLLEEVKR | (SEQ ID NO: 163) |
| | (SEQ ID NO: 296) | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_PAS | MEKKIEQKLISEEDLGSGSSAWSHPQFEKGGGSGGG | X1- |
| 24_NTSM | SGGSAWSHPQFEKGGSGSSGELEEQVMHVLDQVSEL | ELEEQVMHVLDQVSELAHELLHKLTGEELERAAYFN |
| | AHELLHKLTGEELERAAYFNWWATEMMLELIKSDDE | WWATEMMLELIKSDDEREIREIEEEARRILEHLEEL |
| | REIREIEEEARRILEHLEELARKGGASPAAPAPASP | ARK |
| | AAPAPSAPAGGNIDELLMQVTDLIYEALHFAKDEEF | (SEQ ID NO: 101) |
| | QKHAFQLFEKATKAYKNKDKQKLEKVVEELKELLER | -X2- |
| | ILSGGASPAAPAPASPAAPAPSAPAGGDKENVLQKI | NIDELLMQVTDLIYEALHFAKDEEFQKHAFQLFEKA |
| | YEIMKELERLGHAEASMQVSDLIYEFMKTKDENLLE | TKAYKNKDKQKLEKVVEELKELLERILS |
| | EAERLLEEVKR | (SEQ ID NO: 163) |
| | (SEQ ID NO: 297) | -X3- |
| | | DKENVLQKIYEIMKELERLGHAEASMQVSDLIYEFM |
| | | KTKDENLLEEAERLLEEVKR |
| | | (SEQ ID NO: 135) |
| 2-PAS | MEKKIDYKDHDGDYKDHDIDYKDDDDKGSGSSAWSH | X1- |
| 24-3- | PQFEKGGGSGGGSGGSAWSHPQFEKGGSGSSGELEE | ELEEQVMHVLDQVSELAHELLHKLTGEELERAAYFN |
| PAS | QVMHVLDQVSELAHELLHKLTGEELERAAYFNWWAT | WWATEMMLELIKSDDEREIREIEEEARRILEHLEEL |
| 16-1_ | EMMLELIKSDDEREIREIEEEARRILEHLEELARKG | ARK |
| NTS3F | GASPAAPAPASPAAPAPSAPAGGNIDELLMQVTDLI | (SEQ ID NO: 101) |
| | YEALHFAKDEEFQKHAFQLFEKATKAYKNKDKQKLE | -X2- |
| | KVVEELKELLERILSGGASPAAPAPASPAGGDKENV | NIDELLMQVTDLIYEALHFAKDEEFQKHAFQLFEKA |
| | LQKIYEIMKELERLGHAEASMQVSDLIYEFMKTKDE | TKAYKNKDKQKLEKVVEELKELLERILS |
| | NLLEEAERLLEEVKR | (SEQ ID NO: 163) |
| | (SEQ ID NO: 298) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKIDYKDHDGDYKDHDIDYKDDDDKGSGSSAWSH | X1- |
| PAS16-3- | PQFEKGGGSGGGSGGSAWSHPQFEKGGSGSSGELEE | ELEEQVMHVLDQVSELAHELLHKLTGEELERAAYFN |
| PAS16- | QVMHVLDQVSELAHELLHKLTGEELERAAYFNWWAT | WWATEMMLELIKSDDEREIREIEEEARRILEHLEEL |
| 1_NTS | EMMLELIKSDDEREIREIEEEARRILEHLEELARKG | ARK |
| 3F | GASPAAPAPASPAGGNIDELLMQVTDLIYEALHFAK | (SEQ ID NO: 101) |
| | DEEFQKHAFQLFEKATKAYKNKDKQKLEKVVEELKE | -X2- |
| | LLERILSGGASPAAPAPASPAGGDKENVLQKIYEIM | NIDELLMQVTDLIYEALHFAKDEEFQKHAFQLFEKA |
| | KELERLGHAEASMQVSDLIYEFMKTKDENLLEEAER | TKAYKNKDKQKLEKVVEELKELLERILS |
| | LLEEVKR | (SEQ ID NO: 163) |
| | (SEQ ID NO: 299) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKIDYKDHDGDYKDHDIDYKDDDDKGSGSSAWSH | X1- |
| PAS11- | PQFEKGGGSGGGSGGSAWSHPQFEKGGSGSSGELEE | ELEEQVMHVLDQVSELAHELLHKLTGEELERAAYFN |
| 3- | QVMHVLDQVSELAHELLHKLTGEELERAAYFNWWAT | WWATEMMLELIKSDDEREIREIEEEARRILEHLEEL |
| PAS16-1_NTS | EMMLELIKSDDEREIREIEEEARRILEHLEELARKG | ARK |
| 3F | GASPAAPAGGNIDELLMQVTDLIYEALHFAKDEEFQ | (SEQ ID NO: 101) |
| | KHAFQLFEKATKAYKNKDKQKLEKVVEELKELLERI | -X2- |
| | LSGGASPAAPAPASPAGGDKENVLQKIYEIMKELER | NIDELLMQVTDLIYEALHFAKDEEFQKHAFQLFEKA |
| | LGHAEASMQVSDLIYEFMKTKDENLLEEAERLLEEV | TKAYKNKDKQKLEKVVEELKELLERILS |
| | KR | (SEQ ID NO: 163) |
| | (SEQ ID NO: 300) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKIDYKDHDGDYKDHDIDYKDDDDKGSGSSAWSH | X1- |
| PAS24 | PQFEKGGGSGGGSGGSAWSHPQFEKGGSGSSGELEE | ELEEQVMHVLDQVSELAHELLHKLTGEELERAAYFN |
| -3- | QVMHVLDQVSELAHELLHKLTGEELERAAYFNWWAT | WWATEMMLELIKSDDEREIREIEEEARRILEHLEEL |
| PAS11-1_ | EMMLELIKSDDEREIREIEEEARRILEHLEELARKG | ARK |
| NTS | GASPAAPAPASPAAPAPSAPAGGNIDELLMQVTDLI | (SEQ ID NO: 101) |
| 3F | YEALHFAKDEEFQKHAFQLFEKATKAYKNKDKQKLE | -X2- |
| | KVVEELKELLERILSGGASPAAPAGGDKENVLQKIY | NIDELLMQVTDLIYEALHFAKDEEFQKHAFQLFEKA |
| | EIMKELERLGHAEASMQVSDLIYEFMKTKDENLLEE | TKAYKNKDKQKLEKVVEELKELLERILS |
| | AERLLEEVKR | (SEQ ID NO: 163) |
| | (SEQ ID NO: 301) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKIDYKDHDGDYKDHDIDYKDDDDKGSGSSAWSH | X1- |
| PAS16 | PQFEKGGGSGGGSGGSAWSHPQFEKGGSGSSGELEE | ELEEQVMHVLDQVSELAHELLHKLTGEELERAAYFN |
| -3- | QVMHVLDQVSELAHELLHKLTGEELERAAYFNWWAT | WWATEMMLELIKSDDEREIREIEEEARRILEHLEEL |
| PAS11- | EMMLELIKSDDEREIREIEEEARRILEHLEELARKG | ARK |
| 1_NTS | GASPAAPAPASPAGGNIDELLMQVTDLIYEALHFAK | (SEQ ID NO: 101) |
| 3F | DEEFQKHAFQLFEKATKAYKNKDKQKLEKVVEELKE | -X2- |
| | LLERILSGGASPAAPAGGDKENVLQKIYEIMKELER | NIDELLMQVTDLIYEALHFAKDEEFQKHAFQLFEKA |
| | LGHAEASMQVSDLIYEFMKTKDENLLEEAERLLEEV | TKAYKNKDKQKLEKVVEELKELLERILS |
| | KR | (SEQ ID NO: 163) |
| | (SEQ ID NO: 302) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKIDYKDHDGDYKDHDIDYKDDDDKGSGSSAWSH | X1- |
| PAS11 | PQFEKGGGSGGGSGGSAWSHPQFEKGGSGSSGELEE | ELEEQVMHVLDQVSELAHELLHKLTGEELERAAYFN |
| -3- | QVMHVLDQVSELAHELLHKLTGEELERAAYFNWWAT | WWATEMMLELIKSDDEREIREIEEEARRILEHLEEL |
| PAS11- | EMMLELIKSDDEREIREIEEEARRILEHLEELARKG | ARK |
| 1_NTS | GASPAAPAGGNIDELLMQVTDLIYEALHFAKDEEFQ | (SEQ ID NO: 101) |
| 3F | KHAFQLFEKATKAYKNKDKQKLEKVVEELKELLERI | -X2- |
| | LSGGASPAAPAGGDKENVLQKIYEIMKELERLGHAE | NIDELLMQVTDLIYEALHFAKDEEFQKHAFQLFEKA |
| | ASMQVSDLIYEFMKTKDENLLEEAERLLEEVKR | TKAYKNKDKQKLEKVVEELKELLERILS |
| | (SEQ ID NO: 303) | (SEQ ID NO: 163) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| 2_G4 | GSGSSGELEEQVMHVLDQVSELAHELLHKLTGEELE | ELEEQVMHVLDQVSELAHELLHKLTGEELERAAYFN |
| | RAAYFNWWATEMMLELIKSDDEREIREIEEEARRIL | WWATEMMLELIKSDDEREIREIEEEARRILEHLEEL |
| | EHLEELARKGGGGELEEQVMHVLDQVSELAHELLHK | ARK |
| | LTGEELERAAYFNWWATEMMLELIKSDDEREIREIE | (SEQ ID NO: 101) |
| | EEARRILEHLEELARK | -X2- |
| | (SEQ ID NO: 304) | ELEEQVMHVLDQVSELAHELLHKLTGEELERAAYFN |
| | | WWATEMMLELIKSDDEREIREIEEEARRILEHLEEL |
| | | ARK |
| | | (SEQ ID NO: 101) |
| 2- | MEKKISAWSHPQFEKGGGSGGGSGGSAWSHPQFEKG | X1- |
| 2_G2 | GSGSSGELEEQVMHVLDQVSELAHELLHKLTGEELE | ELEEQVMHVLDQVSELAHELLHKLTGEELERAAYFN |
| | RAAYFNWWATEMMLELIKSDDEREIREIEEEARRIL | WWATEMMLELIKSDDEREIREIEEEARRILEHLEEL |
| | EHLEELARKGGELEEQVMHVLDQVSELAHELLHKLT | ARK |
| | GEELERAAYFNWWATEMMLELIKSDDEREIREIEEE | (SEQ ID NO: 101) |
| | ARRILEHLEELARK | -X2- |
| | (SEQ ID NO: 305) | ELEEQVMHVLDQVSELAHELLHKLTGEELERAAYFN |
| | | WWATEMMLELIKSDDEREIREIEEEARRILEHLEEL |
| | | ARK |
| | | (SEQ ID NO: 101) |
| 2- | MEKKISAWSHPQFEKGGGSGGGSGGSAWSHPQFEKG | X1- |
| 3_ | GSGSSGELEEQVMHVLDQVSELAHELLHKLTGEELE | ELEEQVMHVLDQVSELAHELLHKLTGEELERAAYFN |
| PAS | RAAYFNWWATEMMLELIKSDDEREIREIEEEARRIL | WWATEMMLELIKSDDEREIREIEEEARRILEHLEEL |
| 24 | EHLEELARKGGASPAAPAPASPAAPAPSAPAGGNID | ARK |
| | ELLMQVTDLIYEALHFAKDEEFQKHAFQLFEKATKA | (SEQ ID NO: 101) |
| | YKNKDKQKLEKVVEELKELLERILS | -X2- |
| | (SEQ ID NO: 306) | NIDELLMQVTDLIYEALHFAKDEEFQKHAFQLFEKA |
| | | TKAYKNKDKQKLEKVVEELKELLERILS |
| | | (SEQ ID NO: 163) |
| 2- | MEKKISAWSHPQFEKGGGSGGGSGGSAWSHPQFEKG | X1- |
| 3_ | GSGSSGELEEQVMHVLDQVSELAHELLHKLTGEELE | ELEEQVMHVLDQVSELAHELLHKLTGEELERAAYFN |
| PAS | RAAYFNWWATEMMLELIKSDDEREIREIEEEARRIL | WWATEMMLELIKSDDEREIREIEEEARRILEHLEEL |
| 16 | EHLEELARKGGASPAAPAPASPAGGNIDELLMQVTD | ARK |
| | LIYEALHFAKDEEFQKHAFQLFEKATKAYKNKDKQK | (SEQ ID NO: 101) |
| | LEKVVEELKELLERILS | -X2- |
| | (SEQ ID NO: 307) | NIDELLMQVTDLIYEALHFAKDEEFQKHAFQLFEKA |
| | | TKAYKNKDKQKLEKVVEELKELLERILS |
| | | (SEQ ID NO: 163) |
| 2- | MEKKISAWSHPQFEKGGGSGGGSGGSAWSHPQFEKG | X1- |
| 3_ | GSGSSGELEEQVMHVLDQVSELAHELLHKLTGEELE | ELEEQVMHVLDQVSELAHELLHKLTGEELERAAYFN |
| PAS | RAAYFNWWATEMMLELIKSDDEREIREIEEEARRIL | WWATEMMLELIKSDDEREIREIEEEARRILEHLEEL |
| 11 | EHLEELARKGGASPAAPAGGNIDELLMQVTDLIYEA | ARK |
| | LHFAKDEEFQKHAFQLFEKATKAYKNKDKQKLEKVV | (SEQ ID NO: 101) |
| | EELKELLERILS | -X2- |
| | (SEQ ID NO: 308) | NIDELLMQVTDLIYEALHFAKDEEFQKHAFQLFEKA |
| | | TKAYKNKDKQKLEKVVEELKELLERILS |
| | | (SEQ ID NO: 163) |
| 1-2-1 | MEKKISAWSHPQFEKGGGSGGGSGGSAWSHPQFEKG | X1- |
| _PAS | GSGSSGDKENVLQKIYEIMKELERLGHAEASMQVSD | DKENVLQKIYEIMKELERLGHAEASMQVSDLIYEFM |
| 24 | LIYEFMKTKDENLLEEAERLLEEVKRGGASPAAPAP | KTKDENLLEEAERLLEEVKR |
| | ASPAAPAPSAPAGGELEEQVMHVLDQVSELAHELLH | (SEQ ID NO: 135) |
| | KLTGEELERAAYFNWWATEMMLELIKSDDEREIREI | -X2- |
| | EEEARRILEHLEELARKGGASPAAPAPASPAAPAPS | ELEEQVMHVLDQVSELAHELLHKLTGEELERAAYFN |
| | APAGGDKENVLQKIYEIMKELERLGHAEASMQVSDL | WWATEMMLELIKSDDEREIREIEEEARRILEHLEEL |
| | IYEFMKTKDENLLEEAERLLEEVKR | ARK |
| | (SEQ ID NO: 309) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1-ELEEQVMHVLDQVSELAHELLHKLTGEELERAAYFN |
| PAS24 | GSGSSGELEEQVMHVLDQVSELAHELLHKLTGEELE | WWATEMMLELIKSDDEREIREIEEEARRILEHLEEL |
| -3- | RAAYFNWWATEMMLELIKSDDEREIREIEEEARRIL | ARK |
| PAS24- | EHLEELARKGGASPAAPAPASPAAPAPSAPAGGNLD | (SEQ ID NO: 101) |
| 1_NTS | ELHMQMTDLVYEALHFAKDEEFQKHVFQLFEKATKA | -X2- |
| | YKNKDRQKLEKVVEELKELLERLLSGGASPAAPAPA | NLDELHMQMTDLVYEALHFAKDEEFQKHVFQLFEKA |
| | SPAAPAPSAPAGGDKENVLQKIYEIMKELERLGHAE | TKAYKNKDRQKLEKVVEELKELLERLLS |
| | ASMQVSDLIYEFMKTKDENLLEEAERLLEEVKR | (SEQ ID NO: 155) |
| | (SEQ ID NO: 310) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS24 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEEL |
| PAS16 | EHLEELARKGGASPAAPAPASPAAPAPSAPAGGNLD | ARK |
| 1_NTS | ELHMQMTDLVYEALHFAKDEEFQKHVFQLFEKATKA | (SEQ ID NO: 101) |
| | YKNKDRQKLEKVVEELKELLERLLSGGASPAAPAPA | -X2- |
| | SPAGGDKENVLQKIYEIMKELERLGHAEASMQVSDL | NLDELHMQMTDLVYEALHFAKDEEFQKHVFQLFEKA |
| | IYEFMKTKDENLLEEAERLLEEVKR | TKAYKNKDRQKLEKVVEELKELLERLLS |
| | (SEQ ID NO: 311) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS16 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEELARK |
| PAS24 | EHLEELARKGGASPAAPAPASPAGG | (SEQ ID NO: 101) |
| 1_NTS | (SEQ ID NO: 155) | -X2- |
| | GGASPAAPAPASPAAPAPSAPAGGDKENVLQKIYEI | NLDELHMQMTDLVYEALHFAKDEEFQKHVFQLFEKA |
| | MKELERLGHAEASMQVSDLIYEFMKTKDENLLEEAE | TKAYKNKDRQKLEKVVEELKELLERLLS |
| | RLLEEVKR | (SEQ ID NO: 155) |
| | (SEQ ID NO: 312) | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS16 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLE |
| PAS16 | EHLEELARKGGASPAAPAPASPAGGNLDELHMQMTD | ELARK |
| 1_NTS | LVYEALHFAKDEEFQKHVFQLFEKATKAYKNKDRQK | (SEQ ID NO: 101) |
| | LEKVVEELKELLERLLSGGASPAAPAPASPAGGDKE | -X2- |
| | NVLQKIYEIMKELERLGHAEASMQVSDLIYEFMKTK | NLDELHMQMTDLVYEALHFAKDEEFQKHVFQLFEKA |
| | DENLLEEAERLLEEVKR | TKAYKNKDRQKLEKVVEELKELLERLLS |
| | (SEQ ID NO: 313} | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS11 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEEL |
| PAS16 | EHLEELARKGGASPAAPAGGNLDELHMQMTDLVYEA | ARK |
| 1_NTS | LHFAKDEEFQKHVFQLFEKATKAYKNKDRQKLEKVV | (SEQ ID NO: 101) |
| | EELKELLERLLSGGASPAAPAPASPAGGDKENVLQK | -X2- |
| | IYEIMKELERLGHAEASMQVSDLIYEFMKTKDENLL | NLDELHMQMTDLVYEALHFAKDEEFQKHVFQLFEKA |
| | EEAERLLEEVKR | TKAYKNKDRQKLEKVVEELKELLERLLS |
| | (SEQ ID NO: 314) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS24 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEEL |
| PAS11 | EHLEELARKGGASPAAPAPASPAAPAPSAPAGGNLD | ARK |
| 1_NTS | ELHMQMTDLVYEALHFAKDEEFQKHVFQLFEKATKA | (SEQ ID NO: 101) |
| | YKNKDRQKLEKVVEELKELLERLLSGGASPAAPAGG | -X2- |
| | DKENVLQKIYEIMKELERLGHAEASMQVSDLIYEFM | NLDELHMQMTDLVYEALHFAKDEEFQKHVFQLFEKA |
| | KTKDENLLEEAERLLEEVKR | TKAYKNKDRQKLEKVVEELKELLERLLS |
| | (SEQ ID NO: 315) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS16 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEEL |
| PAS11 | EHLEELARKGGASPAAPAPASPAGGNLDELHMQMTD | ARK |
| 1_NTS | LVYEALHFAKDEEFQKHVFQLFEKATKAYKNKDRQK | (SEQ ID NO: 101) |
| | LEKVVEELKELLERLLSGGASPAAPAGGDKENVLQK | -X2- |
| | IYEIMKELERLGHAEASMQVSDLIYEFMKTKDENLL | NLDELHMQMTDLVYEALHFAKDEEFQKHVFQLFEKA |
| | EEAERLLEEVKR | TKAYKNKDRQKLEKVVEELKELLERLLS |
| | (SEQ ID NO: 316) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2- | MEKKISAWSHPQFEKGGGSGGGSGGSAWSHPQFEKG | X1- |
| PAS11 | GSGSSGELEEQVMHVLDQVSELAHELLHKLTGEELE | ELEEQVMHVLDQVSELAHELLHKLTGEELERAAYFN |
| -3- | RAAYFNWWATEMMLELIKSDDEREIREIEEEARRIL | WWATEMMLELIKSDDEREIREIEEEARRILEHLEEL |
| PAS11 | EHLEELARKGGASPAAPAGGNLDELHMQMTDLVYEA | ARK |
| 1_NTS | LHFAKDEEFQKHVFQLFEKATKAYKNKDRQKLEKVV | (SEQ ID NO: 101) |
| | EELKELLERLLSGGASPAAPAGGDKENVLQKIYEIM | -X2- |
| | KELERLGHAEASMQVSDLIYEFMKTKDENLLEEAER | NLDELHMQMTDLVYEALHFAKDEEFQKHVFQLFEKA |
| | LLEEVKR | TKAYKNKDRQKLEKVVEELKELLERLLS |
| | (SEQ ID NO: 317) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3- | MEKKISAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYFN |
| PAS | RAAYFNWWATEMMLELIKSDDEREIREIEEEARRIL | WWATEMMLELIKSDDEREIREIEEEARRILEHLEEL |
| 7 | EHLEELARKGGASAGGNLDELHMQMTDLVYEALHFA | ARK |
| | KDEEFQKHVFQLFEKATKAYKNKDRQKLEKVVEELK | (SEQ ID NO: 101) |
| | ELLERLLSGGASAGGDKENVLQKIYEIMKELERLGH | -X2- |
| | AEASMQVSDLIYEFMKTKDENLLEEAERLLEEVKR | NLDELHMQMTDLVYEALHFAKDEEFQKHVFQLFEKA |
| | (SEQ ID NO: 318) | TKAYKNKDRQKLEKVVEELKELLERLLS |
| | | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3- | MEKKISAWSHPQFEKGGGSGGGSGGSAWSHPQFEKG | X1- |
| 1_GS7 | GSGSSGELEEQVMHVLDQVSELAHELLHKLTGEELE | ELEEQVMHVLDQVSELAHELLHKLTGEELERAAYFNWW |
| | RAAYFNWWATEMMLELIKSDDEREIREIEEEARRIL | ATEMMLELIKSDDEREIREIEEE2ARRILEHLEELARK |
| | EHLEELARKGGSGGSGGNLDELHMQMTDLVYEALHF | (SEQ ID NO: 101) |
| | AKDEEFQKHVFQLFEKATKAYKNKDRQKLEKVVEEL | -X2- |
| | KELLERLLSGGSGGSGGDKENVLQKIYEIMKELERL | NLDELHMQMTDLVYEALHFAKDEEFQKHVFQLFEKA |
| | GHAEASMQVSDLIYEFMKTKDENLLEEAERLLEEVK | TKAYKNKDRQKLEKVVEELKELLERLLS |
| | R | (SEQ ID NO: 155) |
| | (SEQ ID NO: 319) | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3- | | X1- |
| 1_GS5 | MEKKISAWSHPQFEKGGGSGGGSGGSAWSHPQFEKG | ELEEQVMHVLDQVSELAHELLHKLTGEELERAAYFN |
| | GSGSSGELEEQVMHVLDQVSELAHELLHKLTGEELE | WWATEMMLELIKSDDEREIREIEEEARRILEHLEEL |
| | RAAYFNWWATEMMLELIKSDDEREIREIEEEARRIL | ARK |
| | EHLEELARKGGSGGNLDELHMQMTDLVYEALHFAKD | (SEQ ID NO: 101) |
| | EEFQKHVFQLFEKATKAYKNKDRQKLEKVVEELKEL | -X2- |
| | LERLLSGGSGGDKENVLQKIYEIMKELERLGHAEAS | NLDELHMQMTDLVYEALHFAKDEEFQKHVFQLFEKA |
| | MQVSDLIYEFMKTKDENLLEEAERLLEEVKR | TKAYKNKDRQKLEKVVEELKELLERLLS |
| | (SEQ ID NO: 320) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3- | MEKKISAWSHPQFEKGGGSGGGSGGSAWSHPQFEKG | X1- |
| 1_ | GSGSSGELEEQVMHVLDQVSELAHELLHKLTGEELE | ELEEQVMHVLDQVSELAHELLHKLTGEELERAAYFN |
| GS11 | RAAYFNWWATEMMLELIKSDDEREIREIEEEARRIL | WWATEMMLELIKSDDEREIREIEEEARRILEHLEEL |
| | EHLEELARKGGSGGSGGSGGNLDELHMQMTDLVYEA | ARK |
| | LHFAKDEEFQKHVFQLFEKATKAYKNKDRQKLEKVV | (SEQ ID NO: 101) |
| | EELKELLERLLSGGSGGSGGSGGDKENVLQKIYEIM | -X2- |
| | KELERLGHAEASMQVSDLIYEFMKTKDENLLEEAER | NLDELHMQMTDLVYEALHFAKDEEFQKHVFQLFEKA |
| | LLEEVKR | TKAYKNKDRQKLEKVVEELKELLERLLS |
| | (SEQ ID NO: 321) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-2- | MEKKISAWSHPQFEKGGGSGGGSGGSAWSHPQFEKG | X1- |
| 2_ | GSGSSGELEEQVMHVLDQVSELAHELLHKLTGEELE | ELEEQVMHVLDQVSELAHELLHKLTGEELERAAYFN |
| PAS11 | RAAYFNWWATEMMLELIKSDDEREIREIEEEARRIL | WWATEMMLELIKSDDEREIREIEEEARRILEHLEEL |
| | EHLEELARKGGASPAAPAGGELEEQVMHVLDQVSEL | ARK |
| | AHELLHKLTGEELERAAYFNWWATEMMLELIKSDDE | (SEQ ID NO: 101) |
| | REIREIEEEARRILEHLEELARKGGASPAAPAGGEL | -X2- |
| | EEQVMHVLDQVSELAHELLHKLTGEELERAAYFNWW | ELEEQVMHVLDQVSELAHELLHKLTGEELERAAYFN |
| | ATEMMLELIKSDDEREIREIEEEARRILEHLEELAR | WWATEMMLELIKSDDEREIREIEEEARRILEHLEEL |
| | K | ARK |
| | (SEQ ID NO: 322) | (SEQ ID NO: 101) |
| | | -X3- |
| | | ELEEQVMHVLDQVSELAHELLHKLTGEELERAAYFN |
| | | WWATEMMLELIKSDDEREIREIEEEARRILEHLEEL |
| | | ARK |
| | | (SEQ ID NO: 101) |
| 2-2- | MEKKISAWSHPQFEKGGGSGGGSGGSAWSHPQFEKG | X1- |
| 2_ | GSGSSGELEEQVMHVLDQVSELAHELLHKLTGEELE | ELEEQVMHVLDQVSELAHELLHKLTGEELERAAYFN |
| GS11 | RAAYFNWWATEMMLELIKSDDEREIREIEEEARRIL | WWATEMMLELIKSDDEREIREIEEEIARRILEHLEE |
| | EHLEELARKGGSGGSGGSGGELEEQVMHVLDQVSEL | LARK |
| | AHELLHKLTGEELERAAYFNWWATEMMLELIKSDDE | (SEQ ID NO: 101) |
| | REIREIEEEARRILEHLEELARKGGSGGSGGSGGEL | -X2- |
| | EEQVMHVLDQVSELAHELLHKLTGEELERAAYFNWW | ELEEQVMHVLDQVSELAHELLHKLTGEELERAAYFN |
| | ATEMMLELIKSDDEREIREIEEEARRILEHLEELAR | WWATEMMLELIKSDDEREIREIEEEARRILEHLEEL |
| | K | ARK |
| | (SEQ ID NO: 323) | (SEQ ID NO: 101) |
| | | -X3- |
| | | ELEEQVMHVLDQVSELAHELLHKLTGEELERAAYFN |
| | | WWATEMMLELIKSDDEREIREIEEEARRILEHLEEL |
| | | ARK |
| | | (SEQ ID NO: 101) |
| 2-2- | MEKKISAWSHPQFEKGGGSGGGSGGSAWSHPQFEKG | X1- |
| 2_GS8 | GSGSSGELEEQVMHVLDQVSELAHELLHKLTGEELE | ELEEQVMHVLDQVSELAHELLHKLTGEELERAAYFN |
| | RAAYFNWWATEMMLELIKSDDEREIREIEEEARRIL | WWATEMMLELIKSDDEREIREIEEEARRILEHLEEL |
| | EHLEELARKGGSGGSGGELEEQVMHVLDQVSELAHE | ARK |
| | LLHKLTGEELERAAYFNWWATEMMLELIKSDDEREI | (SEQ ID NO: 101) |
| | REIEEEARRILEHLEELARKGGSGGSGGELEEQVMH | -X2- |
| | VLDQVSELAHELLHKLTGEELERAAYFNWWATEMML | ELEEQVMHVLDQVSELAHELLHKLTGEELERAAYFN |
| | ELIKSDDEREIREIEEE1ARRILEHLEELARK | WWATEMMLELIKSDDEREIREIEEEARRILEHLEEL |
| | (SEQ ID NO: 324) | ARK |
| | | (SEQ ID NO: 101) |
| | | -X3- |
| | | ELEEQVMHVLDQVSELAHELLHKLTGEELERAAYFN |
| | | WWATEMMLELIKSDDEREIREIEEEARRILEHLEEL |
| | | ARK |
| | | (SEQ ID NO: 101) |
| 2-2- | MEKKISAWSHPQFEKGGGSGGGSGGSAWSHPQFEKG | X1- |
| 2_GS5 | GSGSSGELEEQVMHVLDQVSELAHELLHKLTGEELE | ELEEQVMHVLDQVSELAHELLHKLTGEELERAAYFN |
| | RAAYFNWWATEMMLELIKSDDEREIREIEEEARRIL | WWATEMMLELIKSDDEREIREIEEEARRILEHLEEL |
| | EHLEELARKGGSGGELEEQVMHVLDQVSELAHELLH | ARK |
| | KLTGEELERAAYFNWWATEMMLELIKSDDEREIREI | (SEQ ID NO: 101) |
| | EEEARRILEHLEELARKGGSGGELEEQVMHVLDQVS | -X2- |
| | ELAHELLHKLTGEELERAAYFNWWATEMMLELIKSD | ELEEQVMHVLDQVSELAHELLHKLTGEELERAAYFN |
| | DEREIREIEEEARRILEHLEELARK | WWATEMMLELIKSDDEREIREIEEEARRILEHLEEL |
| | (SEQ ID NO: 325) | ARK |
| | | (SEQ ID NO: 101) |
| | | -X3- |
| | | ELEEQVMHVLDQVSELAHELLHKLTGEELERAAYFN |
| | | WWATEMMLELIKSDDEREIREIEEEARRILEHLEELA |
| | | RK |
| | | (SEQ ID NO: 101) |
| 2-3-1_ | MEKKISAWSHPQFEKGGGSGGGSGGSAWSHPQFEKG | X1- |
| GGG | GSGSSGELEEQVMHVLDQVSELAHELLHKLTGEELE | ELEEQVMHVLDQVSELAHELLHKLTGEELERAAYFN |
| GS15 | RAAYFNWWATEMMLELIKSDDEREIREIEEEARRIL | WWATEMMLELIKSDDEREIREIEEEARRILEHLEEL |
| | EHLEELARKGGSGGGGSGGGGSGGNLDELHMQMTDL | ARK |
| | VYEALHFAKDEEFQKHVFQLFEKATKAYKNKDRQKL | (SEQ ID NO: 101) |
| | EKVVEELKELLERLLSGGSGGGGSGGGGSGGDKENV | -X2- |
| | LQKIYEIMKELERLGHAEASMQVSDLIYEFMKTKDE | NLDELHMQMTDLVYEALHFAKDEEFQKHVFQLFEKA |
| | NLLEEAERLLEEVKR | TKAYKNKDRQKLEKVVEELKELLERLLS |
| | (SEQ ID NO: 326) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_ | MEKKISAWSHPQFEKGGGSGGGSGGSAWSHPQFEKG | X1- |
| GGG | GSGSSGELEEQVMHVLDQVSELAHELLHKLTGEELE | ELEEQVMHVLDQVSELAHELLHKLTGEELERAAYFNW |
| GS12 | RAAYFNWWATEMMLELIKSDDEREIREIEEEARRIL | WATEMMLELIKSDDEREIREIEEEARRILEHLEELA |
| | EHLEELARKGGSGGGGSGGGGNLDELHMQMTDLVYE | RK |
| | ALHFAKDEEFQKHVFQLFEKATKAYKNKDRQKLEKV | (SEQ ID NO: 101) |
| | VEELKELLERLLSGGSGGGGSGGGGDKENVLQKIYE | -X2- |
| | IMKELERLGHAEASMQVSDLIYEFMKTKDENLLEEA | NLDELHMQMTDLVYEALHFAKDEEFQKHVFQLFEKA |
| | ERLLEEVKR | TKAYKNKDRQKLEKVVEELKELLERLLS |
| | (SEQ ID NO: 327) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_ | MEKKISAWSHPQFEKGGGSGGGSGGSAWSHPQFEKG | X1- |
| GGG | GSGSSGELEEQVMHVLDQVSELAHELLHKLTGEELE | ELEEQVMHVLDQVSELAHELLHKLTGEELERAAYFN |
| GS9 | RAAYFNWWATEMMLELIKSDDEREIREIEEEARRIL | WWATEMMLELIKSDDEREIREIEEEARRILEHLEEL |
| | EHLEELARKGGGGSGGGGNLDELHMQMTDLVYEALH | ARK |
| | FAKDEEFQKHVFQLFEKATKAYKNKDRQKLEKVVEE | (SEQ ID NO: 101) |
| | LKELLERLLSGGGGSGGGGDKENVLQKIYEIMKELE | -X2- |
| | RLGHAEASMQVSDLIYEFMKTKDENLLEEAERLLEE | NLDELHMQMTDLVYEALHFAKDEEFQKHVFQLFEKA |
| | VKR | TKAYKNKDRQKLEKVVEELKELLERLLS |
| | (SEQ ID NO: 328) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| 2-3-1_ | MEKKISAWSHPQFEKGGGSGGGSGGSAWSHPQFEKG | X1- |
| GGG | GSGSSGELEEQVMHVLDQVSELAHELLHKLTGEELE | ELEEQVMHVLDQVSELAHELLHKLTGEELERAAYFN |
| GS7 | RAAYFNWWATEMMLELIKSDDEREIREIEEEARRIL | WWATEMMLELIKSDDEREIREIEEEARRILEHLEEL |
| | EHLEELARKGGGSGGGNLDELHMQMTDLVYEALHFA | ARK |
| | KDEEFQKHVFQLFEKATKAYKNKDRQKLEKVVEELK | (SEQ ID NO: 101) |
| | ELLERLLSGGGSGGGDKENVLQKIYEIMKELERLGH | -X2- |
| | AEASMQVSDLIYEFMKTKDENLLEEAERLLEEVKR | NLDELHMQMTDLVYEALHFAKDEEFQKHVFQLFEKA |
| | (SEQ ID NO: 329) | TKAYKNKDRQKLEKVVEELKELLERLLS |
| | | (SEQ ID NO: 155) |
| | | -X3- |
| | | DKENVLQKIYEIMKELERLG |
| | | (SEQ ID NO: 135) |
| | | HAEASMQVSDLIYEFMKTKDENLLEEAERLLEEVKR |
| 1-1_ | MEKKIGGGDKENVLQKIYEIMKELERLGHAEASMQV | X1- |
| GGG | SDLIYEFMKTKDENLLEEAERLLEEVKRGGSGGGGS | DKENVLQKIYEIMKELERLGHAEASMQVSDLIYEFM |
| GS25 | GGGGSGGGGSGGGGSGGDKENVLQKIYEIMKELERL | KTKDENLLEEAERLLEEVKR |
| | GHAEASMQVSDLIYEFMKTKDENLLEEAERLLEEVK | (SEQ ID NO: 135) |
| | RGGGSGGGSAWSHPQFEKGGGSGGGSGGSAWSHPQF | -X2- |
| | EK | DKENVLQKIYEIMKELERLGHAEASMQVSDLIYEFM |
| | (SEQ ID NO: 330} | KTKDENLLEEAERLLEEVKR |
| | | (SEQ ID NO: 135)-X3 |
| 1-1_ | MEKKIGGGDKENVLQKIYEIMKELERLGHAEASMQV | X1- |
| GGG | SDLIYEFMKTKDENLLEEAERLLEEVKRGGSGGGGS | DKENVLQKIYEIMKELERLGHAEASMQVSDLIYEFM |
| GS20 | GGGGSGGGGSGGDKENVLQKIYEIMKELERLGHAEA | KTKDENLLEEAERLLEEVKR |
| | SMQVSDLIYEFMKTKDENLLEEAERLLEEVKRGGGS | (SEQ ID NO: 135) |
| | GGGSAWSHPQFEKGGGSGGGSGGSAWSHPQFEK | -X2- |
| | (SEQ ID NO: 331) | DKENVLQKIYEIMKELERLGHAEASMQVSDLIYEFM |
| | | KTKDENLLEEAERLLEEVKR |
| | | (SEQ ID NO: 135)-X3 |
| 1-1_ | MEKKIGGGDKENVLQKIYEIMKELERLGHAEASMQV | X1- |
| GGG | SDLIYEFMKTKDENLLEEAERLLEEVKRGGSGGGGS | DKENVLQKIYEIMKELERLGHAEASMQVSDLIYEFM |
| GS15 | GGGGSGGDKENVLQKIYEIMKELERLGHAEASMQVS | KTKDENLLEEAERLLEEVKR |
| | DLIYEFMKTKDENLLEEAERLLEEVKRGGGSGGGSA | (SEQ ID NO: 135) |
| | WSHPQFEKGGGSGGGSGGSAWSHPQFEK | -X2- |
| | (SEQ ID NO: 332) | DKENVLQKIYEIMKELERLGHAEASMQVSDLIYEFM |
| | | KTKDENLLEEAERLLEEVKR |
| | | (SEQ ID NO: 135)-X3 |
| 1-1_ | MEKKIGGGDKENVLQKIYEIMKELERLGHAEASMQV | X1- |
| GGG | SDLIYEFMKTKDENLLEEAERLLEEVKRGGSGGGGS | DKENVLQKIYEIMKELERLGHAEASMQVSDLIYEFM |
| GS10 | GGDKENVLQKIYEIMKELERLGHAEASMQVSDLIYE | KTKDENLLEEAERLLEEVKR |
| | FMKTKDENLLEEAERLLEEVKRGGGSGGGSAWSHPQ | (SEQ ID NO: 135) |
| | FEKGGGSGGGSGGSAWSHPQFEK | -X2- |
| | (SEQ ID NO: 333) | DKENVLQKIYEIMKELERLGHAEASMQVSDLIYEFM |
| | | KTKDENLLEEAERLLEEVKR |
| | | (SEQ ID NO: 135)-X3 |
| 2-1_ | MEKKIGGGELEEQVMHVLDQVSELAHELLHKLTGEE | X1- |
| GGG | LERAAYFNWWATEMMLELIKSDDEREIREIEEEARR | ELEEQVMHVLDQVSELAHELLHKLTGEELERAAYFN |
| GS10 | ILEHLEELARKGGSGGGGSGGDKENVLQKIYEIMKE | WWATEMMLELIKSDDEREIREIEEEARRILEHLEEL |
| | LERLGHAEASMQVSDLIYEFMKTKDENLLEEAERLL | ARK |
| | EEVKRGGGSGGGSAWSHPQFEKGGGSGGGSGGSAWS | (SEQ ID NO: 101) |
| | HPQFEK | -X2- |
| | (SEQ ID NO: 334) | DKENVLQKIYEIMKELERLGHAEASMQVSDLIYEFM |
| | | KTKDENLLEEAERLLEEVKR |
| | | (SEQ ID NO: 135)-X3 |
| 3-1_ | MEKKIGGGNLDELHMQMTDLVYEALHFAKDEEFQKH | X1- |
| GGG | VFQLFEKATKAYKNKDRQKLEKVVEELKELLERLLS | NLDELHMQMTDLVYEALHFAKDEEFQKHVFQLFEKA |
| GS10 | GGSGGGGSGGDKENVLQKIYEIMKELERLGHAEASM | TKAYKNKDRQKLEKVVEELKELLERLLS |
| | QVSDLIYEFMKTKDENLLEEAERLLEEVKRGGGSGG | (SEQ ID NO: 155) |
| | GSAWSHPQFEKGGGSGGGSGGSAWSHPQFEK | -X2- |
| | (SEQ ID NO: 335) | DKENVLQKIYEIMKELERLGHAEASMQVSDLIYEFM |
| | | KTKDENLLEEAERLLEEVKR |
| | | (SEQ ID NO: 135)-X3 |
| 2-3_ | MEKKIGGGELEEQVMHVLDQVSELAHELLHKLTGEE | X1- |
| GGG | LERAAYFNWWATEMMLELIKSDDEREIREIEEEARR | ELEEQVMHVLDQVSELAHELLHKLTGEELERAAYFN |
| GS10 | ILEHLEELARKGGSGGGGSGGNLDELHMQMTDLVYE | WWATEMMLELIKSDDEREIREIEEEARRILEHLEEL |
| | ALHFAKDEEFQKHVFQLFEKATKAYKNKDRQKLEKV | ARK |
| | VEELKELLERLLSGGGSGGGSAWSHPQFEKGGGSGG | (SEQ ID NO: 101) |
| | GSGGSAWSHPQFEK | -X2- |
| | (SEQ ID NO: 336) | NLDELHMQMTDLVYEALHFAKDEEFQKHVFQLFEKA |
| | | TKAYKNKDRQKLEKVVEELKELLERLLS |
| | | (SEQ ID NO: 155)-X3 |
| 3-2_ | MEKKIGGGNLDELHMQMTDLVYEALHFAKDEEFQKH | X1- |
| GGG | VFQLFEKATKAYKNKDRQKLEKVVEELKELLERLLS | NLDELHMQMTDLVYEALHFAKDEEFQKHVFQLFEKA |
| GS10 | GGSGGGGSGGELEEQVMHVLDQVSELAHELLHKLTG | TKAYKNKDRQKLEKVVEELKELLERLLS |
| | EELERAAYFNWWATEMMLELIKSDDEREIREIEEEA | (SEQ ID NO: 155) |
| | RRILEHLEELARKGGGSGGGSAWSHPQFEKGGGSGG | -X2- |
| | GSGGSAWSHPQFEK | ELEEQVMHVLDQVSELAHELLHKLTGEELERAAYFN |
| | (SEQ ID NO: 337) | WWATEMMLELIKSDDEREIREIEEEARRILEHLEEL |
| | | ARK |
| | | (SEQ ID NO: 101)-X3 |
| 2-3-1_ | MEKKIGGGELEEQVMHVLDQVSELAHELLHKLTGEE | X1- |
| GGG | LERAAYFNWWATEMMLELIKSDDEREIREIEEEARR | ELEEQVMHVLDQVSELAHELLHKLTGEELERAAYFN |
| GS10 | ILEHLEELARKGGSGGGGSGGNLDELHMQMTDLVYE | WWATEMMLELIKSDDEREIREIEEEARRILEHLEEL |
| | ALHFAKDEEFQKHVFQLFEKATKAYKNKDRQKLEKV | ARK |
| | VEELKELLERLLSGGSGGGGSGGDKENVLQKIYEIM | (SEQ ID NO: 101) |
| | KELERLGHAEASMQVSDLIYEFMKTKDENLLEEAER | -X2- |
| | LLEEVKRGGGSGGGSAWSHPQFEKGGGSGGGSGGSA | NLDELHMQMTDLVYEALHFAKDEEFQKHVFQLFEKA |
| | WSHPQFEK | TKAYKNKDRQKLEKVVEELKELLERLLS |
| | (SEQ ID NO: 338) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| 3-2-1_ | MEKKIGGGNLDELHMQMTDLVYEALHFAKDEEFQKH | X1- |
| GGG | VFQLFEKATKAYKNKDRQKLEKVVEELKELLERLLS | NLDELHMQMTDLVYEALHFAKDEEFQKHVFQLFEKA |
| GS10 | GGSGGGGSGGELEEQVMHVLDQVSELAHELLHKLTG | TKAYKNKDRQKLEKVVEELKELLERLLS |
| | EELERAAYFNWWATEMMLELIKSDDEREIREIEEEA | (SEQ ID NO: 155) |
| | RRILEHLEELARKGGSGGGGSGGDKENVLQKIYEIM | -X2- |
| | KELERLGHAEASMQVSDLIYEFMKTKDENLLEEAER | ELEEQVMHVLDQVSELAHELLHKLTGEELERAAYFN |
| | LLEEVKRGGGSGGGSAWSHPQFEKGGGSGGGSGGSA | WWATEMMLELIKSDDEREIREIEEEARRILEHLEEL |
| | WSHPQFEK | ARK |
| | (SEQ ID NO: 339) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| 2-3-1_ | MEKKIGGGELEEQVMHVLDQVSELAHELLHKLTGEE | X1- |
| GGG | LERAAYFNWWATEMMLELIKSDDEREIREIEEEARR | ELEEQVMHVLDQVSELAHELLHKLTGEELERAAYFN |
| GS15 | ILEHLEELARKGGSGGGGSGGGGSGGNLDELHMQMT | WWATEMMLELIKSDDEREIREIEEEARRILEHLEEL |
| | DLVYEALHFAKDEEFQKHVFQLFEKATKAYKNKDRQ | ARK |
| | KLEKVVEELKELLERLLSGGSGGGGSGGGGSGGDKE | (SEQ ID NO: 101) |
| | NVLQKIYEIMKELERLGHAEASMQVSDLIYEFMKTK | -X2- |
| | DENLLEEAERLLEEVKRGGGSGGGSAWSHPQFEKGG | NLDELHMQMTDLVYEALHFAKDEEFQKHVFQLFEKA |
| | GSGGGSGGSAWSHPQFEK | TKAYKNKDRQKLEKVVEELKELLERLLS |
| | (SEQ ID NO: 340) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| 3-2- | MEKKIGGGNLDELHMQMTDLVYEALHFAKDEEFQKH | X1- |
| 1_ | VFQLFEKATKAYKNKDRQKLEKVVEELKELLERLLS | NLDELHMQMTDLVYEALHFAKDEEFQKHVFQLFEKA |
| GGG | GGSGGGGSGGGGSGGELEEQVMHVLDQVSELAHELL | TKAYKNKDRQKLEKVVEELKELLERLLS |
| GS15 | HKLTGEELERAAYFNWWATEMMLELIKSDDEREIRE | (SEQ ID NO: 155) |
| | IEEEARRILEHLEELARKGGSGGGGSGGGGSGGDKE | -X2- |
| | NVLQKIYEIMKELERLGHAEASMQVSDLIYEFMKTK | ELEEQVMHVLDQVSELAHELLHKLTGEELERAAYFN |
| | DENLLEEAERLLEEVKRGGGSGGGSAWSHPQFEKGG | WWATEMMLELIKSDDEREIREIEEEARRILEHLEEL |
| | GSGGGSGGSAWSHPQFEK | ARK |
| | (SEQ ID NO: 341) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| LCB3- | MEKKINLDELHMQMTDLVYEALHFAKDEEFQKHVFQ | X1- |
| LCB1_ | LFEKATKAYKNKDRQKLEKVVEELKELLERLLSGGA | NLDELHMQMTDLVYEALHFAKDEEFQKHVFQLFEKA |
| PAS12 | SPAAPAPGGDKENVLQKIYEIMKELERLGHAEASMQ | TKAYKNKDRQKLEKVVEELKELLERLLS |
| | VSDLIYEFMKTKDENLLEEAERLLEEVKRGGSGSSG | (SEQ ID NO: 155) |
| | SAWSHPQFEKGGGSGGGSGGSAWSHPQFEK | -X2- |
| | (SEQ ID NO: 342) | DKENVLQKIYEIMKELERLGHAEASMQVSDLIYEFM |
| | | KTKDENLLEEAERLLEEVKR |
| | | (SEQ ID NO: 135)-X3 |
| AHB2- | MEKKIELEEQVMHVLDQVSELAHELLHKLTGEELER | X1- |
| LCB1_ | AAYFNWWATEMMLELIKSDDEREIREIEEEARRILE | ELEEQVMHVLDQVSELAHELLHKLTGEELERAAYFN |
| PAS12 | HLEELARKGGASPAAPAPGGDKENVLQKIYEIMKEL | WWATEMMLELIKSDDEREIREIEEEARRILEHLEEL |
| | ERLGHAEASMQVSDLIYEFMKTKDENLLEEAERLLE | ARK |
| | EVKRGGSGSSGSAWSHPQFEKGGGSGGGSGGSAWSH | (SEQ ID NO: 101) |
| | PQFEK | -X2- |
| | (SEQ ID NO: 343) | DKENVLQKIYEIMKELERLGHAEASMQVSDLIYEFM |
| | | KTKDENLLEEAERLLEEVKR |
| | | (SEQ ID NO: 135)-X3 |
| AHB2- | MEKKIELEEQVMHVLDQVSELAHELLHKLTGEELER | X1- |
| LCB3_ | AAYFNWWATEMMLELIKSDDEREIREIEEEARRILE | ELEEQVMHVLDQVSELAHELLHKLTGEELERAAYFN |
| PAS12 | HLEELARKGGASPAAPAPGGNLDELHMQMTDLVYEA | WWATEMMLELIKSDDEREIREIEEEARRILEHLEEL |
| | LHFAKDEEFQKHVFQLFEKATKAYKNKDRQKLEKVV | ARK |
| | EELKELLERLLSGGSGSSGSAWSHPQFEKGGGSGGG | (SEQ ID NO: 101) |
| | SGGSAWSHPQFEK | -X2- |
| | (SEQ ID NO: 344) | NLDELHMQMTDLVYEALHFAKDEEFQKHVFQLFEKA |
| | | TKAYKNKDRQKLEKVVEELKELLERLLS |
| | | (SEQ ID NO: 155)-X3 |
| LCB3- | MEKKINLDELHMQMTDLVYEALHFAKDEEFQKHVFQ | X1- |
| AHB2_ | LFEKATKAYKNKDRQKLEKVVEELKELLERLLSGGA | NLDELHMQMTDLVYEALHFAKDEEFQKHVFQLFEKA |
| PAS12 | SPAAPAPGGELEEQVMHVLDQVSELAHELLHKLTGE | TKAYKNKDRQKLEKVVEELKELLERLLS |
| | ELERAAYFNWWATEMMLELIKSDDEREIREIEEEAR | (SEQ ID NO: 155) |
| | RILEHLEELARKGGSGSSGSAWSHPQFEKGGGSGGG | -X2- |
| | SGGSAWSHPQFEK | ELEEQVMHVLDQVSELAHELLHKLTGEELERAAYFN |
| | (SEQ ID NO: 345) | WWATEMMLELIKSDDEREIREIEEEARRILEHLEEL |
| | | ARK |
| | | (SEQ ID NO: 101)-X3 |
| AHB2- | MEKKIELEEQVMHVLDQVSELAHELLHKLTGEELER | X1- |
| LCB3- | AAYFNWWATEMMLELIKSDDEREIREIEEEARRILE | ELEEQVMHVLDQVSELAHELLHKLTGEELERAAYFN |
| LCB1_ | HLEELARKGGASPAAPAPGGNLDELHMQMTDLVYEA | WWATEMMLELIKSDDEREIREIEEEARRILEHLEEL |
| PAS12 | LHFAKDEEFQKHVFQLFEKATKAYKNKDRQKLEKVV | ARK |
| | EELKELLERLLSGGASPAAPAPGGDKENVLQKIYEI | (SEQ ID NO: 101) |
| | MKELERLGHAEASMQVSDLIYEFMKTKDENLLEEAE | -X2- |
| | RLLEEVKRGGSGSSGSAWSHPQFEKGGGSGGGSGGS | NLDELHMQMTDLVYEALHFAKDEEFQKHVFQLFEKA |
| | AWSHPQFEK | TKAYKNKDRQKLEKVVEELKELLERLLS |
| | (SEQ ID NO: 346) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| LCB3- | MEKKINLDELHMQMTDLVYEALHFAKDEEFQKHVFQ | X1- |
| AHB2- | LFEKATKAYKNKDRQKLEKVVEELKELLERLLSGGA | NLDELHMQMTDLVYEALHFAKDEEFQKHVFQLFEKA |
| LCB1_ | SPAAPAPGGELEEQVMHVLDQVSELAHELLHKLTGE | TKAYKNKDRQKLEKVVEELKELLERLLS |
| PAS12 | ELERAAYFNWWATEMMLELIKSDDEREIREIEEEAR | (SEQ ID NO: 155) |
| | RILEHLEELARKGGASPAAPAPGGDKENVLQKIYEI | -X2- |
| | MKELERLGHAEASMQVSDLIYEFMKTKDENLLEEAE | ELEEQVMHVLDQVSELAHELLHKLTGEELERAAYFN |
| | RLLEEVKRGGSGSSGSAWSHPQFEKGGGSGGGSGGS | WWATEMMLELIKSDDEREIREIEEEARRILEHLEEL |
| | AWSHPQFEK | ARK |
| | (SEQ ID NO: 347) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| LCB3- | MEKKINLDELHMQMTDLVYEALHFAKDEEFQKHVFQ | X1- |
| AHB2- | LFEKATKAYKNKDRQKLEKVVEELKELLERLLSGGA | NLDELHMQMTDLVYEALHFAKDEEFQKHVFQLFEKA |
| LCB1_ | SPAAPAPASPAAPAPSAPAGGELEEQVMHVLDQVSE | TKAYKNKDRQKLEKVVEELKELLERLLS |
| PAS24 | LAHELLHKLTGEELERAAYFNWWATEMMLELIKSDD | (SEQ ID NO: 155) |
| | EREIREIEEEARRILEHLEELARKGGASPAAPAPAS | -X2- |
| | PAAPAPSAPAGGDKENVLQKIYEIMKELERLGHAEA | ELEEQVMHVLDQVSELAHELLHKLTGEELERAAYFN |
| | SMQVSDLIYEFMKTKDENLLEEAERLLEEVKRGGSG | WWATEMMLELIKSDDEREIREIEEEARRILEHLEEL |
| | SSGSAWSHPQFEKGGGSGGGSGGSAWSHPQFEK | ARK |
| | (SEQ ID NO: 348) | (SEQ ID NO: 101) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| AHB2v | MEKKIELEEQVMHVLDQVSELAHELLHKLTGEELER | X1- |
| 2- | AAYFNWWATEMMLELIKSDDEREIREIEEEAARILE | ELEEQVMHVLDQVSELAHELLHKLTGEELERAAYFN |
| LCB1 | HLEELARTGGASPAAPAPGGDKENVLQKIYEIMKEL | WWATEMMLELIKSDDEREIREIEEEAARILEHLEEL |
| PAS12 | ERLGHAEASMQVSDLIYEFMKTKDENLLEEAERLLE | ART |
| | EVKRGGSGSSGSAWSHPQFEKGGGSGGGSGGSAWSH | (SEQ ID NO: 164) |
| | PQFEK | -X2- |
| | (SEQ ID NO: 349) | DKENVLQKIYEIMKELERLGHAEASMQVSDLIYEFM |
| | | KTKDENLLEEAERLLEEVKR |
| | | (SEQ ID NO: 135)-X3 |
| AHB2v | MEKKIELEEQVMHVLDQVSELAHELLHKLTGEELER | X1- |
| 2- | AAYFNWWATEMMLELIKSDDEREIREIEEEAARILE | ELEEQVMHVLDQVSELAHELLHKLTGEELERAAYFN |
| LCB3 | HLEELARTGGASPAAPAPGGNLDELHMQMTDLVYEA | WWATEMMLELIKSDDEREIREIEEEAARILEHLEEL |
| PAS12 | LHFAKDEEFQKHVFQLFEKATKAYKNKDRQKLEKVV | ART |
| | EELKELLERLLSGGSGSSGSAWSHPQFEKGGGSGGG | (SEQ ID NO: 164) |
| | SGGSAWSHPQFEK | -X2- |
| | (SEQ ID NO: 350) | NLDELHMQMTDLVYEALHFAKDEEFQKHVFQLFEKA |
| | | TKAYKNKDRQKLEKVVEELKELLERLLS |
| | | (SEQ ID NO: 155)-X3 |
| LCB3- | MEKKINLDELHMQMTDLVYEALHFAKDEEFQKHVFQ | X1- |
| AHB2v | LFEKATKAYKNKDRQKLEKVVEELKELLERLLSGGA | NLDELHMQMTDLVYEALHFAKDEEFQKHVFQLFEKA |
| 2_PAS | SPAAPAPGGELEEQVMHVLDQVSELAHELLHKLTGE | TKAYKNKDRQKLEKVVEELKELLERLLS |
| 12 | ELERAAYFNWWATEMMLELIKSDDEREIREIEEEAA | (SEQ ID NO: 155) |
| | RILEHLEELARTGGSGSSGSAWSHPQFEKGGGSGGG | -X2- |
| | SGGSAWSHPQFEK | ELEEQVMHVLDQVSELAHELLHKLTGEELERAAYFN |
| | (SEQ ID NO: 351) | WWATEMMLELIKSDDEREIREIEEEAARILEHLEEL |
| | | ART |
| | | (SEQ ID NO: 164)-X3 |
| AHB2v | MEKKIELEEQVMHVLDQVSELAHELLHKLTGEELER | X1- |
| 2- | AAYFNWWATEMMLELIKSDDEREIREIEEEAARILE | ELEEQVMHVLDQVSELAHELLHKLTGEELERAAYFN |
| LCB3- | HLEELARTGGASPAAPAPGGNLDELHMQMTDLVYEA | WWATEMMLELIKSDDEREIREIEEEAARILEHLEEL |
| LCB1 | LHFAKDEEFQKHVFQLFEKATKAYKNKDRQKLEKVV | ART |
| PAS12 | EELKELLERLLSGGASPAAPAPGGDKENVLQKIYEI | (SEQ ID NO: 164J |
| | MKELERLGHAEASMQVSDLIYEFMKTKDENLLEEAE | -X2- |
| | RLLEEVKRGGSGSSGSAWSHPQFEKGGGSGGGSGGS | NLDELHMQMTDLVYEALHFAKDEEFQKHVFQLFEKA |
| | AWSHPQFEK | TKAYKNKDRQKLEKVVEELKELLERLLS |
| | (SEQ ID NO: 352) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| LCB3- | MEKKINLDELHMQMTDLVYEALHFAKDEEFQKHVFQ | X1- |
| AHB2v | LFEKATKAYKNKDRQKLEKVVEELKELLERLLSGGA | NLDELHMQMTDLVYEALHFAKDEEFQKHVFQLFEKA |
| 2- | SPAAPAPGGELEEQVMHVLDQVSELAHELLHKLTGE | TKAYKNKDRQKLEKVVEELKELLERLLS |
| LCB1 | ELERAAYFNWWATEMMLELIKSDDEREIREIEEEAA | (SEQ ID NO: 155) |
| PAS12 | RILEHLEELARTGGASPAAPAPGGDKENVLQKIYEI | -X2- |
| | MKELERLGHAEASMQVSDLIYEFMKTKDENLLEEAE | ELEEQVMHVLDQVSELAHELLHKLTGEELERAAYFN |
| | RLLEEVKRGGSGSSGSAWSHPQFEKGGGSGGGSGGS | WWATEMMLELIKSDDEREIREIEEEAARILEHLEEL |
| | AWSHPQFEK | ART |
| | (SEQ ID NO: 353) | (SEQ ID NO: 164) |
| | | -X3- |
| | | DKENVLQKIYEIMKELERLGHAEASMQVSDLIYEFM |
| | | KTKDENLLEEAERLLEEVKR |
| | | (SEQ ID NO: 135)-X4 |
| AHB2v | MEKKISAWSHPQFEKGGGSGGGSGGSAWSHPQFEKG | X1- |
| 2- | GSGSSGELEEQVMHVLDQVSELAHELLHKLTGEELE | ELEEQVMHVLDQVSELAHELLHKLTGEELERAAYFN |
| LCB3- | RAAYFNWWATEMMLELIKSDDEREIREIEEEAARIL | WWATEMMLELIKSDDEREIREIEEEAARILEHLEEL |
| LCB1_ | EHLEELARTGGASPAAPAPASPAAPAPSAPAGGNLD | ART |
| PAS24 | ELHMQMTDLVYEALHFAKDEEFQKHVFQLFEKATKA | (SEQ ID NO: 164) |
| | YKNKDRQKLEKVVEELKELLERLLSGGASPAAPAPA | -X2- |
| | SPAAPAPSAPAGGDKENVLQKIYEIMKELERLGHAE | NLDELHMQMTDLVYEALHFAKDEEFQKHVFQLFEKA |
| | ASMQVSDLIYEFMKTKDENLLEEAERLLEEVKR | TKAYKNKDRQKLEKVVEELKELLERLLS |
| | (SEQ ID NO: 354) | (SEQ ID NO: 155) |
| | | -X3- |
| | | DKENVLQKIYEIMKELERLGHAEASMQVSDLIYEFM |
| | | KTKDENLLEEAERLLEEVKR |
| | | (SEQ ID NO: 135) |
| LCB3- | MEKKINLDELHMQMTDLVYEALHFAKDEEFQKHVFQ | X1- |
| AHB2v | LFEKATKAYKNKDRQKLEKVVEELKELLERLLSGGA | NLDELHMQMTDLVYEALHFAKDEEFQKHVFQLFEKA |
| 2- | SPAAPAPASPAAPAPSAPAGGELEEQVMHVLDQVSE | TKAYKNKDRQKLEKVVEELKELLERLLS |
| LCB1_ | LAHELLHKLTGEELERAAYFNWWATEMMLELIKSDD | (SEQ ID NO: 155) |
| PAS24 | EREIREIEEEAARILEHLEELARTGGASPAAPAPAS | -X2- |
| | PAAPAPSAPAGGDKENVLQKIYEIMKELERLGHAEA | ELEEQVMHVLDQVSELAHELLHKLTGEELERAAYFN |
| | SMQVSDLIYEFMKTKDENLLEEAERLLEEVKRGGSG | WWATEMMLELIKSDDEREIREIEEEAARILEHLEEL |
| | SSGSAWSHPQFEKGGGSGGGSGGSAWSHPQFEK | ART |
| | (SEQ ID NO: 355) | (SEQ ID NO: 164) |
| | | -X3- |
| | | DKENVLQKIYEIMKELERLGHAEASMQVSDLIYEFM |
| | | KTKDENLLEEAERLLEEVKR |
| | | (SEQ ID NO: |

TABLE 8A

| SEQ ID | Name | Sequence |
|---|---|---|
| 51 | 1GS1 | DKENILQKIYEIMKTLDQLGHAEASMQVSDLIYEFMKQGDERLLEEAERLLEEVERG |
| | | GGGSGGGGSGGGGSGGGGSGGGGSGGGGSGGGGSGGGGSGGGGSGGGGSGGGGSGGG |
| | | GSGGGGSGGGGSGGGGSDKENILQKIYEIMKTLDQLGHAEASMQVSDLIYEFMKQGD |
| | | ERLLEEAERLLEEVER |
| 52 | 1PRO1 | DKENILQKIYEIMKTLDQLGHAEASMQVSDLIYEFMKQGDERLLEEAERLLEEVERA |
| | | GSGGSGGSGGSPVPSTPPTPSPSTPPTPSPSPVPSTPPTPSPSTPPTPSPSPVPSTP |
| | | PTPSPSTPPTPSPSASGDKENILQKIYEIMKTLDQLGHAEASMQVSDLIYEFMKQGD |
| | | ERLLEEAERLLEEVER |
| 53 | 3GS3 | NDDELHMQMTDLVYEALHFAKDEEIQKHVFQLFEKATKAYKNKDRQKLEKVVEELKE |
| | | LLERLLSGGGGSGGGGSGGGGSGGGGSGGGGSGGGGSGGGGSGGGGSGGGGSGGGGS |
| | | GGGGSGGGGSGGGGSGGGGSGGGGSNDDELHMQMTDLVYEALHFAKDEEIQKHVFQL |
| | | FEKATKAYKNKDROKLEKVVEELKELLERLLS |
| 54 | 3PRO3 | NDDELHMQMTDLVYEALHFAKDEEIQKHVFQLFEKATKAYKNKDRQKLEKVVEELKE |
| | | LLERLLSAGSGGSGGSGGSPVPSTPPTPSPSTPPTPSPSPVPSTPPTPSPSTPPTPS |
| | | PSPVPSTPPTPSPSTPPTPSPSASGNDDELHMQMTDLVYEALHFAKDEEIQKHVFQL |
| | | FEKATKAYKNKDROKLEKVVEELKELLERLLS |
| 55 | 1GS3 | DKENILQKIYEIMKTLDQLGHAEASMQVSDLIYEFMKQGDERLLEEAERLLEEVERG |
| | | GGGSGGGGSGGGGSGGGGSGGGGSGGGGSGGGGSGGGGSGGGGSGGGGSGGGGSGGG |
| | | GSGGGGSGGGGSGGGGSNDDELHMQMTDLVYEALHFAKDEEIQKHVFQLFEKATKAY |
| | | KNKDRQKLEKVVEELKELLERLLS |
| 56 | 3GS1 | NDDELHMQMTDLVYEALHFAKDEEIQKHVFQLFEKATKAYKNKDRQKLEKVVEELKE |
| | | LLERLLSGGGGSGGGGSGGGGSGGGGSGGGGSGGGGSGGGGSGGGGSGGGGSGGGGS |
| | | GGGGSGGGGSGGGGSGGGGSGGGGSDKENILQKIYEIMKTLDQLGHAEASMQVSDLI |
| | | YEFMKQGDERLLEEAERLLEEVER |
| 58 | 1PRO3 | DKENILQKIYEIMKTLDQLGHAEASMQVSDLIYEFMKQGDERLLEEAERLLEEVERA |
| | | GSGGSGGSGGSPVPSTPPTPSPSTPPTPSPSPVPSTPPTPSPSTPPTPSPSPVPSTP |
| | | PTPSPSTPPTPSPSASGNDDELHMQMTDLVYEALHFAKDEEIQKHVFQLFEKATKAY |
| | | KNKDRQKLEKVVEELKELLERLLS |
| 59 | 3PRO1 | NDDELHMQMTDLVYEALHFAKDEEIQKHVFQLFEKATKAYKNKDRQKLEKVVEELKE |
| | | LLERLLSAGSGGSGGSGGSPVPSTPPTPSPSTPPTPSPSPVPSTPPTPSPSTPPTPS |
| | | PSPVPSTPPTPSPSTPPTPSPSASGDKENILQKIYEIMKTLDQLGHAEASMQVSDLI |
| | | YEFMKQGDERLLEEAERLLEEVER |
| 454 | CSL- | DKEWILQKIYEIMRLLDELGHAEASMRVSDLIYEFMKKGDERLLEEAERLLEEVERG |
| | LCB1- | GGGSGGGGSGGGGSDKEWILQKIYEIMRLLDELGHAEASMRVSDLIYEFMKKGDERL |
| | GS15- | LEEAERLLEEVER |
| | LCB1 | |
| 455 | CSL- | NDDELHMLMTDLVYEALHFAKDEEIKKRVFQLFELADKAYKNNDRQKLEKVVEELKE |
| | LCB3- | LLERLLSGGGGSGGGGSGGGGSNDDELHMLMTDLVYEALHFAKDEEIKKRVFQLFEL |
| | GS15- | ADKAYKNNDRQKLEKVVEELKELLERLLS |
| | LCB3 | |
| 456 | CSL- | DKEWILQKIYEIMRLLDELGHAEASMRVSDLIYEFMKKGDERLLEEAERLLEEVERG |
| | LCB1- | GGGSGGGGSGGGGSGGGGSDKEWILQKIYEIMRLLDELGHAEASMRVSDLIYEFMKK |
| | GS20- | GDERLLEEAERLLEEVER |
| | LCB1 | |
| 457 | CSL- | NDDELHMLMTDLVYEALHFAKDEEIKKRVFQLFELADKAYKNNDRQKLEKVVEELKE |
| | LCB3- | LLERLLSGGGGSGGGGSGGGGSGGGGSNDDELHMLMTDLVYEALHFAKDEEIKKRVF |
| | GS20- | QLFELADKAYKNNDRQKLEKVVEELKELLERLLS |
| | LCB3 | |
| 458 | CSL- | DKEWILQKIYEIMRLLDELGHAEASMRVSDLIYEFMKKGDERLLEEAERLLEEVERG |
| | LCB1- | SSGSGSSGSGSSGSGSSGSDKEWILQKIYEIMRLLDELGHAEASMRVSDLIYEFMKK |
| | GS20- | GDERLLEEAERLLEEVERGSSSGGSSSGGSSSGGSSSGDKEWILQKIYEIMRLLDEL |
| | LCB1- | GHAEASMRVSDLIYEFMKKGDERLLEEAERLLEEVER |
| | GS20- | |
| | LCB1 | |
| 459 | CSL- | NDDELHMLMTDLVYEALHFAKDEEIKKRVFQLFELADKAYKNNDRQKLEKVVEELKE |
| | LCB3- | LLERLLSGSSGSGSSGSGSSGSGSSGSNDDELHMLMTDLVYEALHFAKDEEIKKRVF |
| | GS20- | QLFELADKAYKNNDRQKLEKVVEELKELLERLLSGSSSGGSSSGGSSSGGSSSGNDD |
| | LCB3- | ELHMLMTDLVYEALHFAKDEEIKKRVFQLFELADKAYKNNDRQKLEKVVEELKELLE |
| | GS20- | RLLS |
| | LCB3 | |
| 460 | CSL- | DKEWILQKIYEIMRLLDELGHAEASMRVSDLIYEFMKKGDERLLEEAERLLEEVERG |
| | LCB1- | SAGGSPAGSPTSTGTSTSGDKEWILQKIYEIMRLLDELGHAEASMRVSDLIYEFMKK |
| | XTENx2 | GDERLLEEAERLLEEVER |
| 461 | CSL- | DKEWILQKIYEIMRLLDELGHAEASMRVSDLIYEFMKKGDERLLEEAERLLEEVERG |
| | LCB1- | SAGGSPAGSPTSTGTSGSGDKEWILQKIYEIMRLLDELGHAEASMRVSDLIYEFMKK |
| | XTENx3 | GDERLLEEAERLLEEVERGSAGGSPAGSPTSTGTSGSGDKEWILQKIYEIMRLLDEL |
| | | GHAEASMRVSDLIYEFMKKGDERLLEEAERLLEEVER |
| 462 | CSL- | NDDELHMLMTDLVYEALHFAKDEEIKKRVFQLFELADKAYKNNDRQKLEKVVEELKE |
| | LCB3- | LLERLLSGSAGGSPAGSPTSTGTSGSGNDDELHMLMTDLVYEALHFAKDEEIKKRVF |
| | XTENx2 | QLFELADKAYKNNDRQKLEKVVEELKELLERLLS |
| 463 | CSL- | NDDELHMLMTDLVYEALHFAKDEEIKKRVFQLFELADKAYKNNDRQKLEKVVEELKE |
| | LCB3- | LLERLLSGSAGGSPAGSPTSTGTSGSGNDDELHMLMTDLVYEALHFAKDEEIKKRVF |
| | XTENx3 | QLFELADKAYKNNDRQKLEKVVEELKELLERLLSGSAGGSPAGSPTSTGTSGSGNDD |
| | | ELHMLMTDLVYEALHFAKDEEIKKRVFQLFELADKAYKNNDRQKLEKVVEELKELLE |
| | | RLLS |
| 458 | C- | DKEWILQKIYEIMRLLDELGHAEASMRVSDLIYEFMKKGDERLLEEAERLLEEVERG |
| | LCB1- | SSGSGSSGSGSSGSGSSGSDKEWILQKIYEIMRLLDELGHAEASMRVSDLIYEFMKK |
| | GS20- | GDERLLEEAERLLEEVERGSSSGGSSSGGSSSGGSSSGDKEWILQKIYEIMRLLDEL |
| | LCB1- | GHAEASMRVSDLIYEFMKKGDERLLEEAERLLEEVER |
| | GS20- | |
| | LCB1- | |
| | LS | |
| 459 | C- | NDDELHMLMTDLVYEALHFAKDEEIKKRVFQLFELADKAYKNNDRQKLEKVVEELKE |
| | LCB3- | LLERLLSGSSGSGSSGSGSSGSGSSGSNDDELHMLMTDLVYEALHFAKDEEIKKRVF |
| | GS20- | QLFELADKAYKNNDRQKLEKVVEELKELLERLLSGSSSGGSSSGGSSSGGSSSGNDD |
| | LCB3- | ELHMLMTDLVYEALHFAKDEEIKKRVFQLFELADKAYKNNDRQKLEKVVEELKELLE |
| | GS20- | RLLS |
| | LCB3- | |
| | LS | |
| 464 | CSL- | DKEWILQKIYEIMRLLDELGHAEASMRVSDLIYEFMKKGDERLLEEAERLLEEVERG |
| | LCB1- | GSSAGSPTSTGTSSATPSGSGTGGDKEWILQKIYEIMRLLDELGHAEASMRVSDLIY |
| | XTEN25 | EFMKKGDERLLEEAERLLEEVERGGSSAGSPTSTGTSSATPSGSGTGGDKEWILQKI |
| | x3 | YEIMRLLDELGHAEASMRVSDLIYEFMKKGDERLLEEAERLLEEVER |
| 465 | CSL- | NDDELHMLMTDLVYEALHFAKDEEIKKRVFQLFELADKAYKNNDRQKLEKVVEELKE |
| | LCB3- | LLERLLSGGSSAGSPTSTGTSSATPSGSGTGGNDDELHMLMTDLVYEALHFAKDEEI |
| | XTEN25 | KKRVFQLFELADKAYKNNDRQKLEKVVEELKELLERLLSGGSSAGSPTSTGTSSATP |
| | x3 | SGSGTGGNDDELHMLMTDLVYEALHFAKDEEIKKRVFQLFELADKAYKNNDRQKLEK |
| | | WEELKELLERLLS |
| 466 | LCB1_ | DKENILQKIYEIMKTLEQLGHAEASMQVSDLIYEFMKQGDERLLEEAERLLEEVERG |
| | v1.3_GS_ | GSSGGGSSGGGSSGGGSSGGGSSGDKENILQKIYEIMKTLEQLGHAEASMQVSDLIY |
| | 2X | EFMKQGDERLLEEAERLLEEVER |
| 467 | LCB1_ | DKENILQKIYEIMKTLEQLGHAEASMQVSDLIYEFMKQGDERLLEEAERLLEEVERG |
| | v1.3_XTEN_ | GSSAGSPTSTGTSSATPSGSGTGGDKENILQKIYEIMKTLEQLGHAEASMQVSDLIY |
| | 2X | EFMKQGDERLLEEAERLLEEVER |
| 468 | LCB1_v | DKENILQKIYEIMKTLEQLGHAEASMQVSDLIYEFMKQGDERLLEEAERLLEEVERG |
| | 1.3_EAAAK_ | GSSGEAAAKEAAAKEAAAKGSSGGDKENILQKIYEIMKTLEQLGHAEASMQVSDLIY |
| | 2X | EFMKQGDERLLEEAERLLEEVER |
| 469 | LCB1_ | DKENILQKIYEIMKTLEQLGHAEASMQVSDLIYEFMKQGDERLLEEAERLLEEVERG |
| | v1.3_Pro_ | GSSGPSTPPTPSPSTPPTPSPSPGGSSGDKENILQKIYEIMKTLEQLGHAEASMQVS |
| | 2X | DLIYEFMKQGDERLLEEAERLLEEVER |
| 470 | LCB1_v | DKENILQKIYEIMKTLEQLGHAEASMQVSDLIYEFMKQGDERLLEEAERLLEEVERG |
| | 1.3_Ub_2x | GSSGQIFVKTLTGKTITLEVEPSDTIENVKAKIQDKEGIPPDQQRLIFAGKQLEDGR |
| | | TLSDYNIQKESTLHLVLRLRGGGGSSGDKENILQKIYEIMKTLEQLGHAEASMQVSD |
| | | LIYEFMKQGDERLLEEAERLLEEVER |
| 471 | LCB1_ | DKENILQKIYEIMKTLEQLGHAEASMQVSDLIYEFMKQGDERLLEEAERLLEEVERG |
| | v1.3_XTEN_ | GSSAGSPTSTGTSSATPSGSGTGGDKENILQKIYEIMKTLEQLGHAEASMQVSDLIY |
| | 3X | EFMKQGDERLLEEAERLLEEVERGGSSAGSPTSTGTSSATPSGSGTGGDKENILQKI |
| | | YEIMKTLEQLGHAEASMQVSDLIYEFMKQGDERLLEEAERLLEEVER |
| 472 | LCB1_ | DKENILQKIYEIMKTLEQLGHAEASMQVSDLIYEFMKQGDERLLEEAERLLEEVERG |
| | v1.3_EAAK_ | GSSGEAAAKEAAAKEAAAKGSSGGDKENILQKIYEIMKTLEQLGHAEASMQVSDLIY |
| | 3X | EFMKQGDERLLEEAERLLEEVERGGSSGEAAAKEAAAKEAAAKGSSGGDKENILQKI |
| | | YEIMKTLEQLGHAEASMQVSDLIYEFMKQGDERLLEEAERLLEEVER |
| 473 | LCB1_ | DKENILQKIYEIMKTLEQLGHAEASMQVSDLIYEFMKQGDERLLEEAERLLEEVERG |
| | v1.3_Pro_ | GSSGPSTPPTPSPSTPPTPSPSPGGSSGDKENILQKIYEIMKTLEQLGHAEASMQVS |
| | 3X | DLIYEFMKQGDERLLEEAERLLEEVERGGSSGPSTPPTPSPSTPPTPSPSPGGSSGD |
| | | KENILQKIYEIMKTLEQLGHAEASMQVSDLIYEFMKQGDERLLEEAERLLEEVER |
| 474 | LCB1_ | DKENILQKIYEIMKTLEQLGHAEASMQVSDLIYEFMKQGDERLLEEAERLLEEVERG |
| | v1.3_Ub_ | GSSGQIFVKTLTGKTITLEVEPSDTIENVKAKIQDKEGIPPDQQRLIFAGKQLEDGR |
| | 3X | TLSDYNIQKESTLHLVLRLRGGGGSSGDKENILQKIYEIMKTLEQLGHAEASMQVSD |
| | | LIYEFMKQGDERLLEEAERLLEEVERGGSSGQIFVKTLTGKTITLEVEPSDTIENVK |
| | | AKIQDKEGIPPDQQRLIFAGKQLEDGRTLSDYNIQKESTLHLVLRLRGGGGSSGDKE |
| | | NILQKIYEIMKTLEQLGHAEASMQVSDLIYEFMKQGDERLLEEAERLLEEVER |
| 475 | 1GS1_ | DKENVLQKIYEIMKELERLGHAEASMQVSDLIYEFMKTKDENLLEEAERLLEEVKRG |
| | NTS | GSSGGGSSGGGSSGGGSSGGGSSGDKENVLQKIYEIMKELERLGHAEASMQVSDLIY |
| | | EFMKTKDENLLEEAERLLEEVKR |
| 476 | 1PRP1_ | DKENVLQKIYEIMKELERLGHAEASMQVSDLIYEFMKTKDENLLEEAERLLEEVKRG |
| | NTS | GSSGPSTPPTPSPSTPPTPSPSPGGSSGDKENVLQKIYEIMKELERLGHAEASMQVS |
| | | DLIYEFMKTKDENLLEEAERLLEEVKR |
| 477 | 3GS3_ | NLDELHMQMTDLVYEALHFAKDEEFQKHVFQLFEKATKAYKNKDRQKLEKVVEELKE |
| | NTS | LLERLLSGGSSGGGSSGGGSSGGGSSGGGSSGNLDELHMQMTDLVYEALHFAKDEEF |
| | | QKHVFQLFEKATKAYKNKDRQKLEKWEELKELLERLLS |
| 478 | 3Pro3_ | NLDELHMQMTDLVYEALHFAKDEEFQKHVFQLFEKATKAYKNKDRQKLEKVVEELKE |
| | NTS | LLERLLSGGSSGPSTPPTPSPSTPPTPSPSPGGSSGNLDELHMQMTDLVYEALHFAK |
| | | DEEFQKHVFQLFEKATKAYKNKDRQKLEKWEELKELLERLLS |
| 475 | 1GS1_ | DKENVLQKIYEIMKELERLGHAEASMQVSDLIYEFMKTKDENLLEEAERLLEEVKRG |
| | CTS | GSSGGGSSGGGSSGGGSSGGGSSGDKENVLQKIYEIMKELERLGHAEASMQVSDLIY |
| | | EFMKTKDENLLEEAERLLEEVKR |
| 476 | 1Pro_ | DKENVLQKIYEIMKELERLGHAEASMQVSDLIYEFMKTKDENLLEEAERLLEEVKRG |
| | CTS | GSSGPSTPPTPSPSTPPTPSPSPGGSSGDKENVLQKIYEIMKELERLGHAEASMQVS |
| | | DLIYEFMKTKDENLLEEAERLLEEVKR |
| 477 | 3GS3_ | NLDELHMQMTDLVYEALHFAKDEEFQKHVFQLFEKATKAYKNKDRQKLEKVVEELKE |
| | CTS | LLERLLSGGSSGGGSSGGGSSGGGSSGGGSSGNLDELHMQMTDLVYEALHFAKDEEF |
| | | QKHVFQLFEKATKAYKNKDRQKLEKWEELKELLERLLS |
| 478 | 3Pro3_ | NLDELHMQMTDLVYEALHFAKDEEFQKHVFQLFEKATKAYKNKDRQKLEKVVEELKE |
| | CTS | LLERLLSGGSSGPSTPPTPSPSTPPTPSPSPGGSSGNLDELHMQMTDLVYEALHFAK |
| | | DEEFQKHVFQLFEKATKAYKNKDRQKLEKWEELKELLERLLS |
| 479 | 1GS1GS1_ | DKENVLQKIYEIMKELERLGHAEASMQVSDLIYEFMKTKDENLLEEAERLLEEVKRG |
| | NTS | GSSGGGSSGGGSSGGGSSGGGSSGDKENVLQKIYEIMKELERLGHAEASMQVSDLIY |
| | | EFMKTKDENLLEEAERLLEEVKRGGSSGGGSSGGGSSGGGSSGGGSSGDKENVLQKI |
| | | YEIMKELERLGHAEASMQVSDLIYEFMKTKDENLLEEAERLLEEVKR |
| 480 | 1Pro1Pro1_ | DKENVLQKIYEIMKELERLGHAEASMQVSDLIYEFMKTKDENLLEEAERLLEEVKRG |
| | NTS | GSSGPSTPPTPSPSTPPTPSPSPGGSSGDKENVLQKIYEIMKELERLGHAEASMQVS |
| | | DLIYEFMKTKDENLLEEAERLLEEVKRGGSSGPSTPPTPSPSTPPTPSPSPGGSSGD |
| | | KENVLQKIYEIMKELERLGHAEASMQVSDLIYEFMKTKDENLLEEAERLLEEVKR |
| 481 | 3GS3GS3_ | NLDELHMQMTDLVYEALHFAKDEEFQKHVFQLFEKATKAYKNKDRQKLEKVVEELKE |
| | NTS | LLERLLSGGSSGGGSSGGGSSGGGSSGGGSSGNLDELHMQMTDLVYEALHFAKDEEF |
| | | QKHVFQLFEKATKAYKNKDRQKLEKVVEELKELLERLLSGGSSGGGSSGGGSSGGGS |
| | | SGGGSSGNLDELHMQMTDLVYEALHFAKDEEFQKHVFQLFEKATKAYKNKDRQKLEK |
| | | WEELKELLERLLS |
| 482 | 3Pro3Pro3_ | NLDELHMQMTDLVYEALHFAKDEEFQKHVFQLFEKATKAYKNKDRQKLEKVVEELKE |
| | NTS | LLERLLSGGSSGPSTPPTPSPSTPPTPSPSPGGSSGNLDELHMQMTDLVYEALHFAK |
| | | DEEFQKHVFQLFEKATKAYKNKDRQKLEKVVEELKELLERLLSGGSSGPSTPPTPSP |
| | | STPPTPSPSPGGSSGNLDELHMQMTDLVYEALHFAKDEEFQKHVFQLFEKATKAYKN |
| | | KDRQKLEKWEELKELLERLLS |
| 479 | 1GS1GS1_ | DKENVLQKIYEIMKELERLGHAEASMQVSDLIYEFMKTKDENLLEEAERLLEEVKRG |
| | CTS | GSSGGGSSGGGSSGGGSSGGGSSGDKENVLQKIYEIMKELERLGHAEASMQVSDLIY |
| | | EFMKTKDENLLEEAERLLEEVKRGGSSGGGSSGGGSSGGGSSGGGSSGDKENVLQKI |
| | | YEIMKELERLGHAEASMQVSDLIYEFMKTKDENLLEEAERLLEEVKR |
| 480 | 1Pro1pRO_ | DKENVLQKIYEIMKELERLGHAEASMQVSDLIYEFMKTKDENLLEEAERLLEEVKRG |
| | CTS | GSSGPSTPPTPSPSTPPTPSPSPGGSSGDKENVLQKIYEIMKELERLGHAEASMQVS |
| | | DLIYEFMKTKDENLLEEAERLLEEVKRGGSSGPSTPPTPSPSTPPTPSPSPGGSSGD |
| | | KENVLQKIYEIMKELERLGHAEASMQVSDLIYEFMKTKDENLLEEAERLLEEVKR |
| 481 | 3GS3GS3_ | NLDELHMQMTDLVYEALHFAKDEEFQKHVFQLFEKATKAYKNKDRQKLEKVVEELKE |
| | CTS | LLERLLSGGSSGGGSSGGGSSGGGSSGGGSSGNLDELHMQMTDLVYEALHFAKDEEF |
| | | QKHVFQLFEKATKAYKNKDRQKLEKVVEELKELLERLLSGGSSGGGSSGGGSSGGGS |
| | | SGGGSSGNLDELHMQMTDLVYEALHFAKDEEFQKHVFQLFEKATKAYKNKDRQKLEK |
| | | WEELKELLERLLS |
| 482 | 3Pro3pRO3_ | NLDELHMQMTDLVYEALHFAKDEEFQKHVFQLFEKATKAYKNKDRQKLEKVVEELKE |
| | CTS | LLERLLSGGSSGPSTPPTPSPSTPPTPSPSPGGSSGNLDELHMQMTDLVYEALHFAK |
| | | DEEFQKHVFQLFEKATKAYKNKDRQKLEKVVEELKELLERLLSGGSSGPSTPPTPSP |
| | | STPPTPSPSPGGSSGNLDELHMQMTDLVYEALHFAKDEEFQKHVFQLFEKATKAYKN |
| | | KDRQKLEKWEELKELLERLLS |
| 483 | 1GS3_ | DKENVLQKIYEIMKELERLGHAEASMQVSDLIYEFMKTKDENLLEEAERLLEEVKRG |
| | NTS | GSSGGGSSGGGSSGGGSSGGGSSGNLDELHMQMTDLVYEALHFAKDEEFQKHVFQLF |
| | | EKATKAYKNKDRQKLEKWEELKELLERLLS |
| 484 | 1Pro3_ | DKENVLQKIYEIMKELERLGHAEASMQVSDLIYEFMKTKDENLLEEAERLLEEVKRG |
| | NTS | GSSGPSTPPTPSPSTPPTPSPSPGGSSGNLDELHMQMTDLVYEALHFAKDEEFQKHV |
| | | FQLFEKATKAYKNKDRQKLEKWEELKELLERLLS |
| 485 | 3GS1_ | NLDELHMQMTDLVYEALHFAKDEEFQKHVFQLFEKATKAYKNKDRQKLEKVVEELKE |
| | NTS | LLERLLSGGSSGGGSSGGGSSGGGSSGGGSSGDKENVLQKIYEIMKELERLGHAEAS |
| | | MQVSDLIYEFMKTKDENLLEEAERLLEEVKR |
| 486 | 3Pro1_ | NLDELHMQMTDLVYEALHFAKDEEFQKHVFQLFEKATKAYKNKDRQKLEKVVEELKE |
| | NTS | LLERLLSGGSSGPSTPPTPSPSTPPTPSPSPGGSSGDKENVLQKIYEIMKELERLGH |
| | | AEASMQVSDLIYEFMKTKDENLLEEAERLLEEVKR |
| 483 | 1GS3_ | DKENVLQKIYEIMKELERLGHAEASMQVSDLIYEFMKTKDENLLEEAERLLEEVKRG |
| | CTS | GSSGGGSSGGGSSGGGSSGGGSSGNLDELHMQMTDLVYEALHFAKDEEFQKHVFQLF |
| | | EKATKAYKNKDRQKLEKWEELKELLERLLS |
| 484 | 1Pro3_ | DKENVLQKIYEIMKELERLGHAEASMQVSDLIYEFMKTKDENLLEEAERLLEEVKRG |
| | CTS | GSSGPSTPPTPSPSTPPTPSPSPGGSSGNLDELHMQMTDLVYEALHFAKDEEFQKHV |
| | | PQLFEKATKAYKNKDRQKLEKWEELKELLERLLS |
| 485 | 3GS1_ | NLDELHMQMTDLVYEALHFAKDEEFQKHVFQLFEKATKAYKNKDRQKLEKVVEELKE |
| | CTS | LLERLLSGGSSGGGSSGGGSSGGGSSGGGSSGDKENVLQKIYEIMKELERLGHAEAS |
| | | MQVSDLIYEFMKTKDENLLEEAERLLEEVKR |
| 486 | 3Pro1_ | NLDELHMQMTDLVYEALHFAKDEEFQKHVFQLFEKATKAYKNKDRQKLEKVVEELKE |
| | CTS | LLERLLSGGSSGPSTPPTPSPSTPPTPSPSPGGSSGDKENVLQKIYEIMKELERLGH |
| | | AEASMQVSDLIYEFMKTKDENLLEEAERLLEEVKR |
| 487 | 3- | NLDELHMQMTDLVYEALHFAKDEEFQKHVFQLFEKATKAYKNKDRQKLEKVVEELKE |
| | GS10- | LLERLLSGGGSGGGSGGDKENVLQKIYEIMKELERLGHAEASMQVSDLIYEFMKTKD |
| | 1- | ENLLEEAERLLEEVKR |
| | L_NTS | |
| 488 | 3- | NLDELHMQMTDLVYEALHFAKDEEFQKHVFQLFEKATKAYKNKDRQKLEKVVEELKE |
| | GS10- | LLERLLSGGSSGGGSSGDKENVLQKIYEIMKELERLGHAEASMQVSDLIYEFMKTKD |
| | 1_ | ENLLEEAERLLEEVKR |
| | NTS | |
| 489 | 3- | NLDELHMQMTDLVYEALHFAKDEEFQKHVFQLFEKATKAYKNKDRQKLEKVVEELKE |
| | GS15- | LLERLLSGGSSGGGSSGGGSSGDKENVLQKIYEIMKELERLGHAEASMQVSDLIYEF |
| | 1_NTS | MKTKDENLLEEAERLLEEVKR |
| 490 | 3- | NLDELHMQMTDLVYEALHFAKDEEFQKHVFQLFEKATKAYKNKDRQKLEKVVEELKE |
| | GS20- | LLERLLSGGSSGGGSSGGGSSGGGSSGDKENVLQKIYEIMKELERLGHAEASMQVSD |
| | 1_NTS | LIYEFMKTKDENLLEEAERLLEEVKR |
| 491 | 1- | DKENVLQKIYEIMKELERLGHAEASMQVSDLIYEFMKTKDENLLEEAERLLEEVKRG |
| | GS10- | GSSGGGSSGDKENVLQKIYEIMKELERLGHAEASMQVSDLIYEFMKTKDENLLEEAE |
| | 1_NTS | RLLEEVKR |
| 492 | 1- | DKENVLQKIYEIMKELERLGHAEASMQVSDLIYEFMKTKDENLLEEAERLLEEVKRG |
| | GS15- | GSSGGGSSGGGSSGDKENVLQKIYEIMKELERLGHAEASMQVSDLIYEFMKTKDENL |
| | 1_NTS | LEEAERLLEEVKR |
| 493 | 1- | DKENVLQKIYEIMKELERLGHAEASMQVSDLIYEFMKTKDENLLEEAERLLEEVKRG |
| | GS20- | GSSGGGSSGGGSSGGGSSGDKENVLQKIYEIMKELERLGHAEASMQVSDLIYEFMKT |
| | 1_NTS | KDENLLEEAERLLEEVKR |
| 494 | 2- | ELEEQVMHVLDQVSELAHELLHKLTGEELERAAYFNWWATEMMLELIKSDDEREIRE |
| | 1_GS10 | IEEEARRILEHLEELARKGGSSGGGSSGDKENVLQKIYEIMKELERLGHAEASMQVS |
| | | DLIYEFMKTKDERLLEEAERLLEEVKR |
| 495 | 2- | ELEEQVMHVLDQVSELAHELLHKLTGEELERAAYFNWWATEMMLELIKSDDEREIRE |
| | 1_GS15 | IEEEARRILEHLEELARKGGSSGGGSSGGGSSGDKENVLQKIYEIMKELERLGHAEA |
| | | SMQVSDLIYEFMKTKDERLLEEAERLLEEVKR |
| 496 | 2- | ELEEQVMHVLDQVSELAHBLLHKLTGEELERAAYFNWWATEMMLELIKSDDEREIRE |
| | 1_GS20 | IEEEARRILEHLEELARKGGSSGGGSSGGGSSGGGSSGDKENVLQKIYEIMKELERL |
| | | GHAEASMQVSDLIYEFMKTKDERLLEEAERLLEEVKR |
| 497 | 2-2- | ELEEQVMHVLDQVSELAHELLHKLTGEELERAAYFNWWATEMMLELIKSDDEREIRE |
| | 1_GS10 | IEEEARRILEHLEELARKGGSSGGGSSGELEEQVMHVLDQVSELAHELLHKLTGEEL |
| | | ERAAYFNWWATEMMLELIKSDDEREIREIEEEARRILEHLEELARKGGSSGGGSSGD |
| | | KENVLQKIYEIMKELERLGHAEASMQVSDLIYEFMKTKDERLLEEAERLLEEVKR |
| 498 | 2-2- | ELEEQVMHVLDQVSELAHELLHKLTGEELERAAYFNWWATEMMLELIKSDDEREIRE |
| | 1_GS15 | IEEEARRILEHLEELARKGGSSGGGSSGGGSSGELEEQVMHVLDQVSELAHELLHKL |
| | | TGEELERAAYFNWWATEMMLELIKSDDEREIREIEEEARRILEHLEELARKGGSSGG |
| | | GSSGGGSSGDKENVLQKIYEIMKELERLGHAEASMQVSDLIYEFMKTKDERLLEEAE |
| | | RLLEEVKR |
| 499 | 2-2- | ELEEQVMHVLDQVSELAHELLHKLTGEELERAAYFNWWATEMMLELIKSDDEREIRE |
| | 1_GS20 | IEEEARRILEHLEELARKGGSSGGGSSGGGSSGGGSSGELEEQVMHVLDQVSELAHE |
| | | LLHKLTGEELERAAYFNWWATEMMLELIKSDDEREIREIEEEARRILEHLEELARKG |
| | | GSSGGGSSGGGSSGGGSSGDKENVLQKIYEIMKELERLGHAEASMQVSDLIYEFMKT |
| | | KDERLLEEAERLLEEVKR |
| 500 | 2- | ELEEQVMHVLDQVSELAHBLLHKLTGEELERAAYFNWWATEMMLELIKSDDEREIRE |
| | 2_GS10 | IEEEARRILEHLEELARKGGSSGGGSSGELEEQVMHVLDQVSELAHELLHKLTGEEL |
| | | ERAAYFNWWATEMMLELIKSDDEREIREIEEEARRILEHLEELARK |
| 501 | 2- | ELEEQVMHVLDQVSELAHELLHKLTGEELERAAYFNWWATEMMLELIKSDDEREIRE |
| | 2_GS15 | IEEEARRILEHLEELARKGGSSGGGSSGGGSSGELEEQVMHVLDQVSELAHELLHKL |
| | | TGEELERAAYFNWWATEMMLELIKSDDEREIREIEEEARRILEHLEELARK |
| 502 | 2- | ELEEQVMHVLDQVSELAHELLHKLTGEELERAAYFNWWATEMMLELIKSDDEREIRE |
| | 2_GS20 | IEEEARRILEHLEELARKGGSSGGGSSGGGSSGGGSSGELEEQVMHVLDQVSELAHE |
| | | LLHKLTGEELERAAYFNWWATEMMLELIKSDDEREIREIEEEARRILEHLEELARK |
| 503 | 2-2- | ELEEQVMHVLDQVSELAHELLHKLTGEELERAAYFNVWATEMMLELIKSDDEREIRE |
| | 2_GS10 | IEEEARRILEHLEELARKGGSSGGGSSGELEEQVMHVLDQVSELAHELLHKLTGEEL |
| | | ERAAYFNWWATEMMLELIKSDDEREIREIEEEARRILEHLEELARKGGSSGGGSSGE |
| | | LEEQVMHVLDQVSELAHELLHKLTGEELERAAYFNWWATEMMLELIKSDDEREIREI |
| | | EEEARRILEHLEELARK |
| 504 | 2-2- | ELEEQVMHVLDQVSELAHELLHKLTGEELERAAYFNWWATEMMLELIKSDDEREIRE |
| | 2_GS15 | IEEEARRILEHLEELARKGGSSGGGSSGGGSSGELEEQVMHVLDQVSELAHELLHKL |
| | | TGEELERAAYFNVMATEMMLELIKSDDEREIREIEEEARRILEHLEELARKGGSSGG |
| | | GSSGGGSSGELEEQVMHVLDQVSELAHELLHKLTGEELERAAYFNWWATEMMLELIK |
| | | SDDEREIREIEEEARRILEHLEELARK |
| 505 | 2-2- | ELEEQVMHVLDQVSELAHELLHKLTGEELERAAYFNWWATEMMLELIKSDDEREIRE |
| | 2_GS20 | IEEEARRILEHLEELARKGGSSGGGSSGGGSSGGGSSGELEEQVMHVLDQVSELAHE |
| | | LLHKLTGEELERAAYFNWWATEMMLELIKSDDEREIREIEEEARRILEHLEELARKG |
| | | GSSGGGSSGGGSSGGGSSGELEEQVMHVLDQVSELAHELLHKLTGEELERAAYFNWW |
| | | ATEMMLELIKSDDEREIREIEEEARRILEHLEELARK |
| 506 | AHB2- | ELEEQVMHVLDQVSELAHELLHKLTGEELERAAYFNWWATEMMLELIKSDDEREIRE |
| | AHB2- | IEEEARRILEHLEELARKGGASPAAPAPASPAAPAPSAPAGGELEEQVMHVLDQVSE |
| | PAS | LAHELLHKLTGEELERAAYFNWWATEMMLELIKSDDEREIREIEEEARRILEHLEEL |
| | | ARK |
| 507 | LCB3- | NLDELHMQMTDLVYEALHFAKDEEFQKHVFQLFEKATKAYKNKDRQKLEKVVEELKE |
| | LCB1_ | LLERLLSGGASPAAPAPASPAAPAPSAPAGGDKENVLQKIYEIMKELERLGHAEASM |
| | PAS | QVSDLIYEFMKTKDERLLEEAERLLEEVKR |
| 508 | AHB2- | ELEEQVMHVLDQVSELAHELLHKLTGEELERAAYFNWWATEMMLELIKSDDEREIRE |
| | LCB1_ | IEEEARRILEHLEELARKGGASPAAPAPASPAAPAPSAPAGGDKENVLQKIYEIMKE |
| | PAS | LERLGHAEASMQVSDLIYEFMKTKDERLLEEAERLLEEVKR |
| 509 | AHB2_ | ELEEQVMHVLDQVSELAHELLHKLTGEELERAAYFNWWATEMMLELIKSDDEREIRE |
| | 3x_PAS | IEEEARRILEHLEELARKGGASPAAPAPASPAAPAPSAPAGGELEEQVMHVLDQVSE |
| | | LAHELLHKLTGEELERAAYFNWWATEMMLELIKSDDEREIREIEEEARRILEHLEEL |
| | | ARKGGASPAAPAPASPAAPAPSAPAGGELEEQVMHVLDQVSELAHELLHKLTGEELE |
| | | RAAYFNWWATEMMLELIKSDDEREIREIEEEARRILEHLEELARK |
| 510 | 3-2- | NLDELHMQMTDLVYEALHFAKDEEFQKHVFQLFEKATKAYKNKDRQKLEKVVEELKE |
| | 1_PAS | LLERLLSGGASPAAPAPASPAAPAPSAPAGGELEEQVMHVLDQVSELAHELLHKLTG |
| | | EELERAAYFNWWATEMMLELIKSDDEREIREIEEEARRILEHLEELARKGGASPAAP |
| | | APASPAAPAPSAPAGGDKENVLQKIYEIMKELERLGHAEASMQVSDLIYEFMKTKDE |
| | | RLLEEAERLLEEVKR |
| 511 | 2-3- | ELEEQVMHVLDQVSELAHELLHKLTGEELERAAYFNWWATEMMLELIKSDDEREIRE |
| | 1_PAS | IEEEARRILEHLEELARKGGASPAAPAPASPAAPAPSAPAGGNLDELHMQMTDLVYE |
| | | ALHFAKDEEFQKHVFQLFEKATKAYKNKDRQKLEKVVEELKELLERLLSGGASPAAP |
| | | APASPAAPAPSAPAGGDKENVLQKIYEIMKELERLGHAEASMQVSDLIYEFMKTKDE |
| | | RLLEEAERLLEEVKR |
| 512 | 1-1- | DKENVLQKIYEIMKELERLGHAEASMQVSDLIYEFMKTKDENLLEEAERLLEEVKRG |
| | 1_PAS24 | GASPAAPAPASPAAPAPSAPAGGDKENVLQKIYEIMKELERLGHAEASMQVSDLIYE |
| | | FMKTKDENLLEEAERLLEEVKRGGASPAAPAPASPAAPAPSAPAGGDKENVLQKIYE |
| | | IMKELERLGHAEASMQVSDLIYEFMKTKDENLLEEAERLLEEVKR |
| 509 | 2-2- | ELEEQVMHVLDQVSELAHELLHKLTGEELERAAYFNWWATEMMLELIKSDDEREIRE |
| | 2_PAS24 | IEEEARRILEHLEELARKGGASPAAPAPASPAAPAPSAPAGGELEEQVMHVLDQVSE |
| | | LAHELLHKLTGEELERAAYFNWWATEMMLELIKSDDEREIREIEEEARRILEHLEEL |
| | | ARKGGASPAAPAPASPAAPAPSAPAGGELEEQVMHVLDQVSELAHELLHKLTGEELE |
| | | RAAYFNWWATEMMLELIKSDDEREIREIEEEARRILEHLEELARK |
| 513 | 3-3- | NLDELHMQMTDLVYEALHFAKDEEFQKHVFQLFEKATKAYKNKDRQKLEKVVEELKE |
| | 3_PAS24 | LLERLLSGGASPAAPAPASPAAPAPSAPAGGNLDELHMQMTDLVYEALHFAKDEEFQ |
| | | KHVFQLFEKATKAYKNKDRQKLEKVVEELKELLERLLSGGASPAAPAPASPAAPAPS |
| | | APAGGNLDELHMQMTDLVYEALHFAKDEEFQKHVFQLFEKATKAYKNKDRQKLEKVV |
| | | EELKELLERLLS |
| 514 | 2-2- | ELEEQVMHVLDQVSELAHELLHKLTGEELERAAYFNWWATEMMLELIKSDDEREIRE |
| | 1_PAS24 | IEEEARRILEHLEELARKGGASPAAPAPASPAAPAPSAPAGGELEEQVMHVLDQVSE |
| | | LAHELLHKLTGEELERAAYFNWWATEMMLELIKSDDEREIREIEEEARRILEHLEEL |
| | | ARKGGASPAAPAPASPAAPAPSAPAGGDKENVLQKIYEIMKELERLGHAEASMQVSD |
| | | LIYEFMKTKDENLLEEAERLLEEVKR |
| 515 | 2- | ELEEQVMHVLDQVSELAHELLHKLTGEELERAAYFNWWATEMMLELIKSDDEREIRE |
| | 2_PAS16 | IEEEARRILEHLEELARKGGASPAAPAPASPAGGELEEQVMHVLDQVSELAHELLHK |
| | | LTGEELERAAYFNWWATEMMLELIKSDDEREIREIEEEARRILEHLEELARK |
| 516 | 2- | ELEEQVMHVLDQVSELAHELLHKLTGEELERAAYFNWWATEMMLELIKSDDEREIRE |
| | 2_PAS11 | IEEEARRILEHLEELARKGGASPAAPAGGELEEQVMHVLDQVSELAHELLHKLTGEE |
| | | LERAAYFNWWATEMMLELIKSDDEREIREIEEEARRILEHLEELARK |
| 517 | 3- | NLDELHMQMTDLVYEALHFAKDEEFQKHVFQLFEKATKAYKNKDRQKLEKVVEELKE |
| | 1_PAS16 | LLERLLSGGASPAAPAPASPAGGDKENVLQKIYEIMKELERLGHAEASMQVSDLIYE |
| | | FMKTKDERLLEEAERLLEEVKR |
| 518 | 3- | NLDELHMQMTDLVYEALHFAKDEEFQKHVFQLFEKATKAYKNKDRQKLEKVVEELKE |
| | 1_PAS11 | LLERLLSGGASPAAPAGGDKENVLQKIYEIMKELERLGHAEASMQVSDLIYEFMKTK |
| | | DERLLEEAERLLEEVKR |
| 519 | 2- | ELEEQVMHVLDQVSELAHELLHKLTGEELERAAYFNWWATEMMLELIKSDDEREIRE |
| | 1_PAS16 | IEEEARRILEHLEELARKGGASPAAPAPASPAGGDKENVLOKIYEIMKELERLGHAE |
| | | ASMQVSDLIYEFMKTKDERLLEEAERLLEEVKR |
| 520 | 2- | ELEEQVMHVLDQVSELAHELLHKLTGEELERAAYFNWWATEMMLELIKSDDEREIRE |
| | 1_PAS11 | IEEEARRILEHLEELARKGGASPAAPAGGDKENVLQKIYEIMKELERLGHAEASMQV |
| | | SDLIYEFMKTKDERLLEEAERLLEEVKR |
| 521 | 3v2.3- | NIDELLMQVTDLIYEALHFAKDEEFQKHAFQLFEKATKAYKNKDKQKLEKVVEELKE |
| | 2- | LLERILSGGASPAAPAPASPAAPAPSAPAGGELEEQVMHVLDQVSELAHELLHKLTG |
| | 1_PAS24 | EELERAAYFNWWATEMMLELIKSDDEREIREIEEEARRILEHLEELARKGGASPAAP |
| | | APASPAAPAPSAPAGGDKENVLQKIYEIMKELERLGHAEASMQVSDLIYEFMKTKDE |
| | | RLLEEAERLLEEVKR |
| 522 | 2- | ELEEQVMHVLDQVSELAHELLHKLTGEELERAAYFNWWATEMMLELIKSDDEREIRE |
| | 3v2.3- | IEEEARRILEHLEELARKGGASPAAPAPASPAAPAPSAPAGGNIDELLMQVTDLIYE |
| | 1_PAS24 | ALHFAKDEEFQKHAFQLFEKATKAYKNKDKQKLEKVVEELKELLERILSGGASPAAP |
| | | APASPAAPAPSAPAGGDKENVLQKIYEIMKELERLGHAEASMQVSDLIYEFMKTKDE |
| | | RLLEEAERLLEEVKR |
| 523 | 2-2- | ELEEQVMHVLDQVSELAHELLHKLTGEELERAAYFNWWATEMMLELIKSDDEREIRE |
| | 2_PAS24_ | IEEEAARILEHLEELARTGGASPAAPAPASPAAPAPSAPAGGELEEQVMHVLDQVSE |
| | ompT | LAHELLHKLTGEELERAAYFNWWATEMMLELIKSDDEREIREIEEEAARILEHLEEL |
| | | ARTGGASPAAPAPASPAAPAPSAPAGGELEEQVMHVLDQVSELAHELLHKLTGEELE |
| | | RAAYFNWWATEMMLELIKSDDEREIREIEEEAARILEHLEELART |
| 524 | 1-1- | DKENVLQKIYEIMKELERLGHAEASMQVSDLIYEFMKTKDENLLEEAERLLEEVTRG |
| | 1_PAS24_ | GASPAAPAPASPAAPAPSAPAGGDKENVLOKIYEIMKELERLGHAEASMQVSDLIYE |
| | ompT | FMKTKDENLLEEAERLLEEVTRGGASPAAPAPASPAAPAPSAPAGGDKENVLQKIYE |
| | | IMKELERLGHAEASMQVSDLIYEFMKTKDENLLEEAERLLEEVTR |
| 525 | 3v2.3- | NIDELLMQVTDLIYEALHFAKDEEFQKHAFQLFEKATKAYKNKDKQKLEKVVEELKE |
| | 2- | LLERILSGGASPAAPAPASPAAPAPSAPAGGELEEQVMHVLDQVSELAHELLHKLTG |
| | 1_PAS24_ | EELERAAYFNWWATEMMLELIKSDDEREIREIEEEAARILEHLEELARTGGASPAAP |
| | ompT | APASPAAPAPSAPAGGDKENVLQKIYEIMKELERLGHAEASMQVSDLIYEFMKTKDE |
| | | RLLEEAERLLEEVKR |
| 526 | 2- | ELEEQVMHVLDQVSELAHELLHKLTGEELERAAYFNWWATEMMLELIKSDDEREIRE |
| | 3v2.3- | IEEEAARILEHLEELARTGGASPAAPAPASPAAPAPSAPAGGNIDELLMQVTDLIYE |
| | 1_PAS24_ | ALHFAKDEEFQKHAFOLFEKATKAYKNKDKQKLEKVVEELKELLERILSGGASPAAP |
| | ompT | APASPAAPAPSAPAGGDKENVLQKIYEIMKELERLGHAEASMQVSDLIYEFMKTKDE |
| | | RLLEEAERLLEEVKR |
| 527 | 3v2.3- | NIDELLMQVTDLIYEALHFAKDEEFQKHAFQLFEKATKAYKNKDKQKLEKVVEELKE |
| | 2- | LLERILSGGASPAAPAPASPAAPAPSAPAGGELEEQVMHVLDQVSELAHELLHKLTG |
| | 1_PAS24 | EELERAAYFNWWATEMMLELIKSDDEREIREIEEEARRILEHLEELARKGGASPAAP |
| | | APASPAAPAPSAPAGGDKENVLQKIYEIMKELERLGHAEASMQVSDLIYEFMKTKDE |
| | | NLLEEAERLLEEVKR |
| 528 | 2- | ELEEQVMHVLDQVSELAHELLHKLTGEELERAAYFNWWATEMMLELIKSDDEREIRE |
| | 3v2.3- | IEEEARRILEHLEELARKGGASPAAPAPASPAAPAPSAPAGGNIDELLMQVTDLIYE |
| | 1_PAS24 | ALHFAKDEEFQKHAFQLFEKATKAYKNKDKQKLEKVVEELKELLERILSGGASPAAP |
| | | APASPAAPAPSAPAGGDKENVLQKIYEIMKELERLGHAEASMQVSDLIYEFMKTKDE |
| | | NLLEEAERLLEEVKR |
| 529 | 2- | NIDELLMQVTDLIYEALHFAKDEEFQKHAFQLFEKATKAYKNKDKQKLEKVVEELKE |
| | 3v2.3- | LLERILSGGASPAAPAPASPAAPAPSAPAGGELEEQVMHVLDQVSELAHELLHKLTG |
| | 1_PAS24_ | EELERAAYFNWWATEMMLELIKSDDEREIREIEEEAARILEHLEELARTGGASPAAP |
| | ompT | APASPAAPAPSAPAGGDKENVLQKIYEIMKELERLGHAEASMQVSDLIYEFMKTKDE |
| | | NLLEEAERLLEEVKR |
| 530 | 2- | ELEEQVMHVLDQVSELAHELLHKLTGEELERAAYFNWWATEMMLELIKSDDEREIRE |
| | 3v2.3- | IEEEAARILEHLEELARTGGASPAAPAPASPAAPAPSAPAGGNIDELLMQVTDLIYE |
| | 1_PAS24_ | ALHFAKDEEFQKHAFQLFEKATKAYKNKDKQKLEKVVEELKELLERILSGGASPAAP |
| | ompT | APASPAAPAPSAPAGGDKENVLQKIYEIMKELERLGHAEASMQVSDLIYEFMKTKDE |
| | | NLLEEAERLLEEVKR |
| 538 | 2- | ELEEQVMHVLDQVSELAHELLHKLTGEELERAAYFNWWATEMMLELIKSDDEREIRE |
| | 3v2.3- | IEEEARRILEHLEELARKGGASPAAPAPASPAAPAPSAPAGGNIDELLMQVTDLIYE |
| | 1_PAS24_ | ALHFAKDEEFQKHAFQLFEKATKAYKNKDKQKLEKVVEELKELLERILSGGASPAAP |
| | N- | APASPAAPAPSAPAGGDKENVLQKIYEIMKELERLGHAEASMQVSDLIYEFMKTKDE |
| | His-TEV | NLLEEAERLLEEVKR |
| 530 | 2- | ELEEQVMHVLDQVSELAHELLHKLTGEELERAAYFNWWATEMMLELIKSDDEREIRE |
| | 3v2.3- | IEEEAARILEHLEELARTGGASPAAPAPASPAAPAPSAPAGGNIDELLMQVTDLIYE |
| | 1_PAS24_ | ALHFAKDEEFQKHAFQLFEKATKAYKNKDKQKLEKVVEELKELLERILSGGASPAAP |
| | ompT | APASPAAPAPSAPAGGDKENVLQKIYEIMKELERLGHAEASMQVSDLIYEFMKTKDE |
| | N- | NLLEEAERLLEEVKR |
| | His-TEV | |
| 531 | 2-3- | ELEEQVMHVLDQVSELAHELLHKLTGEELERAAYFNWWATEMMLELIKSDDEREIRE |
| | 1_PAS24_ | IEEEARRILEHLEELARKGGASPAAPAPASPAAPAPSAPAGGNIDELLMQVTDLIYE |
| | NTSF | ALHFAKDEEFQKHAFQLFEKATKAYKNKDKQKLEKVVEELKELLERILSGGASPAAP |
| | | APASPAAPAPSAPAGGDKENVLQKIYEIMKELERLGHAEASMQVSDLIYEFMKTKDE |
| | | NLLEEAERLLEEVKR |
| 531 | 2-3- | ELEEQVMHVLDQVSELAHELLHKLTGEELERAAYFNWWATEMMLELIKSDDEREIRE |
| | 1_PAS24_ | IEEEARRILEHLEELARKGGASPAAPAPASPAAPAPSAPAGGNIDELLMQVTDLIYE |
| | NTS3F | ALHFAKDEEFQKHAFQLFEKATKAYKNKDKQKLEKVVEELKELLERILSGGASPAAP |
| | | APASPAAPAPSAPAGGDKENVLQKIYEIMKELERLGHAEASMQVSDLIYEFMKTKDE |
| | | NLLEEAERLLEEVKR |
| 531 | 2-3- | ELEEQVMHVLDQVSELAHELLHKLTGEELERAAYFNWWATEMMLELIKSDDEREIRE |
| | 1_PAS24_ | IEEEARRILEHLEELARKGGASPAAPAPASPAAPAPSAPAGGNIDELLMQVTDLIYE |
| | NTSM | ALHFAKDEEFQKHAFQLFEKATKAYKNKDKQKLEKVVEELKELLERILSGGASPAAP |
| | | APASPAAPAPSAPAGGDKENVLQKIYEIMKELERLGHAEASMQVSDLIYEFMKTKDE |
| | | NLLEEAERLLEEVKR |
| 532 | 2- | ELEEQVMHVLDQVSELAHELLHKLTGEELERAAYFNWWATEMMLELIKSDDEREIRE |
| | PAS24- | IEEEARRILEHLEELARKGGASPAAPAPASPAAPAPSAPAGGNIDELLMQVTDLIYE |
| | 3- | ALHFAKDEEFQKHAFOLFEKATKAYKNKDKQKLEKVVEELKELLERILSGGASPAAP |
| | PAS16- | APASPAGGDKENVLQKIYEIMKELERLGHAEASMQVSDLIYEFMKTKDENLLEEAER |
| | 1_NTS3F | LLEEVKR |
| 533 | 2- | ELEEQVMHVLDQVSELAHELLHKLTGEELERAAYFNWWATEMMLELIKSDDEREIRE |
| | PAS16- | IEEEARRILEHLEELARKGGASPAAPAPASPAGGNIDELLMQVTDLIYEALHFAKDE |
| | 3- | EFQKHAFQLFEKATKAYKNKDKQKLEKVVEELKELLERILSGGASPAAPAPASPAGG |
| | PAS16-1_ | DKENVLQKIYEIMKELERLGHAEASMQVSDLIYEFMKTKDENLLEEAERLLEEVKR |
| | NTS3F | |
| 534 | 2- | ELEEQVMHVLDQVSELAHELLHKLTGEELERAAYFNWWATEMMLELIKSDDEREIRE |
| | PAS11- | IEEEARRILEHLEELARKGGASPAAPAGGNIDELLMQVTDLIYEALHFAKDEEFQKH |
| | 3- | AFQLFEKATKAYKNKDKQKLEKVVEELKELLERILSGGASPAAPAPASPAGGDKENV |
| | PAS16-1_ | LQKIYEIMKELERLGHAEASMQVSDLIYEFMKTKDENLLEEAERLLEEVKR |
| | NTS3F | |
| 535 | 2- | ELEEQVMHVLDQVSELAHELLHKLTGEELERAAYFNWWATEMMLELIKSDDEREIRE |
| | PAS24- | IEEEARRILEHLEELARKGGASPAAPAPASPAAPAPSAPAGGNIDELLMQVTDLIYE |
| | 3- | ALHFAKDEEFQKHAFQLFEKATKAYKNKDKQKLEKVVEELKELLERILSGGASPAAP |
| | PAS 11-1_ | AGGDKENVLQKIYEIMKELERLGHAEASMQVSDLIYEFMKTKDENLLEEAERLLEEV |
| | NTS3F | KR |
| 536 | 2- | ELEEQVMHVLDQVSELAHELLHKLTGEELERAAYFNWWATEMMLELIKSDDEREIRE |
| | PAS16- | IEEEARRILEHLEELARKGGASPAAPAPASPAGGNIDELLMQVTDLIYEALHFAKDE |
| | 3- | EFQKHAFQLFEKATKAYKNKDKQKLEKVVEELKELLERILSGGASPAAPAGGDKENV |
| | PAS11- | LQKIYEIMKELERLGHAEASMQVSDLIYEFMKTKDENLLEEAERLLEEVKR |
| | 1_NTS3F | |
| 537 | 2- | ELEEQVMHVLDQVSELAHELLHKLTGEELERAAYFNWWATEMMLELIKSDDEREIRE |
| | PAS11- | IEEEARRILEHLEELARKGGASPAAPAGGNIDELLMQVTDLIYEALHFAKDEEFQKH |
| | 3- | AFQLFEKATKAYKNKDKQKLEKVVEELKELLERILSGGASPAAPAGGDKENVLQKIY |
| | PAS11- | EIMKELERLGHAEASMQVSDLIYEFMKTKDENLLEEAERLLEEVKR |
| | 1_NTS3F | |
| 538 | 2-2_G4 | ELEEQVMHVLDQVSELAHELLHKLTGEELERAAYFNWWATEMMLELIKSDDEREIRE |
| | | IEEEARRILEHLEELARKGGGGELEEQVMHVLDQVSELAHELLHKLTGEELERAAYF |
| | | NWWATEMMLELIKSDDEREIREIEEEARRILEHLEELARK |
| 539 | 2-2_G2 | ELEEQVMHVLDQVSELAHELLHKLTGEELERAAYFNWWATEMMLELIKSDDEREIRE |
| | | IEEEARRILEHLEELARKGGELEEQVMHVLDQVSELAHELLHKLTGEELERAAYFNW |
| | | WATEMMLELIKSDDEREIREIEEEARRILEHLEELARK |
| 540 | 2- | ELEEQVMHVLDQVSELAHELLHKLTGEELERAAYFNWWATEMMLELIKSDDEREIRE |
| | 3_PAS24 | IEEEARRILEHLEELARKGGASPAAPAPASPAAPAPSAPAGGNIDELLMQVTDLIYE |
| | | ALHFAKDEEFQKHAFQLFEKATKAYKNKDKQKLEKVVEELKELLERILS |
| 541 | 2- | ELEEQVMHVLDQVSELAHELLHKLTGEELERAAYFNWWATEMMLELIKSDDEREIRE |
| | 3_PAS16 | IEEEARRILEHLEELARKGGASPAAPAPASPAGGNIDELLMQVTDLIYEALHFAKDE |
| | | EFQKHAFQLFEKATKAYKNKDKQKLEKVVEELKELLERILS |
| 542 | 2- | ELEEQVMHVLDQVSELAHELLHKLTGEELERAAYFNWWATEMMLELIKSDDEREIRE |
| | 3_PAS11 | IEEEARRILEHLEELARKGGASPAAPAGGNIDELLMQVTDLIYEALHFAKDEEFQKH |
| | | AFQLFEKATKAYKNKDKOKLEKVVEELKELLERILS |
| 543 | 1-2- | DKENVLQKIYEIMKELERLGHAEASMQVSDLIYEFMKTKDENLLEEAERLLEEVKRG |
| | 1_PAS24 | GASPAAPAPASPAAPAPSAPAGGELEEQVMHVLDQVSELAHELLHKLTGEELERAAY |
| | | FNWWATEMMLELIKSDDEREIREIEEEARRILEHLEELARKGGASPAAPAPASPAAP |
| | | APSAPAGGDKENVLQKIYEIMKELERLGHAEASMQVSDLIYEFMKTKDENLLEEAER |
| | | LLEEVKR |
| 544 | 2- | ELEEQVMHVLDQVSELAHELLHKLTGEELERAAYFNWWATEMMLELIKSDDEREIRE |
| | PAS24- | IEEEARRILEHLEELARKGGASPAAPAPASPAAPAPSAPAGGNLDELHMQMTDLVYE |
| | 3- | ALHFAKDEEFQKHVFQLFEKATKAYKNKDRQKLEKVVEELKELLERLLSGGASPAAP |
| | PAS24- | APASPAGGDKENVLOKIYEIMKELERLGHAEASMQVSDLIYEFMKTKDENLLEEAER |
| | 1_NTS | LLEEVKR |
| 545 | 2- | ELEEQVMHVLDQVSELAHELLHKLTGEELERAAYFNWWATEMMLELIKSDDEREIRE |
| | PAS24- | IEEEARRILEHLEELARKGGASPAAPAPASPAAPAPSAPAGGNLDELHMQMTDLVYE |
| | 3- | ALHFAKDEEFQKHVFQLFEKATKAYKNKDRQKLEKVVEELKELLERLLSGGASPAAP |
| | PAS16- | APASPAAPAPSAPAGGDKENVLQKIYEIMKELERLGHAEASMQVSDLIYEFMKTKDE |
| | 1_NTS | NLLEEAERLLEEVKR |
| 546 | 2- | ELEEQVMHVLDQVSELAHELLHKLTGEELERAAYFNWWATEMMLELIKSDDEREIRE |
| | PAS16- | IEEEARRILEHLEELARKGGASPAAPAPASPAGGNLDELHMQMTDLVYEALHFAKDE |
| | 3- | EFQKHVFQLFEKATKAYKNKDRQKLEKVVEELKELLERLLSGGASPAAPAPASPAAP |
| | PAS24- | APSAPAGGDKENVLQKIYEIMKELERLGHAEASMQVSDLIYEFMKTKDENLLEEAER |
| | 1_NTS | LLEEVKR |
| 547 | 2- | ELEEQVMHVLDQVSELAHELLHKLTGEELERAAYFNWWATEMMLELIKSDDEREIRE |
| | PAS16- | IEEEARRILEHLEELARKGGASPAAPAPASPAGGNLDELHMQMTDLVYEALHFAKDE |
| | 3- | EFQKHVFQLFEKATKAYKNKDRQKLEKVVEELKELLERLLSGGASPAAPAPASPAGG |
| | PAS16- | DKENVLQKIYEIMKELERLGHAEASMQVSDLIYEFMKTKDENLLEEAERLLEEVKR |
| | 1_NTS | |
| 548 | 2- | ELEEQVMHVLDQVSELAHELLHKLTGEELERAAYFNWWATEMMLELIKSDDEREIRE |
| | PAS11- | IEEEARRILEHLEELARKGGASPAAPAGGNLDELHMQMTDLVYEALHFAKDEEFQKH |
| | 3- | VFQLFEKATKAYKNKDRQKLEKVVEELKELLERLLSGGASPAAPAPASPAGGDKENV |
| | PAS16- | LQKIYEIMKELERLGHAEASMQVSDLIYEFMKTKDENLLEEAERLLEEVKR |
| | 1_NTS | |
| 549 | 2- | ELEEQVMHVLDQVSELAHELLHKLTGEELERAAYFNWWATEMMLELIKSDDEREIRE |
| | PAS24- | IEEEARRILEHLEELARKGGASPAAPAPASPAAPAPSAPAGGNLDELHMQMTDLVYE |
| | 3- | ALHFAKDEEFQKHVFQLFEKATKAYKNKDRQKLEKVVEELKELLERLLSGGASPAAP |
| | PAS11- | AGGDKENVLQKIYEIMKELERLGHAEASMQVSDLIYEFMKTKDENLLEEAERLLEEV |
| | 1_NTS | KR |
| 550 | 2- | ELEEQVMHVLDQVSELAHELLHKLTGEELERAAYFNWWATEMMLELIKSDDEREIRE |
| | PAS16- | IEEEARRILEHLEELARKGGASPAAPAPASPAGGNLDELHMQMTDLVYEALHFAKDE |
| | 3- | EFQKHVFQLFEKATKAYKNKDRQKLEKVVEELKELLERLLSGGASPAAPAGGDKENV |
| | PAS11- | LQKIYEIMKELERLGHAEASMQVSDLIYEFMKTKDENLLEEAERLLEEVKR |
| | 1_NTS | |
| 551 | 2- | ELEEQVMHVLDQVSELAHELLHKLTGEELERAAYFNWWATEMMLELIKSDDEREIRE |
| | PAS11- | IEEEARRILEHLEELARKGGASPAAPAGGNLDELHMQMTDLVYEALHFAKDEEFQKH |
| | 3- | VFQLFEKATKAYKNKDRQKLEKVVEELKELLERLLSGGASPAAPAGGDKENVLQKIY |
| | PAS11- | EIMKELERLGHAEASMQVSDLIYEFMKTKDENLLEEAERLLEEVKR |
| | 1_NTS | |
| 552 | 2-3- | ELEEQVMHVLDQVSELAHELLHKLTGEELERAAYFNWWATEMMLELIKSDDEREIRE |
| | 1_PAS7 | IEEEARRILEHLEELARKGGASAGGNLDELHMQMTDLVYEALHFAKDEEFQKHVFQL |
| | | FEKATKAYKNKDRQKLEKVVEELKELLERLLSGGASAGGDKENVLQKIYEIMKELER |
| | | LGHAEASMQVSDLIYEFMKTKDENLLEEAERLLEEVKR |
| 553 | 2-3- | ELEEQVMHVLDQVSELAHELLHKLTGEELERAAYFNWWATEMMLELIKSDDEREIRE |
| | 1_GS7 | IEEEARRILEHLEELARKGGSGGSGGNLDELHMQMTDLVYEALHFAKDEEFQKHVFQ |
| | | LFEKATKAYKNKDRQKLEKVVEELKELLERLLSGGSGGSGGDKENVLQKIYEIMKEL |
| | | ERLGHAEASMQVSDLIYEFMKTKDENLLEEAERLLEEVKR |
| 554 | 2-3- | ELEEQVMHVLDQVSELAHELLHKLTGEELERAAYFNWWATEMMLELIKSDDEREIRE |
| | 1_GS5 | IEEEARRILEHLEELARKGGSGGNLDELHMQMTDLVYEALHFAKDEEFQKHVFQLFE |
| | | KATKAYKNKDROKLEKVVEELKELLERLLSGGSGGDKENVLQKIYEIMKELERLGHA |
| | | EASMQVSDLIYEFMKTKDENLLEEAERLLEEVKR |
| 555 | 2-3- | ELEEQVMHVLDQVSELAHELLHKLTGEELERAAYFNWWATEMMLELIKSDDEREIRE |
| | 1_GS11 | IEEEARRILEHLEELARKGGSGGSGGSGGNLDELHMQMTDLVYEALHFAKDEEFQKH |
| | | VFQLFEKATKAYKNKDROKLEKVVEELKELLERLLSGGSGGSGGSGGDKENVLQKIY |
| | | EIMKELERLGHAEASMQVSDLIYEFMKTKDENLLEEAERLLEEVKR |
| 556 | 2-2- | ELEEQVMHVLDQVSELAHELLHKLTGEELERAAYFNWWATEMMLELIKSDDEREIRE |
| | 2_PAS11 | IEEEARRILEHLEELARKGGASPAAPAGGELEEQVMHVLDQVSELAHELLHKLTGEE |
| | | LERAAYFNWWATEMMLELIKSDDEREIREIEEEARRILEHLEELARKGGASPAAPAG |
| | | GELEEQVMHVLDQVSELAHELLHKLTGEELERAAYFNWWATEMMLELIKSDDEREIR |
| | | EIEEEARRILEHLEELARK |
| 557 | 2-2- | ELEEQVMHVLDQVSELAHELLHKLTGEELERAAYFNWWATEMMLELIKSDDEREIRE |
| | 2_GS11 | IEEEARRILEHLEELARKGGSGGSGGSGGELEEQVMHVLDQVSELAHELLHKLTGEE |
| | | LERAAYFNWWATEMMLELIKSDDEREIREIEEEARRILEHLEELARKGGSGGSGGSG |
| | | GELEEQVMHVLDQVSELAHELLHKLTGEELERAAYFNWWATEMMLELIKSDDEREIR |
| | | EIEEEARRILEHLEELARK |
| 558 | 2-2- | ELEEQVMHVLDQVSELAHELLHKLTGEELERAAYFNWWATEMMLELIKSDDEREIRE |
| | 2_GS8 | IEEEARRILEHLEELARKGGSGGSGGELEEQVMHVLDQVSELAHELLHKLTGEELER |
| | | AAYFNWWATEMMLELIKSDDEREIREIEEEARRILEHLEELARKGGSGGSGGELEEQ |
| | | VMHVLDQVSELAHELLHKLTGEELERAAYFNWWATEMMLELIKSDDEREIREIEEEA |
| | | RRILEHLEELARK |
| 559 | 2-2- | ELEEQVMHVLDQVSELAHELLHKLTGEELERAAYFNWWATEMMLELIKSDDEREIRE |
| | 2_GS5 | IEEEARRILEHLEELARKGGSGGELEEQVMHVLDQVSELAHELLHKLTGEELERAAY |
| | | FNWWATEMMLELIKSDDEREIREIEEEARRILEHLEELARKGGSGGELEEQVMHVLD |
| | | QVSELAHELLHKLTGEELERAAYFNWWATEMMLELIKSDDEREIREIEEEARRILEH |
| | | LEELARK |
| 560 | 2-3- | ELEEQVMHVLDQVSELAHELLHKLTGEELERAAYFNWWATEMMLELIKSDDEREIRE |
| | 1_GGGG | IEEEARRILEHLEELARKGGSGGGGSGGGGSGGNLDELHMQMTDLVYEALHFAKDEE |
| | S15 | FQKHVFQLFEKATKAYKNKDRQKLEKVVEELKELLERLLSGGSGGGGSGGGGSGGDK |
| | | ENVLQKIYEIMKELERLGHAEASMQVSDLIYEFMKTKDENLLEEAERLLEEVKR |
| 561 | 2-3- | ELEEQVMHVLDQVSELAHELLHKLTGEELERAAYFNWWATEMMLELIKSDDEREIRE |
| | 1_GGGG | IEEEARRILEHLEELARKGGSGGGGSGGGGNLDELHMQMTDLVYEALHFAKDEEFQK |
| | S12 | HVFQLFEKATKAYKNKDRQKLEKVVEELKELLERLLSGGSGGGGSGGGGDKENVLQK |
| | | IYEIMKELERLGHAEASMQVSDLIYEFMKTKDENLLEEAERLLEEVKR |
| 562 | 2-3- | ELEEQVMHVLDQVSELAHELLHKLTGEELERAAYFNWWATEMMLELIKSDDEREIRE |
| | 1_GGGG | IEEEARRILEHLEELARKGGGGSGGGGNLDELHMQMTDLVYEALHFAKDEEFQKHVF |
| | S9 | QLFEKATKAYKNKDRQKLEKVVEELKELLERLLSGGGGSGGGGDKENVLQKIYEIMK |
| | | ELERLGHAEASMQVSDLIYEFMKTKDENLLEEAERLLEEVKR |
| 563 | 2-3- | ELEEQVMHVLDQVSELAHELLHKLTGEELERAAYFNWWATEMMLELIKSDDEREIRE |
| | 1_GGGG | IEEEARRILEHLEELARKGGGSGGGNLDELHMQMTDLVYEALHFAKDEEFQKHVFQL |
| | S7 | FEKATKAYKNKDRQKLEKVVEELKELLERLLSGGGSGGGDKENVLQKIYEIMKELER |
| | | LGHAEASMQVSDLIYEFMKTKDENLLEEAERLLEEVKR |
| 564 | 1- | DKENVLQKIYEIMKELERLGHAEASMQVSDLIYEFMKTKDENLLEEAERLLEEVKRG |
| | 1_GGGG | GSGGGGSGGGGSGGGGSGGGGSGGDKENVLQKIYEIMKELERLGHAEASMQVSDLIY |
| | S25 | EFMKTKDENLLEEAERLLEEVKR |
| 565 | 1- | DKENVLQKIYEIMKELERLGHAEASMQVSDLIYEFMKTKDENLLEEAERLLEEVKRG |
| | 1_GGGG | GSGGGGSGGGGSGGGGSGGDKENVLQKIYEIMKELERLGHAEASMQVSDLIYEFMKT |
| | S20 | KDENLLEEAERLLEEVKR |
| 566 | 1- | DKENVLQKIYEIMKELERLGHAEASMQVSDLIYEFMKTKDENLLEEAERLLEEVKRG |
| | 1_GGGG | GSGGGGSGGGGSGGDKENVLQKIYEIMKELERLGHAEASMQVSDLIYEFMKTKDENL |
| | S15 | LEEAERLLEEVKR |
| 567 | 1- | DKENVLQKIYEIMKELERLGHAEASMQVSDLIYEFMKTKDENLLEEAERLLEEVKRG |
| | 1_GGGG | GSGGGGSGGDKENVLQKIYEIMKELERLGHAEASMQVSDLIYEFMKTKDENLLEEAE |
| | S10 | RLLEEVKR |
| 568 | 2- | ELEEQVMHVLDQVSELAHELLHKLTGEELERAAYFNWWATEMMLELIKSDDEREIRE |
| | 1_GGGG | IEEEARRILEHLEELARKGGSGGGGSGGDKENVLQKIYEIMKELERLGHAEASMQVS |
| | S10 | DLIYEFMKTKDENLLEEAERLLEEVKR |
| 569 | 3- | NLDELHMQMTDLVYEALHFAKDEEFQKHVFQLFEKATKAYKNKDRQKLEKVVEELKE |
| | 1_GGGG | LLERLLSGGSGGGGSGGDKENVLQKIYEIMKELERLGHAEASMQVSDLIYEFMKTKD |
| | S10 | ENLLEEAERLLEEVKR |
| 570 | 2- | ELEEQVMHVLDQVSELAHELLHKLTGEELERAAYFNWWATEMMLELIKSDDEREIRE |
| | 3_GGGG | IEEEARRILEHLEELARKGGSGGGGSGGNLDELHMQMTDLVYEALHFAKDEEFQKHV |
| | S10 | FQLFEKATKAYKNKDRQKLEKVVEELKELLERLLS |
| 571 | 3- | NLDELHMQMTDLVYEALHFAKDEEFQKHVFQLFEKATKAYKNKDRQKLEKVVEELKE |
| | 2_GGGG | LLERLLSGGSGGGGSGGELEEQVMHVLDQVSELAHELLHKLTGEELERAAYFNWWAT |
| | S10 | EMMLELIKSDDEREIREIEEEARRILEHLEELARK |
| 572 | 2-3- | ELEEQVMHVLDQVSELAHELLHKLTGEELERAAYFNWWATEMMLELIKSDDEREIRE |
| | 1_GGGG | IEEEARRILEHLEELARKGGSGGGGSGGNLDELHMQMTDLVYEALHFAKDEEFQKHV |
| | S10 | FQLFEKATKAYKNKDRQKLEKVVEELKELLERLLSGGSGGGGSGGDKENVLQKIYEI |
| | | MKELERLGHAEASMQVSDLIYEFMKTKDENLLEEAERLLEEVKR |
| 573 | 3-2- | NLDELHMQMTDLVYEALHFAKDEEFQKHVFQLFEKATKAYKNKDRQKLEKVVEELKE |
| | 1_GGGG | LLERLLSGGSGGGGSGGELEEQVMHVLDQVSELAHELLHKLTGEELERAAYFNWWAT |
| | S10 | EMMLELIKSDDEREIREIEEEARRILEHLEELARKGGSGGGGSGGDKENVLQKIYEI |
| | | MKELERLGHAEASMQVSDLIYEFMKTKDENLLEEAERLLEEVKR |
| 560 | 2-3- | ELEEQVMHVLDQVSELAHELLHKLTGEELERAAYFNWWATEMMLELIKSDDEREIRE |
| | 1_GGGG | IEEEARRILEHLEELARKGGSGGGGSGGGGSGGNLDELHMQMTDLVYEALHFAKDEE |
| | S15 | FQKHVFQLFEKATKAYKNKDROKLEKVVEELKELLERLLSGGSGGGGSGGGGSGGDK |
| | | ENVLQKIYEIMKELERLGHAEASMQVSDLIYEFMKTKDENLLEEAERLLEEVKR |
| 574 | 3-2- | NLDELHMQMTDLVYEALHFAKDEEFQKHVFQLFEKATKAYKNKDRQKLEKVVEELKE |
| | 1_GGGG | LLERLLSGGSGGGGSGGGGSGGELEEQVMHVLDQVSELAHELLHKLTGEELERAAYF |
| | S15 | NWWATEMMLELIKSDDEREIREIEEEARRILEHLEELARKGGSGGGGSGGGGSGGDK |
| | | ENVLQKIYEIMKELERLGHAEASMQVSDLIYEFMKTKDENLLEEAERLLEEVKR |
| 575 | LCB3- | NLDELHMQMTDLVYEALHFAKDEEFQKHVFQLFEKATKAYKNKDRQKLEKVVEELKE |
| | LCB1_ | LLERLLSGGASPAAPAPGGDKENVLQKIYEIMKELERLGHAEASMQVSDLIYEFMKT |
| | PAS12 | KDENLLEEAERLLEEVKR |
| 576 | AHB2- | ELEEQVMHVLDQVSELAHELLHKLTGEELERAAYFNWWATEMMLELIKSDDEREIRE |
| | LCB1_ | IEEEARRILEHLEELARKGGASPAAPAPGGDKENVLQKIYEIMKELERLGHAEASMQ |
| | PAS12 | VSDLIYEFMKTKDENLLEEAERLLEEVKR |
| 577 | AHB2- | ELEEQVMHVLDQVSELAHELLHKLTGEELERAAYFNWWATEMMLELIKSDDEREIRE |
| | LCB3_ | IEEEARRILEHLEELARKGGASPAAPAPGGNLDELHMQMTDLVYEALHFAKDEEFQK |
| | PAS12 | HVFQLFEKATKAYKNKDRQKLEKVVEELKELLERLLS |
| 578 | LCB3- | NLDELHMQMTDLVYEALHFAKDEEFQKHVFQLFEKATKAYKNKDRQKLEKVVEELKE |
| | AHB2_ | LLERLLSGGASPAAPAPGGELEEQVMHVLDQVSELAHELLHKLTGEELERAAYFNWW |
| | PAS12 | ATEMMLELIKSDDEREIREIEEEARRILEHLEELARK |
| 579 | AHB2- | ELEEQVMHVLDQVSELAHELLHKLTGEELERAAYFNWWATEMMLELIKSDDEREIRE |
| | LCB3- | IEEEARRILEHLEELARKGGASPAAPAPGGNLDELHMQMTDLVYEALHFAKDEEFQK |
| | LCB1_ | HVFQLFEKATKAYKNKDROKLEKVVEELKELLERLLSGGASPAAPAPGGDKENVLQK |
| | PAS12 | IYEIMKELERLGHAEASMQVSDLIYEFMKTKDENLLEEAERLLEEVKR |
| 580 | LCB3- | NLDELHMQMTDLVYEALHFAKDEEFQKHVFQLFEKATKAYKNKDRQKLEKVVEELKE |
| | AHB2- | LLERLLSGGASPAAPAPGGELEEQVMHVLDQVSELAHELLHKLTGEELERAAYFNWW |
| | LCB1_ | ATEMMLELIKSDDEREIREIEEEARRILEHLEELARKGGASPAAPAPGGDKENVLQK |
| | PAS12 | IYEIMKELERLGHAEASMQVSDLIYEFMKTKDENLLEEAERLLEEVKR |
| 581 | LCB3- | NLDELHMQMTDLVYEALHFAKDEEFQKHVFQLFEKATKAYKNKDRQKLEKWEELKE |
| | AHB2- | LLERLLSGGASPAAPAPASPAAPAPSAPAGGELEEQVMHVLDQVSELAHELLHKLTG |
| | LCB1_ | EELERAAYFNWWATEMMLELIKSDDEREIREIEEEARRILEHLEELARKGGASPAAP |
| | PAS24 | APASPAAPAPSAPAGGDKENVLQKIYEIMKELERLGHAEASMQVSDLIYEFMKTKDE |
| | | NLLEEAERLLEEVKR |
| 582 | AHB2v2- | ELEEQVMHVLDQVSELAHELLHKLTGEELERAAYFNWWATEMMLELIKSDDEREIRE |
| | LCB1_ | IEEEAARILEHLEELARTGGASPAAPAPGGDKENVLQKIYEIMKELERLGHAEASMQ |
| | PAS12 | VSDLIYEFMKTKDENLLEEAERLLEEVKR |
| 583 | AHB2v2- | ELEEQVMHVLDQVSELAHELLHKLTGEELERAAYFNWWATEMMLELIKSDDEREIRE |
| | LCB3_ | IEEEAARILEHLEELARTGGASPAAPAPGGNLDELHMQMTDLVYEALHFAKDEEFQK |
| | PAS12 | HVFQLFEKATKAYKNKDRQKLEKVVEELKELLERLLS |
| 584 | LCB3- | NLDELHMQMTDLVYEALHFAKDEEFQKHVFQLFEKATKAYKNKDRQKLEKVVEELKE |
| | AHB2v2_ | LLERLLSGGASPAAPAPGGELEEQVMHVLDQVSELAHELLHKLTGEELERAAYFNWW |
| | PAS12 | ATEMMLELIKSDDEREIREIEEEAARILEHLEELART |
| 585 | AHB2v2- | ELEEQVMHVLDQVSELAHELLHKLTGEELERAAYFNWWATEMMLELIKSDDEREIRE |
| | LCB3- | IEEEAARILEHLEELARTGGASPAAPAPGGNLDELHMQMTDLVYEALHFAKDEEFQK |
| | LCB1_ | HVFQLFEKATKAYKNKDRQKLEKVVEELKELLERLLSGGASPAAPAPGGDKENVLQK |
| | PAS12 | IYEIMKELERLGHAEASMQVSDLIYEFMKTKDENLLEEAERLLEEVKR |
| 586 | LCB3- | NLDELHMQMTDLVYEALHFAKDEEFQKHVFQLFEKATKAYKNKDRQKLEKVVEELKE |
| | AHB2v2- | LLERLLSGGASPAAPAPGGELEEQVMHVLDQVSELAHELLHKLTGEELERAAYFNWW |
| | LCB1_ | ATEMMLELIKSDDEREIREIEEEAARILEHLEELARTGGASPAAPAPGGDKENVLQK |
| | PAS12 | IYEIMKELERLGHAEASMOVSDLIYEFMKTKDENLLEEAERLLEEVKR |
| 587 | AHB2v2- | ELEEQVMHVLDQVSELAHELLHKLTGEELERAAYFNWWATEMMLELIKSDDEREIRE |
| | LCB3- | IEEEAARILEHLEELARTGGASPAAPAPASPAAPAPSAPAGGNLDELHMQMTDLVYE |
| | LCB1_ | ALHFAKDEEFQKHVFQLFEKATKAYKNKDRQKLEKVVEELKELLERLLSGGASPAAP |
| | PAS24 | APASPAAPAPSAPAGGDKENVLQKIYEIMKELERLGHAEASMQVSDLIYEFMKTKDE |
| | | NLLEEAERLLEEVKR |
| 588 | LCB3- | NLDELHMQMTDLVYEALHFAKDEEFQKHVFQLFEKATKAYKNKDRQKLEKVVEELKE |
| | AHB2v2- | LLERLLSGGASPAAPAPASPAAPAPSAPAGGELEEQVMHVLDQVSELAHELLHKLTG |
| | LCB1_ | EELERAAYFNWWATEMMLELIKSDDEREIREIEEEAARILEHLEELARTGGASPAAP |
| | PAS24 | APASPAAPAPSAPAGGDKENVLQKIYEIMKELERLGHAEASMQVSDLIYEFMKTKDE |
| | | NLLEEAERLLEEVKR |

In some embodiments, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a genus selected from those recited in the middle column of Table 8. In these embodiments, X1, X2, X3 (when recited in the genus), and X4 (when recited in the genus) may be present or absent, and when present may be any sequence of 1 or more amino acids. By way of example, the genus in the middle column, first row of sequences in Table 8 is X1-(SEQ ID NO:4)-X2-(SEQ ID NO:4). In this embodiment, X2 may be present or absent and, when present, may (for example) comprise an amino acid linker of any suitable length and amino acid composition as deemed appropriate. X1 may be present or absent, and when present may comprise any amino acid residue or residues as deemed appropriate, including but not limited to a leader sequence, a detectable tag, a purification tag, etc.

In another example, the genus in the middle column, last row of sequences in Table 8 is X1-(SEQ ID NO: 155)-X2-(SEQ ID NO: 164)-X3-(SEQ ID NO: 135)-X4. In this embodiment, X2 and X3 may be present or absent and, when present, may (for example) comprise an amino acid linker of any suitable length and amino acid composition as deemed appropriate. X1 and X4 may be present or absent, and when present may comprise any amino acid residue or residues as deemed appropriate, including but not limited to a leader sequence, a detectable tag, a purification tag, secretion signal etc.

In some embodiments, the optional domain that is present between monomer domains is present and may comprise an amino acid linker. Under this embodiment, (a) in the first example above X2 would be present and comprise an amino acid linker of any appropriate length and amino acid composition, and X1 may be present or absent; and (b) in the second example above one or both of X2 and X3 would be present and comprise an amino acid linker of any appropriate length and amino acid composition, and X1 and X4 may independently be present or absent.

In any embodiment or combination of embodiments of the polypeptides disclosed herein, the polypeptide may further comprise one or more additional functional peptide domain. Any such additional functional peptide domain may be used as appropriate for an intended purpose. In various non-limiting embodiments, the additional functional peptide domain may comprise, for example, a targeting domain, a detectable domain, a scaffold domain, a secretion signal, an Fc domain, or a further therapeutic peptide domain. In one embodiment, the additional functional domain comprises an Fc domain, including but not limited to an Fc domain comprising an amino acid sequence comprising the amino acid sequence of SEQ ID NO:64.

Fc domain :
(SEQ ID NO: 64)
EPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In another embodiment, the added functional domain may comprise an oligomerization domain. Any oligomerization domain may be used as suitable to generate an oligomer as suitable for an intended purpose. In one non-limiting embodiment, the oligomerization domain may comprise a homotrimerization domain. Exemplary oligomerization domains may comprises an amino acid sequence selected from the group consisting of SEQ ID NOS:179-189 and 589-594.

1rfo
(SEQ ID NO: 179)
GYIPEAPRDGQAYVRKDGEWVLLSTFL
1na0_int2-R3
(SEQ ID NO: 180)
EEAELAYLLGELAYKLGEYRIAIRAYRIALKRDPNNAEAWYNLGNAYYKQGDYDEAIEYY
QKALELDPNNAEAKQNLGNAKQKQG
1na0_int2
(SEQ ID NO: 181)
EEAELAYLLGELAYKLGEYRIAIRAYRIALKRDPNNAEAWYNLGNAYYKQGDYDEAIEYYQKALEL
DPNNAEAWYNLGNAYYKQGDYDEAIEYYQKALELDPNNAEAKQNLGNAKQKQG
6msr
(SEQ ID NO: 182)
GSEYEIRKALEELKASTAELKRATASLRASTEELKKNPSEDALVENNRLIVEHNA
IIVENNRIIAAVLELIVRAIK
1gcm
(SEQ ID NO: 183)
RMKQIEDKIEEILSKIYHIENEIARIKKLIGER
PRO-2-noHis
(SEQ ID NO: 184)
GSEYEIRKALEELKASTAELKRSTASLRASTEELKKNPSEDALVENNRLIVENNA
IIVENNRIIAAVLELIVRAIK
1na0_3
(SEQ ID NO: 185)
NLAEKMYKAGNAMYRKGQYTIAIIAYTLALLKDPNNAEAWYNLGNAAYKKGEYDEAIEAYQKALELDP
NNAEAWYNLGNAYYKQGDYDEAIEYYQKALELDPNNAEAKQNLGNAKQKQG
4pn9
(SEQ ID NO: 186)
GEIAKSLKEIAKSLKEIAWSLKEIAKSLKG
SB175
(SEQ ID NO: 187)
SEALEELEKALRELKKSTDELERSTEELEKNPSEDALVENNRLIVENNKIIVE
VLRIIAKVLK
SB175.1
(SEQ ID NO: 188)
SPELEKALRELKKSTDELERSTEELEKNGSPEALVENNRLIVENNKIIVEVLR
IIAK
SB175.2
(SEQ ID NO: 189)
SEKALRELKKSTDELERSTEELEKNGSPEALVENNRLIVENNKIIVEVLR
5L6HC3_1
(SEQ ID NO: 589)
SEELRAVADLQRLNIELARKLLEAVARLQELNIDLVRKTSELTDEKTIREE
IRKVKEESKRIVEEAEEEIRRAKEESRKIADESR
36729.2
(SEQ ID NO: 590)
EEAELAYLLGELAYKLGEYRIAIRAYRIALKRDPNNAEAWYNLGNAYYKQG
DYDEAIEYYQKALELDPNNAEAWYNLGNAYYKQGDYDEAIEYYQKALELDP
NNAEAWYNLGNAYYKQGDYDEAIEYYQKALEL
1na0_int2-R3v2
(SEQ ID NO: 591)
EEAELAYLLGELAYKLGEYRIAIRAYRIALKRDPNNAEAWYNLGNAYYKQG
DYDEAIYYQKALELDPNNAEAKQNLGNAKQKQ
1na0_int2-R3v3
(SEQ ID NO: 592)
EEAELAYLLGELAYKLGEYRIAIRAYRIALKRDPNNAEAWYNLGNAYYKQG
DYDEIEYYQKALELDPNNAEAKQNLGNAKQKQ
1na0_int2 v2
(SEQ ID NO: 593)
EEAELAYLLGELAYKLGEYRIAIRAYRIALKRDPNNAEAWYNLGNAYYKQG
DYDEAIYYQKALELDPNNAEAWYNLGNAYYKQGDYDEAIEYYQKALELDPN
NAEAKqNLGNKQKQ
6msrv2
(SEQ ID NO: 594)
EYEIRKALEELKASTAELKRATASLRASTEELKKNPSEDALVENNRLIVEH
NAIIVENRIIAAVLELIVRAIK

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS 356-453 and 595-692 and a genus selected from those recited in the right hand column of Table 9 wherein genus positions X1, X2, X3, and X4 may be present or absent, and when present may be any sequence of 1 or more amino acids. In all embodiments, any N-terminal methionine residues may be present or absent in the polypeptide. In one embodiment, any N-terminal methionine residues are absent in the polypeptide.

TABLE 9
Homotrimer Designs

| | | Annotated: X1, X2, X3, and X4 may be |
|---|---|---|
| | | present or absent, and when present |
| Name | Protein | may be any sequence of 1 or more amino acids |
| AHB2- | MEKKIELEEQVMHVLDQVSELAHELLH | X1- |
| 6GS-1rfo | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSGYIPEAPRDGQAYVRKDGEWVL | K(SEQ ID NO: 101) -X2- |
| | LSTFLGGSGSSGSAWSHPQFEKGGGSG | GYIPEAPRDGQAYVRKDGEWVLLSTFL (SEQ ID |
| | GGSGGSAWSHPQFEK(SEQ ID NO: | NO: 179)-X3 |
| | 356) | |
| AHB2- | MEKKIELEEQVMHVLDQVSELAHELLH | X1- |
| 28GS- | KLTGEELERAAYFNWWATEMMLELIKS | ELEEQVMHVLDQVSELAHELLHKLTGEELERAAYFNW |
| AHB2- | DDEREIREIEEEARRILEHLEELARKG | WATEMMLELIKSDDEREIREIEEEARRILEHLEELAR |
| 6GS-1rfo | SGSGSGSGSGSGSGSGSGSGSGSGSGS | K(SEQ ID NO: 101) -X2- |
| | ELEEQVMHVLDQVSELAHELLHKLTGE | ELEEQVMHVLDQVSELAHELLHKLTGEELERAAYFNW |
| | ELERAAYFNWWATEMMLELIKSDDERE | WATEMMLELIKSDDEREIREIEEEARRILEHLEELAR |
| | IREIEEEARRILEHLEELARKGSGSGS | K(SEQ ID NO: 101) -X3- |
| | GYIPEAPRDGQAYVRKDGEWVLLSTFL | GYIPEAPRDGQAYVRKDGEWVLLSTFL (SEQ ID |
| | GGSGSSGSAWSHPQFEKGGGSGGGSGG | NO: 179) -X4 |
| | SAWSHPQFEK (SEQ ID NO: 357) | |
| AHB2- | MEKKIELEEQVMHVLDQVSELAHELLH | X1- |
| 5GS-1rfo | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGGYIPEAPRDGQAYVRKDGEWVLL | K(SEQ ID NO: 101) -X2- |
| | STFLGGSGSSGSAWSHPQFEKGGGSGG | GYIPEAPRDGQAYVRKDGEWVLLSTFL (SEQ ID |
| | GSGGSAWSHPQFEK (SEQ ID NO: | NO: 179) -X3 |
| | 358) | |
| AHB2- | MEKKIELEEQVMHVLDQVSELAHELLH | X1- |
| 4GS-1rfo | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGYIPEAPRDGQAYVRKDGEWVLLS | K(SEQ ID NO: 101) -X2- |
| | TFLGGSGSSGSAWSHPQFEKGGGSGGG | GYIPEAPRDGQAYVRKDGEWVLLSTFL (SEQ ID |
| | SGGSAWSHPQFEK(SEQ ID NO: | NO: 179)-X3 |
| | 359) | |
| AHB2- | MEKKIELEEQVMHVLDQVSELAHELLH | X1- |
| 3GS-1rfo | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGGYIPEAPRDGQAYVRKDGEWVLLST | K(SEQ ID NO: 101)-X2- |
| | FLGGSGSSGSAWSHPQFEKGGGSGGGS | GYIPEAPRDGQAYVRKDGEWVLLSTFL (SEQ ID |
| | GGSAWSHPQFEK (SEQ ID NO: | NO: 179)-X3 |
| | 360) | |
| AHB2- | MEKKIELEEQVMHVLDQVSELAHELLH | X1- |
| 28GS- | KLTGEELERAAYFNWWATEMMLELIKS | ELEEQVMHVLDQVSELAHELLHKLTGEELERAAYFNW |
| AHB2- | DDEREIREIEEEARRILEHLEELARKG | WATEMMLELIKSDDEREIREIEEEARRILEHLEELAR |
| 3GS-1rfo | SGSGSGSGSGSGSGSGSGSGSGSGSGS | K(SEQ ID NO: 101)-X2- |
| | ELEEQVMHVLDQVSELAHELLHKLTGE | ELEEQVMHVLDQVSELAHELLHKLTGEELERAAYFNW |
| | ELERAAYFNWWATEMMLELIKSDDERE | WATEMMLELIKSDDEREIREIEEEARRILEHLEELAR |
| | IREIEEEARRILEHLEELARKGSGGYI | K(SEQ ID NO: 101)-X3- |
| | PEAPRDGQAYVRKDGEWVLLSTFLGGS | GYIPEAPRDGQAYVRKDGEWVLLSTFL (SEQ ID |
| | GSSGSAWSHPQFEKGGGSGGGSGGSAW | NO: 179)-X4 |
| | SHPQFEK (SEQ ID NO: 361) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| 5L6HC3_1 | DDEREIREIEEEARRILEHLEELARKG | WATEMMLELIKSDDEREIREIEEEARRILEHLEELAR |
| | SGSSEELRAVADLORLNIELARKLLEA | K(SEQ ID NO: 101)-X2- |
| | VARLQELNIDLVRKTSELTDEKTIREE | SEELRAVADLQRLNIELARKLLEAVARLQELNIDLVR |
| | IRKVKEESKRIVEEAEEEIRRAKEESR | KTSELTDEKTIREEIRKVKEESKRIVEEAEEEIRRAK |
| | KIADESRGGSGSSGSAWSHPQFEKGGG | EESRKIADESR (SEQ ID NO: 589) |
| | SGGGSGGSAWSHPQFEK (SEQ ID | |
| | NO: 362) | |
| AHB2- | MEKKIELEEQVMHVLDQVSELAHELLH | X1- |
| 5GS- | KLTGEELERAAYFNWWATEMMLELIKS | ELEEQVMHVLDQVSELAHELLHKLTGEELERAAYFNW |
| 5L6HC3_1 | DDEREIREIEEEARRILEHLEELARKG | WATEMMLELIKSDDEREIREIEEEARRILEHLEELAR |
| | SGSGSEELRAVADLORLNIELARKLLE | K(SEQ ID NO: 101)-X2- |
| | AVARLQELNIDLVRKTSELTDEKTIRE | SEELRAVADLQRLNIELARKLLEAVARLQELNIDLVR |
| | EIRKVKEESKRIVEEAEEEIRRAKEES | KTSELTDEKTIREEIRKVKEESKRIVEEAEEEIRRAK |
| | RKIADESRGGSGSSGSAWSHPQFEKGG | EESRKIADESR (SEQ ID NO: 589) |
| | GSGGGSGGSAWSHPQFEK (SEQ ID | |
| | NO: 363) | |
| AHB2- | MEKKIELEEQVMHVLDQVSELAHELLH | X1- |
| 28GS- | KLTGEELERAAYFNWWATEMMLELIKS | ELEEQVMHVLDQVSELAHELLHKLTGEELERAAYFNW |
| AHB2- | DDEREIREIEEEARRILEHLEELARKG | WATEMMLELIKSDDEREIREIEEEARRILEHLEELAR |
| 4GS- | SGSGSGSGSGSGSGSGSGSGSGSGSGS | K(SEQ ID NO: 101)-X2- |
| 5L6HC3_1 | ELEEQVMHVLDQVSELAHELLHKLTGE | ELEEQVMHVLDQVSELAHELLHKLTGEELERAAYFNW |
| | ELERAAYFNWWATEMMLELIKSDDERE | WATEMMLELIKSDDEREIREIEEEARRILEHLEELAR |
| | IREIEEEARRILEHLEELARKGSGSSE | K(SEQ ID NO: 101)-X3 |
| | ELRAVADLORLNIELARKLLEAVARLQ | |
| | ELNIDLVRKTSELTDEKTIREEIRKVK | |
| | EESKRIVEEAEEEIRRAKEESRKIADE | |
| | SRGGSGSSGSAWSHPQFEKGGGSGGGS | |
| | GGSAWSHPQFEK (SEQ ID NO: | |
| | 364) | |
| AHB2- | MEKKIELEEQVMHVLDQVSELAHELLH | X1- |
| 28GS- | KLTGEELERAAYFNWWATEMMLELIKS | ELEEQVMHVLDQVSELAHELLHKLTGEELERAAYFNW |
| AHB2- | DDEREIREIEEEARRILEHLEELARKG | WATEMMLELIKSDDEREIREIEEEARRILEHLEELAR |
| 5GS- | SGSGSGSGSGSGSGSGSGSGSGSGSGS | K(SEQ ID NO: 101)-X2- |
| 5L6HC3_1 | ELEEQVMHVLDQVSELAHELLHKLTGE | ELEEQVMHVLDQVSELAHELLHKLTGEELERAAYFNW |
| | ELERAAYFNWWATEMMLELIKSDDERE | WATEMMLELIKSDDEREIREIEEEARRILEHLEELAR |
| | IREIEEEARRILEHLEELARKGSGSGS | K(SEQ ID NO: 101)-X3 |
| | EELRAVADLQRLNIELARKLLEAVARL | |
| | QELNIDLVRKTSELTDEKTIREEIRKV | |
| | KEESKRIVEEAEEEIRRAKEESRKIAD | |
| | ESRGGSGSSGSAWSHPQFEKGGGSGGG | |
| | SGGSAWSHPQFEK (SEQ ID NO: | |
| | 365) | |
| LCB3- | MEKKINLDELHMQMTDLVYEALHFAKD | X1- |
| 6GS-1rfo | EEFQKHVFQLFEKATKAYKNKDRQKLE | NLDELHMQMTDLVYEALHFAKDEEFQKHVFQLFEKAT |
| | KVVEELKELLERLLSGSGSGSGYIPEA | KAYKNKDRQKLEKVVEELKELLERLLS (SEQ |
| | PRDGQAYVRKDGEWVLLSTFLGGSGSS | ID NO: 155)-X2- |
| | GSAWSHPQFEKGGGSGGGSGGSAWSHP | GYIPEAPRDGQAYVRKDGEWVLLSTFL (SEQ ID |
| | QFEK (SEQ ID NO: 366) | NO: 179)-X3 |
| LCB3- | MEKKINLDELHMQMTDLVYEALHFAKD | X1- |
| 5GS-1rfo | EEFQKHVFQLFEKATKAYKNKDRQKLE | NLDELHMQMTDLVYEALHFAKDEEFQKHVFQLFEKAT |
| | KVVEELKELLERLLSGSGSGGYIPEAP | KAYKNKDROKLEKVVEELKELLERLLS (SEQ ID |
| | RDGQAYVRKDGEWVLLSTFLGGSGSSG | NO: 155)-X2- |
| | SAWSHPQFEKGGGSGGGSGGSAWSHPQ | GYIPEAPRDGQAYVRKDGEWVLLSTFL (SEQ ID |
| | FEK (SEQ ID NO: 367) | NO: 179)-X3 |
| LCB3- | MEKKINLDELHMQMTDLVYEALHFAKD | X1- |
| 4GS-1rfo | EEFQKHVFQLFEKATKAYKNKDRQKLE | NLDELHMQMTDLVYEALHFAKDEEFQKHVFQLFEKAT |
| | KVVEELKELLERLLSGSGSGYIPEAPR | KAYKNKDROKLEKVVEELKELLERLLS (SEQ ID |
| | DGQAYVRKDGEWVLLSTFLGGSGSSGS | NO: 155)-X2- |
| | AWSHPQFEKGGGSGGGSGGSAWSHPQF | GYIPEAPRDGQAYVRKDGEWVLLSTFL (SEQ ID |
| | EK (SEQ ID NO: 368) | NO: 179)-X3 |
| LCB3- | MEKKINLDELHMQMTDLVYEALHFAKD | X1- |
| 5GS- | EEFQKHVFQLFEKATKAYKNKDRQKLE | NLDELHMQMTDLVYEALHFAKDEEFQKHVFQLFEKAT |
| 5L6HC3_1 | KVVEELKELLERLLSGSGSGSEELRAV | KAYKNKDROKLEKVVEELKELLERLLS (SEQ ID |
| | ADLQRLNIELARKLLEAVARLQELNID | NO: 155)-X2- |
| | LVRKTSELTDEKTIREEIRKVKEESKR | SEELRAVADLQRLNIELARKLLEAVARLQELNIDLVR |
| | IVEEAEEEIRRAKEESRKIADESRGGS | KTSELTDEKTIREEIRKVKEESKRIVEEAEEEIRRAK |
| | GSSGSAWSHPQFEKGGGSGGGSGGSAW | EESRKIADESR (SEQ ID NO: 589) |
| | SHPQFEK (SEQ ID NO: 369) | |
| LCB3- | MEKKINLDELHMQMTDLVYEALHFAKD | X1- |
| 28GS- | EEFQKHVFQLFEKATKAYKNKDRQKLE | NLDELHMQMTDLVYEALHFAKDEEFQKHVFQLFEKAT |
| LCB3- | KVVEELKELLERLLSGSGSGSGSGSGS | KAYKNKDRqKLEKVVEELKELLERLLS (SEQ ID |
| 4GS- | GSGSGSGSGSGSGSGSNLDELHMQMTD | NO: 155)-X2- |
| 5L6HC3_1 | LVYEALHFAKDEEFQKHVFQLFEKATK | NLDELHMQMTDLVYEALHFAKDEEFQKHVFQLFEKAT |
| | AYKNKDRQKLEKVVEELKELLERLLSG | KAYKNKDRQKLEKVVEELKELLERLLS ( SEQ ID |
| | SGSSEELRAVADLQRLNIELARKLLEA | NO: 155)-X3 |
| | VARLQELNIDLVRKTSELTDEKTIREE | |
| | IRKVKEESKRIVEEAEEEIRRAKEESR | |
| | KIADESRGGSGSSGSAWSHPQFEKGGG | |
| | SGGGSGGSAWSHPQFEK (SEQ ID | |
| | NO: 370) | |
| LCB3- | MEKKINLDELHMQMTDLVYEALHFAKD | X1- |
| 28GS- | EEFQKHVFQLFEKATKAYKNKDRQKLE | NLDELHMQMTDLVYEALHFAKDEEFQKHVFQLFEKAT |
| LCB3- | KVVEELKELLERLLSGSGSGSGSGSGS | KAYKNKDROKLEKVVEELKELLERLLS (SEQ ID |
| 5GS- | GSGSGSGSGSGSGSGSNLDELHMQMTD | NO: 155)-X2- |
| 5L6HC3_1 | LVYEALHFAKDEEFQKHVFQLFEKATK | NLDELHMQMTDLVYEALHFAKDEEFQKHVFQLFEKAT |
| | AYKNKDRQKLEKVVEELKELLERLLSG | KAYKNKDRQKLEKVVEELKELLERLLS (SEQ ID |
| | SGSGSEELRAVADLORLNIELARKLLE | NO: 155)-X3 |
| | AVARLQELNIDLVRKTSELTDEKTIRE | |
| | EIRKVKEESKRIVEEAEEEIRRAKEES | |
| | RKIADESRGGSGSSGSAWSHPQFEKGG | |
| | GSGGGSGGSAWSHPQFEK (SEQ ID | |
| | NO: 371) | |
| 36729.2_ | MEKKIEEAELAYLLGELAYKLGEYRIA | X1- |
| LCB1v2.2_ | IRAYRIALKRDPNNAEAWYNLGNAYYK | DKENVLQKIYEIMKELERLGHAEASMQVSDLIYEFMK |
| 6GS | QGDYDEAIEYYQKALELDPNNAEAWYN | TKDENLLEEAERLLEEVKR (SEQ ID NO: |
| | LGNAYYKQGDYDEAIEYYQKALELDPN | 135)-X2- |
| | NAEAWYNLGNAYYKQGDYDEAIEYYQK | EEAELAYLLGELAYKLGEYRIAIRAYRIALKRDPNNA |
| | ALELGSGSGSDKENVLQKIYEIMKELE | EAWYNLGNAYYKQGDYDEAIEYYQKALELDPNNAEAW |
| | RLGHAEASMQVSDLIYEFMKTKDENLL | YNLGNAYYKQGDYDEAIEYYQKALELDPNNAEAWYNL |
| | EEAERLLEEVKRGGSGSSGSAWSHPQF | GNAYYKQGDYDEAIEYYQKALEL (SEQ ID NO: |
| | EKGGGSGGGSGGSAWSHPQFEK (SEQ | 590) |
| | ID NO: 372) | |
| 36729.2_ | MEKKIEEAELAYLLGELAYKLGEYRIA | X1- |
| LCB1v2.2_ | IRAYRIALKRDPNNAEAWYNLGNAYYK | DKENVLQKIYEIMKELERLGHAEASMQVSDLIYEFMK |
| 4GS | QGDYDEAIEYYQKALELDPNNAEAWYN | TKDENLLEEAERLLEEVKR (SEQ ID NO: |
| | LGNAYYKQGDYDEAIEYYQKALELDPN | 135)-X2- |
| | NAEAWYNLGNAYYKQGDYDEAIEYYQK | EEAELAYLLGELAYKLGEYRIAIRAYRIALKRDPNNA |
| | ALELGSGSDKENVLQKIYEIMKELERL | EAWYNLGNAYYKQGDYDEAIEYYQKALELDPNNAEAW |
| | GHAEASMQVSDLIYEFMKTKDENLLEE | YNLGNAYYKQGDYDEAIEYYQKALELDPNNAEAWYNL |
| | AERLLEEVKRGGSGSSGSAWSHPQFEK | GNAYYKQGDYDEAIEYYQKALEL (SEQ ID NO: |
| | GGGSGGGSGGSAWSHPQFEK (SEQ | 590) |
| | ID NO: 373) | |
| 36729.2_ | MEKKIEEAELAYLLGELAYKLGEYRIA | X1- |
| LCB1v2.2_ | IRAYRIALKRDPNNAEAWYNLGNAYYK | DKENVLQKIYEIMKELERLGHAEASMQVSDLIYEFMK |
| 2GS | QGDYDEAIEYYQKALELDPNNAEAWYN | TKDENLLEEAERLLEEVKR (SEQ ID NO: |
| | LGNAYYKQGDYDEAIEYYQKALELDPN | 135)-X2- |
| | NAEAWYNLGNAYYKQGDYDEAIEYYQK | EEAELAYLLGELAYKLGEYRIAIRAYRIALKRDPNNA |
| | ALELGSDKENVLOKIYEIMKELERLGH | EAWYNLGNAYYKQGDYDEAIEYYQKALELDPNNAEAW |
| | AEASMQVSDLIYEFMKTKDENLLEEAE | YNLGNAYYKQGDYDEAIEYYQKALELDPNNAEAWYNL |
| | RLLEEVKRGGSGSSGSAWSHPQFEKGG | GNAYYKQGDYDEAIEYYQKALEL (SEQ ID NO: |
| | GSGGGSGGSAWSHPQFEK (SEQ ID | 590) |
| | NO: 374) | |
| LCB3- | MEKKINLDELHMQMTDLVYEALHFAKD | - |
| PAS24- | EEFQKHVFQLFEKATKAYKNKDRQKLE | X1NLDELHMQMTDLVYEALHFAKDEEFQKHVFQLFEK |
| LCB3- | KVVEELKELLERLLSGGASPAAPAPAS | ATKAYKNKDROKLEKVVEELKELLERLLS (SEQ ID |
| 4GS- | PAAPAPSAPAGGNLDELHMQMTDLVYE | NO: 155)-X2- |
| 5L6HC3_1 | ALHFAKDEEFQKHVFQLFEKATKAYKN | NLDELHMQMTDLVYEALHFAKDEEFQKHVFQLFEKAT |
| | KDRQKLEKVVEELKELLERLLSGSGSS | KAYKNKDRQKLEKVVEELKELLERLLS (SEQ ID |
| | EELRAVADLQRLNIELARKLLEAVARL | NO: 155)-X3 |
| | QELNIDLVRKTSELTDEKTIREEIRKV | |
| | KEESKRIVEEAEEEIRRAKEESRKIAD | |
| | ESRGGSGSSGSAWSHPQFEKGGGSGGG | |
| | SGGSAWSHPQFEK (SEQ ID NO: | |
| | 375) | |
| LCB3- | MEKKINLDELHMQMTDLVYEALHFAKD | X1- |
| PAS24- | EEFQKHVFQLFEKATKAYKNKDRQKLE | NLDELHMQMTDLVYEALHFAKDEEFQKHVFQLFEKAT |
| LCB3- | KVVEELKELLERLLSGGASPAAPAPAS | KAYKNKDROKLEKVVEELKELLERLLS (SEQ ID |
| 5GS- | PAAPAPSAPAGGNLDELHMQMTDLVYE | NO: 155)-X2- |
| 5L6HC3_1 | ALHFAKDEEFQKHVFQLFEKATKAYKN | NLDELHMQMTDLVYEALHFAKDEEFQKHVFQLFEKAT |
| | KDRQKLEKVVEELKELLERLLSGSGSG | KAYKNKDROKLEKVVEELKELLERLLS (SEQ ID |
| | SEELRAVADLORLNIELARKLLEAVAR | NO: 155)-X3 |
| | LQELNIDLVRKTSELTDEKTIREEIRK | |
| | VKEESKRIVEEAEEEIRRAKEESRKIA | |
| | DESRGGSGSSGSAWSHPQFEKGGGSGG | |
| | GSGGSAWSHPQFEK (SEQ ID NO: | |
| | 376) | |
| LCB1- | MEKKIDKENVLQKIYEIMKELERLGHA | X1- |
| 10GS- | EASMQVSDLIYEFMKTKDENLLEEAER | DKENVLQKIYEIMKELERLGHAEASMQVSDLIYEFMK |
| AHB2- | LLEEVKRGGGSGGGSGGELEEQVMHVL | TKDENLLEEAERLLEEVKR (SEQ ID NO: |
| 4GS-1rfo | DQVSELAHELLHKLTGEELERAAYFNW | 135)-X2- |
| | WATEMMLELIKSDDEREIREIEEEARR | ELEEQVMHVLDQVSELAHELLHKLTGEELERAAYFNW |
| | ILEHLEELARKGSGSGYIPEAPRDGQA | WATEMMLELIKSDDEREIREIEEEARRILEHLEELAR |
| | YVRKDGEWVLLSTFLGGSGSSGSAWSH | K(SEQ ID NO: 101)-X3- |
| | PQFEKGGGSGGGSGGSAWSHPQFEK | GYIPEAPRDGQAYVRKDGEWVLLSTFL (SEQ ID |
| | (SEQ ID NO: 377) | NO: 179)-X4 |
| LCB1- | MEKKIDKENVLQKIYEIMKELERLGHA | X1- |
| 28GS- | EASMQVSDLIYEFMKTKDENLLEEAER | DKENVLQKIYEIMKELERLGHAEASMQVSDLIYEFMK |
| AHB2- | LLEEVKRGSGSGSGSGSGSGSGSGSGS | TKDENLLEEAERLLEEVKR (SEQ ID NO: |
| 4GS-1rfo | GSGSGSGSELEEQVMHVLDQVSELAHE | 135)-X2- |
| | LLHKLTGEELERAAYFNWWATEMMLEL | ELEEQVMHVLDQVSELAHELLHKLTGEELERAAYFNW |
| | IKSDDEREIREIEEEARRILEHLEELA | WATEMMLELIKSDDEREIREIEEEARRILEHLEELAR |
| | RKGSGSGYIPEAPRDGQAYVRKDGEWV | K(SEQ ID NO: 101)-X3- |
| | LLSTFLGGSGSSGSAWSHPQFEKGGGS | GYIPEAPRDGQAYVRKDGEWVLLSTFL (SEQ ID |
| | GGGSGGSAWSHPQFEK (SEQ ID | NO: 179)-X4 |
| | NO: 378) | |
| LCB3- | MEKKINLDELHMQMTDLVYEALHFAKD | X1- |
| 10GS- | EEFQKHVFQLFEKATKAYKNKDRQKLE | NLDELHMQMTDLVYEALHFAKDEEFQKHVFQLFEKAT |
| AHB2- | KVVEELKELLERLLSGGGSGGGSGGEL | KAYKNKDROKLEKVVEELKELLERLLS (SEQ ID |
| 4GS-1rfo | EEQVMHVLDQVSELAHELLHKLTGEEL | NO: 155)-X2- |
| | ERAAYFNWWATEMMLELIKSDDEREIR | ELEEQVMHVLDQVSELAHELLHKLTGEELERAAYFNW |
| | EIEEEARRILEHLEELARKGSGSGYIP | WATEMMLELIKSDDEREIREIEEEARRILEHLEELAR |
| | EAPRDGQAYVRKDGEWVLLSTFLGGSG | K(SEQ ID NO: 101)-X3- |
| | SSGSAWSHPQFEKGGGSGGGSGGSAWS | GYIPEAPRDGQAYVRKDGEWVLLSTFL (SEQ ID |
| | HPQFEK (SEQ ID NO: 379) | NO: 179)-X4 |
| LCB3- | MEKKINLDELHMQMTDLVYEALHFAKD | X1- |
| 16GS- | EEFQKHVFQLFEKATKAYKNKDRQKLE | NLDELHMQMTDLVYEALHFAKDEEFQKHVFQLFEKAT |
| AHB2- | KVVEELKELLERLLSGSGSGSGSGSGS | KAYKNKDRQKLEKVVEELKELLERLLS (SEQ ID |
| 4GS-1rfo | GSGSELEEQVMHVLDQVSELAHELLHK | NO: 155)-X2- |
| | LTGEELERAAYFNWWATEMMLELIKSD | ELEEQVMHVLDQVSELAHELLHKLTGEELERAAYFNW |
| | DEREIREIEEEARRILEHLEELARKGS | WATEMMLELIKSDDEREIREIEEEARRILEHLEELAR |
| | GSGYIPEAPRDGQAYVRKDGEWVLLST | K (SEQ ID NO: 101)-X3- |
| | FLGGSGSSGSAWSHPQFEKGGGSGGGS | GYIPEAPRDGQAYVRKDGEWVLLSTFL (SEQ ID |
| | GGSAWSHPQFEK (SEQ ID NO: | NO: 179)-X4 |
| | 380) | |
| 36729.2_ | MEKKISAWSHPQFEKGGGSGGGSGGSA | X1- |
| 6GS_LCB1 | WSHPQFEKGGSGSSGMEEAELAYLLGE | DKENVLQKIYEIMKELERLGHAEASMQVSDLIYEFMK |
| v2.2- | LAYKLGEYRIAIRAYRIALKRDPNNAE | TKDENLLEEAERLLEEVKR (SEQ ID NO: |
| 28GS- | AWYNLGNAYYKQGDYDEAIEYYQKALE | 135)-X2- |
| LCB3 | LDPNNAEAWYNLGNAYYKQGDYDEAIE | NLDELHMQMTDLVYEALHFAKDEEFQKHVFQLFEKAT |
| | YYQKALELDPNNAEAWYNLGNAYYKQG | KAYKNKDRQKLEKVVEELKELLERLLS (SEQ ID |
| | DYDEAIEYYQKALELGSGSGSDKENVL | NO: 155) |
| | QKIYEIMKELERLGHAEASMQVSDLIY | |
| | EFMKTKDENLLEEAERLLEEVKRGSGS | |
| | GSGSGSGSGSGSGSGSGSGSGSGSNLD | |
| | ELHMQMTDLVYEALHFAKDEEFQKHVF | |
| | QLFEKATKAYKNKDRQKLEKVVEELKE | |
| | LLERLLS (SEQ ID NO: 381) | |
| 36729.2_ | MEKKISAWSHPQFEKGGGSGGGSGGSA | X1- |
| 6GS_LCB1 | WSHPQFEKGGSGSSGMEEAELAYLLGE | DKENVLQKIYEIMKELERLGHAEASMQVSDLIYEFMK |
| v2.2- | LAYKLGEYRIAIRAYRIALKRDPNNAE | TKDENLLEEAERLLEEVKR (SEQ ID NO: |
| 9GS-LCB3 | AWYNLGNAYYKQGDYDEAIEYYQKALE | 135)-X2- |
| | LDPNNAEAWYNLGNAYYKOGDYDEAIE | NLDELHMQMTDLVYEALHFAKDEEFQKHVFQLFEKAT |
| | YYQKALELDPNNAEAWYNLGNAYYKQG | KAYKNKDRQKLEKVVEELKELLERLLS (SEQ ID |
| | DYDEAIEYYQKALELGSGSGSDKENVL | NO: 155) |
| | QKIYEIMKELERLGHAEASMQVSDLIY | |
| | EFMKTKDENLLEEAERLLEEVKRGSGS | |
| | GSGSGNLDELHMQMTDLVYEALHFAKD | |
| | EEFQKHVFQLFEKATKAYKNKDRQKLE | |
| | KVVEELKELLERLLS (SEQ ID NO: | |
| | 382) | |
| 36729.2_ | MEKKISAWSHPQFEKGGGSGGGSGGSA | X1- |
| 6GS_LCB1 | WSHPQFEKGGSGSSGMEEAELAYLLGE | DKENVLQKIYEIMKELERLGHAEASMQVSDLIYEFMK |
| v2.2- | LAYKLGEYRIAIRAYRIALKRDPNNAE | TKDENLLEEAERLLEEVKR (SEQ ID NO: |
| 28GS- | AWYNLGNAYYKQGDYDEAIEYYQKALE | 135)-X2- |
| AHB2 | LDPNNAEAWYNLGNAYYKQGDYDEAIE | ELEEQVMHVLDQVSELAHELLHKLTGEELERAAYFNW |
| | YYQKALELDPNNAEAWYNLGNAYYKQG | WATEMMLELIKSDDEREIREIEEEARRILEHLEELAR |
| | DYDEAIEYYQKALELGSGSGSDKENVL | K (SEQ ID NO: 101) |
| | QKIYEIMKELERLGHAEASMQVSDLIY | |
| | EFMKTKDENLLEEAERLLEEVKRGSGS | |
| | GSGSGSGSGSGSGSGSGSGSGSGSELE | |
| | EQVMHVLDQVSELAHELLHKLTGEELE | |
| | RAAYFNWWATEMMLELIKSDDEREIRE | |
| | IEEEARRILEHLEELARK (SEQ ID | |
| | NO: 383) | |
| 36729.2_ | MEKKISAWSHPQFEKGGGSGGGSGGSA | X1- |
| 6GS_LCB1 | WSHPQFEKGGSGSSGMEEAELAYLLGE | DKENVLQKIYEIMKELERLGHAEASMQVSDLIYEFMK |
| v2.2- | LAYKLGEYRIAIRAYRIALKRDPNNAE | TKDENLLEEAERLLEEVKR (SEQ ID NO: |
| 9GS-AHB2 | AWYNLGNAYYKQGDYDEAIEYYQKALE | 135)-X2- |
| | LDPNNAEAWYNLGNAYYKQGDYDEAIE | ELEEQVMHVLDQVSELAHELLHKLTGEELERAAYFNW |
| | YYQKALELDPNNAEAWYNLGNAYYKQG | WATEMMLELIKSDDEREIREIEEEARRILEHLEELAR |
| | DYDEAIEYYQKALELGSGSGSDKENVL | K (SEQ ID NO: 101) |
| | QKIYEIMKELERLGHAEASMQVSDLIY | |
| | EFMKTKDENLLEEAERLLEEVKRGSGS | |
| | GSGSGELEEQVMHVLDQVSELAHELLH | |
| | KLTGEELERAAYFNWWATEMMLELIKS | |
| | DDEREIREIEEEARRILEHLEELARK | |
| | (SEQ ID NO: 384) | |
| LCB1- | MEKKIDKENVLQKIYEIMKELERLGHA | X1- |
| 10GS- | EASMQVSDLIYEFMKTKDENLLEEAER | DKENVLQKIYEIMKELERLGHAEASMQVSDLIYEFMK |
| LCB3- | LLEEVKRGGGSGGGSGGNLDELHMQMT | TKDENLLEEAERLLEEVKR (SEQ ID NO: |
| 4GS-1rfo | DLVYEALHFAKDEEFQKHVFQLFEKAT | 135)-X2- |
| | KAYKNKDRQKLEKVVEELKELLERLLS | NLDELHMQMTDLVYEALHFAKDEEFQKHVFQLFEKAT |
| | GSGSGYIPEAPRDGQAYVRKDGEWVLL | KAYKNKDROKLEKVVEELKELLERLLS (SEQ ID |
| | STFLGGSGSSGSAWSHPQFEKGGGSGG | NO: 155)-X3- |
| | GSGGSAWSHPQFEK (SEQ ID NO: | GYIPEAPRDGQAYVRKDGEWVLLSTFL (SEQ ID |
| | 385) | NO: 179)-X4 |
| LCB1- | MEKKIDKENVLQKIYEIMKELERLGHA | X1- |
| 28GS- | EASMQVSDLIYEFMKTKDENLLEEAER | DKENVLQKIYEIMKELERLGHAEASMQVSDLIYEFMK |
| LCB3- | LLEEVKRGSGSGSGSGSGSGSGSGSGS | TKDENLLEEAERLLEEVKR (SEQ ID NO: |
| 4GS-1rfo | GSGSGSGSNLDELHMQMTDLVYEALHF | 135)-X2- |
| | AKDEEFQKHVFQLFEKATKAYKNKDRQ | NLDELHMQMTDLVYEALHFAKDEEFQKHVFQLFEKAT |
| | KLEKVVEELKELLERLLSGSGSGYIPE | KAYKNKDRQKLEKVVEELKELLERLLS (SEQ ID |
| | APRDGQAYVRKDGEWVLLSTFLGGSGS | NO: 155)-X3 |
| | SGSAWSHPQFEKGGGSGGGSGGSAWSH | GYIPEAPRDGQAYVRKDGEWVLLSTFL (SEQ ID |
| | PQFEK (SEQ ID NO: 386) | NO: 179)-X4 |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| LCB3- | DDEREIREIEEEARRILEHLEELARKG | WATEMMLELIKSDDEREIREIEEEARRILEHLEELAR |
| 4GS-1rfo | GGSGGGSGGNLDELHMQMTDLVYEALH | K (SEQ ID NO: 101)-X2- |
| | FAKDEEFQKHVFQLFEKATKAYKNKDR | NLDELHMQMTDLVYEALHFAKDEEFQKHVFQLFEKAT |
| | QKLEKVVEELKELLERLLSGSGSGYIP | KAYKNKDRQKLEKVVEELKELLERLLS (SEQ ID |
| | EAPRDGQAYVRKDGEWVLLSTFLGGSG | NO: 155)-X3- |
| | SSGSAWSHPQFEKGGGSGGGSGGSAWS | GYIPEAPRDGQAYVRKDGEWVLLSTFL (SEQ ID |
| | HPQFEK (SEQ ID NO: 387) | NO: 179)-X4 |
| AHB2- | MEKKIELEEQVMHVLDQVSELAHELLH | X1- |
| 16GS- | KLTGEELERAAYFNWWATEMMLELIKS | ELEEQVMHVLDQVSELAHELLHKLTGEELERAAYFNW |
| LCB3- | DDEREIREIEEEARRILEHLEELARKG | WATEMMLELIKSDDEREIREIEEEARRILEHLEELAR |
| 4GS-1rfo | SGSGSGSGSGSGSGSNLDELHMQMTDL | K (SEQ ID NO: 101)-X2- |
| | VYEALHFAKDEEFQKHVFQLFEKATKA | NLDELHMQMTDLVYEALHFAKDEEFQKHVFQLFEKAT |
| | YKNKDRQKLEKVVEELKELLERLLSGS | KAYKNKDRQKLEKVVEELKELLERLLS (SEQ ID |
| | GSGYIPEAPRDGQAYVRKDGEWVLLST | NO: 155)-X3- |
| | FLGGSGSSGSAWSHPQFEKGGGSGGGS | GYIPEAPRDGQAYVRKDGEWVLLSTFL (SEQ ID |
| | GGSAWSHPQFEK (SEQ ID NO: | NO: 179)-X4 |
| | 388) | |
| LCB1- | MEKKIDKENVLQKIYEIMKELERLGHA | X1- |
| 9GS- | EASMQVSDLIYEFMKTKDENLLEEAER | DKENVLQKIYEIMKELERLGHAEASMQVSDLIYEFMK |
| LCB3- | LLEEVKRGSGSGSGSGNLDELHMQMTD | TKDENLLEEAERLLEEVKR (SEQ ID NO: |
| 5GS- | LVYEALHFAKDEEFQKHVFQLFEKATK | 135)-X2- |
| 5L6HC3_1 | AYKNKDRQKLEKVVEELKELLERLLSG | NLDELHMQMTDLVYEALHFAKDEEFQKHVFQLFEKAT |
| | SGSGSEELRAVADLQRLNIELARKLLE | KAYKNKDRQKLEKVVEELKELLERLLS (SEQ ID |
| | AVARLQELNIDLVRKTSELTDEKTIRE | NO: 155)-X3 |
| | EIRKVKEESKRIVEEAEEEIRRAKEES | |
| | RKIADESRGGSGSSGSAWSHPQFEKGG | |
| | GSGGGSGGSAWSHPQFEK (SEQ ID | |
| | NO: 389) | |
| AHB2- | MEKKIELEEQVMHVLDQVSELAHELLH | X1- |
| 9GS- | KLTGEELERAAYFNWWATEMMLELIKS | ELEEQVMHVLDQVSELAHELLHKLTGEELERAAYFNW |
| LCB3- | DDEREIREIEEEARRILEHLEELARKG | WATEMMLELIKSDDEREIREIEEEARRILEHLEELAR |
| 5GS- | SGSGSGSGNLDELHMQMTDLVYEALHF | K (SEQ ID NO: 101)-X2- |
| 5L6HC3_1 | AKDEEFQKHVFQLFEKATKAYKNKDRQ | NLDELHMQMTDLVYEALHFAKDEEFQKHVFQLFEKAT |
| | KLEKVVEELKELLERLLSGSGSGSEEL | KAYKNKDROKLEKVVEELKELLERLLS (SEQ ID |
| | RAVADLQRLNIELARKLLEAVARLQEL | NO: 155)-X3 |
| | NIDLVRKTSELTDEKTIREEIRKVKEE | |
| | SKRIVEEAEEEIRRAKEESRKIADESR | |
| | GGSGSSGSAWSHPQFEKGGGSGGGSGG | |
| | SAWSHPQFEK (SEQ ID NO: 390) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| Ina0_int | DDEREIREIEEEARRILEHLEELARKG | WATEMMLELIKSDDEREIREIEEEARRILEHLEELAR |
| 2-R3 | SGSEEAELAYLLGELAYKLGEYRIAIR | K (SEQ ID NO: 101)-X2- |
| | AYRIALKRDPNNAEAWYNLGNAYYKQG | EEAELAYLLGELAYKLGEYRIAIRAYRIALKRDPNNA |
| | DYDEAIYYQKALELDPNNAEAKQNLGN | EAWYNLGNAYYKQGDYDEAIYYQKALELDPNNAEAKQ |
| | AKQKQGGGSGSSGSAWSHPQFEKGGGS | NLGNAKQKQ (SEQ ID NO: 591) |
| | GGGSGGSAWSHPQFEK (SEQ ID NO: | |
| | 391) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| Ina0_int | DDEREIREIEEEARRILEHLEELARKG | WATEMMLELIKSDDEREIREIEEEARRILEHLEELAR |
| 2-R3 | SGSGSEEAELAYLLGELAYKLGEYRIA | K (SEQ ID NO: 101)-X2- |
| | IRAYRIALKRDPNNAEAWYNLGNAYYK | EEAELAYLLGELAYKLGEYRIAIRAYRIALKRDPNNA |
| | QGDYDEIEYYQKALELDPNNAEAKQNL | EAWYNLGNAYYKQGDYDEIEYYQKALELDPNNAEAKQ |
| | GNAKQKQGGGSGSSGSAWSHPQFEKGG | NLGNAKQKQ (SEQ ID NO: _592) |
| | GSGGGSGGSAWSHPQFEK (SEQ ID | |
| | NO: 392) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | SGSEEAELAYLLGELAYKLGEYRIAIR | K (SEQ ID NO: 101)-X2- |
| | AYRIALKRDPNNAEAWYNLGNAYYKQG | EEAELAYLLGELAYKLGEYRIAIRAYRIALKRDPNNA |
| | DYDEAIYYQKALELDPNNAEAWYNLGN | EAWYNLGNAYYKQGDYDEAIYYQKALELDPNNAEAWY |
| | AYYKQGDYDEAIEYYQKALELDPNNAE | NLGNAYYKQGDYDEAIEYYQKALELDPNNAEAKQNLG |
| | AKONLGNKOKQGGGSGSSGSAWSHPQF | NKQKQ (SEQ ID NO: 593) |
| | EKGGGSGGGSGGSAWSHPQFEK (SEQ | |
| | ID NO: 393) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | SGSGSEEAELAYLLGELAYKLGEYRIA | K (SEQ ID NO: 101)-X2- |
| | IRAYRIALKRDPNNAEAWYNLGNAYYK | EEAELAYLLGELAYKLGEYRIAIRAYRIALKRDPNNA |
| | QGDYDEAIYYQKALELDPNNAEAWYNL | EAWYNLGNAYYKQGDYDEAIYYQKALELDPNNAEAWY |
| | GNAYYKQGDYDEAIEYYQKALELDPNN | NLGNAYYKQGDYDEAIEYYQKALELDPNNAEAKQNLG |
| | AEAKQNLGNKOKQGGGSGSSGSAWSHP | NKQKQ (SEQ ID NO: 593) |
| | QFEKGGGSGGGSGGSAWSHPQFEK | |
| | (SEQ ID NO: 394) | |
| AHB2- | MEKKIELEEQVMHVLDQVSELAHELLH | X1- |
| 4GS-6msr | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSEYEIRKALEELKASTAELKRAT | K (SEQ ID NO: 101)-X2- |
| | ASLRASTEELKKNPSEDALVENNRLIV | EYEIRKALEELKASTAELKRATASLRASTEELKKMPS |
| | EHNAIIVENRIIAAVLELIVRAIKGGS | EDALVENMRLIVEHNAIIVENRIIAAVLELIVRAIK |
| | GSSGSAWSHPQFEKGGGSGGGSGGSAW | (SEQ ID NO: 594) |
| | SHPQFEK (SEQ ID NO: 395) | |
| AHB2- | MEKKIELEEQVMHVLDQVSELAHELLH | X1- |
| 6GS-6msr | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSGSEYEIRKALEELKASTAELKR | K (SEQ ID NO: 101)-X2- |
| | ATASLRASTEELKKNPSEDALVENNRL | EYEIRKALEELKASTAELKRATASLRASTEELKKMPS |
| | IVEHNAIIVENRIIAAVLELIVRAIKG | EDALVENMRLIVEHNAIIVENRIIAAVLELIVRAIK |
| | GSGSSGSAWSHPQFEKGGGSGGGSGGS | (SEQ ID NO: 594) |
| | AWSHPQFEK (SEQ ID NO: 396) | |
| 36729.2_ | MEKKISAWSHPQFEKGGGSGGGSGGSA | X1- |
| LCB1v2.2_ | WSHPQFEKGGSGSSGEEAELAYLLGEL | EEAELAYLLGELAYKLGEYRIAIRAYRIALKRDPNNA |
| 6GS | AYKLGEYRIAIRAYRIALKRDPNNAEA | EAWYNLGNAYYKQGDYDEAIEYYQKALELDPNNAEAW |
| | WYNLGNAYYKQGDYDEAIEYYQKALEL | YNLGNAYYKQGDYDEAIEYYQKALELDPNNAEAWYNL |
| | DPNNAEAWYNLGNAYYKQGDYDEAIEY | GNAYYKQGDYDEAIEYYQKALEL (SEQ ID NO: |
| | YQKALELDPNNAEAWYNLGNAYYKQGD | 590)-X2- |
| | YDEAIEYYQKALELGSGSGSDKENVLQ | DKENVLQKIYEIMKELERLGHAEASMQVSDLIYEFMK |
| | KIYEIMKELERLGHAEASMQVSDLIYE | TKDENLLEEAERLLEEVKR (SEQ ID NO: 135) |
| | FMKTKDENLLEEAERLLEEVKR (SEQ | |
| | ID NO: 397) | |
| 36729.2_ | MEKKISAWSHPQFEKGGGSGGGSGGSA | X1- |
| LCB1v2.2_ | WSHPQFEKGGSGSSGEEAELAYLLGEL | EEAELAYLLGELAYKLGEYRIAIRAYRIALKRDPNNA |
| 4GS | AYKLGEYRIAIRAYRIALKRDPNNAEA | EAWYNLGNAYYKQGDYDEAIEYYQKALELDPNNAEAW |
| | WYNLGNAYYKQGDYDEAIEYYQKALEL | YNLGNAYYKQGDYDEAIEYYQKALELDPNNAEAWYNL |
| | DPNNAEAWYNLGNAYYKQGDYDEAIEY | GNAYYKQGDYDEAIEYYQKALEL (SEQ ID NO: |
| | YQKALELDPNNAEAWYNLGNAYYKQGD | 590)-X2- |
| | YDEAIEYYQKALELGSGSDKENVLQKI | DKENVLQKIYEIMKELERLGHAEASMQVSDLIYEFMK |
| | YEIMKELERLGHAEASMQVSDLIYEFM | TKDENLLEEAERLLEEVKR (SEQ ID NO: 135) |
| | KTKDENLLEEAERLLEEVKR (SEQ | |
| | ID NO: 398) | |
| 36729.2_ | MEKKISAWSHPQFEKGGGSGGGSGGSA | X1- |
| LCB1v2.2_ | WSHPQFEKGGSGSSGEEAELAYLLGEL | EEAELAYLLGELAYKLGEYRIAIRAYRIALKRDPNNA |
| 2GS | AYKLGEYRIAIRAYRIALKRDPNNAEA | EAWYNLGNAYYKQGDYDEAIEYYQKALELDPNNAEAW |
| | WYNLGNAYYKOGDYDEAIEYYQKALEL | YNLGNAYYKQGDYDEAIEYYQKALELDPNNAEAWYNL |
| | DPNNAEAWYNLGNAYYKQGDYDEAIEY | GNAYYKQGDYDEAIEYYQKALEL (SEQ ID NO: |
| | YQKALELDPNNAEAWYNLGNAYYKQGD | 590)-X2- |
| | YDEAIEYYQKALELGSDKENVLQKIYE | DKENVLQKIYEIMKELERLGHAEASMQVSDLIYEFMK |
| | IMKELERLGHAEASMQVSDLIYEFMKT | TKDENLLEEAERLLEEVKR(SEQ ID NO: 135) |
| | KDENLLEEAERLLEEVKR (SEQ ID | |
| | NO: 399) | |
| AHB2- | MEKKIELEEQVMHVLDQVSELAHELLH | X1- |
| 8GS- | KLTGEELERAAYFNWWATEMMLELIKS | ELEEQVMHVLDQVSELAHELLHKLTGEELERAAYFNW |
| 1na0_int2- | DDEREIREIEEEARRILEHLEELARKG | WATEMMLELIKSDDEREIREIEEEARRILEHLEELAR |
| R3 | SGSGSGSEEAELAYLLGELAYKLGEYR | K (SEQ ID NO: 101)-X2- |
| | IAIRAYRIALKRDPNNAEAWYNLGNAY | EEAELAYLLGELAYKLGEYRIAIRAYRIALKRDPNNA |
| | YKQGDYDEAIEYYQKALELDPNNAEAK | EAWYNLGNAYYKQGDYDEAIEYYQKALELDPNNAEAK |
| | QNLGNAKQKQGGGSGSSGSAWSHPQFE | QNLGNAKQKQG (SEQ ID NO: 180)-X3 |
| | KGGGSGGGSGGSAWSHPQFEK (SEQ | |
| | ID NO: 400) | |
| AHB2- | MEKKIELEEQVMHVLDQVSELAHELLH | X1- |
| 8GS- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | SGSGSGSEEAELAYLLGELAYKLGEYR | K (SEQ ID NO: 101) -X2- |
| | IAIRAYRIALKRDPNNAEAWYNLGNAY | EEAELAYLLGELAYKLGEYRIAIRAYRIALKRDPNNA |
| | YKQGDYDEAIEYYQKALELDPNNAEAW | EAWYNLGNAYYKQGDYDEAIEYYQKALELDPNNAEAW |
| | YNLGNAYYKQGDYDEAIEYYQKALELD | YNLGNAYYKQGDYDEAIEYYQKALELDPNNAEAKQNL |
| | PNNAEAKQNLGNAKQKQGGGSGSSGSA | GNAKQKQG (SEQ ID NO: 181) -X3 |
| | WSHPQFEKGGGSGGGSGGSAWSHPQFE | |
| | K (SEQ ID NO: 401) | |
| AHB2- | MEKKIELEEQVMHVLDQVSELAHELLH | X1- |
| 8GS-6msr | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSGSGSEYEIRKALEELKASTAEL | K (SEQ ID NO: 101)-X2- |
| | KRATAS LRASTEELKKNPSEDALVENN | GSEYEIRKALEELKASTAELKRATASLRASTEELKKN |
| | RLIVEHNAIIVENNRIIAAVLELIVRA | PSEDALVENNRLIVEHNAIIVENNRIIAAVLELIVRA |
| | IKGGSGSSGSAWSHPQFEKGGGSGGGS | IK (SEQ ID NO: 182)-X3 |
| | GGSAWSHPQFEK (SEQ ID NO: | |
| | 402) | |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| 6msr | DDEREIREIEEEARRILEHLEELARKG | WATEMMLELIKSDDEREIREIEEEARRILEHLEELAR |
| | SGSGSGSGSGSEYEIRKALEELKASTA | K(SEQ ID NO: 101) -X2- |
| | ELKRATASLRASTEELKKNPSEDALVE | GSEYEIRKALEELKASTAELKRATASLRASTEELKKN |
| | NNRLIVEHNAIIVENNRIIAAVLELIV | PSEDALVENNRLIVEHNAIIVENNRIIAAVLELIVRA |
| | RAIKGGSGSSGSAWSHPQFEKGGGSGG | IK(SEQ ID NO: 182)-X3 |
| | GSGGSAWSHPQFEK (SEQ ID NO: | |
| | 403) | |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| 1na0_int2-R3 | DDEREIREIEEEARRILEHLEELARKG | WATEMMLELIKSDDEREIREIEEEARRILEHLEELAR |
| | SGSGSGSGSEEAELAYLLGELAYKLGE | K (SEQ ID NO: 101)-X2- |
| | YRIAIRAYRIALKRDPNNAEAWYNLGN | EEAELAYLLGELAYKLGEYRIAIRAYRIALKRDPNNA |
| | AYYKQGDYDEAIEYYQKALELDPNNAE | EAWYNLGNAYYKQGDYDEAIEYYQKALELDPNNAEAK |
| | AKONLGNAKQKQGGGSGSSGSAWSHPQ | QNLGNAKQKQG (SEQ ID NO: 180)-X3 |
| | FEKGGGSGGGSGGSAWSHPQFEK | |
| | (SEQ ID NO: 404) | |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | SGSGSGSGSEEAELAYLLGELAYKLGE | K (SEQ ID NO: 101)-X2- |
| | YRIAIRAYRIALKRDPNNAEAWYNLGN | EEAELAYLLGELAYKLGEYRIAIRAYRIALKRDPNNA |
| | AYYKQGDYDEAIEYYQKALELDPNNAE | EAWYNLGNAYYKQGDYDEAIEYYQKALELDPNNAEAW |
| | AWYNLGNAYYKQGDYDEAIEYYQKALE | YNLGNAYYKQGDYDEAIEYYQKALELDPNNAEAKQNL |
| | LDPNNAEAKQNLGNAKQKQGGGSGSSG | GNAKQKQG (SEQ ID NO: 181)-X3 |
| | SAWSHPQFEKGGGSGGGSGGSAWSHPQ | |
| | FEK (SEQ ID NO: 405) | |
| AHB2- | MEKKIELEEQVMHVLDQVSELAHELLH | X1- |
| 4GS-1gcm | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSRMKQIEDKIEEILSKIYHIENEIA | K (SEQ ID NO: 101)-X2- |
| | RIKKLIGERGGSGSSGSAWSHPQFEKG | RMKQIEDKIEEILSKIYHIENEIARIKKLIGER |
| | GGSGGGSGGSAWSHPQFEK (SEQ ID | (SEQ ID NO: 183)-X3 |
| | NO: 406) | |
| AHB2- | MEKKIELEEQVMHVLDQVSELAHELLH | X1- |
| 8GS-1gcm | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSGSRMKQIEDKIEEILSKIYHIE | K (SEQ ID NO: 101)-X2- |
| | NEIARIKKLIGERGGSGSSGSAWSHPQ | RMKQIEDKIEEILSKIYHIENEIARIKKLIGER (SEQ |
| | FEKGGGSGGGSGGSAWSHPQFEK | ID NO: 183)-X3 |
| | (SEQ ID NO: 407) | |
| AHB2- | MEKKIELEEQVMHVLDQVSELAHELLH | X1- |
| 4GS-pRO- | KLTGEELERAAYFNWWATEMMLELIKS | ELEEQVMHVLDQVSELAHELLHKLTGEELERAAYFNW |
| 2-noHis | DDEREIREIEEEARRILEHLEELARKG | WATEMMLELIKSDDEREIREIEEEARRILEHLEELAR |
| | SGSGSEYEIRKALEELKASTAELKRST | K (SEQ ID NO: 101)-X2- |
| | ASLRASTEELKKNPSEDALVENNRLIV | GSEYEIRKALEELKASTAELKRSTASLRASTEELKKN |
| | ENNAIIVENNRIIAAVLELIVRAIKGG | PSEDALVENNRLIVENNAIIVENNRIIAAVLELIVRA |
| | SGSSGSAWSHPQFEKGGGSGGGSGGSA | IK (SEQ ID NO: 184)-X3 |
| | WSHPQFEK (SEQ ID NO: 408) | |
| AHB2- | MEKKIELEEQVMHVLDQVSELAHELLH | X1ELEEQVMHVLDQVSELAHELLHKLTGEELERAAYF |
| 8GS-PRO- | KLTGEELERAAYFNWWATEMMLELIKS | NWWATEMMLELIKSDDEREIREIEEEARRILEHLEEL |
| 2-noHis | DDEREIREIEEEARRILEHLEELARKG | ARK (SEQ ID NO: 101)-X2- |
| | SGSGSGSGSEYEIRKALEELKASTAEL | GSEYEIRKALEELKASTAELKRSTASLRASTEELKKN |
| | KRSTASLRASTEELKKNPSEDALVENN | PSEDALVENNRLIVENNAIIVENNRIIAAVLELIVRA |
| | RLIVENNAIIVENNRIIAAVLELIVRA | IK (SEQ ID NO: 184)-X3 |
| | IKGGSGSSGSAWSHPQFEKGGGSGGGS | |
| | GGSAWSHPQFEK (SEQ ID NO: | |
| | 409) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| 1na0_3 | DDEREIREIEEEARRILEHLEELARKG | WATEMMLELIKSDDEREIREIEEEARRILEHLEELAR |
| | SGSGSNLAEKMYKAGNAMYRKGQYTIA | K (SEQ ID NO: 101)-X2- |
| | IIAYTLALLKDPNNAEAWYNLGNAAYK | NLAEKMYKAGNAMYRKGQYTIAIIAYTLALLKDPNNA |
| | KGEYDEAIEAYQKALELDPNNAEAWYN | EAWYNLGNAAYKKGEYDEAI EAYQKALELDPNNAEAW |
| | LGNAYYKQGDYDEAIEYYQKALELDPN | YNLGNAYYKQGDYDEAIEYYQKALELDPNNAEAKQNL |
| | NAEAKONLGNAKQKOGGGSGSSGSAWS | GNAKQKQG (SEQ ID NO: 185)-X3 |
| | HPQFEKGGGSGGGSGGSAWSHPQFEK | |
| | (SEQ ID NO: 410) | |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| 1na0_3 | DDEREIREIEEEARRILEHLEELARKG | WATEMMLELIKSDDEREIREIEEEARRILEHLEELAR |
| | SGSGSGSGSNLAEKMYKAGNAMYRKGQ | K (SEQ ID NO: 101)-X2- |
| | YTIAIIAYTLALLKDPNNAEAWYNLGN | NLAEKMYKAGNAMYRKGQYTIAIIAYTLALLKDPNNA |
| | AAYKKGEYDEAIEAYQKALELDPNNAE | EAWYNLGNAAYKKGEYDEAI EAYQKALELDPNNAEAW |
| | AWYNLGNAYYKQGDYDEAIEYYQKALE | YNLGNAYYKQGDYDEAIEYYQKALELDPNNAEAKQNL |
| | LDPNNAEAKQNLGNAKQKQGGGSGSSG | GNAKQKQG (SEQ ID NO: 185)-X3 |
| | SAWSHPQFEKGGGSGGGSGGSAWSHPQ | |
| | FEK (SEQ ID NO: 411) | |
| AHB2- | MEKKIELEEQVMHVLDQVSELAHELLH | X1- |
| 6GS-4pn9 | KLTGEELERAAYFNWWATEMMLELIKS | ELEEQVMHVLDQVSELAHELLHKLTGEELERAAYFNW |
| | DDEREIREIEEEARRILEHLEELARKG | WATEMMLELIKSDDEREIREIEEEARRILEHLEELAR |
| | SGSGSGEIAKSLKEIAKSLKEIAWSLK | K (SEQ ID NO: 101)-X2- |
| | EIAKSLKGGGSGSSGSAWSHPQFEKGG | GEIAKSLKEIAKSLKEIAWSLKEIAKSLKG (SEQ ID |
| | GSGGGSGGSAWSHPQFEK (SEQ ID | NO: 186)-X3 |
| | NO: 412) | |
| AHB2- | MEKKIELEEQVMHVLDQVSELAHELLH | X1- |
| 10GS- | KLTGEELERAAYFNWWATEMMLELIKS | ELEEQVMHVLDQVSELAHELLHKLTGEELERAAYFNW |
| 4pn9 | DDEREIREIEEEARRILEHLEELARKG | WATEMMLELIKSDDEREIREIEEEARRILEHLEELAR |
| | SGSGSGSGSGEIAKSLKEIAKSLKEIA | K (SEQ ID NO: 101)-X2- |
| | WSLKEIAKSLKGGGSGSSGSAWSHPQF | GEIAKSLKEIAKSLKEIAWSLKEIAKSLKG (SEQ ID |
| | EKGGGSGGGSGGSAWSHPQFEK (SEQ | NO: 186)-X3 |
| | ID NO: 413) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 1na0_int2- | DDEREIREIEEEARRILEHLEELARKG | WATEMMLELIKSDDEREIREIEEEARRILEHLEELAR |
| R3 | GSGGEEAELAYLLGELAYKLGEYRIAI | K (SEQ ID NO: 101)-X2- |
| | RAYRIALKRDPNNAEAWYNLGNAYYKQ | EEAELAYLLGELAYKLGEYRIAIRAYRIALKRDPNNA |
| | GDYDEAIEYYQKALELDPNNAEAKQNL | EAWYNLGNAYYKQGDYDEAIEYYQKALELDPNNAEAK |
| | GNAKOKQGGGSGSSGSAWSHPQFEKGG | QNLGNAKQKQG (SEQ ID NO: 180)-X3 |
| | GSGGGSGGSAWSHPQFEK (SEQ ID | |
| | NO: 414) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 1na0_int2- | DDEREIREIEEEARRILEHLEELARKG | WATEMMLELIKSDDEREIREIEEEARRILEHLEELAR |
| R3 | GGSGGGEEAELAYLLGELAYKLGEYRI | K (SEQ ID NO: 101)-X2- |
| | AIRAYRIALKRDPNNAEAWYNLGNAYY | EEAELAYLLGELAYKLGEYRIAIRAYRIALKRDPNNA |
| | KQGDYDEAIEYYQKALELDPNNAEAKQ | EAWYNLGNAYYKQGDYDEAIEYYQKALELDPNNAEAK |
| | NLGNAKQKQGGGSGSSGSAWSHPQFEK | QNLGNAKQKQG (SEQ ID NO: 180)-X3 |
| | GGGSGGGSGGSAWSHPQFEK (SEQ ID | |
| | NO: 415) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 1na0_int2- | DDEREIREIEEEARRILEHLEELARKG | WATEMMLELIKSDDEREIREIEEEARRILEHLEELAR |
| R3 | GGGSGGGGEEAELAYLLGELAYKLGEY | K (SEQ ID NO: 101)-X2- |
| | RIAIRAYRIALKRDPNNAEAWYNLGNA | EEAELAYLLGELAYKLGEYRIAIRAYRIALKRDPNNA |
| | YYKQGDYDEAIEYYQKALELDPNNAEA | EAWYNLGNAYYKQGDYDEAIEYYQKALELDPNNAEAK |
| | KQNLGNAKQKQGGGSGSSGSAWSHPQF | QNLGNAKQKQG (SEQ ID NO: 180)-X3 |
| | EKGGGSGGGSGGSAWSHPQFEK (SEQ | |
| | ID NO: 416) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | GSGGEEAELAYLLGELAYKLGEYRIAI | K (SEQ ID NO: 101)-X2- |
| | RAYRIALKRDPNNAEAWYNLGNAYYKQ | EEAELAYLLGELAYKLGEYRIAIRAYRIALKRDPNNA |
| | GDYDEAIEYYQKALELDPNNAEAWYNL | EAWYNLGNAYYKQGDYDEAIEYYQKALELDPNNAEAW |
| | GNAYYKQGDYDEAIEYYQKALELDPNN | YNLGNAYYKQGDYDEAIEYYQKALELDPNNAEAKQNL |
| | AEAKQNLGNAKQKQGGGSGSSGSAWSH | GNAKQKQG (SEQ ID NO: 181)-X3 |
| | PQFEKGGGSGGGSGGSAWSHPQFEK | |
| | (SEQ ID NO: 417) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | GGSGGGEEAELAYLLGELAYKLGEYRI | K (SEQ ID NO: 101)-X2- |
| | AIRAYRIALKRDPNNAEAWYNLGNAYY | EEAELAYLLGELAYKLGEYRIAIRAYRIALKRDPNNA |
| | KQGDYDEAIEYYQKALELDPNNAEAWY | EAWYNLGNAYYKQGDYDEAIEYYQKALELDPNNAEAW |
| | NLGNAYYKQGDYDEAIEYYQKALELDP | YNLGNAYYKQGDYDEAIEYYQKALELDPNNAEAKQNL |
| | NNAEAKQNLGNAKQKQGGGSGSSGSAW | GNAKQKOG (SEQ ID NO: 181)-X3 |
| | SHPQFEKGGGSGGGSGGSAWSHPQFEK | |
| | (SEQ ID NO: 418) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 1na0_int2 | DDEREIREIEEEARRILEHLEELARKG | WATEMMLELIKSDDEREIREIEEEARRILEHLEELAR |
| | GGGSGGGGEEAELAYLLGELAYKLGEY | K (SEQ ID NO: 101)-X2- |
| | RIAIRAYRIALKRDPNNAEAWYNLGNA | EEAELAYLLGELAYKLGEYRIAIRAYRIALKRDPNNA |
| | YYKQGDYDEAIEYYQKALELDPNNAEA | EAWYNLGNAYYKQGDYDEAIEYYQKALELDPNNAEAW |
| | WYNLGNAYYKQGDYDEAIEYYQKALEL | YNLGNAYYKQGDYDEAIEYYQKALELDPNNAEAKQNL |
| | DPNNAEAKQNLGNAKQKQGGGSGSSGS | GNAKQKQG (SEQ ID NO: 181)-X3 |
| | AWSHPQFEKGGGSGGGSGGSAWSHPQF | |
| | EK (SEQ ID NO: 419) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 6msr | DDEREIREIEEEARRILEHLEELARKG | WATEMMLELIKSDDEREIREIEEEARRILEHLEELAR |
| | GSGGGSEYEIRKALEELKASTAELKRA | K (SEQ ID NO: 101)-X2- |
| | TASLRASTEELKKNPSEDALVENNRLI | GSEYEIRKALEELKASTAELKRATASLRASTEELKKN |
| | VEHNAIIVENNRIIAAVLELIVRAIKG | PSEDALVENNRLIVEHNAIIVENNRIIAAVLELIVRA |
| | GSGSSGSAWSHPQFEKGGGSGGGSGGS | IK (SEQ ID NO: 182)-X3 |
| | AWSHPQFEK (SEQ ID NO: 420) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 6msr | DDEREIREIEEEARRILEHLEELARKG | WATEMMLELIKSDDEREIREIEEEARRILEHLEELAR |
| | GGSGGGGSEYEIRKALEELKASTAELK | K (SEQ ID NO: 101)-X2- |
| | RATASLRASTEELKKNPSEDALVENNR | GSEYEIRKALEELKASTAELKRATASLRASTEELKKN |
| | LIVEHNAIIVENNRIIAAVLELIVRAI | PSEDALVENNRLIVEHNAIIVENNRIIAAVLELIVRA |
| | KGGSGSSGSAWSHPQFEKGGGSGGGSG | IK (SEQ ID NO: 182)-X3 |
| | GSAWSHPQFEK (SEQ ID NO: 421) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 6msr | DDEREIREIEEEARRILEHLEELARKG | WATEMMLELIKSDDEREIREIEEEARRILEHLEELAR |
| | GGGSGGGGGSEYEIRKALEELKASTAE | K (SEQ ID NO: 101)-X2- |
| | LKRATASLRASTEELKKNPSEDALVEN | GSEYEIRKALEELKASTAELKRATASLRASTEELKKN |
| | NRLIVEHNAIIVENNRIIAAVLELIVR | PSEDALVENNRLIVEHNAIIVENNRIIAAVLELIVRA |
| | AIKGGSGSSGSAWSHPQFEKGGGSGGG | IK (SEQ ID NO: 182)-X3 |
| | SGGSAWSHPQFEK (SEQ ID NO: | |
| | 422) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 1gcm | DDEREIREIEEEARRILEHLEELARKG | WATEMMLELIKSDDEREIREIEEEARRILEHLEELAR |
| | GSGGRMKQIEDKIEEILSKIYHIENEI | K (SEQ ID NO: 101)-X2- |
| | ARIKKLIGERGGSGSSGSAWSHPQFEK | RMKQIEDKIEEILSKIYHIENEIARIKKLIGER (SEQ |
| | GGGSGGGSGGSAWSHPQFEK (SEQ | ID NO: 183)-X3 |
| | ID NO: 423) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 1gcm | DDEREIREIEEEARRILEHLEELARKG | WATEMMLELIKSDDEREIREIEEEARRILEHLEELAR |
| | GGSGGGRMKQIEDKIEEILSKIYHIEN | K (SEQ ID NO: 101)-X2- |
| | EIARIKKLIGERGGSGSSGSAWSHPQF | RMKQIEDKIEEILSKIYHIENEIARIKKLIGER (SEQ |
| | EKGGGSGGGSGGSAWSHPQFEK (SEQ | ID NO: 183)-X3 |
| | ID NO: 424) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 1gcm | DDEREIREIEEEARRILEHLEELARKG | WATEMMLELIKSDDEREIREIEEEARRILEHLEELAR |
| | GGGSGGGGRMKQIEDKIEEILSKIYHI | K (SEQ ID NO: 101)-X2- |
| | ENEIARIKKLIGERGGSGSSGSAWSHP | RMKQIEDKIEEILSKIYHIENEIARIKKLIGER (SEQ |
| | QFEKGGGSGGGSGGSAWSHPQFEK | ID NO: 183)-X3 |
| | (SEQ ID NO: 425) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| pRO-2- | DDEREIREIEEEARRILEHLEELARKG | WATEMMLELIKSDDEREIREIEEEARRILEHLEELAR |
| noHis | GSGGGSEYEIRKALEELKASTAELKRS | K (SEQ ID NO: 101)-X2- |
| | TASLRASTEELKKNPSEDALVENNRLI | GSEYEIRKALEELKASTAELKRSTASLRASTEELKKN |
| | VENNAIIVENNRIIAAVLELIVRAIKG | PSEDALVENNRLIVENNAIIVENNRIIAAVLELIVRA |
| | GSGSSGSAWSHPQFEKGGGSGGGSGGS | IK (SEQ ID NO: 184)-X3 |
| | AWSHPQFEK (SEQ ID NO: 426) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| PRO-2- | DDEREIREIEEEARRILEHLEELARKG | WATEMMLELIKSDDEREIREIEEEARRILEHLEELAR |
| noHis | GGSGGGGSEYEIRKALEELKASTAELK | K (SEQ ID NO: 101)-X2- |
| | RSTASLRASTEELKKNPSEDALVENNR | GSEYEIRKALEELKASTAELKRSTASLRASTEELKKN |
| | LIVENNAIIVENNRIIAAVLELIVRAI | PSEDALVENNRLIVENNAIIVENNRIIAAVLELIVRA |
| | KGGSGSSGSAWSHPQFEKGGGSGGGSG | IK (SEQ ID NO: 184)-X3 |
| | GSAWSHPQFEK (SEQ ID NO: 427) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| pRO-2- | DDEREIREIEEEARRILEHLEELARKG | WATEMMLELIKSDDEREIREIEEEARRILEHLEELAR |
| noHis | GGGSGGGGGSEYEIRKALEELKASTAE | K (SEQ ID NO: 101)-X2- |
| | LKRSTASLRASTEELKKNPSEDALVEN | GSEYEIRKALEELKASTAELKRSTASLRASTEELKKN |
| | NRLIVENNAIIVENNRIIAAVLELIVR | PSEDALVENNRLIVENNAIIVENNRIIAAVLELIVRA |
| | AIKGGSGSSGSAWSHPQFEKGGGSGGG | IK (SEQ ID NO: 184)-X3 |
| | SGGSAWSHPQFEK (SEQ ID NO: | |
| | 428) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 1na0_3 | DDEREIREIEEEARRILEHLEELARKG | WATEMMLELIKSDDEREIREIEEEARRILEHLEELAR |
| | GSGGNLAEKMYKAGNAMYRKGQYTIAI | K (SEQ ID NO: 101)-X2- |
| | IAYTLALLKDPNNAEAWYNLGNAAYKK | NLAEKMYKAGNAMYRKGQYTIAIIAYTLALLKDPNNA |
| | GEYDEAIEAYQKALELDPNNAEAWYNL | EAWYNLGNAAYKKGEYDEAI EAYQKALELDPNNAEAW |
| | GNAYYKQGDYDEAIEYYQKALELDPNN | YNLGNAYYKQGDYDEAIEYYQKALELDPNNAEAKQNL |
| | AEAKONLGNAKOKQGGGSGSSGSAWSH | GNAKQKQG (SEQ ID NO: 185)-X3 |
| | PQFEKGGGSGGGSGGSAWSHPQFEK | |
| | (SEQ ID NO: 429) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 1na0_3 | DDEREIREIEEEARRILEHLEELARKG | WATEMMLELIKSDDEREIREIEEEARRILEHLEELAR |
| | GGSGGGNLAEKMYKAGNAMYRKGQYTI | K (SEQ ID NO: 101)-X2- |
| | AIIAYTLALLKDPNNAEAWYNLGNAAY | NLAEKMYKAGNAMYRKGQYTIAIIAYTLALLKDPNNA |
| | KKGEYDEAIEAYQKALELDPNNAEAWY | EAWYNLGNAAYKKGEYDEAIEAYQKALELDPNNAEAW |
| | NLGNAYYKQGDYDEAIEYYQKALELDP | YNLGNAYYKQGDYDEAIEYYQKALELDPNNAEAKQNL |
| | NNAEAKQNLGNAKQKQGGGSGSSGSAW | GNAKQKQG (SEQ ID NO: 185)-X3 |
| | SHPQFEKGGGSGGGSGGSAWSHPQFEK | |
| | (SEQ ID NO: 430) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 1na0_3 | DDEREIREIEEEARRILEHLEELARKG | WATEMMLELIKSDDEREIREIEEEARRILEHLEELAR |
| | GGGSGGGGNLAEKMYKAGNAMYRKGQY | K (SEQ ID NO: 101)-X2- |
| | TIAIIAYTLALLKDPNNAEAWYNLGNA | NLAEKMYKAGNAMYRKGQYTIAIIAYTLALLKDPNNA |
| | AYKKGEYDEAIEAYQKALELDPNNAEA | EAWYNLGNAAYKKGEYDEAIEAYQKALELDPNNAEAW |
| | WYNLGNAYYKQGDYDEAIEYYQKALEL | YNLGNAYYKQGDYDEAIEYYQKALELDPNNAEAKQNL |
| | DPNNAEAKQNLGNAKQKQGGGSGSSGS | GNAKQKQG (SEQ ID NO: 185)-X3 |
| | AWSHPQFEKGGGSGGGSGGSAWSHPQF | |
| | EK (SEQ ID NO: 431) | |
| AHB2- | MEKKIELEEQVMHVLDQVSELAHELLH | X1- |
| GGGGS5- | KLTGEELERAAYFNWWATEMMLELIKS | ELEEQVMHVLDQVSELAHELLHKLTGEELERAAYFNW |
| 4pn9 | DDEREIREIEEEARRILEHLEELARKG | WATEMMLELIKSDDEREIREIEEEARRILEHLEELAR |
| | GSGGGEIAKSLKEIAKSLKEIAWSLKE | K (SEQ ID NO: 101)-X2- |
| | IAKSLKGGGSGSSGSAWSHPQFEKGGG | GEIAKSLKEIAKSLKEIAWSLKEIAKSLKG (SEQ ID |
| | SGGGSGGSAWSHPQFEK (SEQ ID | NO: 186)-X3 |
| | NO: 432) | |
| AHB2- | MEKKIELEEQVMHVLDQVSELAHELLH | X1- |
| GGGGS7- | KLTGEELERAAYFNWWATEMMLELIKS | ELEEQVMHVLDQVSELAHELLHKLTGEELERAAYFNW |
| 4pn9 | DDEREIREIEEEARRILEHLEELARKG | WATEMMLELIKSDDEREIREIEEEARRILEHLEELAR |
| | GGSGGGGEIAKSLKEIAKSLKEIAWSL | K (SEQ ID NO: 101)-X2- |
| | KEIAKSLKGGGSGSSGSAWSHPQFEKG | GEIAKSLKEIAKSLKEIAWSLKEIAKSLKG (SEQ |
| | GGSGGGSGGSAWSHPQFEK (SEQ ID | ID NO: 186)-X3 |
| | NO: 433) | |
| AHB2- | MEKKIELEEQVMHVLDQVSELAHELLH | X1- |
| GGGGS9- | KLTGEELERAAYFNWWATEMMLELIKS | ELEEQVMHVLDQVSELAHELLHKLTGEELERAAYFNW |
| 4pn9 | DDEREIREIEEEARRILEHLEELARKG | WATEMMLELIKSDDEREIREIEEEARRILEHLEELAR |
| | GGGSGGGGGEIAKSLKEIAKSLKEIAW | K (SEQ ID NO: 101)-X2- |
| | SLKEIAKSLKGGGSGSSGSAWSHPQFE | GEIAKSLKEIAKSLKEIAWSLKEIAKSLKG (SEQ ID |
| | KGGGSGGGSGGSAWSHPQFEK (SEQ | NO: 186)-X3 |
| | ID NO: 434) | |
| AHB2- | MEKKIELEEQVMHVLDQVSELAHELLH | X1- |
| 2GS- | KLTGEELERAAYFNWWATEMMLELIKS | ELEEQVMHVLDQVSELAHELLHKLTGEELERAAYFNW |
| SB175 | DDEREIREIEEEARRILEHLEELARKG | WATEMMLELIKSDDEREIREIEEEARRILEHLEELAR |
| | GSEALEELEKALRELKKSTDELERSTE | K (SEQ ID NO: 101)-X2- |
| | ELEKNPSEDALVENNRLIVENNKIIVE | SEALEELEKALRELKKSTDELERSTEELEKNPSEDAL |
| | VLRIIAKVLKGGSGSSGSAWSHPQFEK | VENNRLIVENNKIIVEVLRIIAKVLK (SEQ ID |
| | GGGSGGGSGGSAWSHPQFEK (SEQ ID | NO: 187)-X3 |
| | NO: 435) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| SB175 | DDEREIREIEEEARRILEHLEELARKG | WATEMMLELIKSDDEREIREIEEEARRILEHLEELAR |
| | GGSSEALEELEKALRELKKSTDELERS | K (SEQ ID NO: 101)-X2- |
| | TEELEKNPSEDALVENNRLIVENNKII | SEALEELEKALRELKKSTDELERSTEELEKNPSEDAL |
| | VEVLRIIAKVLKGGSGSSGSAWSHPQF | VENNRLIVENNKIIVEVLRIIAKVLK (SEQ ID |
| | EKGGGSGGGSGGSAWSHPQFEK (SEQ | NO: 187)-X3 |
| | ID NO: 436) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| SB175 | DDEREIREIEEEARRILEHLEELARKG | WATEMMLELIKSDDEREIREIEEEARRILEHLEELAR |
| | GGGSGSEALEELEKALRELKKSTDELE | K (SEQ ID NO: 101)-X2- |
| | RSTEELEKNPSEDALVENNRLIVENNK | SEALEELEKALRELKKSTDELERSTEELEKNPSEDAL |
| | IIVEVLRIIAKVLKGGSGSSGSAWSHP | VENNRLIVENNKIIVEVLRIIAKVLK (SEQ ID |
| | QFEKGGGSGGGSGGSAWSHPQFEK | NO: 187)-X3 |
| | (SEQ ID NO: 437) | |
| AHB2- | MEKKIELEEQVMHVLDQVSELAHELLH | X1- |
| 2GS- | KLTGEELERAAYFNWWATEMMLELIKS | ELEEQVMHVLDQVSELAHELLHKLTGEELERAAYFNW |
| SB175.1 | DDEREIREIEEEARRILEHLEELARKG | WATEMMLELIKSDDEREIREIEEEARRILEHLEELAR |
| | GSPELEKALRELKKSTDELERSTEELE | K (SEQ ID NO: 101)-X2- |
| | KNGSPEALVENNRLIVENNKIIVEVLR | SPELEKALRELKKSTDELERSTEELEKNGSPEALVEN |
| | IIAKGGSGSSGSAWSHPQFEKGGGSGG | NRLIVENNKIIVEVLRIIAK (SEQ ID NO: 188)- |
| | GSGGSAWSHPQFEK (SEQ ID NO: | X3 |
| | 438) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| SB175.1 | DDEREIREIEEEARRILEHLEELARKG | WATEMMLELIKSDDEREIREIEEEARRILEHLEELAR |
| | GGSSPELEKALRELKKSTDELERSTEE | K (SEQ ID NO: 101)-X2- |
| | LEKNGSPEALVENNRLIVENNKIIVEV | SPELEKALRELKKSTDELERSTEELEKNGSPEALVEN |
| | LRIIAKGGSGSSGSAWSHPQFEKGGGS | NRLIVENNKIIVEVLRIIAK (SEQ ID NO: 188)- |
| | GGGSGGSAWSHPQFEK (SEQ ID NO: | X3 |
| | 439) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| SB175.1 | DDEREIREIEEEARRILEHLEELARKG | WATEMMLELIKSDDEREIREIEEEARRILEHLEELAR |
| | GGGSGSPELEKALRELKKSTDELERST | K (SEQ ID NO: 101)-X2- |
| | EELEKNGSPEALVENNRLIVENNKIIV | SPELEKALRELKKSTDELERSTEELEKNGSPEALVEN |
| | EVLRIIAKGGSGSSGSAWSHPQFEKGG | NRLIVENNKIIVEVLRIIAK (SEQ ID NO: 188)- |
| | GSGGGSGGSAWSHPQFEK (SEQ ID | X3 |
| | NO: 440) | |
| AHB2- | MEKKIELEEQVMHVLDQVSELAHELLH | X1- |
| 2GS- | KLTGEELERAAYFNWWATEMMLELIKS | ELEEQVMHVLDQVSELAHELLHKLTGEELERAAYFNW |
| SB175.2 | DDEREIREIEEEARRILEHLEELARKG | WATEMMLELIKSDDEREIREIEEEARRILEHLEELAR |
| | GSEKALRELKKSTDELERSTEELEKNG | K (SEQ ID NO: 101)-X2- |
| | SPEALVENNRLIVENNKIIVEVLRGGS | SEKALRELKKSTDELERSTEELEKNGSPEALVENNRL |
| | GSSGSAWSHPQFEKGGGSGGGSGGSAW | IVENNKIIVEVLR (SEQ ID NO: 189)-X3 |
| | SHPQFEK (SEQ ID NO: 441) | |
| AHB2- | MEKKIELEEQVMHVLDQVSELAHELLH | X1- |
| 4GS- | KLTGEELERAAYFNWWATEMMLELIKS | ELEEQVMHVLDQVSELAHELLHKLTGEELERAAYFNW |
| SB175.2 | DDEREIREIEEEARRILEHLEELARKG | WATEMMLELIKSDDEREIREIEEEARRILEHLEELAR |
| | GGSSEKALRELKKSTDELERSTEELEK | K (SEQ ID NO: 101)-X2- |
| | NGSPEALVENNRLIVENNKIIVEVLRG | SEKALRELKKSTDELERSTEELEKNGSPEALVENNRL |
| | GSGSSGSAWSHPQFEKGGGSGGGSGGS | IVENNKIIVEVLR (SEQ ID NO: 189)-X3 |
| | AWSHPQFEK (SEQ ID NO: 442) | |
| AHB2- | MEKKIELEEQVMHVLDQVSELAHELLH | X1- |
| 6GS- | KLTGEELERAAYFNWWATEMMLELIKS | ELEEQVMHVLDQVSELAHELLHKLTGEELERAAYFNW |
| SB175.2 | DDEREIREIEEEARRILEHLEELARKG | WATEMMLELIKSDDEREIREIEEEARRILEHLEELAR |
| | GGGSGSEKALRELKKSTDELERSTEEL | K-X2- |
| | EKNGSPEALVENNRLIVENNKIIVEVL | SEKALRELKKSTDELERSTEELEKNGSPEALVENNRL |
| | RGGSGSSGSAWSHPQFEKGGGSGGGSG | IVENNKIIVEVLR (SEQ ID NO: 189)-X3 |
| | GSAWSHPQFEK (SEQ ID NO: 443) | |
| SB175- | MEKKISAWSHPQFEKGGGSGGGSGGSA | X1- |
| 6GS- | WSHPQFEKGGSGSSGSEALEELEKALR | SEALEELEKALRELKKSTDELERSTEELEKNPSEDAL |
| LCB1v2.2 | ELKKSTDELERSTEELEKNPSEDALVE | VENNRLIVENNKIIVEVLRIIAKVLK (SEQ ID |
| | NNRLIVENNKIIVEVLRIIAKVLKGGG | NO: 187)-X2- |
| | GSGDKENVLQKIYEIMKELERLGHAEA | DKENVLQKIYEIMKELERLGHAEASMQVSDLIYEFMK |
| | SMQVSDLIYEFMKTKDENLLEEAERLL | TKDENLLEEAERLLEEVKR (SEQ ID NO: 135) |
| | EEVKR (SEQ ID NO: 444) | |
| SB175- | MEKKISAWSHPQFEKGGGSGGGSGGSA | X1- |
| 10GS- | WSHPQFEKGGSGSSGSEALEELEKALR | SEALEELEKALRELKKSTDELERSTEELEKNPSEDAL |
| LCB1v2.2 | ELKKSTDELERSTEELEKNPSEDALVE | VENNRLIVENNKIIVEVLRIIAKVLK (SEQ ID |
| | NNRLIVENNKIIVEVLRIIAKVLKGGG | NO: 187)-X2- |
| | GSGGGGSDKENVLQKIYEIMKELERLG | DKENVLQKIYEIMKELERLGHAEASMQVSDLIYEFMK |
| | HAEASMQVSDLIYEFMKTKDENLLEEA | TKDENLLEEAERLLEEVKR (SEQ ID NO: 135) |
| | ERLLEEVKR (SEQ ID NO: 445) | |
| SB175.1- | MEKKISAWSHPQFEKGGGSGGGSGGSA | X1- |
| 6GS- | WSHPQFEKGGSGSSGSPELEKALRELK | SPELEKALRELKKSTDELERSTEELEKNGSPEALVEN |
| LCB1v2.2 | KSTDELERSTEELEKNGSPEALVENNR | NRLIVENNKIIVEVLRIIAK (SEQ ID NO: 188)- |
| | LIVENNKIIVEVLRIIAKGGGGSGDKE | X2- |
| | NVLQKIYEIMKELERLGHAEASMQVSD | DKENVLQKIYEIMKELERLGHAEASMQVSDLIYEFMK |
| | LIYEFMKTKDENLLEEAERLLEEVKR | TKDENLLEEAERLLEEVKR (SEQ ID NO: 135) |
| | (SEQ ID NO: 446) | |
| SB175.1- | MEKKISAWSHPQFEKGGGSGGGSGGSA | X1- |
| 10GS- | WSHPQFEKGGSGSSGSPELEKALRELK | SPELEKALRELKKSTDELERSTEELEKNGSPEALVEN |
| LCB1v2.2 | KSTDELERSTEELEKNGSPEALVENNR | NRLIVENNKIIVEVLRIIAK (SEQ ID NO: 188)- |
| | LIVENNKIIVEVLRIIAKGGGGSGGGG | X2 |
| | SDKENVLQKIYEIMKELERLGHAEASM | DKENVLQKIYEIMKELERLGHAEASMQVSDLIYEFMK |
| | QVSDLIYEFMKTKDENLLEEAERLLEE | TKDENLLEEAERLLEEVKR (SEQ ID NO: 135) |
| | VKR (SEQ ID NO: 447) | |
| LCB3- | MEKKINLDELHMQMTDLVYEALHFAKD | X1- |
| 8GS- | EEFQKHVFQLFEKATKAYKNKDRQKLE | NLDELHMQMTDLVYEALHFAKDEEFQKHVFQLFEKAT |
| SB175 | KVVEELKELLERLLSGGGGSGGGSEAL | KAYKNKDROKLEKVVEELKELLERLLS (SEQ ID |
| | EELEKALRELKKSTDELERSTEELEKN | NO: 155)-X2- |
| | PSEDALVENNRLIVENNKIIVEVLRII | SEALEELEKALRELKKSTDELERSTEELEKNPSEDAL |
| | AKVLKGGASPAAPAGGSAWSHPQFEKG | VENNRLIVENNKIIVEVLRIIAKVLK (SEQ ID |
| | GGSGGGSGGSAWSHPQFEK (SEQ ID | NO: 187)-X3 |
| | NO: 448) | |
| LCB3- | MEKKINLDELHMQMTDLVYEALHFAKD | X1- |
| 6GS- | EEFQKHVFQLFEKATKAYKNKDRQKLE | NLDELHMQMTDLVYEALHFAKDEEFQKHVFQLFEKAT |
| SB175 | KVVEELKELLERLLSGGGGSGSEALEE | KAYKNKDROKLEKVVEELKELLERLLS (SEQ ID |
| | LEKALRELKKSTDELERSTEELEKNPS | NO: 155)-X2- |
| | EDALVENNRLIVENNKIIVEVLRILAK | SEALEELEKALRELKKSTDELERSTEELEKNPSEDAL |
| | VLKGGASPAAPAGGSAWSHPQFEKGGG | VENNRLIVENNKIIVEVLRIIAKVLK (SEQ ID |
| | SGGGSGGSAWSHPQFEK (SEQ ID | NO: 187)-X3 |
| | NO: 449) | |
| LCB3- | MEKKINLDELHMQMTDLVYEALHFAKD | X1- |
| 4GS- | EEFQKHVFQLFEKATKAYKNKDRQKLE | NLDELHMQMTDLVYEALHFAKDEEFQKHVFQLFEKAT |
| SB175 | KVVEELKELLERLLSGGGSSEALEELE | KAYKNKDRQKLEKVVEELKELLERLLS (SEQ ID |
| | KALRELKKSTDELERSTEELEKNPSED | NO: 155)-X2- |
| | ALVENNRLIVENNKIIVEVLRIIAKVL | SEALEELEKALRELKKSTDELERSTEELEKNPSEDAL |
| | KGGASPAAPAGGSAWSHPQFEKGGGSG | VENNRLIVENNKIIVEVLRIIAKVLK (SEQ ID |
| | GGSGGSAWSHPQFEK (SEQ ID NO: | NO: 187)-X3 |
| | 450) | |
| LCB3- | MEKKINLDELHMQMTDLVYEALHFAKD | X1- |
| 2GS- | EEFQKHVFQLFEKATKAYKNKDRQKLE | NLDELHMQMTDLVYEALHFAKDEEFQKHVFQLFEKAT |
| SB175 | KVVEELKELLERLLSGGSEALEELEKA | KAYKNKDROKLEKVVEELKELLERLLS (SEQ ID |
| | LRELKKSTDELERSTEELEKNPSEDAL | NO: 155)-X2- |
| | VENNRLIVENNKIIVEVLRIIAKVLKG | SEALEELEKALRELKKSTDELERSTEELEKNPSEDAL |
| | GASPAAPAGGSAWSHPQFEKGGGSGGG | VENNRLIVENNKIIVEVLRIIAKVLK (SEQ |
| | SGGSAWSHPQFEK (SEQ ID NO: | ID NO: 187)-X3 |
| | 451) | |
| AHB2v1- | MEKKIELEEQVMHVLDQVSELAHELLH | X1- |
| 2GS- | KLTGEELERAAYFNWWATEMMLELIKS | ELEEQVMHVLDQVSELAHELLHKLTGEELERAAYFNW |
| SB175 | DDEREIREIEEEARRILEHLEELARKG | WATEMMLELIKSDDEREIREIEEEARRILEHLEELAR |
| | GSEALEELEKALRELKKSTDELERSTE | K (SEQ ID NO: 101)-X2- |
| | ELEKNPSEDALVENNRLIVENNKIIVE | SEALEELEKALRELKKSTDELERSTEELEKNPSEDAL |
| | VLRIIAKVLKGGSGSSGSAWSHPQFEK | VENNRLIVENNKIIVEVLRIIAKVLK (SEQ ID |
| | GGGSGGGSGGSAWSHPQFEK (SEQ ID | NO: 187)-X3 |
| | NO: 452) | |
| AHB2v2- | MEKKIELEEQVMHVLDQVSELAHELLH | X1- |
| 2GS- | KLTGEELERAAYFNWWATEMMLELIKS | ELEEQVMHVLDQVSELAHELLHKLTGEELERAAYFNW |
| SB175 | DDEREIREIEEEAARILEHLEELARTG | WATEMMLELIKSDDEREIREIEEEAARILEHLEELAR |
| | GSEALEELEKALRELKKSTDELERSTE | T (SEQ ID NO: 164)-X2- |
| | ELEKNPSEDALVENNRLIVENNKIIVE | SEALEELEKALRELKKSTDELERSTEELEKNPSEDAL |
| | VLRIIAKVLKGGSGSSGSAWSHPQFEK | VENNRLIVENNKIIVEVLRIIAKVLK (SEQ ID |
| | GGGSGGGSGGSAWSHPQFEK (SEQ ID | NO: 187)-X3 |
| | NO: 453) | |

TABLE 9A

| SEQ | | |
|---|---|---|
| ID | Name | Sequence |
| 595 | AHB2-6GS- | ELEEQVMHVLDQVSELAHELLHKLTGEELERAAYFNWWATEMMLELIKSDDEREIRE |
| | 1rfo | IEEEARRILEHLEELARKGSGSGSGYIPEAPRDGQAYVRKDGEWVLLSTFL |
| 596 | AHB2-28GS- | ELEEQVMHVLDQVSELAHELLHKLTGEELERAAYFNWWATEMMLELIKSDDEREIRE |
| | AHB2-6GS- | IEEEARRILEHLEELARKGSGSGSGSGSGSGSGSGSGSGSGSGSGSELEEQVMHVLD |
| | 1rfo | QVSELAHELLHKLTGEELERAAYFNWWATEMMLELIKSDDEREIREIEEEARRILEH |
| | | LEELARKGSGSGSGYIPEAPRDGQAYVRKDGEWVLLSTFL |
| 597 | AHB2-5GS- | ELEEQVMHVLDQVSELAHELLHKLTGEELERAAYFNWWATEMMLELIKSDDEREIRE |
| | 1rfo | IEEEARRILEHLEELARKGSGSGGYIPEAPRDGQAYVRKDGEWVLLSTFL |
| 598 | AHB2-4GS- | ELEEQVMHVLDQVSELAHELLHKLTGEELERAAYFNWWATEMMLELIKSDDEREIRE |
| | 1rfo | IEEEARRILEHLEELARKGSGSGYIPEAPRDGQAYVRKDGEWVLLSTFL |
| 599 | AHB2-3GS- | ELEEQVMHVLDQVSELAHELLHKLTGEELERAAYFNWWATEMMLELIKSDDEREIRE |
| | 1rfo | IEEEARRILEHLEELARKGSGGYIPEAPRDGQAYVRKDGEWVLLSTFL |
| 600 | AHB2-28GS- | ELEEQVMHVLDQVSELAHELLHKLTGEELERAAYFNWWATEMMLELIKSDDEREIRE |
| | AHB2-3GS- | IEEEARRILEHLEELARKGSGSGSGSGSGSGSGSGSGSGSGSGSGSELEEQVMHVLD |
| | 1rfo | QVSELAHELLHKLTGEELERAAYFNWWATEMMLELIKSDDEREIREIEEEARRILEH |
| | | LEELARKGSGGYIPEAPRDGQAYVRKDGEWVLLSTFL |
| 601 | AHB2-4GS- | ELEEQVMHVLDQVSELAHELLHKLTGEELERAAYFNWWATEMMLELIKSDDEREIRE |
| | 5L6HC3_1 | IEEEARRILEHLEELARKGSGSSEELRAVADLQRLNIELARKLLEAVARLQELNIDL |
| | | VRKTSELTDEKTIREEIRKVKEESKRIVEEAEEEIRRAKEESRKIADESR |
| 602 | AHB2-5GS- | ELEEQVMHVLDQVSELAHELLHKLTGEELERAAYFNWWATEMMLELIKSDDEREIRE |
| | 5L6HC3_1 | IEEEARRILEHLEELARKGSGSGSEELRAVADLQRLNIELARKLLEAVARLQELNID |
| | | LVRKTSELTDEKTIREEIRKVKEESKRIVEEAEEEIRRAKEESRKIADESR |
| 603 | AHB2-28GS- | ELEEQVMHVLDQVSELAHELLHKLTGEELERAAYFNWWATEMMLELIKSDDEREIRE |
| | AHB2-4GS- | IEEEARRILEHLEELARKGSGSGSGSGSGSGSGSGSGSGSGSGSGSELEEQVMHVLD |
| | 5L6HC3_1 | QVSELAHELLHKLTGEELERAAYFNWWATEMMLELIKSDDEREIREIEEEARRILEH |
| | | LEELARKGSGSSEELRAVADLQRLNIELARKLLEAVARLQELNIDLVRKTSELTDEK |
| | | TIREEIRKVKEESKRIVEEAEEEIRRAKEESRKIADESR |
| 604 | AHB2-28GS- | ELEEQVMHVLDQVSELAHELLHKLTGEELERAAYFNWWATEMMLELIKSDDEREIRE |
| | AHB2-5GS- | IEEEARRILEHLEELARKGSGSGSGSGSGSGSGSGSGSGSGSGSGSELEEQVMHVLD |
| | 5L6HC3_1 | QVSELAHELLHKLTGEELERAAYFNWWATEMMLELIKSDDEREIREIEEEARRILEH |
| | | LEELARKGSGSGSEELRAVADLQRLNIELARKLLEAVARLQELNIDLVRKTSELTDE |
| | | KTIREEIRKVKEESKRIVEEAEEEIRRAKEESRKIADESR |
| 605 | LCB3-6GS- | NLDELHMQMTDLVYEALHFAKDEEFQKHVFQLFEKATKAYKNKDRQKLEKVVEELKE |
| | 1rfo | LLERLLSGSGSGSGYIPEAPRDGQAYVRKDGEWVLLSTFL |
| 606 | LCB3-5GS- | NLDELHMQMTDLVYEALHFAKDEEFQKHVFQLFEKATKAYKNKDRQKLEKVVEELKE |
| | 1rfo | LLERLLSGSGSGGYIPEAPRDGQAYVRKDGEWVLLSTFL |
| 607 | LCB3-4GS- | NLDELHMQMTDLVYEALHFAKDEEFQKHVFQLFEKATKAYKNKDRQKLEKVVEELKE |
| | 1rfo | LLERLLSGSGSGYIPEAPRDGQAYVRKDGEWVLLSTFL |
| 608 | LCB3-5GS- | NLDELHMQMTDLVYEALHFAKDEEFQKHVFQLFEKATKAYKNKDRQKLEKVVEELKE |
| | 5L6HC3_1 | LLERLLSGSGSGSEELRAVADLQRLNIELARKLLEAVARLQELNIDLVRKTSELTDE |
| | | KTIREEIRKVKEESKRIVEEAEEEIRRAKEESRKIADESR |
| 609 | LCB3-28GS- | NLDELHMQMTDLVYEALHFAKDEEFQKHVFQLFEKATKAYKNKDRQKLEKVVEELKE |
| | LCB3-4GS- | LLERLLSGSGSGSGSGSGSGSGSGSGSGSGSGSGSNLDELHMQMTDLVYEALHFAKD |
| | 5L6HC3_1 | EEFQKHVFQLFEKATKAYKNKDRQKLEKVVEELKELLERLLSGSGSSEELRAVADLQ |
| | | RLNIELARKLLEAVARLQELNIDLVRKTSELTDEKTIREEIRKVKEESKRIVEEAEE |
| | | EIRRAKEESRKIADESR |
| 610 | LCB3-28GS- | NLDELHMQMTDLVYEALHFAKDEEFQKHVFQLFEKATKAYKNKDRQKLEKVVEELKE |
| | LCB3-5GS- | LLERLLSGSGSGSGSGSGSGSGSGSGSGSGSGSGSNLDELHMQMTDLVYEALHFAKD |
| | 5L6HC3_1 | EEFQKHVFQLFEKATKAYKNKDRQKLEKVVEELKELLERLLSGSGSGSEELRAVADL |
| | | QRLNIELARKLLEAVARLQELNIDLVRKTSELTDEKTIREEIRKVKEESKRIVEEAE |
| | | EEIRRAKEESRKIADESR |
| 611 | 36729.2_LCB | EEAELAYLLGELAYKLGEYRIAIRAYRIALKRDPNNAEAWYNLGNAYYKQGDYDEAI |
| | 1v2.2_6GS | EYYQKALELDPNNAEAWYNLGNAYYKQGDYDEAIEYYQKALELDPNNAEAWYNLGNA |
| | | YYKQGDYDEAIEYYQKALELGSGSGSDKENVLQKIYEIMKELERLGHAEASMQVSDL |
| | | IYEFMKTKDENLLEEAERLLEEVKR |
| 612 | 36729.2_LCB | EEAELAYLLGELAYKLGEYRIAIRAYRIALKRDPNNAEAWYNLGNAYYKQGDYDEAI |
| | 1v2.2_4GS | EYYQKALELDPNNAEAWYNLGNAYYKQGDYDEAIEYYQKALELDPNNAEAWYNLGNA |
| | | YYKQGDYDEAIEYYQKALELGSGSDKENVLQKIYEIMKELERLGHAEASMQVSDLIY |
| | | EFMKTKDENLLEEAERLLEEVKR |
| 613 | 36729.2_LCB | EEAELAYLLGELAYKLGEYRIAIRAYRIALKRDPNNAEAWYNLGNAYYKQGDYDEAI |
| | 1v2.2_2GS | EYYQKALELDPNNAEAWYNLGNAYYKQGDYDEAIEYYQKALELDPNNAEAWYNLGNA |
| | | YYKQGDYDEAIEYYQKALELGSDKENVLQKIYEIMKELERLGHAEASMQVSDLIYEF |
| | | MKTKDENLLEEAERLLEEVKR |
| 614 | LCB3-PAS24- | NLDELHMQMTDLVYEALHFAKDEEFQKHVFQLFEKATKAYKNKDRQKLEKVVEELKE |
| | LCB3-4GS- | LLERLLSGGASPAAPAPASPAAPAPSAPAGGNLDELHMQMTDLVYEALHFAKDEEFQ |
| | 5L6HC3_1 | KHVFQLFEKATKAYKNKDRQKLEKVVEELKELLERLLSGSGSSEELRAVADLQRLNI |
| | | ELARKLLEAVARLQELNIDLVRKTSELTDEKTIREEIRKVKEESKRIVEEAEEEIRR |
| | | AKEESRKIADESR |
| 615 | LCB3-PAS24- | NLDELHMQMTDLVYEALHFAKDEEFQKHVFQLFEKATKAYKNKDRQKLEKVVEELKE |
| | LCB3-5GS- | LLERLLSGGASPAAPAPASPAAPAPSAPAGGNLDELHMQMTDLVYEALHFAKDEEFQ |
| | 5L6HC3_1 | KHVFQLFEKATKAYKNKDRQKLEKVVEELKELLERLLSGSGSGSEELRAVADLQRLN |
| | | IELARKLLEAVARLQELNIDLVRKTSELTDEKTIREEIRKVKEESKRIVEEAEEEIR |
| | | RAKEESRKIADESR |
| 616 | LCB1-10GS- | DKENVLQKIYEIMKELERLGHAEASMQVSDLIYEFMKTKDENLLEEAERLLEEVKRG |
| | AHB2-4GS- | GGSGGGSGGELEEQVMHVLDQVSELAHELLHKLTGEELERAAYFNWWATEMMLELIK |
| | 1rfo | SDDEREIREIEEEARRILEHLEELARKGSGSGYIPEAPRDGQAYVRKDGEWVLLSTF |
| | | L |
| 617 | LCB1-28GS- | DKENVLQKIYEIMKELERLGHAEASMQVSDLIYEFMKTKDENLLEEAERLLEEVKRG |
| | AHB2-4GS- | SGSGSGSGSGSGSGSGSGSGSGSGSGSELEEQVMHVLDQVSELAHELLHKLTGEELE |
| | 1rfo | RAAYFNWWATEMMLELIKSDDEREIREIEEEARRILEHLEELARKGSGSGYIPEAPR |
| | | DGQAYVRKDGEWVLLSTFL |
| 618 | LCB3-10GS- | NLDELHMQMTDLVYEALHFAKDEEFQKHVFQLFEKATKAYKNKDRQKLEKVVEELKE |
| | AHB2-4GS- | LLERLLSGGGSGGGSGGELEEQVMHVLDQVSELAHELLHKLTGEELERAAYFNWWAT |
| | 1rfo | EMMLELIKSDDEREIREIEEEARRILEHLEELARKGSGSGYIPEAPRDGQAYVRKDG |
| | | EWVLLSTFL |
| 619 | LCB3-16GS- | MLDELHMQMTDLVYEALHFAKDEEFQKHVFQLFEKATKAYKNKDRQKLEKVVEELKE |
| | AHB2-4GS- | LLERLLSGSGSGSGSGSGSGSGSELEEQVMHVLDQVSELAHELLHKLTGEELERAAY |
| | 1rfo | FNWWATEMMLELIKSDDEREIREIEEEARRILEHLEELARKGSGSGYIPEAPRDGQA |
| | | YVRKDGEWVLLSTFL |
| 620 | 36729.2_6GS_ | EEAELAYLLGELAYKLGEYRIAIRAYRIALKRDPNNAEAWYNLGNAYYKQGDYDEAI |
| | LCB1v2.2- | EYYQKALELDPNNAEAWYNLGNAYYKQGDYDEAIEYYQKALELDPNNAEAWYNLGNA |
| | 28GS-LCB3 | YYKQGDYDEAIEYYQKALELGSGSGSDKENVLQKIYEIMKELERLGHAEASMQVSDL |
| | | IYEFMKTKDENLLEEAERLLEEVKRGSGSGSGSGSGSGSGSGSGSGSGSGSGSNLDE |
| | | LHMQMTDLVYEALHFAKDEEFQKHVFQLFEKATKAYKNKDRQKLEKVVEELKELLER |
| | | LLS |
| 621 | 36729.2_6GS_ | EEAELAYLLGELAYKLGEYRIAIRAYRIALKRDPNNAEAWYNLGNAYYKQGDYDEAI |
| | LCB1v2.2- | EYYQKALELDPNNAEAWYNLGNAYYKQGDYDEAIEYYQKALELDPNNAEAWYNLGNA |
| | 9GS-LCB3 | YYKQGDYDEAIEYYQKALELGSGSGSDKENVLQKIYEIMKELERLGHAEASMQVSDL |
| | | IYEFMKTKDENLLEEAERLLEEVKRGSGSGSGSGNLDELHMQMTDLVYEALHFAKDE |
| | | EFQKHVFQLFEKATKAYKNKDRQKLEKVVEELKELLERLLS |
| 622 | 36729.2_6GS_ | EEAELAYLLGELAYKLGEYRIAIRAYRIALKRDPNNAEAWYNLGNAYYKQGDYDEAI |
| | LCB1v2.2- | EYYQKALELDPNNAEAWYNLGNAYYKQGDYDEAIEYYQKALELDPNNAEAWYNLGNA |
| | 28GS-AHB2 | YYKQGDYDEAIEYYQKALELGSGSGSDKENVLQKIYEIMKELERLGHAEASMQVSDL |
| | | IYEFMKTKDENLLEEAERLLEEVKRGSGSGSGSGSGSGSGSGSGSGSGSGSGSELEE |
| | | QVMHVLDQVSELAHELLHKLTGEELERAAYFNWWATEMMLELIKSDDEREIREIEEE |
| | | ARRILEHLEELARK |
| 623 | 36729.2_6GS_ | EEAELAYLLGELAYKLGEYRIAIRAYRIALKRDPNNAEAWYNLGNAYYKQGDYDEAI |
| | LCB1v2.2- | EYYQKALELDPNNAEAWYNLGNAYYKQGDYDEAIEYYQKALELDPNNAEAWYNLGNA |
| | 9GS-AHB2 | YYKQGDYDEAIEYYQKALELGSGSGSDKENVLQKIYEIMKELERLGHAEASMQVSDL |
| | | IYEFMKTKDENLLEEAERLLEEVKRGSGSGSGSGELEEQVMHVLDQVSELAHELLHK |
| | | LTGEELERAAYFNWWATEMMLELIKSDDEREIREIEEEARRILEHLEELARK |
| 624 | LCB1-10GS- | DKENVLQKIYEIMKELERLGHAEASMQVSDLIYEFMKTKDENLLEEAERLLEEVKRG |
| | LCB3-4GS- | GGSGGGSGGNLDELHMQMTDLVYEALHFAKDEEFQKHVFQLFEKATKAYKNKDRQKL |
| | 1rfo | EKVVEELKELLERLLSGSGSGYIPEAPRDGQAYVRKDGEWVLLSTFL |
| 625 | LCB1-28GS- | DKENVLQKIYEIMKELERLGHAEASMQVSDLIYEFMKTKDENLLEEAERLLEEVKRG |
| | LCB3-4GS- | SGSGSGSGSGSGSGSGSGSGSGSGSGSNLDELHMQMTDLVYEALHFAKDEEFQKHVF |
| | 1rfo | QLFEKATKAYKNKDRQKLEKVVEELKELLERLLSGSGSGYIPEAPRDGQAYVRKDGE |
| | | WVLLSTFL |
| 626 | AHB2-10GS- | ELEEQVMHVLDQVSELAHELLHKLTGEELERAAYFNWWATEMMLELIKSDDEREIRE |
| | LCB3-4GS- | IEEEARRILEHLEELARKGGGSGGGSGGNLDELHMQMTDLVYEALHFAKDEEFQKHV |
| | 1rfo | FQLFEKATKAYKNKDRQKLEKVVEELKELLERLLSGSGSGYIPEAPRDGQAYVRKDG |
| | | EWVLLSTFL |
| 627 | AHB2-16GS- | ELEEQVMHVLDQVSELAHELLHKLTGEELERAAYFNWWATEMMLELIKSDDEREIRE |
| | LCB3-4GS- | IEEEARRILEHLEELARKGSGSGSGSGSGSGSGSNLDELHMQMTDLVYEALHFAKDE |
| | 1rfo | EFQKHVFQLFEKATKAYKNKDRQKLEKVVEELKELLERLLSGSGSGYIPEAPRDGQA |
| | | YVRKDGEWVLLSTFL |
| 628 | LCB1-9GS- | DKENVLQKIYEIMKELERLGHAEASMQVSDLIYEFMKTKDENLLEEAERLLEEVKRG |
| | LCB3-5GS- | SGSGSGSGNLDELHMQMTDLVYEALHFAKDEEFQKHVFQLFEKATKAYKNKDRQKLE |
| | 5L6HC3_1 | KVVEELKELLERLLSGSGSGSEELRAVADLQRLNIELARKLLEAVARLQELNIDLVR |
| | | KTSELTDEKTIREEIRKVKEESKRIVEEAEEEIRRAKEESRKIADESR |
| 629 | AHB2-9GS- | ELEEQVMHVLDQVSELAHELLHKLTGEELERAAYFNWWATEMMLELIKSDDEREIRE |
| | LCB3-5GS- | IEEEARRILEHLEELARKGSGSGSGSGNLDELHMQMTDLVYEALHFAKDEEFQKHVF |
| | 5L6HC3_1 | QLFEKATKAYKNKDRQKLEKVVEELKELLERLLSGSGSGSEELRAVADLQRLNIELA |
| | | RKLLEAVARLQELNIDLVRKTSELTDEKTIREEIRKVKEESKRIVEEAEEEIRRAKE |
| | | ESRKIADESR |
| 630 | AHB2-4GS- | ELEEQVMHVLDQVSELAHELLHKLTGEELERAAYFNWWATEMMLELIKSDDEREIRE |
| | 1na0_int2- | IEEEARRILEHLEELARKGSGSEEAELAYLLGELAYKLGEYRIAIRAYRIALKRDPN |
| | R3 | NAEAWYNLGNAYYKQGDYDEAIYYQKALELDPNNAEAKQNLGNAKQKQ |
| 631 | AHB2-6GS- | ELEEQVMHVLDQVSELAHELLHKLTGEELERAAYFNWWATEMMLELIKSDDEREIRE |
| | 1na0_int2- | IEEEARRILEHLEELARKGSGSGSEEAELAYLLGELAYKLGEYRIAIRAYRIALKRD |
| | R3 | PNNAEAWYNLGNAYYKQGDYDEIEYYQKALELDPNNAEAKQNLGNAKQKQ |
| 632 | AHB2-4GS- | ELEEQVMHVLDQVSELAHELLHKLTGEELERAAYFNWWATEMMLELIKSDDEREIRE |
| | 1na0_int2 | IEEEARRILEHLEELARKGSGSEEAELAYLLGELAYKLGEYRIAIRAYRIALKRDPN |
| | | NAEAWYNLGNAYYKQGDYDEAIYYQKALELDPNNAEAWYNLGNAYYKQGDYDEAIEY |
| | | YQKALELDPNNAEAKQNLGNKQKQ |
| 633 | AHB2-6GS- | ELEEQVMHVLDQVSELAHELLHKLTGEELERAAYFNWWATEMMLELIKSDDEREIRE |
| | 1na0_int2 | IEEEARRILEHLEELARKGSGSGSEEAELAYLLGELAYKLGEYRIAIRAYRIALKRD |
| | | PNNAEAWYNLGNAYYKQGDYDEAIYYQKALELDPNNAEAWYNLGNAYYKQGDYDEAI |
| | | EYYQKALELDPNNAEAKQNLGNKQKQ |
| 634 | AHB2-4GS- | ELEEQVMHVLDQVSELAHELLHKLTGEELERAAYFNWWATEMMLELIKSDDEREIRE |
| | 6msr | IEEEARRILEHLEELARKGSGSGSEYEIRKALEELKASTAELKRATASLRASTEELK |
| | | KNPSEDALVENNRLIVEHNAIIVENRIIAAVLELIVRAIK |
| 635 | AHB2-6GS- | ELEEQVMHVLDQVSELAHELLHKLTGEELERAAYFNWWATEMMLELIKSDDEREIRE |
| | 6msr | IEEEARRILEHLEELARKGSGSGSGSEYEIRKALEELKASTAELKRATASLRASTEE |
| | | LKKNPSEDALVENNRLIVEHNAIIVENRIIAAVLELIVRAIK |
| 636 | 36729.2_ | EEAELAYLLGELAYKLGEYRIAIRAYRIALKRDPNNAEAWYNLGNAYYKQGDYDEAI |
| | LCB1v2.2_6GS | EYYQKALELDPNNAEAWYNLGNAYYKQGDYDEAIEYYQKALELDPNNAEAWYNLGNA |
| | | YYKQGDYDEAIEYYQKALELGSGSGSDKENVLQKIYEIMKELERLGHAEASMQVSDL |
| | | IYEFMKTKDENLLEEAERLLEEVKR |
| 637 | 36729.2_ | EEAELAYLLGELAYKLGEYRIAIRAYRIALKRDPNNAEAWYNLGNAYYKQGDYDEAI |
| | LCB1v2.2_4GS | EYYQKALELDPNNAEAWYNLGNAYYKQGDYDEAIEYYQKALELDPNNAEAWYNLGNA |
| | | YYKQGDYDEAIEYYQKALELGSGSDKENVLQKIYEIMKELERLGHAEASMQVSDLIY |
| | | EFMKTKDENLLEEAERLLEEVKR |
| 638 | 36729.2_ | EEAELAYLLGELAYKLGEYRIAIRAYRIALKRDPNNAEAWYNLGNAYYKQGDYDEAI |
| | LCB1v2.2_2GS | EYYQKALELDPNNAEAWYNLGNAYYKQGDYDEAIEYYQKALELDPNNAEAWYNLGNA |
| | | MKTKDENLLEEAERLLEEVKR |
| 639 | AHB2-8GS- | ELEEQVMHVLDQVSELAHELLHKLTGEELERAAYFNWWATEMMLELIKSDDEREIRE |
| | 1na0_int2- | IEEEARRILEHLEELARKGSGSGSGSEEAELAYLLGELAYKLGEYRIAIRAYRIALK |
| | R3 | RDPNNAEAWYNLGNAYYKQGDYDEAIEYYQKALELDPNNAEAKQNLGNAKQKQG |
| 640 | AHB2-8GS- | ELEEQVMHVLDQVSELAHELLHKLTGEELERAAYFNWWATEMMLELIKSDDEREIRE |
| | 1na0_int2 | IEEEARRILEHLEELARKGSGSGSGSEEAELAYLLGELAYKLGEYRIAIRAYRIALK |
| | | RDPNNAEAWYNLGNAYYKQGDYDEAIEYYQKALELDPNNAEAWYNLGNAYYKQGDYD |
| | | EAIEYYQKALELDPNNAEAKQNLGNAKQKQG |
| 641 | AHB2-8GS- | ELEEQVMHVLDQVSELAHELLHKLTGEELERAAYFNWWATEMMLELIKSDDEREIRE |
| | 6msr | IEEEARRILEHLEELARKGSGSGSGSGSEYEIRKALEELKASTAELKRATASLRAST |
| | | EELKKNPSEDALVENNRLIVEHNAIIVENNRIIAAVLELIVRAIK |
| 642 | AHB2-10GS- | ELEEQVMHVLDQVSELAHELLHKLTGEELERAAYFNWWATEMMLELIKSDDEREIRE |
| | 6msr | IEEEARRILEHLEELARKGSGSGSGSGSGSEYEIRKALEELKASTAELKRATASLRA |
| | | STEELKKNPSEDALVENNRLIVEHNAIIVENNRIIAAVLELIVRAIK |
| 643 | AHB2-10GS- | ELEEQVMHVLDQVSELAHELLHKLTGEELERAAYFNWWATEMMLELIKSDDEREIRE |
| | 1na0_int2- | IEEEARRILEHLEELARKGSGSGSGSGSEEAELAYLLGELAYKLGEYRIAIRAYRIA |
| | R3 | LKRDPNNAEAWYNLGNAYYKQGDYDEAIEYYQKALELDPNNAEAKQNLGNAKQKQG |
| 644 | AHB2-10GS- | ELEEQVMHVLDQVSELAHELLHKLTGEELERAAYFNWWATEMMLELIKSDDEREIRE |
| | 1na0_int2 | IEEEARRILEHLEELARKGSGSGSGSGSEEAELAYLLGELAYKLGEYRIAIRAYRIA |
| | | LKRDPNNAEAWYNLGNAYYKQGDYDEAIEYYQKALELDPNNAEAWYNLGNAYYKQGD |
| | | YDEAIEYYQKALELDPNNAEAKQNLGNAKQKQG |
| 645 | AHB2-4GS- | ELEEQVMHVLDQVSELAHELLHKLTGEELERAAYFNWWATEMMLELIKSDDEREIRE |
| | 1gcm | IEEEARRILEHLEELARKGSGSRMKQIEDKIEEILSKIYHIENEIARIKKLIGER |
| 646 | AHB2-8GS- | ELEEQVMHVLDQVSELAHELLHKLTGEELERAAYFNWWATEMMLELIKSDDEREIRE |
| | 1gcm | IEEEARRILEHLEELARKGSGSGSGSRMKQIEDKIEEILSKIYHIENEIARIKKLIG |
| | | ER |
| 647 | AHB2-4GS- | ELEEQVMHVLDQVSELAHELLHKLTGEELERAAYFNWWATEMMLELIKSDDEREIRE |
| | pRO-2-noHis | IEEEARRILEHLEELARKGSGSGSEYEIRKALEELKASTAELKRSTASLRASTEELK |
| | | KNPSEDALVENNRLIVENNAIIVENNRIIAAVLELIVRAIK |
| 648 | AHB2-8GS- | ELEEQVMHVLDQVSELAHELLHKLTGEELERAAYFNWWATEMMLELIKSDDEREIRE |
| | pRO-2-noHis | IEEEARRILEHLEELARKGSGSGSGSGSEYEIRKALEELKASTAELKRSTASLRAST |
| | | EELKKNPSEDALVENNRLIVENNAIIVENNRIIAAVLELIVRAIK |
| 649 | AHB2-6GS- | ELEEQVMHVLDQVSELAHELLHKLTGEELERAAYFNWWATEMMLELIKSDDEREIRE |
| | 1na0_3 | IEEEARRILEHLEELARKGSGSGSNLAEKMYKAGNAMYRKGQYTIAIIAYTLALLKD |
| | | PNNAEAWYNLGNAAYKKGEYDEAIEAYQKALELDPNNAEAWYNLGNAYYKQGDYDEA |
| | | IEYYQKALELDPNNAEAKQNLGNAKQKQG |
| 650 | AHB2-10GS- | ELEEQVMHVLDQVSELAHELLHKLTGEELERAAYFNWWATEMMLELIKSDDEREIRE |
| | 1na0_3 | IEEEARRILEHLEELARKGSGSGSGSGSNLAEKMYKAGNAMYRKGQYTIAIIAYTLA |
| | | LLKDPNNAEAWYNLGNAAYKKGEYDEAIEAYQKALELDPNNAEAWYNLGNAYYKQGD |
| | | YDEAIEYYQKALELDPNNAEAKQNLGNAKQKQG |
| 651 | AHB2-6GS- | ELEEQVMHVLDQVSELAHELLHKLTGEELERAAYFNWWATEMMLELIKSDDEREIRE |
| | 4pn9 | IEEEARRILEHLEELARKGSGSGSGEIAKSLKEIAKSLKEIAWSLKEIAKSLKG |
| 652 | AHB2-10GS- | ELEEQVMHVLDQVSELAHELLHKLTGEELERAAYFNWWATEMMLELIKSDDEREIRE |
| | 4pn9 | IEEEARRILEHLEELARKGSGSGSGSGSGEIAKSLKEIAKSLKEIAWSLKEIAKSLK |
| | | G |
| 653 | AHB2- | ELEEQVMHVLDQVSELAHELLHKLTGEELERAAYFNWWATEMMLELIKSDDEREIRE |
| | GGGGS5- | IEEEARRILEHLEELARKGGSGGEEAELAYLLGELAYKLGEYRIAIRAYRIALKRDP |
| | 1na0_int2- | NNAEAWYNLGNAYYKQGDYDEAIEYYQKALELDPNNAEAKQNLGNAKQKQG |
| | R3 | |
| 654 | AHB2- | ELEEQVMHVLDQVSELAHELLHKLTGEELERAAYFNWWATEMMLELIKSDDEREIRE |
| | GGGGS7- | IEEEARRILEHLEELARKGGGSGGGEEAELAYLLGELAYKLGEYRIAIRAYRIALKR |
| | 1na0_int2- | DPNNAEAWYNLGNAYYKQGDYDEAIEYYQKALELDPNNAEAKQNLGNAKQKQG |
| | R3 | |
| 655 | AHB2- | ELEEQVMHVLDQVSELAHELLHKLTGEELERAAYFNWWATEMMLELIKSDDEREIRE |
| | 1na0_int2- | IEEEARRILEHLEELARKGGGGSGGGGEEAELAYLLGELAYKLGEYRIAIRAYRIAL |
| | R3 | KRDPNNAEAWYNLGNAYYKQGDYDEAIEYYQKALELDPNNAEAKQNLGNAKQKQG |
| 656 | GGGGS9- | IEEEARRILEHLEELARKGGGGSGGGGEEAELAYLLGELAYKLGEYRIAIRAYRIAL |
| | AHB2- | ELEEQVMHVLDQVSELAHELLHKLTGEELERAAYFNWWATEMMLELIKSDDEREIRE |
| | GGGGS5- | IEEEARRILEHLEELARKGGSGGEEAELAYLLGELAYKLGEYRIAIRAYRIALKRDP |
| | 1na0_int2 | NNAEAWYNLGNAYYKQGDYDEAIEYYQKALELDPNNAEAWYNLGNAYYKQGDYDEAI |
| | | EYYQKALELDPNNAEAKQNLGNAKQKQG |
| 657 | AHB2- | ELEEQVMHVLDQVSELAHELLHKLTGEELERAAYFNWWATEMMLELIKSDDEREIRE |
| | GGGGS7- | IEEEARRILEHLEELARKGGGSGGGEEAELAYLLGELAYKLGEYRIAIRAYRIALKR |
| | 1na0_int2 | DPNNAEAWYNLGNAYYKQGDYDEAIEYYQKALELDPNNAEAWYNLGNAYYKQGDYDE |
| | | AIEYYQKALELDPNNAEAKQNLGNAKQKQG |
| 658 | AHB2- | ELEEQVMHVLDQVSELAHELLHKLTGEELERAAYFNWWATEMMLELIKSDDEREIRE |
| | GGGGS9- | IEEEARRILEHLEELARKGGGGSGGGGEEAELAYLLGELAYKLGEYRIAIRAYRIAL |
| | 1na0_int2 | KRDPNNAEAWYNLGNAYYKQGDYDEAIEYYQKALELDPNNAEAWYNLGNAYYKQGDY |
| | | DEAIEYYQKALELDPNNAEAKQNLGNAKQKQG |
| 659 | AHB2- | ELEEQVMHVLDQVSELAHELLHKLTGEELERAAYFNWWATEMMLELIKSDDEREIRE |
| | GGGGS5-6msr | IEEEARRILEHLEELARKGGSGGGSEYEIRKALEELKASTAELKRATASLRASTEEL |
| | | KKNPSEDALVENNRLIVEHNAIIVENNRIIAAVLELIVRAIK |
| 660 | AHB2- | ELEEQVMHVLDQVSELAHELLHKLTGEELERAAYFNWWATEMMLELIKSDDEREIRE |
| | GGGGS7-6msr | IEEEARRILEHLEELARKGGGSGGGGSEYEIRKALEELKASTAELKRATASLRASTE |
| | | ELKKNPSEDALVENNRLIVEHNAIIVENNRIIAAVLELIVRAIK |
| 661 | AHB2- | ELEEQVMHVLDQVSELAHELLHKLTGEELERAAYFNWWATEMMLELIKSDDEREIRE |
| | GGGGS9-6msr | IEEEARRILEHLEELARKGGGGSGGGGGSEYEIRKALEELKASTAELKRATASLRAS |
| | | TEELKKNPSEDALVENNRLIVEHNAIIVENNRIIAAVLELIVRAIK |
| 662 | AHB2- | ELEEQVMHVLDQVSELAHELLHKLTGEELERAAYFNWWATEMMLELIKSDDEREIRE |
| | GGGGS5-1gcm | IEEEARRILEHLEELARKGGSGGRMKQIEDKIEEILSKIYHIENEIARIKKLIGER |
| 663 | AHB2- | ELEEQVMHVLDQVSELAHELLHKLTGEELERAAYFNWWATEMMLELIKSDDEREIRE |
| | GGGGS7-1gcm | IEEEARRILEHLEELARKGGGSGGGRMKQIEDKIEEILSKIYHIENEIARIKKLIGE |
| | | R |
| 664 | AHB2- | ELEEQVMHVLDQVSELAHELLHKLTGEELERAAYFNWWATEMMLELIKSDDEREIRE |
| | GGGGS9-1gcm | IEEEARRILEHLEELARKGGGGSGGGGRMKQIEDKIEEILSKIYHIENEIARIKKLI |
| | | GER |
| 665 | AHB2- | ELEEQVMHVLDQVSELAHELLHKLTGEELERAAYFNWWATEMMLELIKSDDEREIRE |
| | GGGGS5-pRO- | IEEEARRILEHLEELARKGGSGGGSEYEIRKALEELKASTAELKRSTASLRASTEEL |
| | 2-noHis | KKNPSEDALVENNRLIVENNAIIVENNRIIAAVLELIVRAIK |
| 666 | AHB2- | IEEEARRILEHLEELARKGGSGGGSEYEIRKALEELKASTAELKRSTASLRASTEEL |
| | GGGGS7-pRO- | ELEEQVMHVLDQVSELAHELLHKLTGEELERAAYFNWWATEMMLELIKSDDEREIRE |
| | 2-noHis | IEEEARRILEHLEELARKGGGSGGGGSEYEIRKALEELKASTAELKRSTASLRASTE |
| | | ELKKNPSEDALVENNRLIVENNAIIVENNRIIAAVLELIVRAIK |
| 667 | AHB2- | ELEEQVMHVLDQVSELAHELLHKLTGEELERAAYFNWWATEMMLELIKSDDEREIRE |
| | GGGGS9-pRO- | IEEEARRILEHLEELARKGGGGSGGGGGSEYEIRKALEELKASTAELKRSTASLRAS |
| | 2-noHis | TEELKKNPSEDALVENNRLIVENNAIIVENNRIIAAVLELIVRAIK |
| 668 | AHB2- | ELEEQVMHVLDQVSELAHELLHKLTGEELERAAYFNWWATEMMLELIKSDDEREIRE |
| | GGGGS5- | IEEEARRILEHLEELARKGGSGGNLAEKMYKAGNAMYRKGQYTIAIIAYTLALLKDP |
| | 1na0_3 | NNAEAWYNLGNAAYKKGEYDEAIEAYQKALELDPNNAEAWYNLGNAYYKQGDYDEAI |
| | | EYYQKALELDPNNAEAKQNLGNAKQKQG |
| 669 | AHB2- | ELEEQVMHVLDQVSELAHELLHKLTGEELERAAYFNWWATEMMLELIKSDDEREIRE |
| | GGGGS7- | IEEEARRILEHLEELARKGGGSGGGNLAEKMYKAGNAMYRKGQYTIAIIAYTLALLK |
| | 1na0_3 | DPNNAEAWYNLGNAAYKKGEYDEAIEAYQKALELDPNNAEAWYNLGNAYYKQGDYDE |
| | | AIEYYQKALELDPNNAEAKQNLGNAKQKQG |
| 670 | AHB2- | ELEEQVMHVLDQVSELAHELLHKLTGEELERAAYFNWWATEMMLELIKSDDEREIRE |
| | GGGGS9- | IEEEARRILEHLEELARKGGGGSGGGGNLAEKMYKAGNAMYRKGQYTIAIIAYTLAL |
| | 1na0_3 | LKDPNNAEAWYNLGNAAYKKGEYDEAIEAYQKALELDPNNAEAWYNLGNAYYKQGDY |
| | | DEAIEYYQKALELDPNNAEAKQNLGNAKQKQG |
| 671 | AHB2- | ELEEQVMHVLDQVSELAHELLHKLTGEELERAAYFNWWATEMMLELIKSDDEREIRE |
| | GGGGS5-4pn9 | IEEEARRILEHLEELARKGGSGGGEIAKSLKEIAKSLKEIAWSLKEIAKSLKG |
| 672 | AHB2- | ELEEQVMHVLDQVSELAHELLHKLTGEELERAAYFNWWATEMMLELIKSDDEREIRE |
| | GGGGS7-4pn9 | IEEEARRILEHLEELARKGGGSGGGGEIAKSLKEIAKSLKEIAWSLKEIAKSLKG |
| 673 | AHB2- | ELEEQVMHVLDQVSELAHELLHKLTGEELERAAYFNWWATEMMLELIKSDDEREIRE |
| | GGGGS9-4pn9 | IEEEARRILEHLEELARKGGGGSGGGGGEIAKSLKEIAKSLKEIAWSLKEIAKSLKG |
| 674 | AHB2-2GS- | ELEEQVMHVLDQVSELAHELLHKLTGEELERAAYFNWWATEMMLELIKSDDEREIRE |
| | SB175 | IEEEARRILEHLEELARKGGSEALEELEKALRELKKSTDELERSTEELEKNPSEDAL |
| | | VENNRLIVENNKIIVEVLRIIAKVLK |
| 675 | AHB2-4GS- | ELEEQVMHVLDQVSELAHELLHKLTGEELERAAYFNWWATEMMLELIKSDDEREIRE |
| | SB175 | IEEEARRILEHLEELARKGGGSSEALEELEKALRELKKSTDELERSTEELEKNPSED |
| | | ALVENNRLIVENNKIIVEVLRIIAKVLK |
| 676 | AHB2-6GS- | ELEEQVMHVLDQVSELAHELLHKLTGEELERAAYFNWWATEMMLELIKSDDEREIRE |
| | SB175 | IEEEARRILEHLEELARKGGGGSGSEALEELEKALRELKKSTDELERSTEELEKNPS |
| | | EDALVENNRLIVENNKIIVEVLRIIAKVLK |
| 677 | AHB2-2GS- | ELEEQVMHVLDQVSELAHELLHKLTGEELERAAYFNWWATEMMLELIKSDDEREIRE |
| | SB175.1 | IEEEARRILEHLEELARKGGSPELEKALRELKKSTDELERSTEELEKNGSPEALVEN |
| | | NRLIVENNKIIVEVLRIIAK |
| 678 | AHB2-4GS- | ELEEQVMHVLDQVSELAHELLHKLTGEELERAAYFNWWATEMMLELIKSDDEREIRE |
| | SB175.1 | IEEEARRILEHLEELARKGGGSSPELEKALRELKKSTDELERSTEELEKNGSPEALV |
| | | ENNRLIVENNKIIVEVLRILAK |
| 679 | AHB2-6GS- | ELEEQVMHVLDQVSELAHELLHKLTGEELERAAYFNWWATEMMLELIKSDDEREIRE |
| | SB175.1 | IEEEARRILEHLEELARKGGGGSGSPELEKALRELKKSTDELERSTEELEKNGSPEA |
| | | LVENNRLIVENNKIIVEVLRILAK |
| 680 | AHB2-2GS- | ELEEQVMHVLDQVSELAHELLHKLTGEELERAAYFNWWATEMMLELIKSDDEREIRE |
| 41 | SB175.2 | IEEEARRILEHLEELARKGGSEKALRELKKSTDELERSTEELEKNGSPEALVENNRL |
| | | IVENNKIIVEVLR |
| 681 | AHB2-4GS- | ELEEQVMHVLDQVSELAHELLHKLTGEELERAAYFNWWATEMMLELIKSDDEREIRE |
| | SB175.2 | IEEEARRILEHLEELARKGGGSSEKALRELKKSTDELERSTEELEKNGSPEALVENN |
| | | RLIVENNKIIVEVLR |
| 682 | AHB2-6GS- | ELEEQVMHVLDQVSELAHELLHKLTGEELERAAYFNWWATEMMLELIKSDDEREIRE |
| | SB175.2 | IEEEARRILEHLEELARKGGGGSGSEKALRELKKSTDELERSTEELEKNGSPEALVE |
| | | NNRLIVENNKIIVEVLR |
| 683 | SB175-6GS- | SEALEELEKALRELKKSTDELERSTEELEKNPSEDALVENNRLIVENNKIIVEVLRI |
| | LCB1v2.2 | IAKVLKGGGGSGDKENVLQKIYEIMKELERLGHAEASMQVSDLIYEFMKTKDENLLE |
| | | EAERLLEEVKR |
| 684 | SB175-10GS- | SEALEELEKALRELKKSTDELERSTEELEKNPSEDALVENNRLIVENNKIIVEVLRI |
| | LCB1v2.2 | IAKVLKGGGGSGGGGSDKENVLQKIYEIMKELERLGHAEASMQVSDLIYEFMKTKDE |
| | | NLLEEAERLLEEVKR |
| 685 | SB175.1- | SPELEKALRELKKSTDELERSTEELEKNGSPEALVENNRLIVENNKIIVEVLRIIAK |
| | 6GS- | GGGGSGDKENVLQKIYEIMKELERLGHAEASMQVSDLIYEFMKTKDENLLEEAERLL |
| | LCB1v2.2 | EEVKR |
| 686 | SB175.1- | SPELEKALRELKKSTDELERSTEELEKNGSPEALVENNRLIVENNKIIVEVLRIIAK |
| | 10GS- | GGGGSGGGGSDKENVLQKIYEIMKELERLGHAEASMQVSDLIYEFMKTKDENLLEEA |
| | LCB1v2.2 | ERLLEEVKR |
| 687 | LCB3-8GS- | NLDELHMQMTDLVYEALHFAKDEEFQKHVFQLFEKATKAYKNKDRQKLEKVVEELKE |
| | SB175 | LLERLLSGGGGSGGGSEALEELEKALRELKKSTDELERSTEELEKNPSEDALVENNR |
| | | LIVENNKIIVEVLRIIAKVLK |
| 688 | LCB3-6GS- | NLDELHMQMTDLVYEALHFAKDEEFQKHVFQLFEKATKAYKNKDRQKLEKVVEELKE |
| | SB175 | LLERLLSGGGGSGSEALEELEKALRELKKSTDELERSTEELEKNPSEDALVENNRLI |
| | | VENNKIIVEVLRIIAKVLK |
| 689 | LCB3-4GS- | NLDELHMQMTDLVYEALHFAKDEEFQKHVFQLFEKATKAYKNKDRQKLEKVVEELKE |
| | SB175 | LLERLLSGGGSSEALEELEKALRELKKSTDELERSTEELEKNPSEDALVENNRLIVE |
| | | NNKIIVEVLRIIAKVLK |
| 690 | LCB3-2GS- | NLDELHMQMTDLVYEALHFAKDEEFQKHVFQLFEKATKAYKNKDRQKLEKVVEELKE |
| | SB175 | KIIVEVLRIIAKVLK |
| | | LLERLLSGGSEALEELEKALRELKKSTDELERSTEELEKNPSEDALVENNRLIVENN |
| 691 | AHB2v1-2GS- | ELEEQVMHVLDQVSELAHELLHKLTGEELERAAYFNWWATEMMLELIKSDDEREIRE |
| | SB175 | IEEEARRILEHLEELARKGGSEALEELEKALRELKKSTDELERSTEELEKNPSEDAL |
| | | VENNRLIVENNKIIVEVLRIIAKVLK |
| 692 | AHB2v2-2GS- | ELEEQVMHVLDQVSELAHELLHKLTGEELERAAYFNWWATEMMLELIKSDDEREIRE |
| | SB175 | IEEEAARILEHLEELARTGGSEALEELEKALRELKKSTDELERSTEELEKNPSEDAL |
| | | VENNRLIVENNKIIVEVLRIIAKVLK |

In some embodiments, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of a genus selected from those recited in the middle column of Table 9. In these embodiments, X1, X2, X3 (when recited in the genus), and X4 (when recited in the genus) may be present or absent, and when present may be any sequence of 1 or more amino acids, as described above for embodiments listed in Table 8. In some embodiments, the optional domain that is present between monomer domains is present and may comprise an amino acid linker, as described above for embodiments listed in Table 8.

In another embodiment, the polypeptides comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to an amino acid sequence comprising the amino acid sequence selected from the group consisting of SEQ ID NOS:693 to 701, wherein any N-terminal methionine residue may be absent or present, and wherein residues in parentheses may be present or absent (preferably absent) and are not considered in determining percent identity. In one embodiment, the N-terminal methionine residue is absent and the optional residues are absent.

TABLE 9

| Name | Sequence |
|---|---|
| C389_AHB2v1_ | MELEEQVMHVLDQVSELAHELLHKLTGEELERAAYFNWWATEMMLELIKSDDEREIREIEEEARR |
| 2GS-SB175 | ILEHLEELARKGGSEALEELEKALRELKKSTDELERSTEELEKNPSEDALVENNRLIVENNKIIV |
| | EVLRIIAKVLK (SEQ ID NO: 693) |
| AHB2v2_12PAS_ | MELEEQVMHVLDQVSELAHELLHKLTGEELERAAYFNWWATEMMLELIKSDDEREIREIEEEAAR |
| LCB3v2.2_12 | ILEHLEELARTGGASPAAPAPGGNLDELHMQMTDLVYEALHFAKDEEFQLHVFQLFEKATKAYKN |
| PAS_LCB1v2.2 | KDRQKLEKVVEELKELLERLLSGGASPAAPAPGGDKENVLQKIYEIMKELERLGHAEASMQVSDL |
| | IYEFMKTKDENLLEEAERLLEEVKR (SEQ ID NO: 694) |
| AHB2v2_24PAS_ | MELEEQVMHVLDQVSELAHELLHKLTGEELERAAYFNWWATEMMLELIKSDDEREIREIEEEAAR |
| LCB3v2.2_24 | ILEHLEELARTGGASPAAPAPASPAAPAPSAPAGGNLDELHMQMTDLVYEALHFAKDEEFQKHVF |
| PAS_LCB1v2.2 | QLFEKATKAYKNKDRQKLEKVVEELKELLERLLSGGASPAAPAPASPAAPAPSAPAGGDKENVLQ |
| | ILEHLEELARTGGASPAAPAPGGNLDELHMQMTDLVYEALHFAKDEEFQKHVFQLFEKATKAYKN |
| | KIYEIMKELERLGHAEASMQVSDLIYEFMKTKDENLLEEAERLLEEVKR (SEQ ID NO: 695) |
| C398_SB175_ | MELEEQVMHVLDQVSELAHELLHKLTGEELERAAYFNWWATEMMLELIKSDDEREIREIEEEARR |
| 6GS_LCB1v2.2 | ILEHLEELARKGGGSSEALEELEKALRELKKSTDELERSTEELEKNPSEDALVENNRLIVENNKI |
| | IVEVLRIIAKVLK (SEQ ID NO: 696) |
| AHB2v1_10GS_ | MELEEQVMHVLDQVSELAHELLHKLTGEELERAAYFNWWATEMMLELIKSDDEREIREIEEEARR |
| LCB3v2.2_ | ILEHLEELARKGGGSGGGSGGNLDELHMQMTDLVYEALHFAKDEEFQKHVFQLFEKATKAYKNKD |
| 10GS_LCB1v2.2 | RQKLEKVVEELKELLERLLSGGGSGGGSGGDKENVLQKIYEIMKELERLGHAEASMQVSDLIYEF |
| | MKTKDENLLEEAERLLEEVKR (SEQ ID NO: 697) |
| C390-AHB2v1- | MELEEQVMHVLDQVSELAHELLHKLTGEELERAAYFNWWATEMMLELIKSDDEREIREIEEEARR |
| 4GS-SB175 | ILEHLEELARKGGGSSEALEELEKALRELKKSTDELERSTEELEKNPSEDALVENNRLIVENNKI |
| | IVEVLRIIAKVLK (SEQ ID NO: 696) |
| C326-AHB2v1- | MELEEQVMHVLDQVSELAHELLHKLTGEELERAAYFNWWATEMMLELIKSDDEREIREIEEEARR |
| 4GS-1rfo | ILEHLEELARKGSGSGYIPEAPRDGQAYVRKDGEWVLLSTFL (SEQ ID NO: 698) |
| AHB2v1-10GS- | (MSHHHHHHHHSENLYFQSGG)ELEEQVMHVLDQVSELAHELLHKLTGEELERAAYFNWWATEMM |
| LCB3_v2.3- | LELIKSDDEREIREIEEEARRILEHLEELARKGGGSGGGSGGNIDELLMQVTDLIYEALHFAKDE |
| 10GS- | EFQKHAFQLFEKATKAYKNKDKQKLEKVVEELKELLERILSGGGSGGGSGGDKENVLQKIYEIMK |
| LCB1_v2.2 | ELERLGHAEASMQVSDLIYEFMKTKDENLLEEAERLLEEVKR (SEQ ID NO: 699) |
| with His Tev | |
| tag | |
| LCB1v3 with | (MSHHHHHHHHSENLYFQGGG)DKENILQKIYEIMKTLEQLGHAEASMQVSDLIYEFMKQGDER |
| His Tev tag | LLEEAERLLEEVER (SEQ ID NO: 700) |
| LCB1-Fc9 | DKENILQKIYEIMKTLDQLGHAEASMQVSDLIYEFMKQGDERLLEEAERLLEEVERGGSGSGGSGSGSG |
| With signal | GSEPKSSDKTHTCPPCPAPELLGGPSVFLFPPKPKDTLMISRTPEVTCVVVDVSHEDPEVKFNWYVDGV |
| sequence | EVHNAKTKPREEQYNSTYRVVSVLTVLHQDWLNGKEYKCKVSNKALPAPIEKTISKAKGQPREPQVYTL |
| leader | PPSRDELTKNQVSLTCLVKGFYPSDIAVEWESNGQPENNYKTTPPVLDSDGSFFLYSKLTVDKSRWQQG |
| removed | NVFSCSVMHEALHNHYTQKSLSLSPGK (SEQ ID NO: 701) |

The polypeptide of any embodiment or combination of embodiments described here may further be linked to a stabilization domain to promote increased residency time upon administration to a subject. Any suitable stabilization domain may be used for an intended purpose. Exemplary stabilization domains include, but are not limited to, polyethylene glycol (PEG), albumin, hydroxyethyl starch (HES), conformationally disordered polypeptide sequence composed of the amino acids Pro, Ala, and/or Ser (‘PASylation’), and/or a mucin diffusivity polypeptide composed of amino acids Lys and Ala, with or without Glu. Non-limiting embodiments of such mucin diffusivity polypeptides include, but are not limited to:

Mucin domain:
(SEQ ID NO: 61)
AKAKAKAKAKAKAKAKAKAKGG;
(SEQ ID NO: 62)
GGAKAKAKAKAKAKAKAKAKAK

Exemplary polypeptides of these embodiments may, for example, comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 65-96, wherein in embodiments where a secretion signal is present (MARAWIFFLLCLAGRALA; SEQ ID NO:63) it can be replaced with any other secretion signal.

>Mucin_LCB1_v1.1_Cys_
AKAKAKAKAKAKAKAKAKAKGGDKENILQKIYEIMKTLDQLGHAEASMQVSDLIYEFMKQGDERLLEEAERLLEEVERC
(SEQ ID NO: 65)
>Mucin_LCB1_v1.3
AKAKAKAKAKAKAKAKAKAKGGDKENILQKIYEIMKTLEQLGHAEASMQVSDLIYEFMKQGDERLLEEAERLLEEVER (SEQ
ID NO: 66)
>LCB1 v1.3 Mucin
DKENILQKIYEIMKTLEQLGHAEASMQVSDLIYEFMKQGDERLLEEAERLLEEVERGGAKAKAKAKAKAKAKAKAKAK SEQ
ID NO: 67)
FC Fusions (Bold = Secretion Signal, underline = LCB, Yellow is the GS Linker,
Green is Fc)
>LCB1-Fc1 (BM40-LCB1-GS4-Fc-Opt-WT) (The LCB1 sequences = LCB1-1 of first provisional)
MARAWIFFLLCLAGRALADKEWILQKIYEIMRLLDELGHAEASMRVSDLIYEFMKKGDERLLEEAERLLEEVERGSGSEPKSS
DKTHTCPPCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK (SEQ ID NO: 68)
>LCB1-Fc2 (BM40-LCB1-GS15-Fc-Opt-WT) (The LCB1 sequence = LCB1-1 of first provisional)
MARAWIFFLLCLAGRALADKEWILQKIYEIMRLLDELGHAEASMRVSDLIYEFMKKGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 69)
>LCB1-Fc3 (BM40-Fc-Opt-GS15-2-LCB1-WT) (The LCB1 sequence = LCB1-1 of first provisional)
MARAWIFFLLCLAGRALAEPKSSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVMHEALHNHYTQKSLSLSPG
KSGGSGSGSGGSGSGS DKEWILQKIYEIMRLLDELGHAEASMRVSDLIYEFMKKGDERLLEEAERLLEEVER (SEQ ID
NO: 70)
>LCB1-Fc4 (BM40-LCB1-GS15-Fc-Opt-GS15-2-LCB1-WT) (The LCB1 sequences = LCB1-1 of
first provisional)
MARAWIFFLLCLAGRALADKEWILQKIYEIMRLLDELGHAEASMRVSDLIYEFMKKGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGKSGGSGSGSGGS
GSGSDKEWILQKIYEIMRLLDELGHAEASMRVSDLIYEFMKKGDERLLEEAERLLEEVER (SEQ ID NO: 71)
>LCB1-Fc5 (BM40-LCB1-GS4-Fc-Opt-Q38) (The LCB1 sequence = LCB1-3 of first provisional)
MARAWIFFLLCLAGRALADKEWILQKIYEIMRLLDELGHAEASMRVSDLIYEFMKQGDERLLEEAERLLEEVERGSGSEPKSS
DKTHTCPPCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGK (SEQ ID NO: 72)
>LCB1-Fc6 (BM40-LCB1-GS15-Fc-Opt-Q38) (The LCB1 sequence = LCB1-3 of first provisional)
MARAWIFFLLCLAGRALADKEWILQKIYEIMRLLDELGHAEASMRVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 73)
>LCB1-Fc7 (BM40-Fc-Opt-GS15-2-LCB1-Q38) (The LCB1 sequence = LCB1-3 of first provisional)
MARAWIFFLLCLAGRALAEPKSSDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVMHEALHNHYTQKSLSLSPG
KSGGSGSGSGGSGSGSDKEWILQKIYEIMRLLDELGHAEASMRVSDLIYEFMKQGDERLLEEAERLLEEVER (SEQ ID
NO: 74)
>LCB1-Fc8 (BM40-LCB1-Q38-GS15-Fc-Opt-GS15-2-LCB1-Q38) (The LCB1 sequences = LCB1-3
of first provisional)
MARAWIFFLLCLAGRALADKEWILQKIYEIMRLLDELGHAEASMRVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGKSGGSGSGSGGS
GSGSDKEWILQKIYEIMRLLDELGHAEASMRVSDLIYEFMKQGDERLLEEAERLLEEVER (SEQ ID NO: 75)
>LCB1-6M-Fc9 (BM40-LCB1-6M -4N, 14K, 15T, 18Q, 27Q, 38Q-GS15-Fc-Opt) (The LCB1
sequence = LCB1_v1.1 of this provisional)
MARAWIFFLLCLAGRALADKENILQKIYEIMKTLDQLGHAEASMQVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 76)
>LCB1-6M-Ngly-Fc10 (BM40-LCB1-6M-Ngly -4N, 14K, 15T, 18Q, 27N, 38Q-GS15-Fc-Opt) (The
LCB1 sequence = LCB1-5 of the original provisional = LCB1_v1.1 = LCB1-4 with N-link
Glycosylation)
MARAWIFFLLCLAGRALADKENILQKIYEIMKTLDQLGHAEASMNVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 77)
>LCB3-6M-Fc11 (BM40-LCB3-6M -8Q, 26Q, 28H, 35K, 37T, 43K-GS15-Fc-Opt) (LCB sequence is
the same as LCB3-3 of First Provisional)
MARAWIFFLLCLAGRALANDDELHMQMTDLVYEALHFAKDEEIQKHVFQLFEKATKAYKNKDRQKLEKVVEELKELLERLLSG
GSGSGGSGSGSGGSEPKSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSLSLSPGK
(SEQ ID NO: 78)
>LCB3-6M-NGly-Fc12 (BM40-LCB3-6M-Ngly -8Q, 26Q, 28H, 35N, 37T, 43K-GS15-Fc-Opt) (LCB
sequence is the same as LCB3-4 of First Provisional which is LCB3-3 with N-link
Glycosylation)
MARAWIFFLLCLAGRALANDDELHMQMTDLVYEALHFAKDEEIQKHVFQLFENATKAYKNKDRQKLEKVVEELKELLERLLSG
GSGSGGSGSGSGGSEPKSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFSCSVMHEALHNHYTQKSLSLSPGK
(SEQ ID NO: 79)
>LCB1-6M-GPGcP-Fc13 (BM40-LCB1-6M -4N, 14K, 15T, 18Q, 27Q, 38Q-GPGcP-Fc-Opt) (LCB
sequence is the same as LCB3-3 of First Provisional)
MARAWIFFLLCLAGRALADKENILQKIYEIMKTLDQLGHAEASMQVSDLIYEFMKQGDERLLEEAERLLEEVERAGSGGSGGS
GGSPVPSTPPTPSPSTPPTPSPSGGSGNSSGSGGSPVPSTPPTPSPSTPPTPSPSASEPKSSDKTHTCPPCPAPELLGGPSVF
LFPPKPKDTLMISRTPEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK (SEQ ID NO: 80)
>LCB3-6M-GPGcP-Fc14 (BM40-LCB3-6M -8Q, 26Q, 28H, 35K, 37T, 43K-GPGcP-Fc-Opt) (LCB
sequence is the same as LCB3-3 of First Provisional)
MARAWIFFLLCLAGRALANDDELHMQMTDLVYEALHFAKDEEIQKHVFQLFEKATKAYKNKDRQKLEKVVEELKELLERLLSA
GSGGSGGSGGSPVPSTPPTPSPSTPPTPSPSGGSGNSSGSGGSPVPSTPPTPSPSTPPTPSPSASEPKSSDKTHTCPPCPAPE
LLGGPSVFLFPPKPKDTLMISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK (SEQ ID NO: 81)
>LCB1-6M-GS30-Fc15 (BM40-LCB1-6M -4N, 14K, 15T, 18Q, 27Q, 38Q-GS30-Fc-Opt) LCB1-4
MARAWIFFLLCLAGRALADKENILQKIYEIMKTLDQLGHAEASMQVSDLIYEFMKQGDERLLEEAERLLEEVERGGGSGGGGS
GGGGSGGGGSGGGGSGGGGSGEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNHYTQKSLSL
SPGK (SEQ ID NO: 82)
>LCB3-6M-GS30-Fc16 (BM40-LCB3-6M -8Q, 26Q, 28H, 35K, 37T, 43K-GS30-Fc-Opt) (LCB
sequence is the same as LCB3-3 of First Provisional)
MARAWIFFLLCLAGRALANDDELHMQMTDLVYEALHFAKDEEIQKHVFQLFEKATKAYKNKDRQKLEKVVEELKELLERLLSG
GGSGGGGSGGGGSGGGGSGGGGSGGGGSGEPKSSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQGNVFSCSVMHEALHNH
YTQKSLSLSPGK (SEQ ID NO: 83)
>LCB1-v1.3-Fc17 (BM40-LCB1-v1.3 -4N, 14K, 15T, 17E, 18Q, 27Q, 38Q-GS15-Fc-Opt)
MARAWIFFLLCLAGRALADKENILQKIYEIMKTLEQLGHAEASMQVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 84)
>LCB1-v1.3-Ngly-Fc18 (BM40-LCB1-v1.3 Ngly -4N, 14K, 15T, 17E, 18Q, 27N, 38Q-GS15-Fc-
Opt) (>LCB1_v1.5 (= LCB1_v1.3 with N-link Glycosylation)
MARAWIFFLLCLAGRALADKENILQKIYEIMKTLEQLGHAEASMNVSDLIYEFMKQGDERLLEEAERLLEEVERGGSGSGGSG
SGSGGSEPKSSDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK (SEQ ID
NO: 85)
>LCB1-v1.3-GPGcP-Fc19 (BM40-LCB1-v1.3-Fc19 -4N, 14K, 15T, 17E, 18Q, 27Q, 38Q-GPGcP-Fc-
Opt)
MARAWIFFLLCLAGRALADKENILQKIYEIMKTLEQLGHAEASMQVSDLIYEFMKQGDERLLEEAERLLEEVERAGSGGSGGS
GGSPVPSTPPTPSPSTPPTPSPSGGSGNSSGSGGSPVPSTPPTPSPSTPPTPSPSASEPKSSDKTHTCPPCPAPELLGGPSVF
LFPPKPKDTLMISRTPEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK (SEQ ID NO: 86)
>LCB1-v1.3-GS30-Fc20 (BM40-LCB1-v1.3 -4N, 14K, 15T, 17E, 18Q, 27Q, 38Q -GS30-Fc-Opt)
MARAWIFFLLCLAGRALADKENILQKIYEIMKTLEQLGHAEASMQVSDLIYEFMKQGDERLLEEAERLLEEVERGGGSGGGGS
GGGGSGGGGSGGGGSGGGGSGEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSCSVMHEALHNHYTQKSLSL
SPGK (SEQ ID NO: 87)
Fc21-LCB1v2.2-FcIgG1_WT
MARAWIFFLLCLAGRALADKENVLQKIYEIMKELERLGHAEASMQVSDLIYEFMKTKDENL
LEEAERLLEEVKRGGSGSGGSGSGSGGSEPKSSDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEA
LHNHYTQKSLSLSPGK (SEQ ID NO: 88)
Fc22-LCB1v2.2-FcIgG3_WT
MARAWIFFLLCLAGRALANIDELLMQVTDLIYEALHFAKDEEFQKHAFQLFEKATKAYKNK
LEEAERLLEEVKRGGSGSGGSGSGSGGSEPKSSDKTHTCPRCPAPELLGGPSVFLFPPKPK
DTLMISRTPEVTCVVVDVSHEDPEVQFKWYVDGVEVHNAKTKPREEQFNSTFRVVSVLTVL
HQDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHEA
LHNRFTQKSLSLSPGK (SEQ ID NO: 89)
Fc23_LCB3_v2.3-10GS-LCB1_v2.2_FcIgG1_WT
MARAWIFFLLCLAGRALANIDELLMQVTLDIYEALHFAKDEEFQKHAFQLFEKATKAYKNK
LIYEFMKTKDENLLEEAERLLEEVKRGGSGSGGSGSGSGGSEPKSSDKTHTCPPCPAPELL
GGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQ
QGNVFSCSVMHEALHNHYTQKSLSLSPGK (SEQ ID NO: 90)
Fc2_AHB2-10GS-LCB3_v2.3-10GS-LCB1_v2.2_FcIgG1_WT
MARAWIFFLLCLAGRALAELEEQVMHVLDQVSELAHELLHKLTGEELERAAYFNWWATEMM
LELIKSDDEREIREIEEEARRILEHLEELARKGGGSGGGSGGNIDELLMQVTDLIYEALHF
AKDEEFQKHAFQLFEKATKAYKNKDKQKLEKVVEELKELLERILSGGGSGGGSGGDKENVL
QKIYEIMKELERLGHAEASMQVSDLIYEFMKTKDENLLEEAERLLEEVKRGGSGSGGSGSG
SGGSEPKSSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK (SEQ ID
NO: 91)
Fc25_LCB3_v2.3-10GS-AHB2-10GS-LCB1_v2.2_FcIgG1_WT
MARAWIFFLLCLAGRALANIDELLMQVTDLIYEALHFAKDEEFQKHAFQLFEKATKAYKNK
DKQKLEKVVEELKELLERILSGGGSGGGSGGELEEQVMHVLDQVSELAHELLHKLTGEELE
RAAYFNWWATEMMLELIKSDDEREIREIEEEARRILEHLEELARKGGGSGGGSGGDKENVL
SGGSEPKSSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK (SEQ ID
NO: 92)
Fc26_LCB3_v2.3-10GS-LCB1_v2.2_FcIgG1_GA
MARAWIFFLLCLAGRALANIDELLMQVTDLIYEALHFAKDEEFQKHAFQLFEKATKAYKNK
LIYEFMKTKDENLLEEAERLLEEVKRGGSGSGGSGSGSGGSEPKSSDKTHTCPPCPAPELL
AGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQ
QGNVFSCSVMHEALHNHYTQKSLSLSPGK (SEQ ID NO: 93)
Fc27_LCB3_v2.3-10GS-LCB1_v2.2_FcIgG1_GRLR
MARAWIFFLLCLAGRALANIDELLMQVTDLIYEALHFAKDEEFQKHAFQLFEKATKAYKNK
DKQKLEKVVEELKELLERILSGGGSGGGSGGDKENVLQKIYEIMKELERLGHAEASMQVSD
LIYEFMKTKDENLLEEAERLLEEVKRGGSGSGGSGSGSGGSEPKSSDKTHTCPPCPAPELL
RGPSVFLFPPKPKDTLMISRTPEVTCVVVDVSHEDPEVKFNWYVDGVEVHNAKTKPREEQY
NSTYRVVSVLTVLHQDWLNGKEYKCKVSNKARPAPIEKTISKAKGQPREPQVYTLPPSRDE
LTKNQVSLTCLVKGFYPSDIAVEWESNGQPENNYKTTPPVLDSDGSFFLYSKLTVDKSRWQ
QGNVFSCSVMHEALHNHYTQKSLSLSPGK (SEQ ID NO: 94)
Fc28_LCB3_v2.3-10GS-LCB1_v2.2_FcIgG1_GAALIE
MARAWIFFLLCLAGRALANIDELLMQVTDLIYEALHFAKDEEFQKHAFQLFEKATKAYKNK
LIYEFMKTKDENLLEEAERLLEEVKRGGSGSGGSGSGSGGSEPKSSDKTHTCPPCPAPELL
AGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPLPEEKTISKAKGQPREPQVYTLPPSRDE
LTKNQVSLTCLVKGFYPSDIAVEWESNGQPENNYKTTPPVLDSDGSFFLYSKLTVDKSRWQ
QGNVFSCSVMHEALHNHYTQKSLSLSPGK (SEQ ID NO: 95)
Fc29_LCB3_v2.3-10GS-LCB1_v2.2_FcIgG1_GAALIELS
MARAWIFFLLCLAGRALANIDELLMQVTDLIYEALHFAKDEEFQKHAFQLFEKATKAYKNK
DKQKLEKVVEELKELLERILSGGGSGGGSGGDKENVLQKIYEIMKELERLGHAEASMQVSD
LIYEFMKTKDENLLEEAERLLEEVKRGGSGSGGSGSGSGGSEPKSSDKTHTCPPCPAPELL
AGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPLPEEKTISKAKGQPREPQVYTLPPSRDE
LTKNQVSLTCLVKGFYPSDIAVEWESNGQPENNYKTTPPVLDSDGSFFLYSKLTVDKSRWQ
QGNVFSCSVLHEALHSHYTQKSLSLSPGK (SEQ ID NO: 96)

The disclosure further provides oligomers of the polypeptide of any embodiment or combination of embodiments herein. In one embodiment, the oligomers are oligomers of polypeptides disclosed herein that comprise oligomerization domains. In one embodiment, the oligomer comprises a trimer, including but not limited to a homotrimer.

In another embodiment, the disclosure provides compositions, comprising 2, 3, 4, or more copies of the polypeptide of any embodiment or combination of embodiments herein attached to a support, including but not limited to a polypeptide particle support, such as a nanoparticle or virus like particle.

As disclosed herein, the polypeptides bind to the SARS-COV-2 Spike glycoprotein, and thus are useful (for example), as therapeutics to treat SARS-COV-2 infection. In one embodiment, the polypeptides bind to the SARS-COV-2 Spike glycoprotein with an affinity of at least 10 nM, measured as described in the attached examples.

In another aspect, the disclosure provides nucleic acids encoding a polypeptide of the disclosure. The nucleic acid sequence may comprise RNA (such as mRNA) or DNA. Such nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention.

In another aspect, the disclosure provides expression vectors comprising the nucleic acid of any embodiment or combination of embodiments of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

In one aspect, the present disclosure provides cells comprising the polypeptide, the composition, the nucleic acid, and/or the expression vector of any embodiment or combination of embodiments of the disclosure, wherein the cells can be either prokaryotic or eukaryotic, such as mammalian cells. In one embodiment the cells may be transiently or stably transfected with the nucleic acids or expression vectors of the disclosure. Such transfection of expression vectors into prokaryotic and eukaryotic cells can be accomplished via any technique known in the art. A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. In other embodiments, the polypeptides may be produced via any other suitable technique, including but not limited to using cell-free protein synthesis (or in vitro transcription and translation).

In another aspect, the disclosure provides pharmaceutical compositions/vaccines comprising

(a) the polypeptide, the nucleic acid, the expression vector, and/or the host cell of any embodiment or combination of embodiments herein; and

(b) a pharmaceutically acceptable carrier.

The compositions may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The polypeptide, the nucleic acid, the expression vector, and/or the host cell may be the sole active agent in the composition, or the composition may further comprise one or more other agents suitable for an intended use.

In a further aspect, the disclosure provides methods for treating a severe acute respiratory syndrome (SARS) coronavirus infection (including SARS-Co-V and SARS-COV-2), comprising administering to a subject in need thereof an amount of the polypeptide, the nucleic acid, the expression vector, the host cell, the oligomer, the composition, and/or the pharmaceutical composition of any of the preceding claims, effective to treat the infection. In one embodiment, the SARS coronavirus comprises SARS-COV-2.

In another aspect, the disclosure provides methods for limiting development of a severe acute respiratory syndrome (SARS) coronavirus infection (including SARS-Co-V and SARS-COV-2), comprising administering to a subject in need thereof an amount of the polypeptide, the nucleic acid, the expression vector, the host cell, the oligomer, the composition, and/or the pharmaceutical composition of any of the preceding claims, effective to treat the infection. In one embodiment, the SARS coronavirus comprises SARS-COV-2.

The polypeptide, the nucleic acid, the expression vector, the host cell, and/or the pharmaceutical composition may be administered via any suitable administrative route as deemed appropriate by attending medical personnel. In one embodiment, the polypeptide, the nucleic acid, the expression vector, the host cell, the oligomer, the composition, and/or the pharmaceutical composition is administered intra-nasally. In another embodiment, the polypeptide, the nucleic acid, the expression vector, the host cell, the oligomer, the composition, and/or the pharmaceutical composition is administered systemically.

When the method comprises treating a SARS coronavirus infection, the one or more polypeptides, nucleic acids, expression vectors, host cells, and/or pharmaceutical compositions are administered to a subject that has already been diagnosed as having a SARS coronavirus infection. As used herein, “treat” or “treating” means accomplishing one or more of the following: (a) reducing severity of symptoms of the infection in the subject; (b) limiting increase in symptoms in the subject; (c) increasing survival; (d) decreasing the duration of symptoms; (e) limiting or preventing development of symptoms; and (f) decreasing the need for hospitalization and/or the length of hospitalization for treating the infection.

When the method comprises limiting development of SARS coronavirus infection, the one or more polypeptides, nucleic acids, expression vectors, host cells, and/or pharmaceutical compositions are administered prophylactically to a subject that is not known to have a SARS coronavirus infection, but may be at risk of such an infection. As used herein, “limiting” means to limit development of a SARS coronavirus infection in subjects at risk of such infection, which may be any subject.

The subject may be any subject, such as a human subject

Exemplary symptoms of SARS-COV-2 infection include, but are not limited to, fever, fatigue, cough, shortness of breath, chest pressure and/or pain, loss or diminution of the sense of smell, loss or diminution of the sense of taste, and respiratory issues including but not limited to pneumonia, bronchitis, severe acute respiratory syndrome (SARS), and upper and lower respiratory tract infections.

As used herein, an “effective amount” refers to an amount of the composition that is effective for treating and/or limiting SARS-COV-2 infection. The polypeptide, composition, nucleic acid, or composition of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

The disclosure also provides methods for designing polypeptides that bind to the receptor binding site (RBD) of SARS-Cov-2, wherein the methods comprise steps as described in the examples that follow. Such methods may comprise the steps of polypeptide design (as described in any embodiment or combination of embodiments in the examples), cell-free synthesis, and evaluation for SARS-Cov-2 RBD binding using any suitable technique.

### EXAMPLES

### Summary

Effective therapeutics for SARS-COV-2 are needed. We sought to use computational protein design to generate high affinity binders to the receptor binding site (RBD) of SARS-Cov-2 that block the interaction with the Ace2 receptor required for cell entry. We generated small protein scaffolds with shape complementary to the Ace2 binding site on the RBD using two strategies: first, scaffolds were built around the helix in Ace2 that makes the majority of the interactions with the RBD, and second, diverse de novo designed scaffolds less than 65 residues in length were docked against this region. In both cases, the scaffold residues at the RBD interface were then optimized for high affinity binding and those in the remainder of the protein, for folding to the target structure and stability. The 50,000 designs predicted to bind most strongly to the virus were encoded in large oligonucleotide arrays, and screened using yeast surface display for binding to the RBD with fluorescence activated cell sorting; deep sequencing of the population before and after sorting identified hundreds of designs that bind the target. The binding modes of the highest affinity (most enriched by sorting) binders were confirmed by high resolution sequence mapping, and the affinities were further increased by combining 1-4 beneficial substitutions. Eight of the optimized designs with different binding sites surrounding the Ace2 interface on the RBD, and completely different sequences, were found to express at high levels in E coli, and to bind the RBD with Kd's ranging from 100 pM to 10 nM. The designs blocked infection of vero-6 cells by live virus with IC50's ranging from 10 nM to 20 pM. The polypeptides are thus useful, for example, in both intra-nasal and systemic SARS-COV-2 therapeutics, and, more generally, our results demonstrate the power of computational protein design for rapidly generating potential therapeutic candidates against pandemic threats.

SARS-COV-2 infection is thought to often start in the nose, with virus replicating there for several before spreading to the broader respiratory system. Delivery of a high concentration of a viral inhibitor into the nose and into the respiratory system generally could therefore potentially provide prophylactic protection, and therapeutic efficacy early in infection, and could be particularly useful for health care workers and others coming into frequent contact with infected individuals. A number of monoclonal antibodies are in development as systemic SARS-COV-2 therapeutics, but these compounds are not ideal for intranasal delivery as antibodies are large and often not extremely stable molecules, and the density of binding sites is low (two per 150 Kd antibody); the Fc domain provides little added benefit. More desirable would be protein inhibitory with the very high affinity for the virus of the monoclonals, but with higher stability and very much smaller size to maximize the density of inhibitory domains and enable direct delivery into the respiratory system through nebulization.

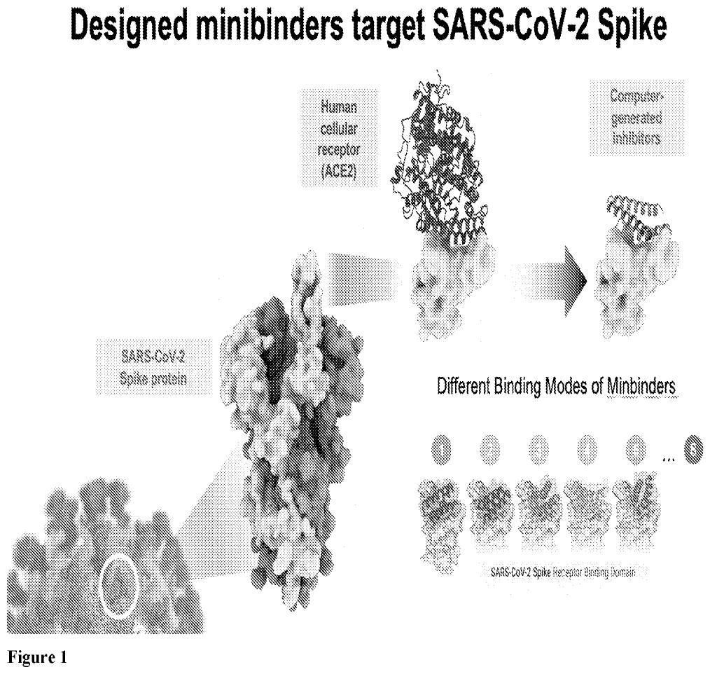

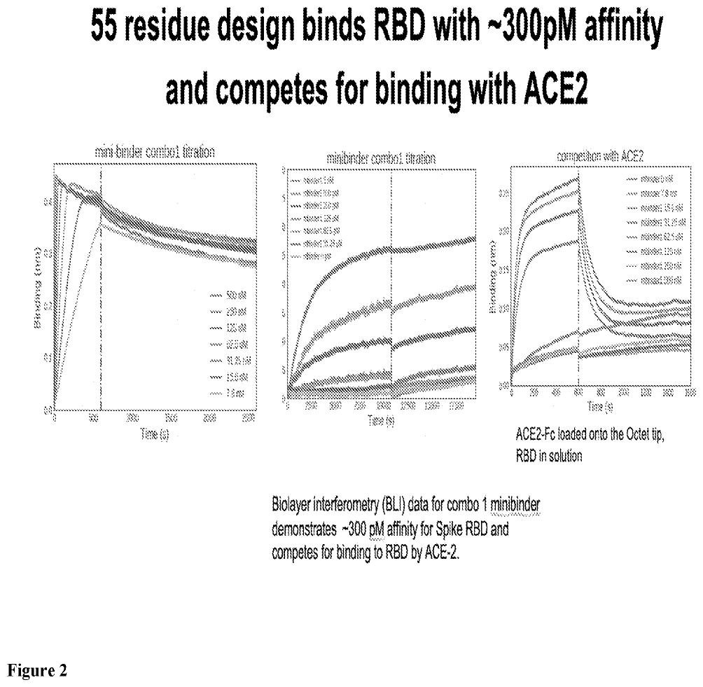

We set out to de novo design high affinity binders to the RBD that compete with Ace2 binding. We explored two strategies: first we attempted to scaffold the alpha helix in Ace2 that makes the majority of the interactions with the RBD in a small designed protein that makes additional interactions with the RBD to attain higher affinity, and second, we sought to design binders completely from scratch that do not incorporate any known binding interaction with the RBD. An advantage of the second approach is that the range of possibilities for design is much larger, and so potentially higher affinity binding modes can be identified. For the first approach, we used the Rosetta™ blue print builder to generate small proteins which incorporate the Ace2 helix and for the second approach, RIF docking and design using large miniprotein libraries. The designs interact with distinct regions of the RBD surface surrounding the Ace2 binding sites (FIG. 1). Designs for approach 1, and approach 2, were encoded in long oligonucleotides, and screened for binding to fluorescently tagged RBD on the yeast cell surface. Deep sequencing identified 3 Ace2 helix scaffolded designs (approach 1), and 150 de novo interface designs (approach 2) that were clearly enriched following FACS sorting for RBD binding. Designs were expressed in E. coli and purified, and many were found to be have soluble expression and to bind RBD in biolayer interferometry experiments and could effectively compete with ACE-2 for binding to RBD (example shown in FIG. 2). Based on BLI data (e.g. See FIG. 2) the RBD binding affinities of minibinders are: LCB1<1 nM, LCB3<1 nM. The affinities of LCB2, LCB4, LCB5, LCB6, LCB7, LCB8 range from 1˜20 nM, with relative strength of different binders being LCB4>LCB2>LCB9=LCB5>LCB6>LCB7.

To determine whether the designs binding the RBD through the designed interfaces, site saturation libraries in which every residue in each design was substituted with each of the 20 amino acids one at a time were constructed, and subjected to FACS sorting for RBD binding. Deep sequencing showed that the binding interface residues and protein core residues were conserved in many of the designs for which such site saturation libraries (SSM's) were constructed (SSMs were used to define allowable positions for amino acid changes in Table 1). For most of the designs, a small number of substitutions were enriched in the FACS sorting, suggesting they increase binding affinity for RBD. For the highest affinity of the approach 1 designs, and 8 of the approach 2 designs, combinatorial libraries incorporating these substitutions were constructed and again screened for binding with FACS; because of the very high binding affinity the concentrations used in the sorting were as low as 20 pM. Each library converged on a small number of closely related sequences, and for each design, one of the optimized variants was expressed in E. coli and purified.

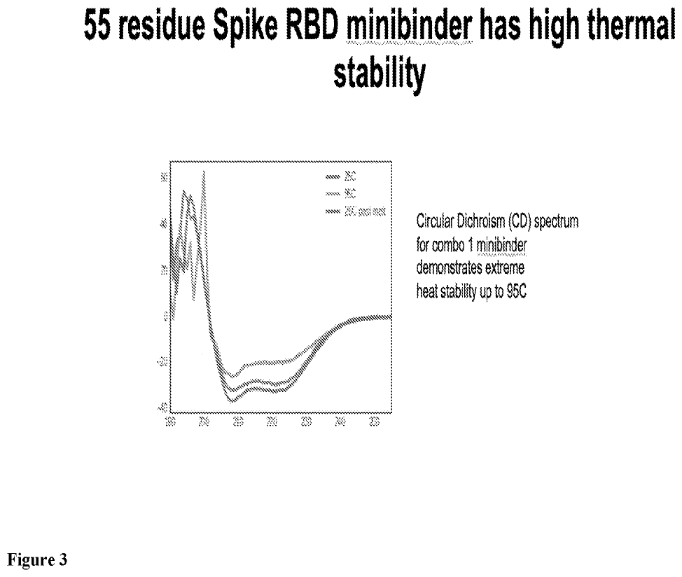

The binding of the 8 optimized designs with different binding modes to RBD (FIG. 1) was investigated by biolayer interferometry. For a number of the designs, the Kd's ranged from 1-20 nM, and for the remainder, the Kd's were below 1 nM, too strong to measure reliably with this technique (See FIG. 2). Circular dichroism spectra of the designs were consistent with the design models, and the designs retained full binding activity after a number of days at room temperature (FIG. 3).

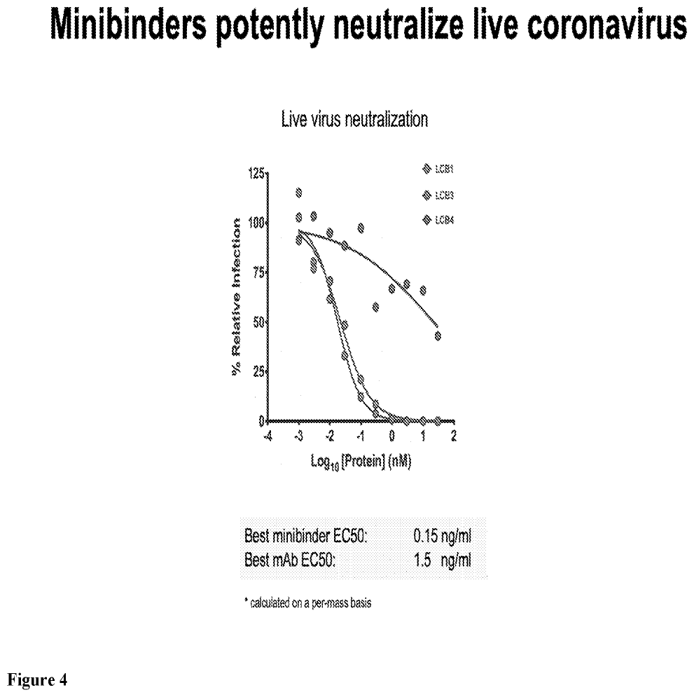

We investigated the ability of the designs to block infection of human cells by live virus. 100 FFU of SARS-COV-2 was added to 2.5-3×104 vero cells in the presence of varying amounts of the designed binders. Details are provided in the legend to FIG. 4. We observed potent inhibition of infection for all of the designs with IC50's ranging from 1 nM to 0.02 nM.

Details on specific designs are provided in Table 10.

TABLE 10

| | | | | | Estimated | | Estimated | |
|---|---|---|---|---|---|---|---|---|
| | | | | | Level of | Estimated | Kd for | SARS-CoV-2 |
| | | | | | Soluble | Kd for | Spike | Neutralization |
| Construct | | Expression | | Expression | Production | RBD | (nM) | IC50 |
| Name | Seq ID | Host | Vector | Method | (mg/L) | (nM) | Avidity | (nM) |
| LCB1-1 | (SEQ ID | E. coli | pET | Autoinduction | 10 | 0.2 | 0.1 | 0.01 |
| | NO: 1) | | | 37 C. | | | | |
| LCB1-2 | (SEQ ID | E. coli | pET | Autoinduction | 10 | 0.2 | 0.1 | 0.01 |
| | NO: 2) | | | 37 C. | | | | |
| LCB1-3 | (SEQ ID | E. coli | pET | Autoinduction | 10 | 0.2 | 0.1 | 0.01 |
| | NO: 3) | | | 37 C. | | | | |
| LCB1-4 | (SEQ ID | E. coli | pET | Autoinduction | 10 | 0.2 | 0.1 | 0.01 |
| | NO: 4) | | | 37 C. | | | | |
| LCB1-5 | (SEQ ID | E. coli | pET | Autoinduction | 10 | 0.2 | 0.1 | 0.01 |
| | NO: 5) | | | 37 C. | | | | |
| LCB1_v1.1_Cys | (SEQ ID | E. coli | pET | Autoinduction | 10 | 0.2 | 0.1 | 0.01 |
| | NO: 6) | | | 37 C. | | | | |
| LCB1_v1.2 | (SEQ ID | E. coli | pET | Autoinduction | 10 | 0.2 | 0.1 | 0.01 |
| | NO: 7) | | | 37 C. | | | | |
| LCB1_v1.3 | (SEQ ID | E. coli | pET | Autoinduction | 10 | 0.2 | 0.1 | 0.01 |
| | NO: 8) | | | 37 C. | | | | |
| LCB1_v1.4 | (SEQ ID | E. coli | pET | Autoinduction | 10 | 0.2 | 0.1 | 0.01 |
| | NO: 9) | | | 37 C. | | | | |
| LCB1_v1.5 | (SEQ ID | E. coli | pET | Autoinduction | 10 | 0.2 | 0.1 | 0.01 |
| (LCB1_v1.3 | NO: 10) | | | 37 C. | | | | |
| with N-link | | | | | | | | |
| Glycosylation) | | | | | | | | |
| LCB2-1 | (SEQ ID | E. coli | pET | Autoinduction | 10 | 0.2 | 0.1 | 0.01 |
| | NO: 11) | | | 37 C. | | | | |
| LCB2-2 | (SEQ ID | E. coli | pET | Autoinduction | 10 | 0.2 | 0.1 | 0.01 |
| | NO: 12) | | | 37 C. | | | | |
| LCB3-1 | (SEQ ID | E. coli | pET | Autoinduction | 10 | 0.2 | 0.1 | 0.01 |
| | NO: 13) | | | 37 C. | | | | |
| LCB3-2 | (SEQ ID | E. coli | pET | Autoinduction | 10 | 0.2 | 0.1 | 0.01 |
| | NO: 14) | | | 37 C. | | | | |
| LCB3-3 | (SEQ ID | E. coli | pET | Autoinduction | 10 | 0.2 | 0.1 | 0.01 |
| | NO: 15) | | | 37 C. | | | | |
| LCB3-4 | (SEQ ID | E. coli | pET | Autoinduction | 10 | 0.2 | 0.1 | 0.01 |
| | NO: 16) | | | 37 C. | | | | |
| LCB3_v1.1 | (SEQ ID | E. coli | pET | Autoinduction | 10 | 0.2 | 0.1 | 0.01 |
| | NO: 17) | | | 37 C. | | | | |
| LCB3_v1.2 | (SEQ ID | E. coli | pET | Autoinduction | 10 | 0.2 | 0.1 | 0.01 |
| | NO: 15) | | | 37 C. | | | | |
| LCB3_v1.3 | (SEQ ID | E. coli | pET | Autoinduction | 10 | 0.2 | 0.1 | 0.01 |
| | NO: 19) | | | 37 C. | | | | |
| LCB3_v1.4 | (SEQ ID | E. coli | pET | Autoinduction | 10 | 0.2 | 0.1 | 0.01 |
| | NO: 20) | | | 37 C. | | | | |
| LCB3_v1.5 | (SEQ ID | E. coli | pET | Autoinduction | 10 | 0.2 | 0.1 | 0.01 |
| | NO: 21) | | | 37 C. | | | | |
| LCB3-4 | (SEQ ID | E. coli | pET | Autoinduc ion | 10 | 0.2 | 0.1 | 0.01 |
| | NO: 16) | | | 37 C. | | | | |
| LCB4-1 | (SEQ ID | E. coli | pET | Autoinduction | 10 | 0.2 | 0.1 | 0.01 |
| | NO: 23) | | | 37 C. | | | | |
| LCB4-2 | (SEQ ID | E. coli | pET | Autoinduction | 10 | 0.2 | 0.1 | 0.01 |
| | NO: 24) | | | 37 C. | | | | |
| LCB5-1 | (SEQ ID | E. coli | pET | Autoinduction | 10 | 0.2 | 0.1 | 0.01 |
| | NO:25) | | | 37 C. | | | | |
| LCB5-2 | (SEQ ID | E. coli | pET | Autoinduction | 10 | 0.2 | 0.1 | 0.01 |
| | NO: 26) | | | 37 C. | | | | |
| LCB6-1 | (SEQ ID | E. coli | pET | Autoinduction | 10 | 0.2 | 0.1 | 0.01 |
| | NO: 27) | | | 37 C. | | | | |
| LCB6-2 | (SEQ ID | E. coli | pET | Autoinduction | 10 | 0.2 | 0.1 | 0.01 |
| | NO: 28) | | | 37 C. | | | | |
| LCB7-1 | (SEQ ID | E. coli | pET | Autoinduction | 10 | 0.2 | 0.1 | 0.01 |
| | NO: 29) | | | 37 C. | | | | |
| LCB7-2 | (SEQ ID | E. coli | pET | Autoinduction | 10 | 0.2 | 0.1 | 0.01 |
| | NO: 30) | | | 37 C. | | | | |
| LCB8-1 | (SEQ ID | E. coli | pET | Autoinduction | 10 | 0.2 | 0.1 | 0.01 |
| | NO: 31) | | | 37 C. | | | | |
| LCB8-2 | (SEQ ID | E. coli | pET | Autoinduction | 10 | 0.2 | 0.1 | 0.01 |
| | NO: 32) | | | 37 C. | | | | |
| AHB1-1 | (SEQ ID | E. coli | pET | Autoinduction | 10 | 0.2 | 0.1 | 0.01 |
| | NO: 33) | | | 37 C. | | | | |
| AHB1-2 | (SEQ ID | E. coli | pET | Autoinduction | 10 | 0.2 | 0.1 | 0.01 |
| | NO: 34) | | | 37 C. | | | | |
| AHB2-1 | (SEQ ID | E. coli | pET | Autoinduction | 10 | 0.2 | 0.1 | 0.01 |
| | NO: 100) | | | 37 C. | | | | |
| AHB2-2 | (SEQ ID | E. coli | pET | Autoinduction | 10 | 0.2 | 0.1 | 0.01 |
| | NO: 101) | | | 37 C. | | | | |
| LCB1-6GS- | (SEQ ID | E. coli | pET | Autoinduction | 10 | 0.2 | 0.1 | 0.01 |
| LCB1 | NO: 47) | | | 37 C. | | | | |
| LCB1-12GS- | (SEQ ID | E. coli | pET | Autoinduction | 10 | 0.2 | 0.1 | 0.01 |
| LCB1 | NO: 48) | | | 37 C. | | | | |
| LCB1-24GS- | (SEQ ID | E. coli | pET | Autoinduction | 10 | 0.2 | 0.1 | 0.01 |
| LCB1 | NO: 49) | | | 37 C. | | | | |
| LCB1-36GS- | (SEQ ID | E. coli | pET | Autoinduction | 10 | 0.2 | 0.1 | 0.01 |
| LCB1 | NO: 50) | | | 37 C. | | | | |
| LCB1_v1.1- | (SEQ ID | E. coli | pET | Autoinduction | 10 | 0.2 | 0.1 | 0.01 |
| GSLCB1_v1.1 | NO: 51) | | | 37 C. | | | | |
| (1GS1) | | | | | | | | |
| LCB1_v1.1- | (SEQ ID | E. coli | pET | Autoinduction | 10 | 0.2 | 0.1 | 0.01 |
| PRO- | NO: 52) | | | 37 C. | | | | |
| LCB1_v1.1 | | | | | | | | |
| (1PRO1) | | | | | | | | |
| LCB3_v1.2- | (SEQ ID | E. coli | pET | Autoinduction | 10 | 0.2 | 0.1 | 0.01 |
| GS3- | NO: 53) | | | 37 C. | | | | |
| LCB3_v1.2 | | | | | | | | |
| (3GS3) | | | | | | | | |
| LCB3_v1.2- | (SEQ ID | E. coli | pET | Autoinduction | 10 | 0.2 | 0.1 | 0.01 |
| PRO- | NO: 54) | | | 37 C. | | | | |
| LCB3_v1.2 | | | | | | | | |
| (3PRO3) | | | | | | | | |
| LCB1_v1.1- | (SEQ ID | E. coli | pET | Autoinduction | 10 | 0.2 | 0.1 | 0.01 |
| GS- | NO: 55) | | | 37 C. | | | | |
| LCB3_v1.2 | | | | | | | | |
| (1GS3) | | | | | | | | |
| LCB3_v1.2- | (SEQ ID | E. coli | pET | Autoinduction | 10 | 0.2 | 0.1 | 0.01 |
| GS- | NO: 56) | | | 37 C. | | | | |
| LCB1_v1.1 | | | | | | | | |
| (3GS1) | | | | | | | | |
| LCB3_v1.2- | (SEQ ID | E. coli | pET | Autoinduction | 10 | 0.2 | 0.1 | 0.01 |
| 10GS- | NO: 57) | | | 37 C. | | | | |
| LCB1_v1.1 | | | | | | | | |
| (LCB3-GS10- | | | | | | | | |
| LCB1) | | | | | | | | |
| LCB1_v1.1- | (SEQ ID | E. coli | pET | Autoinduction | 10 | 0.2 | 0.1 | 0.01 |
| PRO- | NO: 58) | | | 37 C. | | | | |
| LCB3_v1.2 | | | | | | | | |
| (1PRO3) | | | | | | | | |
| LCB3_v1.2- | (SEQ ID | E. coli | pET | Autoinduction | 10 | 0.2 | 0.1 | 0.01 |
| PRO- | NO: 59) | | | 37 C. | | | | |
| LCB1_v1.1 | | | | | | | | |
| (3PRO1) | | | | | | | | |
| (5_LCB1_linker14) | (SEQ ID | E. coli | pET | Autoinduction | 20.38 | 0.2 | 0.1 | 0.01141 |
| | NO: 60) | | | 37 C. | | | | |
| Mucin_LCB1_ | (SEQ ID | E. coli | pET | Autoinduction | 10 | 0.2 | 0.1 | 0.01 |
| v1.1_Cys | NO: 65) | | | 37 C. | | | | |
| Mucin_LCB1_ | (SEQ ID | E. coli | pET | Autoinduction | 10 | 0.2 | 0.1 | 0.01 |
| v1.3 | NO: 66) | | | 37 C. | | | | |
| LCB1_v1.3_Mucin | (SEQ ID | E. coli | pET | Autoinduction | 10 | 0.2 | 0.1 | 0.01 |
| | NO: 67) | | | 37 C. | | | | |
| LCB1-Fc1 | (SEQ ID | Human | pCMVR | Transient | 12 | 0.2 | 0.1 | 0.01 |
| (BM40-LCB1- | NO: 68) | 293 | | Transfection | | | | |
| GS4-Fc-Opt- | | cells | | 37 C. | | | | |
| WT) (The | | | | | | | | |
| LCB1 | | | | | | | | |
| sequences = | | | | | | | | |
| LCB1-1 of | | | | | | | | |
| first | | | | | | | | |
| provisional) | | | | | | | | |
| LCB1-Fc2 | (SEQ ID | Human | pCMVR | Transfection | 12 | 0.2 | 0.1 | 0.01 |
| (BM40-LCB1- | NO: 69) | 293 | | Transient | | | | |
| GS15-Fc-Opt- | | cells | | 37 C. | | | | |
| WT) (The LCB1 | | | | | | | | |
| sequence = | | | | | | | | |
| LCB1-1 of | | | | | | | | |
| first | | | | | | | | |
| provisional) | | | | | | | | |
| LCB1-Fc3 | (SEQ ID | Human | pCMVR | Transient | 12 | 0.2 | 0.1 | 0.01 |
| (BM40-Fc- | NO: 70) | 293 | | Transfection | | | | |
| Opt-GS15-2- | | cells | | 37 C. | | | | |
| LCB1-WT) | | | | | | | | |
| (The LCB1 | | | | | | | | |
| sequence = | | | | | | | | |
| LCB1-1 of | | | | | | | | |
| first | | | | | | | | |
| provisional) | | | | | | | | |
| LCB1-Fc4 | (SEQ ID | Human | pCMVR | Transient | 2 | 0.2 | 0.1 | 0.01 |
| (BM40-LCB1- | NO: 71) | 293 | | Transfection | | | | |
| GS15-Fc-Opt- | | cells | | 37 C. | | | | |
| GS15-2-LCB1- | | | | | | | | |
| WT) (The | | | | | | | | |
| LCB1 | | | | | | | | |
| sequences = | | | | | | | | |
| LCB1-1 of | | | | | | | | |
| first | | | | | | | | |
| provisional) | | | | | | | | |
| LCB1-Fc5 | (SEQ ID | Human | pCMVR | Transient | 6 | 0.2 | 0.1 | 0.01 |
| (BM40-LCB1- | NO: 72) | 293 | | Transfection | | | | |
| GS4-Fc-Opt- | | cells | | 37 C. | | | | |
| Q38) (The | | | | | | | | |
| LCB1 | | | | | | | | |
| sequence = | | | | | | | | |
| LCB1-3 of | | | | | | | | |
| first | | | | | | | | |
| provisional) | | | | | | | | |
| LCB1-Fc6 | (SEQ ID | Human | pCMVR | Transient | 5 | 0.2 | 0.1 | 0.01 |
| (BM40-LCB1- | NO: 73) | 293 | | Transfection | | | | |
| GS15-Fc-Opt- | | cells | | 37 C. | | | | |
| Q38) (The | | | | | | | | |
| LCB1 | | | | | | | | |
| sequence = | | | | | | | | |
| LCB1-3 of | | | | | | | | |
| first | | | | | | | | |
| provisional) | | | | | | | | |
| LCB1-Fc7 | (SEQ ID | Human | pCMVR | Transient | 10 | 0.2 | 0.1 | 0.01 |
| (BM40-Fc- | NO: 74) | 293 | | Transfection | | | | |
| Opt-GS15-2- | | cells | | 37 C. | | | | |
| LCB1-Q38) | | | | | | | | |
| (The LCB1 | | | | | | | | |
| sequence = | | | | | | | | |
| LCB1-3 of | | | | | | | | |
| first | | | | | | | | |
| provisional) | | | | | | | | |
| LCB1-Fc8 | (SEQ ID | Human | pCMVR | Transient | 1 | 0.2 | 0.1 | 0.01 |
| (BM40-LCB1- | NO: 75) | cells | | Transfection | | | | |
| Q38-GS15-Fc- | | 293 | | 37 C. | | | | |
| Opt-GS15-2- | | | | | | | | |
| LCB1- | | | | | | | | |
| 238) (The | | | | | | | | |
| LCB1 | | | | | | | | |
| sequences = | | | | | | | | |
| LCB1-3 of | | | | | | | | |
| first | | | | | | | | |
| provisional) | | | | | | | | |
| LCB1-6M-Fc9 | (SEQ ID | Human | pCMVR | Transient | 20 | 0.2 | 0.1 | 0.01 |
| (BM40-LCB1- | NO: 76) | 293 | | Transfection | | | | |
| 6M- | | cells | | 37 C. | | | | |
| 4N, 14K, 15T, | | | | | | | | |
| 18Q, 27Q, 38Q- | | | | | | | | |
| GS15-Fc-Opt) | | | | | | | | |
| LCB1-6M- | (SEQ ID | Human | pCMVR | Transient | 20 | 0.2 | 0.1 | 0.01 |
| Ng1y-Fc10 | NO: 77) | 293 | | Transfection | | | | |
| (BM40-LCB1- | | cells | | 37 C. | | | | |
| 6M-Ng1y- | | | | | | | | |
| 4N, 14K, 15T, | | | | | | | | |
| 18Q, 27N, 38Q- | | | | | | | | |
| GS15-Fc-Opt) | | | | | | | | |
| (The LCB1 | | | | | | | | |
| sequence = | | | | | | | | |
| LCB1-5 of | | | | | | | | |
| the original | | | | | | | | |
| provisional = | | | | | | | | |
| LCB1_v1.1 = | | | | | | | | |
| LCB1-4 | | | | | | | | |
| with N-link | | | | | | | | |
| Glycosylation) | | | | | | | | |
| LCB3-6M-Fc11 | (SEQ ID | Human | pCMVR | Transient | 5 | 0.2 | 0.1 | 0.01 |
| (BM40-LCB3- | NO: 78) | 293 | | Transfection | | | | |
| 6M | | cells | | 37 C. | | | | |
| 8Q, 26Q, 28H, | | | | | | | | |
| 35K, 37T, 43K- | | | | | | | | |
| GS15-Fc-Opt) | | | | | | | | |
| (LCB | | | | | | | | |
| sequence is | | | | | | | | |
| the same as | | | | | | | | |
| LCB3-3 of | | | | | | | | |
| First | | | | | | | | |
| Provisional) | | | | | | | | |
| LCB3-6M- | (SEQ ID | Human | pCMVR | Transient | 5 | 0.2 | 0.1 | 0.01 |
| NG1y-Fc12 | NO: 79) | 293 | | Transfection | | | | |
| (BM40-LCB3- | | cells | | 37 C. | | | | |
| 6M-Ng1y- | | | | | | | | |
| 8Q, 26Q, 28H, | | | | | | | | |
| 35N, 37T, 43K- | | | | | | | | |
| GS15-Fc-Opt) | | | | | | | | |
| (LCB | | | | | | | | |
| sequence is | | | | | | | | |
| the same as | | | | | | | | |
| LCB3-4 of | | | | | | | | |
| First | | | | | | | | |
| Provisional | | | | | | | | |
| which is | | | | | | | | |
| LCB3-3 with | | | | | | | | |
| N-link | | | | | | | | |
| Glycosylation) | | | | | | | | |
| LCB1-6M- | (SEQ ID | Human | pCMVR | Transient | 2 | 0.2 | 0.1 | 0.01 |
| GPGcP-Fc13 | NO: 80) | cells | | Transfection | | | | |
| (BM40-LCB1- | | 293 | | 37 C. | | | | |
| 6M- | | | | | | | | |
| 4N, 14K, 15T, | | | | | | | | |
| 18Q, 27Q, 38Q- | | | | | | | | |
| GPGcP-Fc- | | | | | | | | |
| Opt) (LCB | | | | | | | | |
| sequence is | | | | | | | | |
| the same as | | | | | | | | |
| LCB3-3 of | | | | | | | | |
| First | | | | | | | | |
| Provisional) | | | | | | | | |
| LCB3-6M- | (SEQ ID | Human | pCMVR | Transient | 2 | 0.2 | 0.1 | 0.01 |
| GPGcP-Fc14 | NO: 81) | 293 | | Transfection | | | | |
| BM40-LCB3- | | cells | | 37 C. | | | | |
| 6M- | | | | | | | | |
| 8Q, 26Q, 28H, | | | | | | | | |
| 35K, 37T, 43K- | | | | | | | | |
| GPGcP-Fc- | | | | | | | | |
| Opt) (LCB | | | | | | | | |
| sequence is | | | | | | | | |
| the same as | | | | | | | | |
| LCB3-3 of | | | | | | | | |
| First | | | | | | | | |
| Provisional) | | | | | | | | |
| LCB1-6M- | (SEQ ID | Human | pCMVR | Transient | 1 | 0.2 | 0.1 | 0.01 |
| (BM40-LCB1- | NO: 82) | 293 | | Transfection | | | | |
| 6M- | | cells | | 37 C. | | | | |
| 4N, 14K, 15T, | | | | | | | | |
| 18Q, 27Q, 38Q- | | | | | | | | |
| GS30-Fc- | | | | | | | | |
| Opt) LCB1-4 | | | | | | | | |
| GS30-Fc15 | | | | | | | | |
| LCB3-6M- | (SEQ ID | Human | pCMVR | 37 C. | 1 | 0.2 | 0.1 | 0.01 |
| GS30-Fc16 | NO: 83) | 293 | | Transient | | | | |
| (BM40-LCB3- | | cells | | Transfection | | | | |
| 6M- | | | | | | | | |
| 8Q, 26Q, 28H, | | | | | | | | |
| 35K, 37T, 43K | | | | | | | | |
| GS30-Fc-Opt) | | | | | | | | |
| (LCB | | | | | | | | |
| sequence is | | | | | | | | |
| the same as | | | | | | | | |
| LCB3-3 of | | | | | | | | |
| First | | | | | | | | |
| Provisional) | | | | | | | | |
| LCB1-v1.3- | (SEQ ID | Human | pCMVR | Transient | 5 | 0.2 | 0.1 | 0.01 |
| Fc17 (BM40- | NO: 84) | 293 | | Transfection | | | | |
| LCB1-v1.3- | | cells | | 37 C. | | | | |
| 4N, 14K, 15T, | | | | | | | | |
| 17E, 18Q, 27Q, | | | | | | | | |
| 38Q-GS15-Fc- | | | | | | | | |
| Opt) | | | | | | | | |
| LCB1-v1.3- | (SEQ ID | Human | pCMVR | Transient | 5 | 0.2 | 0.1 | 0.01 |
| Ng1y-Fc18 | NO: 85) | 293 | | Transfection | | | | |
| (BM40-LCB1- | | cells | | 37 C. | | | | |
| v1.3 Ng1y- | | | | | | | | |
| 4N, 14K, 15T, | | | | | | | | |
| 17E, 18Q, 27N, | | | | | | | | |
| 38Q-GS15-Fc- | | | | | | | | |
| Opt) | | | | | | | | |
| (>LCB1_v1.5 | | | | | | | | |
| (=LCB1_v1.3 | | | | | | | | |
| with N-link | | | | | | | | |
| Glycosylation) | | | | | | | | |
| LCB1-v1.3- | (SEQ ID | Human | pCMVR | Transient | 5 | 0.2 | 0.1 | 0.01 |
| GPGcP-Fc19 | NO: 86) | 293 | | Transfection | | | | |
| (BM40-LCB1- | | cells | | 37 C. | | | | |
| v1.3-Fc19 | | | | | | | | |
| 4N, 14K, 15T, | | | | | | | | |
| 17E, 18Q, 27Q, | | | | | | | | |
| 38Q-GPGcP-Fc- | | | | | | | | |
| Opt) | | | | | | | | |
| LCB1-v1.3- | (SEQ ID | Human | pCMVR | Transient | 5 | 0.2 | 0.1 | 0.01 |
| GS30-Fc20 | NO: 87) | 293 | | Transfection | | | | |
| (BM40-LCB1- | | cells | | 37 C. | | | | |
| v1.3- | | | | | | | | |
| 4N, 14K, 15T, | | | | | | | | |
| 17E, 18Q, 27Q, | | | | | | | | |
| 38Q-GS30-Fc- | | | | | | | | |
| Opt) | | | | | | | | |

The designed binders have several advantages over antibodies as potential therapeutics. Together, they span a range of binding modes, and in combination viral escape would be quite unlikely. The retention of activity after extended time at elevated temperatures suggests they would not require a cold chain. The designs are 20 fold smaller than a full antibody molecule, and hence in an equal mass have 20 fold more potential neutralizing sites, increasing the potential efficacy of a locally administered drug. The cost of goods and the ability to scale to very high production should be lower for the much simpler miniproteins, which unlike antibodies, do not require expression in mammalian cells for proper folding. The small size and high stability should make them amenable to direct delivery into the respiratory system by nebulization. Immunogenicity is a potential problem with any foreign molecule, but for previously characterized small de novo designed proteins little or no immune response has been observed, perhaps because the high solubility and stability together with the small size makes presentation on dendritic cells less likely.

### REFERENCES

1. Yuan M, Wu NC, Zhu X, Lee C D, So RTY, Lv H, Mok CKP, Wilson IA: A highly conserved cryptic epitope in the receptor binding domains of SARS-CoV-2 and SARS-COV. Science 2020, 368(6491):630-633.
2. Case J B, Rothlauf P W, Chen R E, Liu Z, Zhao H, Kim AS, Bloyet L-M, Zeng Q, Tahan S, Droit L et al: Neutralizing antibody and soluble ACE2 inhibition of a replication-competent VSV-SARS-COV-2 and a clinical isolate of SARS-COV-2. bioRxiv 2020:2020.2005.2018.102038.

Ultrapotent Miniproteins Targeting the Receptor-Binding Domain Protect Against SARS-CoV-2 Infection and Disease

Despite the introduction of public health measures and spike protein-based vaccines to mitigate the COVID-19 pandemic, SARS-COV-2 infections and deaths continue to rise. Here, we investigated the capacity of modified versions of one lead binder, LCB1, to protect against SARS-COV-2-mediated lung disease in human ACE2-expressing transgenic mice. Systemic administration of LCB1-Fc reduced viral burden, diminished immune cell infiltration and inflammation, and completely prevented lung disease and pathology. A single intranasal dose of LCB1v1.3 reduced SARS-COV-2 infection in the lung even when given as many as five days before or two days after virus inoculation. Importantly, LCB1v1.3 protected in vivo against a historical strain (WA1/2020), an emerging B.1.1.7 strain, and a strain encoding key E484K and N501Y spike protein substitutions. These data support the use of LCB1v1.3 for prevention or treatment of SARS-COV-2 infection.

Severe Acute Respiratory Syndrome Coronavirus 2 (SARS-COV-2), the cause of the Coronavirus Disease 2019 (COVID-19) pandemic, has resulted in global disease, suffering, and economic hardship. Despite implementation of public health measures, SARS-COV-2 transmission persists principally through human-to-human spread (Day, 2020; Li et al., 2020; Stand1 et al., 2020). SARS-COV-2-induced clinical manifestations range from asymptomatic infection to severe pneumonia, multi-organ failure, and death. Although the underlying mechanisms that dictate disease severity are poorly understood, the immunocompromised, the elderly, and those with specific comorbidities (e.g., history of cardiovascular disease, diabetes, or obesity) are at increased risk for poor outcome (Zhou et al., 2020).

Here, using a stringent model of SARS-COV-2 disease pathogenesis in human ACE2 (hACE2)-expressing transgenic mice (Golden et al., 2020; Winkler et al., 2020a), we evaluated the efficacy in vivo of exemplary miniprotein binder, LCB1. For our in vivo experiments, we evaluated two versions of LCB1: (a) an Fc-modified bivalent form, LCB1-hIgG-Fc9 (LCB1-Fc) that should extend half-life in vivo and engage effector arms of the immune system; and (b) a further optimized, monomeric form of LCB1 lacking an Fc domain, LCB1v1.3. Intraperitoneal administration of LCB1-Fc at one day pre- or post SARS-CoV-2 exposure conferred substantial protection including an absence of weight loss, reductions in viral burden approaching the limit of detection, and inhibition of lung inflammation and pathology. Intranasal delivery of LCB1v1.3 conferred protection as many as five days before or two days after SARS-COV-2 inoculation. Dosing experiments revealed that LCB1v1.3 retained efficacy at pharmacologically attainable concentrations and was weakly immunogenic. Most importantly, LCB1v1.3 protected animals against the currently emerging B.1.1.7 United Kingdom variant and a SARS-COV-2 strain encoding key spike substitutions E484K and N501Y present in both the South Africa (B.1.351) and Brazil (B.1.1.248) variants of concern. Overall, these studies establish LCB1-Fc and LCB1v1.3 as possible treatments to prevent or mitigate SARS-COV-2 disease.

Results

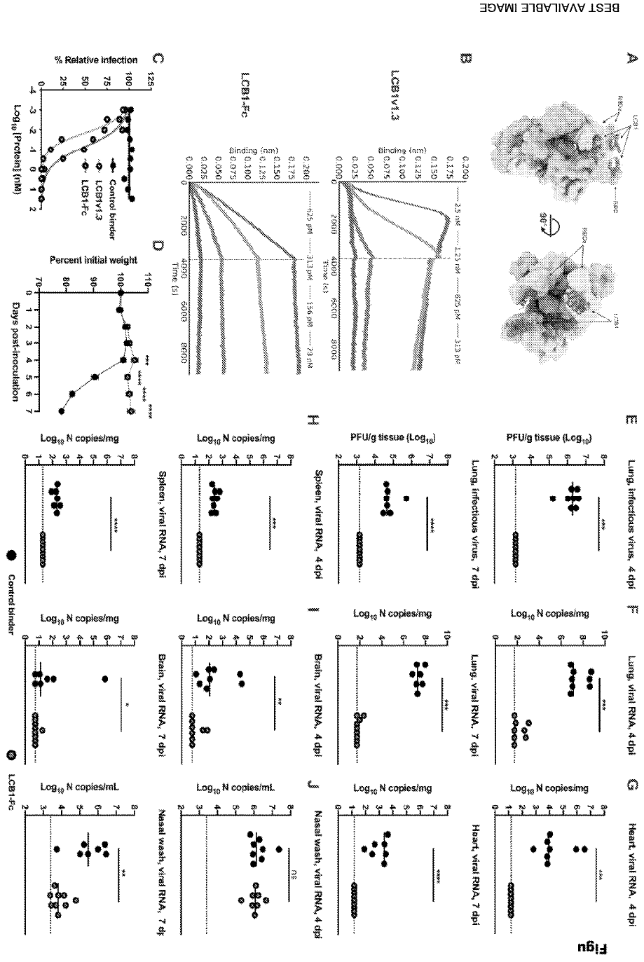

LCB1v1.3 prophylaxis limits viral burden and clinical disease. We modified LCB1 to generate two versions for in vivo testing: (a) we introduced polar mutations into LCB1 to increase expression yield and solubility without altering RBD binding (LCB1v1.3) and (b) we modified LCB1 by fusing it to a human IgG1 Fc domain (LCB1-Fc) to enhance bioavailability. LCB1v1.3 and LCB1-Fc bound avidly to a single RBD within the S trimer (FIG. 5A) with dissociation constants (KD) of less than 625 and 156 pM, respectively (FIG. 5B). LCB1v1.3 and LCB1-Fc also potently neutralized an authentic SARS-COV-2 isolate (2019n-CoV/USA_WA1/2020 [WA1/2020]) (EC₅₀ of 14.4 and 71.8 pM, respectively; FIG. 5C).

To determine the protective potential of these miniproteins against SARS-COV-2, we utilized K18 human hACE2-expressing transgenic mice, which develop severe lung infection and disease after intranasal inoculation of SARS-COV-2 (Golden et al., 2020; Winkler et al., 2020a). In prophylaxis studies, a single 250 μg (10 mg/kg) dose of LCB1-Fc administered by intraperitoneal injection (i.p.) one day prior to intranasal (i.n.) inoculation with 10³ PFU of SARS-COV-2 WA1/2020 prevented weight loss compared to animals given a control protein (influenza A virus hemagglutinin minibinder) designed using similar computational methods (FIG. 5D). After LCB1-Fc prophylaxis, infectious virus was not detected in the lungs at 4- or 7-days post-infection (dpi), whereas high levels were observed in animals administered control protein (FIG. 5E, top and bottom). Similarly, viral RNA levels in the lung, heart, spleen, and brain of LCB1-Fc treated animals were at or near the limit of detection of the assay at 4 or 7 dpi (FIG. 5F-I). LCB1-Fc treatment had no effect on viral RNA levels in nasal wash samples obtained at 4 dpi (FIG. 5J), results that are similar to a recent study of a neutralizing human antibody in hamsters (Zhou et al., 2021). However, viral RNA levels were reduced at 7 dpi, suggesting that LCB1-Fc treatment accelerated viral clearance or prevented spread in the upper respiratory tract.

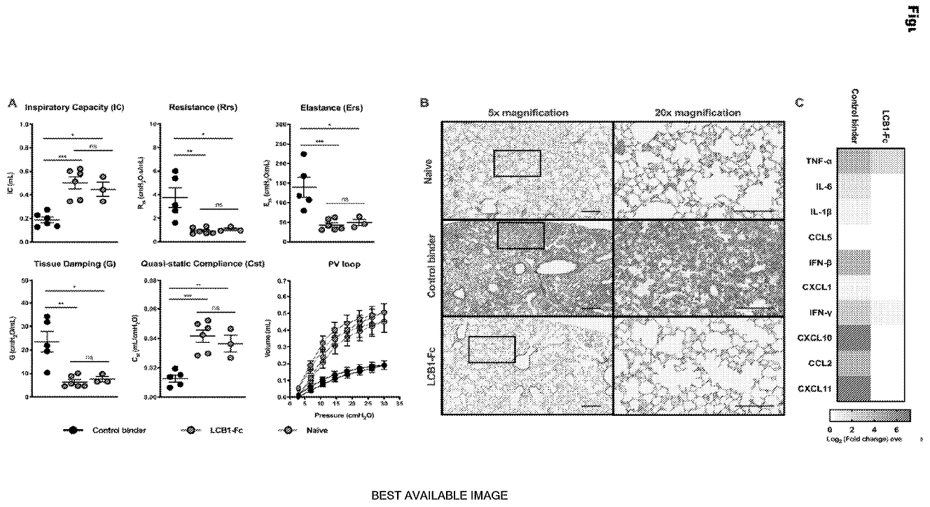

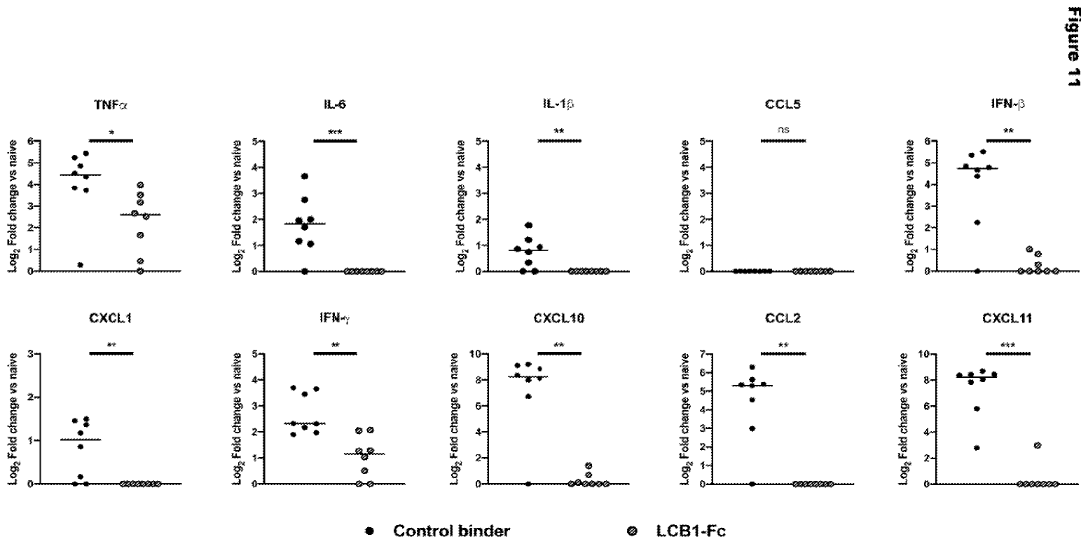

Diffuse alveolar damage, inflammation, and pneumonia are manifestations of COVID-19 lung disease, culminating in respiratory failure and a requirement for mechanical ventilation (Johnson et al., 2020; Kordzadeh-Kermani et al., 2020). We evaluated the capacity of LCB1-Fc to prevent the compromised lung function seen after SARS-COV-2 infection of K18-hACE2 mice (Winkler et al., 2020a). At 7 dpi, mechanical ventilation tests of lung biomechanics in animals treated with LCB1-Fc showed no difference from naïve animals (FIG. 6A), whereas mice receiving the control binder protein showed decreased inspiratory capacity and lung compliance as well as increased pulmonary resistance, elastance, and tissue damping, all consistent with compromised lung function. These biophysical properties resulted in disparate pressure-volume loops between control binder and LCB1-Fc treated or naïve animals. We also assessed the effect of LCB1-Fc treatment on SARS-COV-2-induced lung pathology. Lung sections of animals collected at 7 dpi with SARS-COV-2 showed widespread inflammation characterized by a cellular infiltrate and airspace consolidation in control protein-treated but not LCB1-Fc treated or naïve mice (FIG. 6B). At 4 dpi, inflammatory cytokine and chemokine RNA signatures in the lung were absent in LCB1-Fc treated but not control binder treated animals, suggesting that LCB1-Fc treatment prevents virus infection and inflammation in the lung (FIGS. 6C and 11).

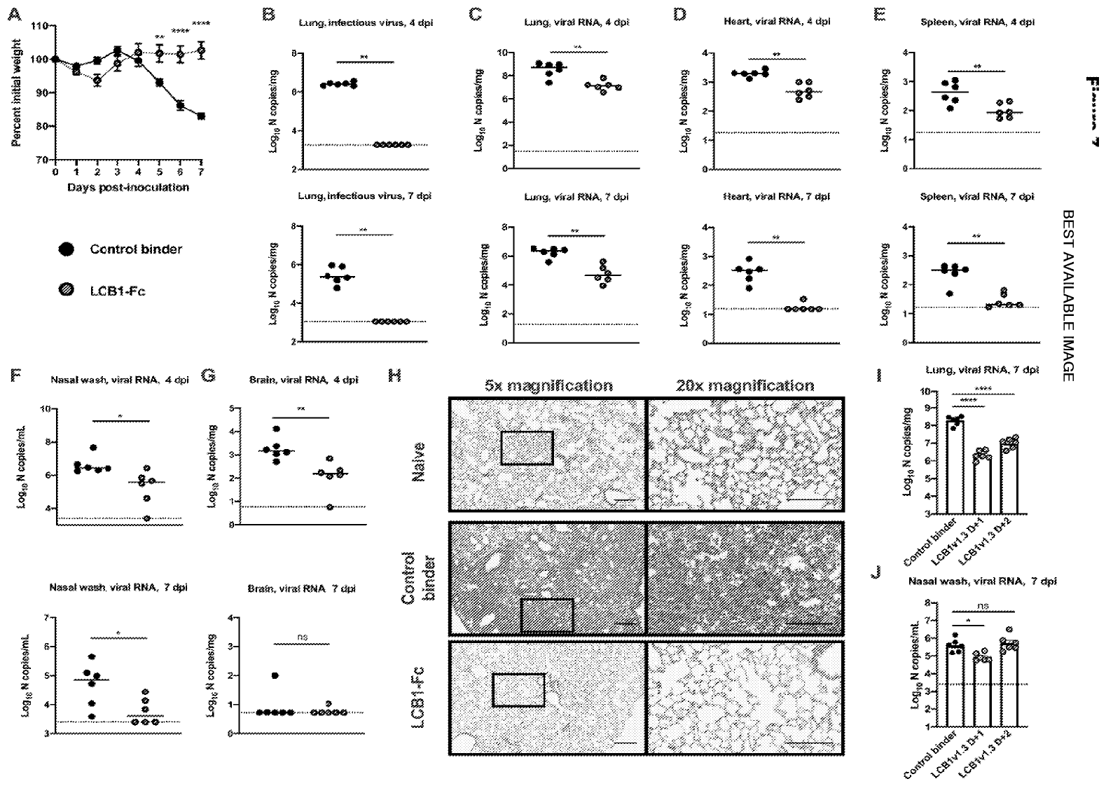

Post-exposure therapy with anti-RBD binders reduces viral burden. To evaluate its efficacy in a post-exposure setting, we administered LCB1-Fc by i.p. injection at 1 dpi. Therapy with LCB1-Fc prevented weight loss (FIG. 7A) and reduced viral burden in all tested tissues at 4 and 7 dpi (FIG. 7B-G). Infectious virus was not recovered from the lungs of LCB1-Fc treated animals collected at either timepoint. Lung sections confirmed that therapy with LCB1-Fc improved pathological outcome (FIG. 7H). At 7 dpi, immune cell infiltrates were absent in the lung sections of LCB1-Fc treated but not control binder-treated animals.

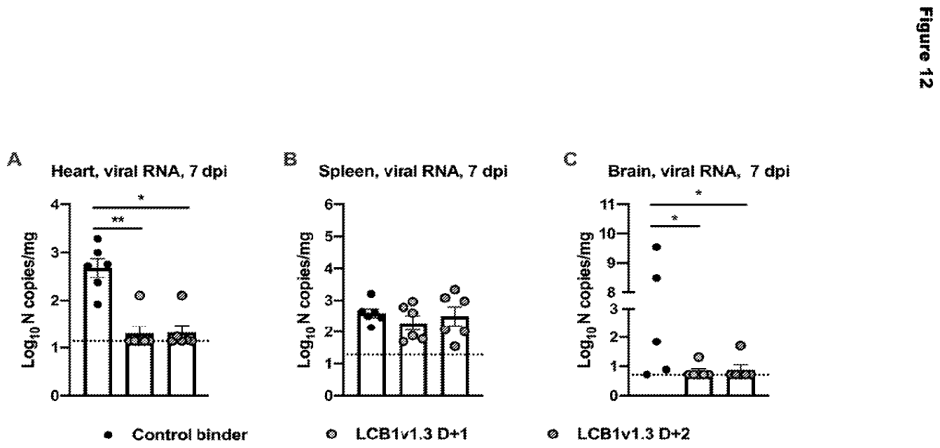

We next tested the efficacy of LCB1v1.3 as an i.n.-delivered post-exposure therapy. I.n. delivery, might enable self-administration of an anti-SARS-CoV-2 biological drug. Indeed, miniprotein inhibitors against influenza virus have shown efficacy as a nasal mist (Chevalier et al., 2017). For these studies, we used LCB1v1.3 because it can bind an increased number of RBD molecules for a given mass dose, resulting in increased neutralization activity (FIG. 5C). Whereas high levels of SARS-COV-2 RNA were detected in the lungs and other peripheral tissues of control binder-treated animals at 7 dpi, infection was reduced in animals receiving LCB 1v1.3 by i.n. administration at D+1 or D+2 after inoculation with SARS-COV-2 (FIGS. 7I and 12). Levels of viral RNA were reduced in the nasal washes of animals receiving LCB1v1.3 after treatment at D+1 but not D+2 compared to control binder-treated animals (FIG. 7J).

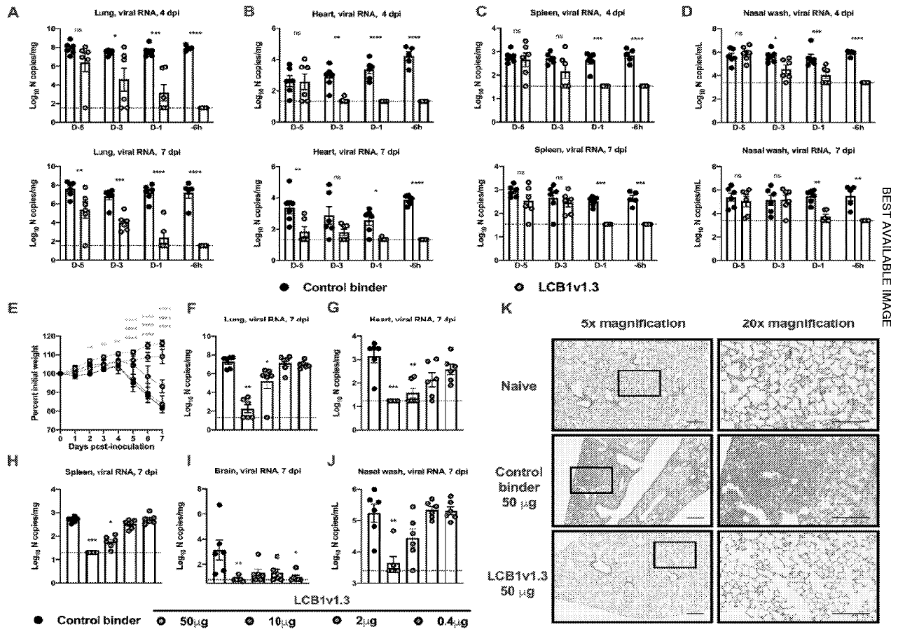

Intranasal delivery of LCB1v1.3 confers protection against SARS-CoV-2 when administered up to 5 days before infection. We next evaluated the durability of LCB1v1.3 administered via i.n. prophylaxis. At 5 days, 3 days, 1 day, or 6 hours prior to inoculation with 10³ PFU of SARS-COV-2, K18-hACE2 transgenic mice received a single 50 μg i.n. dose of LCB1v1.3 or the control binder. At 4 or 7 dpi, viral burden in tissues was determined by RT-qPCR. As expected, protection by LCB1v1.3 was better when administered closer to the time of SARS-COV-2 exposure, as reflected by greater reductions in viral load and weight loss (FIG. 8A-D and S3). However, even mice receiving LCB1v1.3 five days prior to inoculation and collected at 7 dpi showed reduced viral RNA levels in the lung compared to control binder treated animals. Regardless of the collection timepoint, lung viral RNA levels were reduced in animals receiving LCB1v1.3 three days prior to inoculation with SARS-CoV-2.

We tested a range of i.n. doses LCB1v1.3 for efficacy (FIG. 8E-J). Treatment with as little as 2 μg (0.1 mg/kg) of LCB1v1.3 prevented SARS-COV-2-induced weight loss. Doses between 2 and 10 μg (0.1 to 0.5 mg/kg) of LCB1v1.3 reduced viral RNA levels in the lung, heart, and spleen at 7 dpi relative to control binder-treated animals. Moreover, animals receiving a 50 μg dose of LCB1v1.3 showed minimal, if any, lung inflammation (FIG. 8K). Collectively, these results indicate that even low doses of LCB1v1.3, when administered via an i.n. route prior to exposure, can limit SARS-COV-2 infection and disease in the stringent K18-hACE transgenic mouse model of pathogenesis.

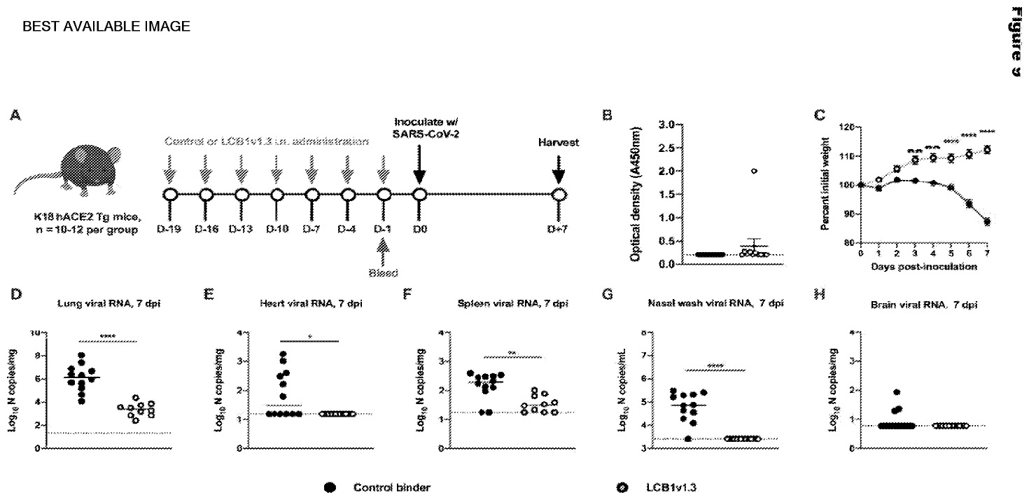

LCB1v1.3 is weakly immunogenic and retains protective activity after repeated dosing. We treated K18-hACE2 transgenic mice with 50 μg of control binder or LCB1v1.3 every three days for a total of 18 days (FIG. 9A). At this time, we collected sera and assessed the presence of anti-LCB1v1.3 antibodies. Only 1 of 10 mice developed IgG antibodies against LCB1v1.3 (FIG. 9B). To determine if repeated dosing affected LCB1v1.3-mediated protection, we challenged the cohort with 10³ PFU of SARS-COV-2. Again, substantial protection against weight loss (FIG. 9C) and viral infection in the lung and other organs was observed in all animals receiving LCB1v1.3 (FIG. 9D-H).

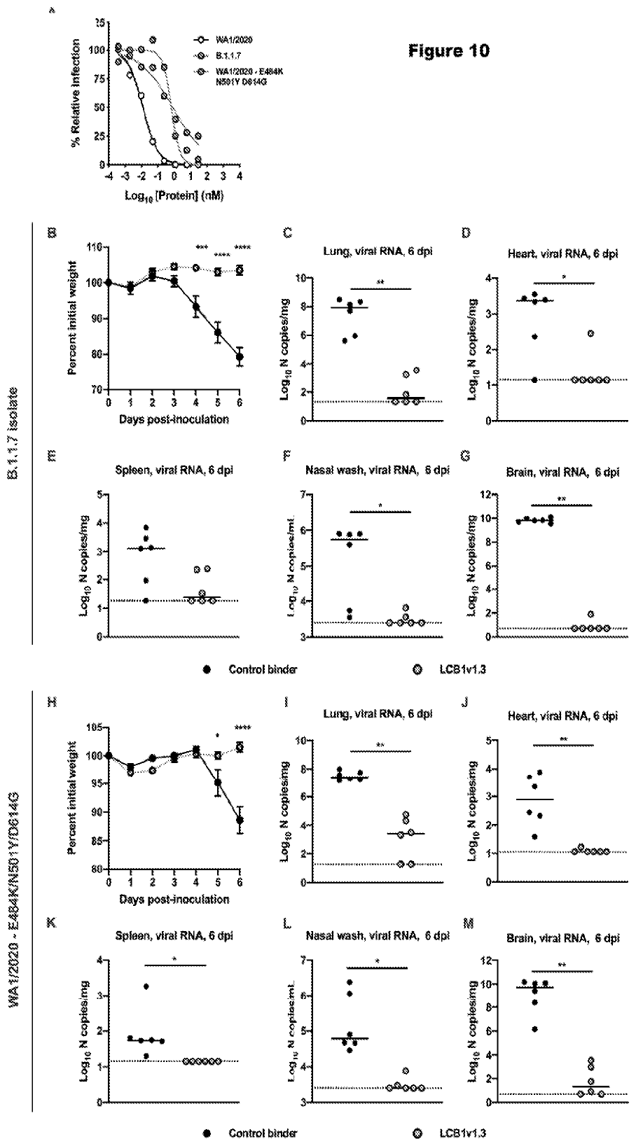

LCB1v1.3 protects against emerging SARS-COV-2 variants. We evaluated the activity of LCB1v1.3 against a B.1.1.7 isolate containing deletions at 69-70 and 144-145, and substitutions at N501Y, A570D, D614G, and P681H, and against a recombinant WA1/2020 strain containing key substitutions present in the B.1.351 and B.1.248 variant strains at residues E484K, N501Y, and D614G (Xie et al., 2021a). Although the neutralizing activity of LCB1v1.3 against the B.1.1.7 and E484K/N501Y/D614G strains was approximately 45 to 50-fold lower than for the WA1/2020 strain, the EC₅₀ values still were ˜800 pM and 667 pM, respectively (FIG. 10A). To determine whether LCB1v1.3 could protect in vivo against SARS-CoV-2 strains with concerning spike protein substitutions, we treated K18-hACE2 transgenic mice with a single i.n. 50 μg dose of LCB1v1.3 or control binder one day prior to inoculation with 10³ PFU of B.1.1.7 or E484K/N501/D614G SARS-COV-2. Notably, LCB1v1.3 treatment before challenge with either variant strain protected against weight loss (FIGS. 10B and 10H) and viral infection in all tissues collected at 6 dpi (FIGS. 10C-G and 101-M). Thus, LCB1v1.3 is effective against both circulating and emerging strains of SARS-COV-2.

### DISCUSSION

Here, using the stringent K18-hACE2 mouse model of SARS-COV-2 pathogenesis, we show that LCB1-Fc prevented SARS-COV-2 infection and disease when administered one day before or after virus inoculation. Lung biomechanics of mice treated with LCB1-Fc mirrored those of naïve animals in all parameters tested.

We also evaluated the efficacy of LCB1v1.3, an optimized, monomeric form of LCB1 without an Fc domain. A single i.n. dose of LCB1v1.3 reduced viral burden when administered as many as five days before or two days after SARS-COV-2 infection. Our i.n. delivery approach is unique. I.n. therapy of SARS-COV-2 has been reported only with type I interferon in a hamster model of disease (Hoagland et al., 2021) and efficacy was limited. The K18-hACE2 mouse model recapitulates several aspects of severe COVID-19, including lung inflammation and reduced pulmonary function (Golden et al., 2020; Winkler et al., 2020a). Since K18-hACE2 mice are highly vulnerable to infection, the therapeutic window of treatment is limited (Winkler et al., 2020b) and for our miniproteins, might only curb viral infection. Importantly, our data demonstrate that LCB1v1.3 binder treatment before or after infection limited immune cell infiltration and lung inflammation, which prevented tissue damage and compromise of respiratory function. As part of our proof-of-principle studies for a nasal prophylaxis, we observed little immunogenicity of LCB1v1.3, suggesting that repeated dosing may be possible.

Although several antibody-based therapies demonstrate promise against SARS-COV-2, and a few have been granted EUA status, viral evolution could jeopardize these interventions as evidenced by the emerging variants in the United Kingdom (B.1.1.7), South Africa (B.1.351), Brazil (B.1.248), and elsewhere. Indeed, we and others have observed that many monoclonal and polyclonal antibodies showed reduced neutralization activity against several of these variant strains (Chen et al., 2021; Wang et al., 2021a; Wang et al., 2021b; Wibmer et al., 2021; Xie et al., 2021b). In comparison, LCB1v1.3 showed efficacy against historical (WA1/2020) and emerging (B.1.1.7 and E484K/N501Y/D614G) SARS-COV-2 strains. Based on the cryo-EM structure of the parent LCB1 binder in complex with SARS-CoV-2 RBD (Cao et al., 2020), only the N501Y mutation is expected to affect binding. While we observed a decrease in the neutralizing activity of LCB1v1.3 against the emerging variants, EC 50 values were still less than 800 pM, suggesting substantial potency was retained.

Compared to other potential SARS-COV-2 antibody-based treatments, miniproteins have several benefits: (a) due to their smaller size, they can bind each protomer of a single trimeric spike, resulting in greater potency for a given dose; (b) they can be manufactured cost-effectively; and (c) they can be mixed using linker proteins to generate multimerized constructs that limit resistance.

Experimental Model and Subject Details

Cells and viruses. Vero E6 (CRL-1586, American Type Culture Collection (ATCC), Vero CCL81 (ATCC), Vero-furin (Mukherjee et al., 2016), and Vero-hACE2-TMPRSS2 (a gift of A. Creanga and B. Graham, NIH) were cultured at 37° C. in Dulbecco's Modified Eagle medium (DMEM) supplemented with 10% fetal bovine serum (FBS), 10 mM HEPES pH 7.3, 1 mM sodium pyruvate, 1× non-essential amino acids, and 100 U/ml of penicillin-streptomycin. Additionally, Vero-hACE2-TMPRSS2 cells were cultured in the presence of 5 μg/mL puromycin. The WA1/202 (2019n-CoV/USA_WA1/2020) isolate of SARS-COV-2 was obtained from the US Centers for Disease Control (CDC). The B.1.1.7 and WA1/2020 E484K/N501Y/D614G viruses have been described previously (Chen et al., 2021; Xie et al., 2021a). Infectious stocks were propagated by inoculating Vero CCL81 or Vero-hACE2-TMPRSS2 cells. Supernatant was collected, aliquoted, and stored at −80° C. All work with infectious SARS-COV-2 was performed in Institutional Biosafety Committee-approved BSL3 and A-BSL3 facilities at Washington University School of Medicine using positive pressure air respirators and protective equipment.

Mouse experiments. Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01).

Virus inoculations were performed under anesthesia that was induced and maintained with ketamine hydrochloride and xylazine, and all efforts were made to minimize animal suffering.

Heterozygous K18-hACE c57BL/6J mice (strain: 2B6·Cg-Tg(K18-ACE2)₂Prlmn/J) were obtained from The Jackson Laboratory. Animals were housed in groups and fed standard chow diets. Mice of different ages and both sexes were administered 10³ PFU of SARS-COV-2 via intranasal administration.

### Method Details

Miniprotein production. LCB1-Fc was synthesized and cloned by GenScript into pCMVR plasmid, with kanamycin resistance. Plasmids were transformed into the NEB 5-alpha strain of E. coli (New England Biolabs) to recover DNA for transient transfection into Expi293F mammalian cells. Expi293F cells were grown in suspension using Expi293F expression medium (Life Technologies) at 33° C., 70% humidity, and 8% CO2 rotating at 150 rpm. The cultures were transfected using PEI-MAX (Polyscience) with cells grown to a density of 3×106 cells per mL and cultivated for 3 days. Supernatants were clarified by centrifugation (5 min at 4000×g, addition of PDADMAC solution to a final concentration of 0.0375% (Sigma Aldrich, #409014), and a second spin (5 min at 4000×g). Clarified supernatants were purified using a MabSelect PrismA™ 2.6×5 cm column (Cytiva) on an AKTA Avant150 FPLC (Cytiva). Bound protein was washed with five column volumes of 20 mM NaPO₄ and 150 mM NaCl pH 7.2, then five column volumes of 20 mM NaPO₄ and 1 M NaCl pH 7.4, and eluted with three column volumes of 100 mM glycine at pH 3.0. The eluate was neutralized with 2 M Tris base to a final concentration of 50 mM. SDS-PAGE was used to assess protein purity. The protein was passed through a 0.22 μm filter and stored at 4° C. until use.

LCB1v1.3 with polar mutations (4N, 14K, 15T, 17E, 18Q, 27Q, 38Q) relative to the original LCB1 was cloned into a pet29b vector. LCB1v1.3 was expressed in Lemo21(DE3) (NEB) in terrific broth media and grown in 2 L baffled shake flasks. Bacteria were propagated at 37° C. to an O.D.600 of ˜0.8, and then induced with 1 mM IPTG. Expression temperature was reduced to 18° C., and the cells were shaken for ˜16 h. The cells were harvested and lysed using heat treatment and incubated at 80° C. for 10 min with stirring. Lysates were clarified by centrifugation at 24,000×g for 30 min and applied to a 2.6×10 cm Ni Sepharose™ 6 FF column (Cytiva) for purification by IMAC on an AKTA Avant150 FPLC system (Cytiva). Proteins were eluted over a linear gradient of 30 mM to 500 mM imidazole in a buffer of 50 mM Tris pH 8.0 and 500 mM NaCl. Peak fractions were pooled, concentrated in 10 kDa MWCO centrifugal filters (Millipore), sterile filtered (0.22 μm) and applied to either a Superdex™ 200 Increase 10/300, or HiLoad S200 pg GL SEC column (Cytiva) using 50 mM phosphate pH 7.4, 150 mM NaCl buffer. After size exclusion chromatography, bacterial-derived components were tested to confirm low levels of endotoxin.

Biolayer interferometry. Biolayer interferometry data were collected using an Octet™ RED96 (ForteBio) and processed using the instrument's integrated software. Briefly, biotinylated RBD (Acro Biosystems) was loaded onto streptavidin-coated biosensors (SA ForteBio) at 20 nM in binding buffer (10 mM HEPES (pH 7.4), 150 mM NaCl, 3 mM EDTA, 0.05% surfactant P20, and 0.5% non-fat dry milk) for 360 s. Analyte proteins (LCB1v1.3 or LCB1-Fc) were diluted from concentrated stocks into binding buffer. After baseline measurement in the binding buffer alone, the binding kinetics were monitored by dipping the biosensors in wells containing the target protein at the indicated concentration (association step) for 3,600 s and then dipping the sensors back into baseline/buffer (dissociation) for 7,200 s.

Plaque assay. Vero-furin cells (Mukherjee et al., 2016) were seeded at a density of 2.5×105 cells per well in flat-bottom 12-well tissue culture plates. The following day, medium was removed and replaced with 200 μL of 10-fold serial dilutions of the material to be titrated, diluted in DMEM+2% FBS, and plates incubated at 37° C. with rocking at regular intervals. One hour later, 1 mL of methylcellulose overlay was added. Plates were incubated at 37° C. for 72 h, then fixed with 4% paraformaldehyde (final concentration) in PBS for 20 min. Fixed cell monolayers were stained with 0.05% (w/v) crystal violet in 20% methanol and washed twice with distilled, deionized water.

Measurement of viral burden. Tissues were weighed and homogenized with zirconia beads in a MagNA Lyser™ instrument (Roche Life Science) in 1,000 μL of DMEM media supplemented with 2% heat-inactivated FBS. Tissue homogenates were clarified by centrifugation at 10,000 rpm for 5 min and stored at −80° C. RNA was extracted using the MagMax mirVana™ Total RNA isolation kit (Thermo Scientific) on a Kingfisher Flex extraction robot (Thermo Scientific). RNA was reverse transcribed and amplified using the TaqMan™ RNA-to-CT 1-Step Kit (ThermoFisher). Reverse transcription was carried out at 48° C. for 15 min followed by 2 min at 95° C. Amplification was accomplished over 50 cycles as follows: 95° C. for 15 s and 60° C. for 1 min. Copies of SARS-COV-2 N gene RNA in samples were determined using a previously published assay (Case et al., 2020; Hassan et al., 2020). Briefly, a TaqMan™ assay was designed to target a highly conserved region of the N gene (Forward primer: ATGCTGCAATCGTGCTACAA (SEQ ID NO: 190); Reverse primer: GACTGCCGCCTCTGCTC (SEQ ID NO: 191); Probe: /56-FAM/TCAAGGAAC/ZEN/AACATTGCCAA/3IABKFQ/) (SEQ ID NO: 192). This region was included in an RNA standard to allow for copy number determination down to 10 copies per reaction. The reaction mixture contained final concentrations of primers and probe of 500 and 100 nM, respectively.

Cytokine and chemokine mRNA measurements. RNA was isolated from lung homogenates as described above. cDNA was synthesized from DNAse-treated RNA using the High-Capacity cDNA Reverse Transcription kit (Thermo Scientific) with the addition of RNase inhibitor following the manufacturer's protocol. Cytokine and chemokine expression was determined using TaqMan™ Fast Universal PCR master mix (Thermo Scientific) with commercial primers/probe sets specific for IFN-g (IDT: Mm.PT.58.41769240), IL-6 (Mm.PT.58.10005566), IL-1b (Mm.PT.58.41616450), Tnfa (Mm.PT.58.12575861), CXCL10 (Mm.PT.58.43575827), CCL2 (Mm.PT.58.42151692), CCL5 (Mm.PT.58.43548565), CXCL11(Mm.PT.58.10773148.g), Ifnb (Mm.PT.58.30132453.g), CXCLI (Mm.PT.58.42076891) and results were normalized to GAPDH (Mm.PT.39a.1) levels. Fold change was determined using the 2^{-ΔΔCt} method comparing treated mice to naïve controls.

Lung Pathology. Animals were euthanized before harvest and fixation of tissues. The left lung was first tied off at the left main bronchus and collected for viral RNA analysis. The right lung was inflated with approximately 1.2 mL of 10% neutral buffered formalin using a 3-mL syringe and catheter inserted into the trachea. Tissues were embedded in paraffin, and sections were stained with hematoxylin and eosin. Slides were scanned using a Hamamatsu NanoZoomer™ slide scanning system, and images were viewed using NDP view software (ver.1.2.46).

Respiratory mechanics. Mice were anesthetized with ketamine/xylazine (100 mg/kg and 10 mg/kg, i.p., respectively). The trachea was isolated via dissection of the neck area and cannulated using an 18-gauge blunt metal cannula (typical resistance of 0.18 cmH₂O·s/mL), which was secured in place with a nylon suture. The mouse then was connected to the flexiVent™ computer-controlled piston ventilator (SCIREQ Inc.) via the cannula, which was attached to the FX adaptor Y-tubing. Mechanical ventilation was initiated, and mice were given an additional 100 mg/kg of ketamine and 0.1 mg/mouse of the paralytic pancuronium bromide via intraperitoneal route to prevent breathing against the ventilator and during measurements. Mice were ventilated using default settings for mice, which consisted in a positive end expiratory pressure at 3 cm H₂O, a 10 mL/kg tidal volume (Vt), a respiratory rate at 150 breaths per minute (bpm), and a fraction of inspired oxygen (FiO₂) of 0.21 (i.e., room air). Respiratory mechanics were assessed using the forced oscillation technique, as previously described (McGovern et al., 2013), using the latest version of the flexiVent™ operating software (flexiWare v8.1.3). Pressure-volume loops and measurements of inspiratory capacity also were performed.

Neutralization assay. Serial dilutions of binder proteins were incubated with 10² focus-forming units (FFU) of SARS-COV-2 for 1 h at 37° C. Binder-virus complexes were added to Vero E6 (WA1/2020) or Vero-hACE2-TMPRSS2 (B.1.1.7 and WA1/2020 E484K/N501Y/D614G) cell monolayers in 96-well plates and incubated at 37° C. for 1 h. Subsequently, cells were overlaid with 1% (w/v) methylcellulose in MEM supplemented with 2% FBS. Plates were harvested 24-30 h later by removing overlays and fixed with 4% PFA in PBS for 20 min at room temperature. Plates were washed and sequentially incubated with an oligoclonal pool of SARS2-2, SARS2-11, SARS2-16, SARS2-31, SARS2-38, SARS2-57, and SARS2-71 anti-spike protein antibodies (Zhou et al., 2021) and HRP-conjugated goat anti-mouse IgG in PBS supplemented with 0.1% saponin and 0.1% bovine serum albumin. SARS-COV-2-infected cell foci were visualized using TrueBlue™ peroxidase substrate (KPL) and quantitated on an ImmunoSpot™ microanalyzer (Cellular Technologies). Data were processed using Prism™ S software (GraphPad Prism™ 8.0).

ELISA. C-terminal biotinylated LCB1.1v3 was immobilized on streptavidin-coated plates (RayBiotech #7C-SCP-1) at 2.5 μg/mL in 100 μL total volume per well and incubated at 4° C. overnight. Plates were washed with wash buffer (TBS+0.1% (w/v) BSA+0.05% (v/v) Tween20) and blocked with 200 μL/well blocking buffer (TBS+2% (w/v) BSA+0.05% (v/v) Tween20) for 1 h at room temperature. Plates were rinsed with wash buffer using 200 μL/well, and 100 μL of 1:100 diluted sera samples in blocking buffer were added to respective wells. For a positive control, Fc-RBD was serially diluted 1:5 starting at 240 ng/ml in 100 μL of blocking buffer. All samples were incubated for 1 h at room temperature. Plates were washed using 200 μL/well of wash buffer. For the serum samples, HRP-conjugated horse anti-mouse IgG antibody (Vector Laboratories #PI-2000-1) was diluted 1:200 in blocking buffer, and 100 μL was incubated in each well at room temperature for 30 min. For the positive control, HRP-conjugated mouse anti-human IgG antibody (Invitrogen #05-4220) was diluted 1:500 in blocking buffer, and 100 μL was incubated in each well at room temperature for 30 min. Plates were rinsed with wash buffer, and 100 μL of TMB (SeraCare) was added to each well for 2 min. The reaction was quenched by adding 100 μL of 1N HCl. Optical densities were determined at 450 nm on a Synergy Neo21M plate reader (BioTek Instruments).

Quantification and Statistical Analysis

Statistical significance was assigned when P values were <0.05 using Prism™ Version 8 (GraphPad). Tests, number of animals, median values, and statistical comparison groups are indicated in each of the Figure legends. Analysis of weight change was determined by two-way ANOVA. Changes in functional parameters or immune parameters were compared to control binder-treated animals and analyzed by one-way ANOVA with multiple comparisons tests. Statistical analyses of viral burden between two groups were determined by Mann-Whitney test.

### REFERENCES

Cao, L. X., Goreshnik, I., Coventry, B., Case, J. B., Miller, L., Kozodoy, L., Chen, R. E., Carter, L., Walls, A. C., Park, Y. J., et al. (2020). De novo design of picomolar SARS-COV-2 miniprotein inhibitors. Science 370.
Case, J. B., Bailey, A. L., Kim, A. S., Chen, R. E., and Diamond, M. S. (2020). Growth, detection, quantification, and inactivation of SARS-COV-2. Virology 548, 39-48. Chen, R., Zhang, X., Case, J. B., Winkler, E., Liu, Y., Vanblargan, L., Liu, J., Errico, J., Xie, X., Suryadevara, N., et al. (2021). Resistance of SARS-COV-2 variants to neutralization by monoclonal and serum-derived polyclonal antibodies (Research Square).
Chevalier, A., Silva, D. A., Rocklin, G. J., Hicks, D. R., Vergara, R., Murapa, P., Bernard, S. M., Zhang, L., Lam, K. H., Yao, G., et al. (2017). Massively parallel de novo protein design for targeted therapeutics. Nature 550, 74-79.
Day, M. (2020). Covid-19: identifying and isolating asymptomatic people helped eliminate virus in Italian village. BMJ 368, m1165.
Galloway, S. E., Paul, P., MacCannell, D. R., Johansson, M. A., Brooks, J. T., MacNeil, A., Slayton, R. B., Tong, S., Silk, B. J., Armstrong, G. L., et al. (2021). Emergence of SARS-COV-2 B.1.1.7 Lineage-United States, Dec. 29, 2020-Jan. 12, 2021. MMWR Morb Mortal Wkly Rep 70, 95-99.
Golden, J. W., Cline, C. R., Zeng, X., Garrison, A. R., Carey, B. D., Mucker, E. M., White, L. E., Shamblin, J. D., Brocato, R. L., Liu, J., et al. (2020). Human angiotensin-converting enzyme 2 transgenic mice infected with SARS-COV-2 develop severe and fatal respiratory disease. JCI Insight 5.
Hassan, A. O., Case, J. B., Winkler, E. S., Thackray, L. B., Kafai, N. M., Bailey, A. L., McCune, B. T., Fox, J. M., Chen, R. E., Alsoussi, W. B., et al. (2020). A SARS-COV-2 Infection Model in Mice Demonstrates Protection by Neutralizing Antibodies. Cell 182, 744-753 e744. Hoagland, D. A., Møller, R., Uhl, S. A., Oishi, K., Frere, J., Golynker, I., Horiuchi, S., Panis, M., Blanco-Melo, D., Sachs, D., et al. (2021). Leveraging the antiviral type I interferon system as a first line of defense against SARS-COV-2 pathogenicity. Immunity.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020). SARS-COV-2 Cell Entry Depends on ACE2 and TMPRSS2 and Is Blocked by a Clinically Proven Protease Inhibitor. Cell 181, 271.
Jeyanathan, M., Afkhami, S., Smaill, F., Miller, M. S., Lichty, B. D., and Xing, Z. (2020). Immunological considerations for COVID-19 vaccine strategies. Nat Rev Immunol 20, 615-632.
Johnson, K. D., Harris, C., Cain, J. K., Hummer, C., Goyal, H., and Perisetti, A. (2020). Pulmonary and Extra-Pulmonary Clinical Manifestations of COVID-19. Front Med (Lausanne) 7, 526.
Kang, T. H., and Jung, S. T. (2019). Boosting therapeutic potency of antibodies by taming Fc domain functions. Exp Mol Med 51, 1-9.
Kordzadeh-Kermani, E., Khalili, H., and Karimzadeh, I. (2020). Pathogenesis, clinical manifestations and complications of coronavirus disease 2019 (COVID-19). Future Microbiol 15, 1287-1305.
Krammer, F. (2020). SARS-COV-2 vaccines in development. Nature 586, 516-527. Letko, M., Marzi, A., and Munster, V. (2020). Functional assessment of cell entry and receptor usage for SARS-COV-2 and other lineage B betacoronaviruses. Nat Microbiol 5, 562.
Leung, K., Shum, M. H., Leung, G. M., Lam, T. T., and Wu, J. T. (2021). Early transmissibility assessment of the N501Y mutant strains of SARS-COV-2 in the United Kingdom, October to November 2020. Euro Surveill 26.
Li, R., Pei, S., Chen, B., Song, Y., Zhang, T., Yang, W., and Shaman, J. (2020). Substantial undocumented infection facilitates the rapid dissemination of novel coronavirus (SARS-COV-2). Science 368, 489-493.
Matsuyama, S., Nao, N., Shirato, K., Kawase, M., Saito, S., Takayama, I., Nagata, N., Sekizuka, T., Katoh, H., Kato, F., et al. (2020). Enhanced isolation of SARS-COV-2 by TMPRSS2-expressing cells. Proc Natl Acad Sci USA 117, 7001-7003.
McGovern, T. K., Robichaud, A., Fereydoonzad, L., Schuessler, T. F., and Martin, J. G. (2013). Evaluation of respiratory system mechanics in mice using the forced oscillation technique. J Vis Exp, e50172.
Mukherjee, S., Sirohi, D., Dowd, K. A., Chen, Z., Diamond, M. S., Kuhn, R. J., and Pierson, T. C. (2016). Enhancing dengue virus maturation using a stable furin over-expressing cell line. Virology 497, 33-40.
Schafer, A., Muecksch, F., Lorenzi, J. C. C., Leist, S. R., Cipolla, M., Bournazos, S., Schmidt, F., Maison, R. M., Gazumyan, A., Martinez, D. R., et al. (2021). Antibody potency, effector function, and combinations in protection and therapy for SARS-COV-2 infection in vivo. J Exp Med 218.
Standl, F., Jöckel, K. H., Brune, B., Schmidt, B., and Stang, A. (2020). Comparing SARS-CoV-2 with SARS-COV and influenza pandemics. Lancet Infect Dis.
Tegally, H., Wilkinson, E., Giovanetti, M., Iranzadeh, A., Fonseca, V., Giandhari, J., Doolabh, D., Pillay, S., San, E. J., Msomi, N., et al. (2020). Emergence and rapid spread of a new severe acute respiratory syndrome-related coronavirus 2 (SARS-COV-2) lineage with multiple spike mutations in South Africa (Cold Spring Harbor Laboratory).
Voloch, C. M., Silva F, R. D., De Almeida, L. G. P., Cardoso, C. C., Brustolini, O. J., Gerber, A. L., Guimarães, A. P. D. C., Mariani, D., Costa, R. M. D., Ferreira, O. C., et al. (2020). Genomic characterization of a novel SARS-COV-2 lineage from Rio de Janeiro, Brazil (Cold Spring Harbor Laboratory).
Wang, P., Nair, M. S., Liu, L., Iketani, S., Luo, Y., Guo, Y., Wang, M., Yu, J., Zhang, B., Kwong, P. D., et al. (2021a). Antibody Resistance of SARS-COV-2 Variants B.1.351 and B.1.1.7 (Cold Spring Harbor Laboratory).
Wang, Z., Schmidt, F., Weisblum, Y., Muecksch, F., Barnes, C. O., Finkin, S., Schaefer-Babajew, D., Cipolla, M., Gaebler, C., Lieberman, J. A., et al. (2021b). mRNA vaccine-elicited antibodies to SARS-COV-2 and circulating variants. Nature.
Wibmer, C. K., Ayres, F., Hermanus, T., Madzivhandila, M., Kgagudi, P., Lambson, B. E., Vermeulen, M., Van Den Berg, K., Rossouw, T., Boswell, M., et al. (2021). SARS-COV-2 501Y. V2 escapes neutralization by South African COVID-19 donor plasma (Cold Spring Harbor Laboratory).
Winkler, E. S., Bailey, A. L., Kafai, N. M., Nair, S., McCune, B. T., Yu, J., Fox, J. M., Chen, R. E., Earnest, J. T., Keeler, S. P., et al. (2020a). SARS-COV-2 infection of human ACE2-transgenic mice causes severe lung inflammation and impaired function. Nat Immunol 21, 1327-1335.
Winkler, E. S., Gilchuk, P., Yu, J., Bailey, A. L., Chen, R. E., Zost, S. J., Jang, H., Huang, Y., Allen, J. D., Case, J. B., et al. (2020b). Human neutralizing antibodies against SARS-COV-2 require intact Fc effector functions and monocytes for optimal therapeutic protection. Cell. In press.
Xie, X., Liu, Y., Liu, J., Zhang, X., Zou, J., Fontes-Garfias, C. R., Xia, H., Swanson, K. A., Cutler, M., Cooper, D., et al. (2021a). Neutralization of SARS-COV-2 spike 69/70 deletion, E484K and N501Y variants by BNT162b2 vaccine-elicited sera. Nature Medicine.
Xie, X., Liu, Y., Liu, J., Zhang, X., Zou, J., Fontes-Garfias, C. R., Xia, H., Swanson, K. A., Cutler, M., Cooper, D., et al. (2021b). Neutralization of SARS-COV-2 spike 69/70 deletion, E484K and N501Y variants by BNT162b2 vaccine-elicited sera. Nat Med.
Zhou, D., Fuk-Woo Chan, J., Zhou, B., Zhou, R., Li, S., Shan, S., Liu, L., Zhang, A. J., Chen, S. J., Chung-Sing Chan, C., et al. (2021). Robust SARS-COV-2 Infection in Nasal Turbinates after Treatment with Systemic Neutralizing Antibodies. Cell Host & Microbe.
Zhou, F., Yu, T., Du, R., Fan, G., Liu, Y., Liu, Z., Xiang, J., Wang, Y., Song, B., Gu, X., et al. (2020). Clinical course and risk factors for mortality of adult inpatients with COVID-19 in Wuhan, China: a retrospective cohort study. Lancet 395, 1054-1062.

Multivalent Designs

Escape variants of SARS-COV-2 are threatening to considerably prolong the COVID-19 pandemic. Here we develop multivalent minibinders as potential prophylactic and therapeutic agents to address this problem. We designed multivalent minibinders containing three copies of a minibinder (self-assembled homotrimer), or three linked distinct minibinders (multi-domain fusion) targeting different sites, geometrically matched to the spike trimer and optimized their composition using a rapid cell-free expression and evaluation workflow. The optimized designs have greatly slowed dissociation rates from the SARS-COV-2-S-glycoprotein with complex half-lives of more than two weeks. Cryo-EM of the structures reveal that both homotrimer and fusion minibinder constructs can engage all three RBDs on a single spike protein. The top trimeric and fusion candidates neutralize the wild-type SARS-CoV-2 virus in addition to the B.1.1.7, B.1.351, B.1.1.28 variants of concern with IC₅₀s in the low pM range. Additionally, the top homotrimer candidate provided prophylactic protection in a human ACE2-expressing transgenic mice against the same variant strains. Our approach highlights the utility of computational protein design coupled to rapid experimental prototyping to design potent multivalent inhibitors that can broadly neutralize widely circulating variants of concern.

We sought to develop multivalent versions of our computationally designed miniproteins that block the SARS-COV-2 receptor binding domain (RBD) interaction with its host receptor ACE2. In principle, the small size of the designed minibinders enables simultaneous engagement of multiple RBDs within a single spike protein trimer. We hypothesized that this multivalent binding would lead to ultra-high affinity inhibitors that are more resistant to escape mutations than their monomeric counterparts. The resulting avidity from these multivalent interactions could ameliorate the effects of mutations that would escape individual domains. Additionally, single proteins containing domains targeting multiple distinct epitopes or containing different sets of contacts with the target epitope could further increase the robustness of the designs to mutational escape. Starting with the LCB1, AHB2, and LCB3 minibinders (hereafter referred to as M1, M2, and M3 respectively; Table 11) and their known binding modes we pursued two parallel strategies for designing multivalent inhibitors, self-assembled homotrimers and multi-domain fusions.

TABLE 11
List of abbreviations used to describe
multivalent minibinders

| ID | Protein |
|---|---|
| M1 | LCB1_v2.2 |
| M2 | AHB2 |
| M3 | LCB3_v2.2 |
| F23-P12 | AHB2_v2-PAS12-LCB3_v2.2 |
| F31-P12 | LCB3_v2.2-PAS12-LCB1_v2.2 |
| F231- | AHB2_v2-PAS12-LCB3_v2.2-PAS12- |
| P12 | LCB1_v2.2 |
| F231- | AHB2_v2-PAS24-LCB3_v2.2-PAS24- |
| P24 | LCB1_v2.2 |
| F23-G10 | AHB2_v2-GS10-LCB3_v2.2 |
| F31-F10 | LCB3_v2.2-GS10-LCB1_v2.2 |
| F231- | AHB2_v2-GS10-LCB3_v2.2-GS10- |
| G10 | LCB1_v2.2 |
| H1-1 | SB175-6GS-LCB1_v2.2 |
| H2-0 | AHB2-4GS-1rfo |
| H2-1 | AHB2-2GS-SB175 |
| H3-1 | LCB3_v2.2-6GS-SB175 |

To enable rapid prototyping of the designed proteins, we developed a cell-free DNA assembly and protein expression workflow enabling a greatly shortened design-build-test cycle better matched to the urgency of a pandemic. The workflow combines a cell-free DNA assembly step utilizing Gibson assembly followed by PCR to generate linear expression templates that are used to drive cell-free protein synthesis (CFPS). The developed workflow allows us to translate synthetic DNA to purified protein in as little as 6 hours, is easily scaled to high-throughput formats (e.g., 96- or 384-well plates), and is amenable to automated liquid handling. Furthermore, we coupled this cell-free workflow to an AlphaLISA protein-protein interaction (PPI) competition assay to enable comparison of dissociation rates of the designed proteins against either the monomeric RBD or the trimeric hexapro SARS-COV-2-S-glycoprotein (S6P). Because multivalency largely only impacts the dissociation rate constant of the interaction, we reasoned that an in-solution off-rate screen would enable us to distinguish mono- from multi-valent binding. The resulting workflow can evaluate hundreds of candidate multivalent proteins per week.

Design and validation of multivalent binders

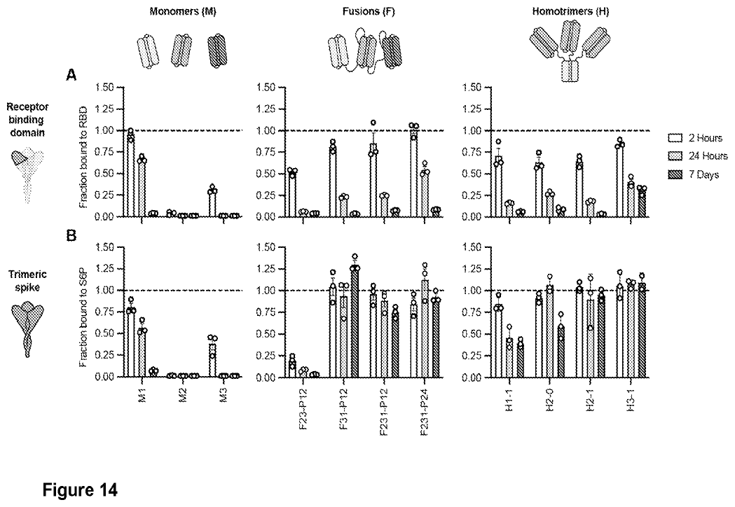

In the first strategy, we designed self-assembling trimeric versions of the M1, M2, and M3 miniproteins geometrically matched to the three RBDs in the spike trimer (hereafter referred to as H[binding domain #]-[homotrimer #]; for example, H₁-1 represents a homotrimer of M1 with homotrimerization domain 1, Table 11). We designed, expressed, and evaluated more than one hundred different proteins containing various homotrimerization domains and linker lengths using our cell-free expression and multivalency screen workflow. We identified versions of each homotrimer that showed slow dissociation rates potentially indicating multivalent binding (FIG. 14).

In the second strategy, we generated two- and three-domain fusions of the M1, M2 and M3 binding domains separated by flexible linkers (hereafter referred to as F[binding domain #s]-[linker]; for example, F231-P12 represents a fusion of M2 to M3 to M1 all separated by a PAS12 linker, Table 11). We evaluated a range of linker lengths chosen to span the distances between the termini of the domains when bound to the “open” and “closed” states of the RBD. We again expressed and evaluated more than one hundred different designs varying binding domain connectivity and linker length to optimize multivalency. Several identified two- and three-domain fusions show slow dissociation rates comparable to the homo-trimeric constructs described above (FIG. 14).

The best candidates from each strategy showed little to no dissociation after 14 days of with competitor, with further measurements being limited by the stability of S6P. From these data we estimate the complex has a dissociation rate constant of slower than 1 ×10⁻⁷ s⁻¹. To our knowledge, these are the slowest measured dissociation rate constant for a synthetic protein-protein interactions ever reported.

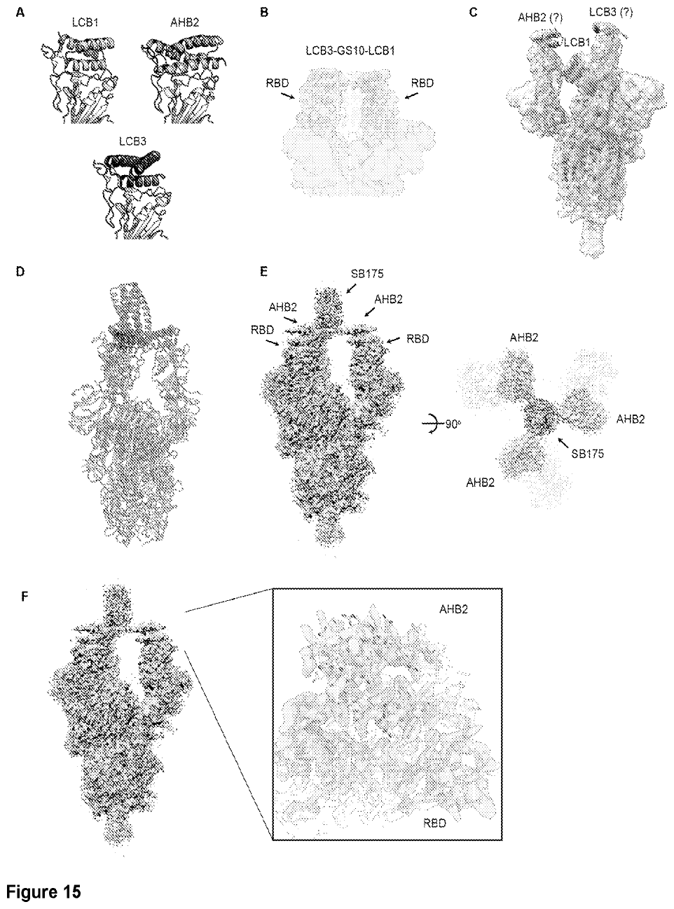

We next used single particle cryo-electron microscopy (cryo-EM) to characterize the complex between S6P and the top candidate minibinders constructs (FIG. 15). The Cryo-EM structures of the H₂-1, F31-G10, and the F231-P24 constructs were determined at resolutions of 2.6, 4.5, and 3.9 Å respectively. H₂-1 was found to simultaneously engage all three RBDs, causing all three RBDs to adopt the open state. The design model accurately closely matches the observed structure. F31-G10 bound to two RBDs, both appearing to adopt the open conformation upon binding. The structure indicates this linker length enabled simultaneous binding of two RBDs in their native state. The third free RBD adopted either the open or closed conformation in the structure. F231-P24 bound to three RBDs, with M1 binding a closed conformation RBD and M2 and M3 binding to open conformation RBDs. This suggests the linker length is sufficiently long enough to enable all three binding domains to simultaneously engage all three RBDs without significant distortion of the native state. In both F31-G10 and F231-P24 the maps are highly suggestive of multivalent binding, though the flexible linkers yield no density in the EM map to confirm linkage of the domains.

Multivalent Minibinders Neutralize Widely Circulating SARS-CoV-2 Variants

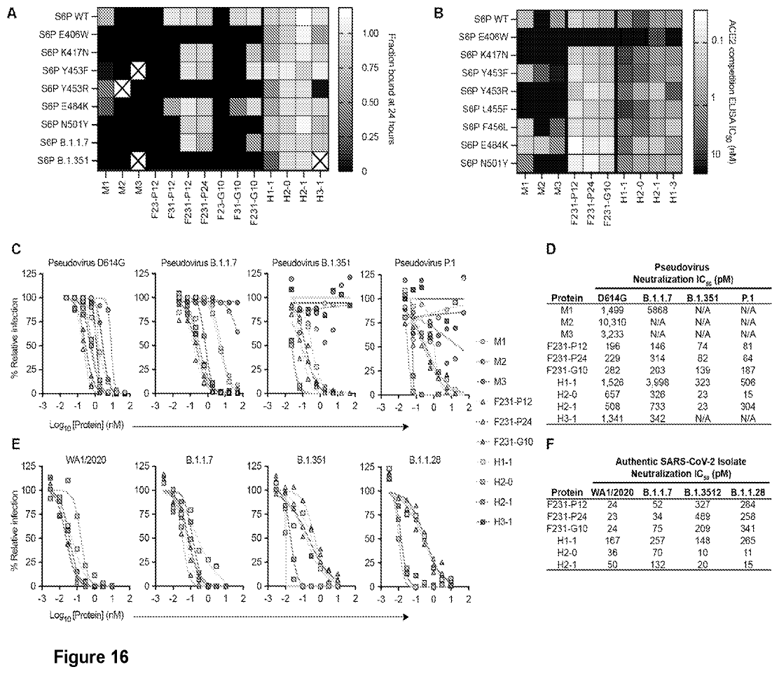

We next sought to determine ability of the multivalent constructs to neutralize SARS-CoV-2 variants. We screened the off rate of the best multivalent minibinders against a panel of mutant spike proteins (FIG. 16). The homotrimers showed the most mutational resistance, with the H₂ homotrimers showing little dissociation after 24 hours against any of the mutant spikes. The two-domain fusions showed little increased resilience to the tested point mutants. The three-domain fusions showed considerably more consistent binding to the tested point mutants, though some still impacted binding.

We additionally evaluated the potency of these proteins via neutralization assays against both a SARS-COV-2 HIV pseudovirus in addition to authentic SARS-COV-2 isolates (FIG. 16). The H2-0 and H2-1 homotrimers consistently performed the best across all constructs tested, with IC₅₀s in the low pM range. The three-domain fusions also performed well, with IC₅₀s in the sub nM range for all tested variants. The greater neutralization breadth of the H₂ homotrimers likely reflects the closer mimicking if the ACE2 binding site by the M2 monomer, a unique advantage enabled by protein design.

Multivalent Minibinders Resist Viral Escape

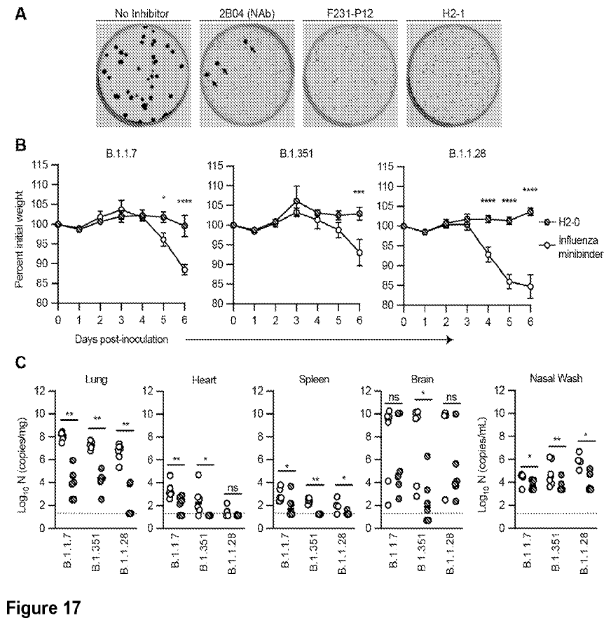

In addition to evaluating the top candidate's ability to neutralize currently circulating SARS-COV-2 mutants, we also tested the ability of the inhibitors to resist escape viral escape (FIG. 17). To do this, plaque assays were performed with a VSV-SARS-COV-2 chimera virus were replicated on Vero E6 cells. To select mutants that were resistant to the inhibitor, the inhibitor was included in the overlay to halt replication of non-resistant viruses. In positive control neutralizing antibody (2B04), multiple escape mutants were selected per plate. For both F231-P12 and H₂-1 no escape mutants were isolated in 35 replicate wells of each inhibitor.

H₂-0 Provides Prophylactic Protection in Human ACE2-Expressing Transgenic Mice

To determine the ability of our multivalent minibinders to protect in an in vivo model, we evaluated them as a pre-exposure prophylactic treatment in human ACE2-expressing transgenic mice (FIG. 17). A single 50 μg dose of H₂-0 was administered intranasally (i.n.) one day prior to inoculation with 10³ focus forming units of SARS-COV-2 Variants B.1.1.7, B1.351, B.1.1.24. In all cases, i.n. administration of H₂-0 protected the mice against SARS-CoV-2-induced weight loss. At 6 days post infection viral burden was determined via RT-qPCR in a variety in tissues. Notably, viral loads in the lungs were reduced in all cases. These results indicate that H₂-0 given via i.n. administration can provide prophylactic protection against SARS-COV-2 infection in a relevant mouse model.

### CONCLUSIONS

We anticipate that the cell-free protein expression and evaluation workflow will find utility in many different applications where the evaluation of individual protein variants is the limiting process step. In addition, our developed multivalency screen will accelerate the ability of researchers to develop multivalent protein therapeutics.

The designed protein constructs could have a number of advantages over monoclonal antibodies for preventing and treating COVID-19 infection. 1) direct administration into respiratory system, 2) low cost of goods and amenability to very large-scale production, 3) high stability and lack of need for cold chain, and 4) very broad resistance to escape mutants in single compounds. More generally, designed high affinity multivalent minibinders could provide a powerful platform for combating viral pandemics.

1. A polypeptide comprising an amino acid sequence at least 50% identical to the amino acid sequence selected from the group consisting of SEQ ID NOS: 1-17, 19-21, 23-34 and 100-101, wherein the polypeptide binds to SARS-COV-2 Spike glycoprotein receptor binding domain (RBD).

2. The polypeptide of claim 1, wherein amino acid substitutions relative to the reference polypeptide amino acid sequence are selected from the exemplary amino acid substitutions provided in Table 1, and/or wherein interface residues are identical to those in the reference polypeptide or are conservatively substituted relative to interface residues in the reference polypeptide.

3.-4. (canceled)

5. The polypeptide of claim 1, comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-10 and 102-136.

6. The polypeptide of claim 5, wherein the polypeptide comprises an amino acid substitution relative to the amino acid sequence of SEQ ID NO:1 at 1, 2, 3, 4, 5, 6, 7, 8, 9, 10, 11, 12, 13, 14, 15, 16, 17, or all 18 residues selected from the group consisting of 2, 4, 5, 14, 15, 17, 18, 27, 28, 32, 37, 38, 39, 41, 42, 49, 52, and 55, optionally wherein the substitutions are selected from the substitutions listed in Table 4, either individually or in combinations in a given row.

7. (canceled)

8. The polypeptide of any claim 1, comprising an amino acid sequence at least 50% identical to the amino acid sequence selected from the group consisting of SEQ ID NOS: 13-17, 19-21 and 137-163.

9. The polypeptide of claim 8, wherein the polypeptide comprises an amino acid substitution relative to the amino acid sequence of SEQ ID NO:13 at 1, 2, 3, 4, 5, 6, 7, 8, 9, 10, 11, 12, 13, 14, 15, 16, 17, 18, 19, or all 20 residues selected from the group consisting 2, 6, 8, 9, 13, 14, 19, 22, 25, 26, 28, 29, 34, 35, 37, 40, 43, 45, 49, and 62, optionally wherein the substitutions are selected from the substitutions listed in Table 6, either individually or in combinations in a given row.

10. (canceled)

11. The polypeptide of claim 1, comprising an amino acid sequence at least 50% identical to the amino acid sequence selected from the group consisting of SEQ ID NOS:33-34 and 100-101 and 164, optionally wherein the polypeptide comprises an amino acid substitution relative to the amino acid sequence of SEQ ID NO:101 at or both residues selected from the group consisting 63 and 75.

12.-15. (canceled)

16. The polypeptide of claim 1, comprising two or more copies of the amino acid sequence at least 50%; identical to the amino acid sequence selected from the group consisting of SEQ ID NOS: 1-17, 19-21, 23-34 and 100-101.

17.-25. (canceled)

26. The polypeptide of claim 16, wherein the polypeptide comprises the formula Z1-Z2-Z3, wherein:
Z1 comprises an amino acid sequence at least 50% identical to the amino acid sequence selected from the group consisting of SEQ ID NOS: 1-17, 19-21, 23-34 and 100-164;
Z2 comprises an optional amino acid linker; and
Z3 comprises an amino acid sequence at least 50% identical to the amino acid sequence selected from the group consisting of SEQ ID NOS: 1-17, 19-21, 23-34 and 100-164;
wherein Z1 and Z3 may be identical or different.

27.-34. (canceled)

35. The polypeptide of claim 26, wherein the polypeptide comprises the formula B1-B2-Z1-Z2-Z3-B3-B4, wherein:
Z1, Z2, and Z3 are as defined in claim 26;
B2 and B3 comprise optional amino acid linkers; and
one or both of B1 and B4 independently comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS: 1-17, 19-21, 23-34 and 100-164, wherein one of B1 and B4 may be absent.

36.-45. (canceled)

46. The polypeptide of claim 1, comprising an amino acid sequence at least 50% identical to the amino acid sequence selected from the group consisting of SEQ ID NOS:47-60,193-355, and 454-588 and a genus selected from those recited in the right hand column of Table 8 wherein genus positions X1, X2, X3, and X4 may be present or absent, and when present may be any sequence of 1 or more amino acids and wherein any N-terminal methionine residues may be present or absent in the polypeptide, and wherein residues in parentheses may be present or absent and are not considered in determining percent identity.

47. The polypeptide of claim 1, further comprising an additional functional peptide domain.

48.-53. (canceled)

54. The polypeptide of claim 1, wherein the polypeptide is linked to a stabilization domain.

55. The polypeptide of claim 1, comprising an amino acid sequence at least 50% identical to the amino acid sequence selected from the group consisting of SEQ ID NOS: 65-96, wherein in embodiments where a secretion signal is present (MARAWIFFLLCLAGRALA; SEQ ID NO:63) it can be replaced with any other secretion signal.

56. (canceled)

57. A nucleic acid encoding the polypeptide of claim 1.

58. An expression vector comprising the nucleic acid of claim 57 operatively linked to a promoter.

59. A host cell comprising the expression vector of claim 58.

60. An oligomer of the polypeptide of claim 1, or a composition, comprising 2 or more copies of the polypeptide of claim 1 attached to a support.

61.-62. (canceled)

63. A pharmaceutical composition, comprising the polypeptide of claim 1, and a pharmaceutically acceptable carrier.

64. A method for treating or limiting development of a severe acute respiratory syndrome (SARS) coronavirus infection (including SARS-Co-V and SARS-COV-2), comprising administering to a subject in need thereof an amount of the polypeptide of claim 1 effective to treat or limit development of the infection.

65.-72. (canceled)
